(19) United States
(12) Patent Application Publication
(10) Pub. No.: US 2019/0359653 A1
(43) Pub. Date: Nov. 28, 2019

(54) SELECTIVE PEPTIDE INHIBITORS OF FRIZZLED

(71) Applicant: Genentech, Inc., South San Francisco, CA (US)

(72) Inventors: Aaron Hugh NILE, San Bruno, CA (US); Yingnan ZHANG, Fremont, CA (US); Li-juan ZHOU, Belmont, CA (US); Rami HANNOUSH, San Mateo, CA (US)

(21) Appl. No.: 16/292,099

(22) Filed: Mar. 4, 2019

Related U.S. Application Data
(63) Continuation of application No. PCT/US2017/050841, filed on Sep. 8, 2017.
(60) Provisional application No. 62/385,848, filed on Sep. 9, 2016.
(60) Provisional application No. 62/419,331, filed on Nov. 8, 2016.

(51) Int. Cl. C07K 7/08; C07K 7/06; A61K 47/54

(52) U.S. Cl. CPC C07K 7/08; A61K 38/00; A61K 47/543; C07K 7/06

(57) ABSTRACT

Provided are ligands comprising a non-naturally occurring peptide that binds a cysteine rich domain (CRD) of the Frizzled7 (FZD7) receptor. Additionally, provided are therapeutic methods of using such ligands, as well as compositions comprising such ligands.

### CROSS REFERENCE TO RELATED APPLICATIONS

This application is a continuation of International Application No. PCT/US2017/050841, filed on Sep. 8, 2017, which claims the priority benefit of U.S. Provisional Application Ser. No. 62/385,848, filed Sep. 9, 2016 and of U.S. Provisional Application Ser. No. 62/419,331, filed Nov. 8, 2016, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392036901SEQLIST.txt, date recorded: Feb. 15, 2019, size: 50 KB).

### BACKGROUND OF THE INVENTION

The Wnt signaling pathway's association with carcinogenesis began as a result of early observations and experiments in certain murine mammary tumors. Wnt-1 proto-oncogene (Int-1) was originally identified from mammary tumors induced by mouse mammary tumor virus (MMTV) due to an insertion of a viral DNA sequence. Nusse et al., Cell 1982; 31: 99-109. The result of such viral integration was unregulated expression of Int-1 resulting in the formation of tumors. Vanooyen, A. et al., Cell 1984; 39: 233-240; Nusse, R. et al., Nature 1984; 307: 131-136; Tsukamoto et al., Cell 1988; 55: 619-625. Subsequent sequence analysis demonstrated that the Int-1 was a mammalian homolog of the Drosophila gene Wingless (Wg), which was implicated in development, and the terms were then combined to create “Wnt” to identify this family of proteins.

The human Wnt gene family of secreted ligands has now grown to at least 19 members (e.g., Wnt-1 (RefSeq.: NM.sub.-005430), Wnt-2 (RefSeq.: NM.sub.-003391), Wnt-2B (Wnt-13) (RefSeq.: NM.sub.-004185), Wnt-3 (RefSeq.: NM.sub.-030753), Wnt3a (RefSeq.: NM.sub.-033131), Wnt-4 (RefSeq.: NM.sub.-030761), Wnt-5A (RefSeq.: NM.sub.-003392), Wnt-5B (RefSeq.: NM.sub.-032642), Wnt-6 (RefSeq.: NM.sub.-006522), Wnt-7A (RefSeq.: NM.sub.-004625), Wnt-7B (RefSeq.: NM.sub.-058238), Wnt-8A (RefSeq.: NM.sub.-058244), Wnt-8B (RefSeq.: NM.sub.-003393), Wnt-9A (Wnt-14) (RefSeq.: NM.sub.-003395), Wnt-9B (Wnt-15) (RefSeq.: NM.sub.-003396), Wnt-10A (RefSeq.: NM.sub.-025216), Wnt-10B (RefSeq.: NM.sub.-003394), Wnt-11 (RefSeq.: NM.sub.-004626), Wnt-16 (RefSeq.: NM.sub.-016087)). Each member has varying degrees of sequence identity but all contain 23-24 conserved cysteine residues which show highly conserved spacing. McMahon, A P et al., Trends Genet. 1992; 8: 236-242; Miller, J R. Genome Biol. 2002; 3(1): 3001.1-3001.15. The Wnt proteins are small (i.e., 39-46 kD) acylated, secreted glycoproteins which play key roles in both embryogenesis and mature tissues. During embryological development, the expression of Wnt proteins is important in patterning through control of cell proliferation and determination of stem cell fate. The Wnt molecules are also palmitoylated, and thus are more hydrophobic than would be otherwise predicted by analysis of the amino acid sequence alone. Willert, K. et al, Nature 2003; 423: 448-52. The site or sites of palmitoylation are also believed to be essential for function.

The Wnt proteins act as ligands to activate the Frizzled (FRZ) family seven-pass transmembrane receptors. Ingham, P. W. Trends Genet. 1996; 12: 382-384; Yang-Snyder, J. et al., Curr. Biol. 1996; 6: 1302-1306; Bhanot, P. et al., Nature 1996; 382: 225-230. There are ten known members of the FRZ family (e.g., FRZ1-Frz10), each characterized by the presence of a cysteine rich domain (CRD). Huang et al., Genome Biol. 2004; 5: 234.1-234.8. There is a great degree of promiscuity between the various Wnt-Frizzled interactions, but Wnt-FRZ binding must also incorporate the LDL receptor related proteins (LRP5 or LRP6) and the membrane and the cytoplasmic protein Disheveled (Dsh) to form an active signaling complex.

The binding of Wnt to Frizzled can activate signaling via either the canonical Wnt signaling pathway, thereby resulting in stabilization and increased transcriptional activity of .beta.-catenin [Peifer, M. et al., Development 1994; 120: 369-380; Papkoff, J. et al, Mol. Cell Biol. 1996; 16: 2128-2134] or non-canonical signaling, such as through the Wnt/planar cell polarity (Wnt/PCP) or Wnt-calcium (Wnt/Ca.sup.2+) pathway. Veeman, M. T. et al., Dev. Cell 2003; 5: 367-377. FZD7, one of the FRZ receptors, is upregulated in diverse human cancers and is able to regulate Wnt signaling activity even in cancer cells which have mutations to down-stream signal transducers. Thus, there is a need to develop selective inhibitors of FZD7 for a variety of therapeutic applications, such as cancer. The present invention meets this and other needs.

### BRIEF SUMMARY OF THE INVENTION

In certain embodiments, provided is a ligand comprising a non-naturally occurring peptide that binds to a cysteine rich domain (CRD) of the Frizzled 7 (FZD7) receptor. In certain embodiments according to (or as applied to) any of the embodiments above, the non-naturally occurring peptide specifically binds the CRD of FZD7. In certain embodiments according to (or as applied to) any of the embodiments above the non-naturally occurring peptide does not bind to a CRD of a FZD receptor selected from the group consisting of: FZD3, FZD4, FZD5, FZD6, FZD8, FZD9, or FZD10. In certain embodiments according to (or as applied to) any of the embodiments above, the non-naturally occurring peptide further binds FZD1 and FZD2. In certain embodiments according to (or as applied to) any of the embodiments above, the non-naturally occurring peptide does not bind to a CRD of a FZD receptor selected from the group consisting of: FZD1, FZD2, FZD3, FZD4, FZD5, FZD6, FZD8, FZD9, or FZD10.

In certain embodiments according to (or as applied to) any of the embodiments above, the non-naturally occurring peptide is linear. In certain embodiments according to (or as applied to) any of the embodiments above, the non-naturally occurring peptide is cyclic. In certain embodiments according to (or as applied to) any of the embodiments above, the non-naturally occurring peptide is between 8-16 amino acids in length. In certain embodiments according to (or as applied to) any of the embodiments above, the non-naturally occurring peptide is between 11-14 amino acids in length.

In certain embodiments according to (or as applied to) any of the embodiments above, wherein the FZD7 is hFZD7. In certain embodiments according to (or as applied to) any of the embodiments above, he non-naturally occurring peptide specifically binds a binding region of hFZD7 CRD comprising at least three amino acids selected from the group consisting of: Leu81, His84, Gln85, Tyr87, Pro88, Phe138, and Phe140.

In certain embodiments according to (or as applied to) any of the embodiments above, the non-naturally occurring peptide comprises an amino acid sequence set forth in:

(SEQ ID NO: 100)
X₁X₂X₃DDLX₄X₅WCHVMY

wherein each of X₁-X₃ is no amino acid, any amino acid, or an unnatural amino acid, and wherein each of X₄-X₅ is any amino acid or an unnatural amino acid. In certain embodiments according to (or as applied to) any of the embodiments above, X₁ is L, X₂ is P, X₃ is S, X₄ is E, and X₅ is F. In certain embodiments according to (or as applied to) any of the embodiments above, the non-naturally occurring peptide comprises the amino acid sequence set forth in LPSDDLEFWCHVMY (SEQ ID NO: 13). In certain embodiments according to (or as applied to) any of the embodiments above, X₁ is no amino acid, X₂ is no amino acid, X₃ is S, X₄ is E, and X₅ is F. In certain embodiments according to (or as applied to) any of the embodiments above, the non-naturally occurring peptide comprises the amino acid sequence set forth in SDDLEFWCHVMY (SEQ ID NO: 99). In certain embodiments according to (or as applied to) any of the embodiments above, the N-terminal amine of the non-naturally occurring peptide is acetylated, the C-terminal carboxyl group of the non-naturally occurring peptide is amidated, or the N-terminal amine of the peptide is acetylated and the C-terminal carboxyl group of the peptide is amidated.

In certain embodiments according to (or as applied to) any of the embodiments above, the non-naturally occurring peptide enhances the binding of a Wnt to the CRD of the FZD7 receptor. In certain embodiments according to (or as applied to) any of the embodiments above, the FZD7 receptor is an hFZD7 receptor.

In certain embodiments according to (or as applied to) any of the embodiments above, the non-naturally occurring peptide comprises an amino acid sequence set forth in:

(SEQ ID NO: 114)
SDDLEFWCHVXY

wherein X is any amino acid, or an unnatural amino acid. In certain embodiments according to (or as applied to) any of the embodiments above, the unnatural amino acid is selected from the group consisting of: 2-amino-3-decyloxy-propionic acid, a derivative of lysine comprising octanoic acid coupled at epsilon amino group, aminodecanoic acid, 2-aminodecanoic acid, a derivative of lysine comprising decanoic acid coupled at epsilon amino group, and 6-hydroxy-L-norleucine.

In certain embodiments according to (or as applied to) any of the embodiments above, the non-naturally occurring peptide comprises an amino acid sequence set forth in:

(SEQ ID NO: 115)
SDDXEFWCHVMY

wherein X is any amino acid, or an unnatural amino acid, and wherein the unnatural amino acid is selected from the group consisting of: L-homoleucine, L-homophenylalanine, and a derivative of lysine comprising octanoic acid coupled at epsilon amino group.

In certain embodiments according to (or as applied to) any of the embodiments above, the non-naturally occurring peptide comprises an amino acid sequence set forth in any one of SEQ ID NOs: 1-31 and 39-99. In certain embodiments according to (or as applied to) any of the embodiments above, the non-naturally occurring peptide comprises an amino acid sequence set forth in any one of SEQ ID Nos: 32-38.

In certain embodiments according to (or as applied to) any of the embodiments above, the non-naturally occurring peptide inhibits Wnt signaling with an IC₅₀ of 120 nM or less. In certain embodiments according to (or as applied to) any of the embodiments above, the non-naturally occurring peptide has an EC₅₀ value of 90 nM or less.

In certain embodiments according to (or as applied to) any of the embodiments above, the non-naturally occurring peptide is conjugated to a lipid. In certain embodiments according to (or as applied to) any of the embodiments above, lipid is a long chain fatty acid (LCFA). In certain embodiments according to (or as applied to) any of the embodiments above, the lipid is a short chain fatty acid (SCFA). In certain embodiments according to (or as applied to) any of the embodiments above, the fatty acid comprises an aromatic tail.

In certain embodiments according to (or as applied to) any of the embodiments above, the non-naturally occurring peptide in the ligand is dimerized. In certain embodiments according to (or as applied to) any of the embodiments above, the non-naturally occurring peptide is dimerized by way of disulfide bond. In certain embodiments according to (or as applied to) any of the embodiments above, the non-naturally occurring peptide is dimerized by way of chemical linker.

In certain embodiments, provided is a composition comprising the ligand according to (or as applied to) any of the embodiments above and a pharmaceutically acceptable carrier.

In certain embodiments, provided is a method of inhibiting Wnt signaling in a cell, comprising contacting the cell with the ligand or composition according to (or as applied to) any of the embodiments above.

In certain embodiments, provided is a method of inhibiting stem cell proliferation, comprising contacting a stem cell with the ligand or composition according to (or as applied to) any of the embodiments above. In certain embodiments according to (or as applied to) any of the embodiments above, the stem cell is an intestinal stem cell. In certain embodiments according to (or as applied to) any of the embodiments above, the stem cell is a cancer stem cell. In certain embodiments according to (or as applied to) any of the embodiments above, the cancer stem cell is a colon cancer stem cell, a pancreatic cancer stem cell, a non-small cell lung cancer stem cell, a cancer stem cell comprising a mutation in RNF43, a cancer stem cell characterized by USP6 overexpression, or a cancer stem cell characterized by gene fusions involving R-spondin (RSPO) family members

In certain embodiments, provided is a method of killing a cancer cell comprising contacting the cancer cell with the ligand or composition according to (or as applied to) any of the embodiments above. In certain embodiments according to (or as applied to) any of the embodiments above, the cancer cell is a colon cancer cell, a pancreatic cancer cell, a non-small cell lung cancer cell, a cancer cell comprising a mutation in RNF43, a cancer cell characterized by USP6 overexpression, or a cancer cell characterized by gene fusions involving R-spondin (RSPO) family members.

In certain embodiments, provided is a method treating cancer in a subject, comprising administering an effective amount of the ligand or composition according to (or as applied to) any of the embodiments above. In certain embodiments according to (or as applied to) any of the embodiments above, the cancer is colon cancer, pancreatic cancer, non-small cell lung cancer (NSCLC), a cancer characterized by a mutation in RNF43, a cancer characterized by USP6 overexpression, or a cancer characterized by gene fusions involving R-spondin (RSPO) family members.

In certain embodiments, provided is a use of the ligand or composition according to (or as applied to) any of the embodiments above in the manufacture of a medicament for treating cancer. In certain embodiments according to (or as applied to) any of the embodiments above, the cancer is colon cancer, pancreatic cancer, non-small cell lung cancer (NSCLC), a cancer characterized by a mutation in RNF43, a cancer characterized by USP6 overexpression, or a cancer characterized by gene fusions involving R-spondin (RSPO) family members.

In certain embodiments, provided is a composition comprising the ligand or composition according to (or as applied to) any of the embodiments above for use in treating cancer in a subject. In certain embodiments according to (or as applied to) any of the embodiments above, the cancer is colon cancer, pancreatic cancer, non-small cell lung cancer (NSCLC), or a cancer characterized by a mutation in RNF43, a cancer characterized by USP6 overexpression, or a cancer characterized by gene fusions involving R-spondin (RSPO) family members.

In certain embodiments, provided is a kit for treating cancer, comprising: (a) ligand or composition according to (or as applied to) any of the embodiments above, and (b) and instructions for administering the ligand to a subject that has cancer. In certain embodiments according to (or as applied to) any of the embodiments above, the cancer is colon cancer, pancreatic cancer, non-small cell lung cancer (NSCLC), a cancer characterized by a mutation in RNF43, a cancer characterized by USP6 overexpression, or a cancer characterized by gene fusions involving R-spondin (RSPO) family members.

In certain embodiments, provided is a ligand comprising the non-naturally occurring peptide set forth in LPSDDLEFWSHVMY (SEQ ID NO: 113).

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 provides a structural depiction of the interaction between Wnt8 and mouse FZD8 CRD displaying sequence conservation between human FZD CRDs onto the surface of mFZD8 CRD.

FIG. 2A shows the results of experiments performed to determine the effect of peptides Fz7-21 and Fz-21S on β-catenin signaling in HEK293-TB cells that were stimulated with 50 ng/ml recombinant Wnt3a.

FIG. 2B shows the results of experiments performed to determine the effect of peptides Fz7-21 and Fz-21S on Wnt-stimulated β-catenin signaling in HEK293-TB cells that were transfected with 5 ng pCDNA3.2-Wnt3a or 25 ng pCDNA3.2-Wnt3a.

FIG. 2C shows the results of experiments performed to determine the effect of peptides Fz7-21 and Fz-21S on Wnt-stimulated β-catenin signaling in HEK293-TB cells that were transfected with 5 ng pCDNA3.2-Wnt1 or 25 ng pCDNA3.2-Wnt1.

FIG. 2D shows the results of experiments performed to determine the effect of an a Fz7-21-derived peptide containing a D-Cys stereoisomer at position 10 on Wnt-stimulated β-catenin signaling in HEK293-TB cells that were transfected with 5 ng pCDNA3.2-Wnt1 or 25 ng pCDNA3.2-Wnt3 a.

FIG. 2E shows the results of experiments performed to determine the effect of peptides Fz7-21 and Fz-21S on receptor-independent β-catenin signaling in HEK293-TB cells that were treated with 6-BIO.

FIG. 3A provides the results of experiments that were performed to determine the EC50 values for binding of 5FAM-Fz7-21 or 5FAM-Fz7-21S to hFZD1 CRD-Fc.

FIG. 3B provides the results of experiments that were performed to determine the EC50 values for binding of 5FAM-Fz7-21 or 5FAM-Fz7-21S to mFZD2 CRD-Fc.

FIG. 3C provides the results of experiments that were performed to determine the EC50 values for binding of 5FAM-Fz7-21 or 5FAM-Fz7-21S to hFZD4 CRD-Fc.

FIG. 3D provides the results of experiments that were performed to determine the EC50 values for binding of 5FAM-Fz7-21 or 5FAM-Fz7-21S to hFZD5 CRD-Fc.

FIG. 3E provides the results of experiments that were performed to determine the EC50 values for binding of 5FAM-Fz7-21 or 5FAM-Fz7-21S to hFZD7 CRD-Fc.

FIG. 3F provides the results of experiments that were performed to determine the EC50 values for binding of 5FAM-Fz7-21 or 5FAM-Fz7-21S to mFZD7 CRD-Fc.

FIG. 3G provides the results of experiments that were performed to determine the EC50 values for binding of 5FAM-Fz7-21 or 5FAM-Fz7-21S to hFZD8 CRD-Fc.

FIG. 3H provides the results of experiments that were performed to determine the EC50 values for binding of 5FAM-Fz7-21 or 5FAM-Fz7-21S to mFZD9 CRD-Fc.

FIG. 3I provides the results of experiments that were performed to determine the EC50 values for binding of 5FAM-Fz7-21 or 5FAM-Fz7-21S to hFZD10 CRD-Fc.

FIG. 4A provides a representative fluorescence trace of 5FAM-Fz7-21 incubated with various FZD CRD-Fc proteins prior to analysis by FSEC.

FIG. 4B provides a representative fluorescence trace of 5FAM-Fz7-21S incubated with various FZD CRD-Fc proteins prior to analysis by FSEC.

FIG. 4C provides a quantification of fluorescence intensity (area under curve, AUC) for FIGS. 4A and 4B.

FIG. 5A provides a representative fluorescence trace of 5FAM-Fz7-21 incubated with various human sFRP proteins prior to analysis by FSEC.

FIG. 5B provides a representative fluorescence trace of 5FAM-Fz7-21S incubated with various sFRP proteins prior to analysis by FSEC.

FIG. 5C provides a quantification of fluorescence intensity (area under curve, AUC) for FIGS. 5A and 5B.

FIG. 6A provides a size exclusion chromatography profile of purified recombinant hFZD CRD.

FIG. 6B shows an SDS-PAGE of pooled hFZD7 CRD from FIG. 6A.

FIG. 6C provides the results of experiments that were performed to assess whether Fz7-21 induces mulimerization of hFZD7 CRD.

FIG. 6D provides a zoom-in view (1.5 mL to 2.0 mL range) of FIG. 6C.

FIG. 7 provides a cladogram showing the evolutionary conservation between human FZD cysteine-rich domains (CRDs) with 5FAM-Fz7-21 or 5FAM-Fz7-21S binding activity.

FIG. 8A shows a ribbon representation of crystal structure of apo hFZD7 CRD dimer.

FIG. 8B shows a surface representation of lipid-binding cavity that bridges the apo hFZD7 CRD dimer interface.

FIG. 8C shows the crystal structure of hFZD7 CRD bound to Fz7-21.

FIG. 8D shows a surface representation of the hydrophobic cavity mapped onto the structure of hFZD7 CRD bound to Fz7-21.

FIG. 8E shows a top view surface representation of the crystal structure of hFZD7 CRD bound to Fz7-21.

FIG. 8F shows a side view surface representation of the crystal structure of hFZD7 CRD bound to Fz7-21.

FIG. 9A shows a surface representation of the lipid-binding groove of hFZD4.

FIG. 9B shows a surface representation of the lipid-binding groove of mFZD8.

FIG. 9C shows the superimposition of FZD CRDs.

FIG. 10A provides a schematic of hFZD7 CRD fused to Fz7-21 through a linker (SEQ ID NO: 136).

FIG. 10B shows a size-exclusion chromatography profile of purified hFZD7 CRD-Fz7-21 fusion construct.

FIG. 11A provides a diagram of intramolecular interactions in the Fz7-21 dimer.

FIG. 11B provides a diagram of intramolecular interactions in the Fz7-21 dimer in which individual interactions are depicted by lines.

FIG. 11C provides a depiction of the solvent accessible surfaces, rendered as a gradient on the Fz7-21 dimer structure.

FIG. 12A provides a ribbon representation of the structure of apo hFZD7 CRD, highlighting the 16° angle at the dimer interface.

FIG. 12B provides a ribbon representation of the structure of hFZD7 CRD bound to Fz7-21, highlighting the 90° angle at the dimer interface.

FIG. 13 highlights select Fz7-21/hFZD7 CRD interactions within the crystal structure of hFZD7 CRD bound to Fz7-21.

FIG. 14 provides an alignment of the amino acid sequences of the CRDs of CRDs of hFZD7 (SEQ ID NO: 137), hFZD2 (SEQ ID NO: 138), hFZD1 (SEQ ID NO: 139), hFZD5 (SEQ ID NO: 140), mFZD8 (SEQ ID NO: 141), hFZD4 (SEQ ID NO: 142), hFZD9 (SEQ ID NO: 143), hFZD10 (SEQ ID NO: 144), hFZD6 (SEQ ID NO: 145) and hFZD3 (SEQ ID NO: 146).

FIG. 15 shows the results of experiments that were performed to assess the effect of dFz7-21, dFz7-21-L6A, dFz7-21-W9A, dFz7-21-M13A, dFz7-21-Y14A, and dFz7-21Δ2 on Wnt-stimulated β-catenin signaling in HEK293-TB cells.

FIG. 16A provides a NOSEY connectivity plot of Fz7-21

FIG. 16B shows a representative NMR solution structure of dFz7-21 based on superimposition of the 20 lowest energy NMR structures of dFz7-21 (amino acid side chains are shown as lines).

FIG. 16C shows a 2D NOESY plot for dFz7-21.

FIG. 16D shows a 2D NOESY plot for Fz7-21S.

FIG. 16E shows 1D NMR spectra of Fz7-21, Fz7-21S and dFz7-21 peptides.

FIG. 17A shows the effect of treatment with DMSO on the morphologies of representative mouse intestinal organoids.

FIG. 17B shows the effect of treatment with Fz7-21S on the morphologies of representative mouse intestinal organoids.

FIG. 17C shows the effect of treatment with anti-Lrp6 blocking antibody on the morphologies of representative mouse intestinal organoids.

FIG. 17D shows the effect of treatment with 200 μM dimerized Fz7-21 on the morphologies of representative mouse intestinal organoids.

FIG. 17E shows the effect of treatment with 100 μM dimerized Fz7-21 on the morphologies of representative mouse intestinal organoids.

FIG. 17F shows the effect of treatment with 10 μM dimerized Fz7-21 on the morphologies of representative mouse intestinal organoids.

FIG. 17G shows the effect of treatment with 1 μM dimerized Fz7-21 on the morphologies of representative mouse intestinal organoids.

FIG. 18 provides a quantification of organoid stem cell (SC) potential after peptide treatment.

FIG. 19A provides the results of experiments that were performed to assess the effect of treatment with dFz7-21 or Fz7-21S on Lrg5 expression in mouse intestinal organoids.

FIG. 19B provides the results of experiments that were performed to assess the effect of treatment with dFz7-21 or Fz7-21S on Asc12 expression in mouse intestinal organoids.

FIG. 19C provides the results of experiments that were performed to assess the effect of treatment with dFz7-21 or Fz7-21S on Axin2 expression in mouse intestinal organoids.

FIG. 20A provides the results of experiments that were performed to assess the effect of treatment with dFz7-21 or Fz7-21S on Lrg5 expression in intestinal epithelia collected from mice.

FIG. 20B provides the results of experiments that were performed to assess the effect of treatment with dFz7-21 or Fz7-21Son Asc12 expression in intestinal epithelia collected from mice.

FIG. 20C provides the results of experiments that were performed to assess the effect of treatment with dFz7-21 or Fz7-21Son Axin2 expression in intestinal epithelia collected from mice.

FIG. 21A Shows the results of experiments performed to determine the effect of peptide dFz7-21Δ2.M13Adp on β-catenin signaling in HEK293-TB cells that were stimulated with 50 ng/ml recombinant Wnt3a.

FIG. 21B Shows the results of experiments performed to determine the effect of peptide dFz7-21Δ2.M13Tbh on β-catenin signaling in HEK293-TB cells that were stimulated with 50 ng/ml recombinant Wnt3a.

FIG. 21C Shows the results of experiments performed to determine the effect of peptide dFz7-21Δ2.M13K(C8) on β-catenin signaling in HEK293-TB cells that were stimulated with 50 ng/ml recombinant Wnt3a.

FIG. 21D Shows the results of experiments performed to determine the effect of peptide dFz7-21Δ2.L6Hof on β-catenin signaling in HEK293-TB cells that were stimulated with 50 ng/ml recombinant Wnt3a.

FIG. 21E Shows the results of experiments performed to determine the effect of peptide dFz7-21Δ2.M13C8 on β-catenin signaling in HEK293-TB cells that were stimulated with 50 ng/ml recombinant Wnt3a.

FIG. 21F Shows the results of experiments performed to determine the effect of peptide dFz7-21Δ2.M13K(C10) on β-catenin signaling in HEK293-TB cells that were stimulated with 50 ng/ml recombinant Wnt3a.

FIG. 21G Shows the results of experiments performed to determine the effect of peptide dFz7-21Δ2.L6Hol on β-catenin signaling in HEK293-TB cells that were stimulated with 50 ng/ml recombinant Wnt3a.

FIG. 21H Shows the results of experiments performed to determine the effect of peptide dFz7-21Δ2.L6KC(8) on β-catenin signaling in HEK293-TB cells that were stimulated with 50 ng/ml recombinant Wnt3a.

FIG. 21I Shows the results of experiments performed to determine the effect of peptide dFz7-21Δ2.M13K(C12) on β-catenin signaling in HEK293-TB cells that were stimulated with 50 ng/ml recombinant Wnt3a.

FIG. 21J Shows the results of experiments performed to determine the effect of peptide dFz7-21Δ2.M13K(C14) on β-catenin signaling in HEK293-TB cells that were stimulated with 50 ng/ml recombinant Wnt3a.

FIG. 21K Shows the results of experiments performed to determine the effect of peptide dFz7-21Δ2.M13K(C16) on β-catenin signaling in HEK293-TB cells that were stimulated with 50 ng/ml recombinant Wnt3a.

FIG. 22A shows the results of experiments that were performed to assess the binding of Wnt5a to FZD1 CRD, FZD2 CRD, FZD4 CRD, and FZD7 CRD in the presence of Fz7-21 at peptide concentrations below 10 μM.

FIG. 22B shows the results of experiments that were performed to assess the binding of Wnt5a to FZD1 CRD, FZD2 CRD, FZD4 CRD, and FZD7 CRD in the presence of dFz7-21 at peptide concentrations below 10 μM.

FIG. 22C shows the results of experiments that were performed to assess the binding of Wnt5a to FZD1 CRD, FZD2 CRD, FZD4 CRD, and FZD7 CRD in the presence of Fz7-21S at peptide concentrations below 10 μM.

FIG. 22D shows the results of experiments that were performed to assess the binding of Wnt3a to FZD1 CRD, FZD2 CRD, FZD4 CRD, and FZD7 CRD in the presence of Fz7-21 at peptide concentrations below 10 μM.

FIG. 22E shows the results of experiments that were performed to assess the binding of Wnt3a to FZD1 CRD, FZD2 CRD, FZD4 CRD, and FZD7 CRD in the presence of dFz7-21 at peptide concentrations below 10 μM.

FIG. 22F shows the results of experiments that were performed to assess the binding of Wnt3a to FZD1 CRD, FZD2 CRD, FZD4 CRD, and FZD7 CRD in the presence of Fz7-21S at peptide concentrations below 10 μM.

FIG. 23A shows the results of experiments that were performed to assess the binding of Wnt5a to FZD7 CRD in the presence of, dFz7-21, Fz7-21S, or dFz7-21Δ2 at peptide concentrations below 10 μM.

FIG. 23B shows the results of experiments that were performed to assess the binding of Wnt5a to FZD7 CRD in the presence of Fz7-21S, dFz7-21, or M13Adp at peptide concentrations below 10 μM.

FIG. 23C shows the results of experiments that were performed to assess the binding of Wnt5a to FZD7 CRD in the presence of Fz7-21S, dFz7-21, or M13Tbh at peptide concentrations below 10 μM.

FIG. 23D shows the results of experiments that were performed to assess the binding of Wnt5a to FZD7 CRD in the presence of Fz7-21S, dFz7-21, or M13K(C8) at peptide concentrations below about 0.5 μM.

FIG. 23E shows the results of experiments that were performed to assess the binding of Wnt5a to FZD7 CRD in the presence of Fz7-21S, dFz7-21, or L6Hof at peptide concentrations below 10 μM.

FIG. 23F shows the results of experiments that were performed to assess the binding of Wnt5a to FZD7 CRD in the presence of Fz7-21S, dFz7-21, or M13C8 at peptide concentrations below 10 μM.

FIG. 23G shows the results of experiments that were performed to assess the binding of Wnt5a to FZD7 CRD in the presence of Fz7-21S, dFz7-21, or M13K(C10) at peptide concentrations below 10 μM.

FIG. 23H shows the results of experiments that were performed to assess the binding of Wnt5a to FZD7 CRD in the presence of Fz7-21S, dFz7-21, or L6Hol at peptide concentrations below 10 μM.

FIG. 23I shows the results of experiments that were performed to assess the binding of Wnt5a to FZD7 CRD in the presence of Fz7-21S, dFz7-21, or L6K(C8) at peptide concentrations below 10 μM.

FIG. 23J shows the results of experiments that were performed to assess the binding of Wnt5a to FZD7 CRD in the presence of Fz7-21S, dFz7-21, M13K(C8), M13K(C10), M13K(C12), M13K(C14), or M13K(C16) at peptide concentrations below 10 μM.

FIG. 23K shows the results of experiments that were performed to assess the binding of Wnt5a to FZD4 CRD in the presence of Fz7-21S, dFz7-21, dFz7-21Δ2.M13Adp, dFz7-21Δ2.M13Tbh, dFz7-21Δ2.M13K(C8), dFz7-21Δ2.L6Hof, dFz7-21Δ2.M13C8, dFz7-21Δ2.M13K(C10), dFz7-21Δ2 (Q519), dFz7-21Δ2.L6Hol, or dFz7-21Δ2.L6KC(8) at peptide concentrations below 10 μM.

FIG. 23L shows the results of experiments that were performed to assess the binding of Wnt5a to FZD4 CRD in the presence of Fz7-21S, dFz7-21, M13K(C8), M13K(C10), M13K(C12), M13K(C14), or M13K(C16) at peptide concentrations below 10 μM

FIG. 24A shows molecular weight (MW) standards analyzed by UV absorption were plotted as a function of elution volume (Ve) over void volume (Vo). Values represent the mean±s.e.m. of three independent experiments.

FIG. 24B shows the observed molecular weights of FZD CRD-Fc proteins bound to 5FAM-Fz7-21 (gray circles) vs. the predicted FZD CRD-Fc tetrameric MW (black squares).

FIG. 24C shows a native PAGE (4-16%) of different FZD CRD-Fc proteins used (˜2 μg).

FIG. 25A shows a ribbon representation of apo hFZD7 CRD crystal structure with a schematic of full length FZD7 illustrating the CRD placement within FZD7.

FIG. 25B shows a ribbon representation of the structure of hFZD7 CRD bound to Fz7-21.

FIG. 25C provides a zoomed-in side view of the hydrophobic cavity in apo hFZD7 CRD.

FIG. 25D provides a top view from of FIG. 25C.

FIG. 25E provides a zoomed-in side view of hFZD7 CRD bound to Fz7-21.

FIG. 25F shows the top view of FIG. 25E.

FIG. 26 shows a superimposition of the 20 lowest energy NMR structures of dFz7-21.

FIG. 27A shows the associated molecular weight standards used to determine the MW of hFZD7 CRD-GS.

FIG. 27B shows an SDS-PAGE of the fusion protein in FIG. 10B under reducing conditions.

FIG. 27C shows a bright field image of crystals obtained from the fusion protein in FIG. 10B.

FIG. 28A provides a superimposition of hFZD7 CRD protomers bound to their respective C24 fatty acids.

FIG. 28B shows the structure from FIG. 28A without ribbon representation displaying residues proximal to the C24 fatty acid. The C24 fatty acid carbons are numbered sequentially starting from the carboxylic acid headgroup (C1) to the w-carbon (C24). Water, crystallization cofactors, and glycans are hidden for clarity.

FIG. 28C provides a superimposition of apo hFZD7 CRD (ribbon representation) with hFZD7 CRD (ribbon representation) bound to C24 fatty acid (ball and stick representation).

FIG. 29A provides the crystal structure of hFZD7 CRD dimer (ribbon representation) in complex with C24 fatty acid (ball and stick representation).

FIG. 29B provides a surface representation of the hydrophobic cavity mapped onto hFZD7 CRD (ribbon representation) structure in complex with C24 fatty acid (black; ball and stick representation).

FIG. 29C provides a zoomed-in view of FIG. 29B, displaying residues proximal to the hydrophobic cavity (gray, chain A; white, chain B; C24 fatty acid, black; ball and stick representation). C24 fatty acid carbons are numbered sequentially, starting from the carboxylic acid headgroup (C1) to the w-carbon (C24). The base of the lipid-binding cavity is between C9 and C13.

FIG. 29D shows the superimposition of the lipid-binding cavities of apo-hFZD7 CRD (PDB ID 5T44; light gray, stick representation) and hFZD7 CRD (gray, stick representation) bound to C24 fatty acid (black, space filling model). Hydrogen bonding interactions are displayed (black; dashed lines).

FIG. 30A shows a superimposition of smoothened CRD (gray; ribbon representation; PDB ID#5KZV) bound to 20(S)-hydroxycholesterol (gray; stick representation) with hFZD7 CRD (white; ribbon representation) bound to C24 fatty acid (white; stick representation; r.m.s deviation=8.3 Å over 108 atom pairs). Zoomed-in inserts highlight residues in close proximity to ligand hydroxyl groups and hydrogen bonding interactions are displayed (black; dashed lines).

FIG. 30B shows a superimposition of smoothened CRD (gray; ribbon representation; PDB ID#5KZV) bound to 20(S)-hydroxycholesterol (gray; stick representation) with hFZD5 CRD (tan, chain A; ribbon representation) bound to C16:1 fatty acids (darker gray; stick representation; r.m.s deviation is 4.6 Å over 109 atom pairs). Zoomed-in inserts highlight residues in close proximity to ligand hydroxyl groups and hydrogen bonding interactions are displayed (black; dashed lines).

FIG. 31 shows the structure of human (h) FZD5 CRD (chain A, light gray; chain B, medium gray; ribbon representation) bound to C16:ln-7 fatty acid (black or dark gray; ball and stick representation). Each C16: In-7 fatty acid within chain A has 50% occupancy due to symmetry mate averaging.

FIG. 32A shows a crystal structure of hFZD5 CRD homodimer (chain A; ribbon representation) in complex with C16:ln-7 fatty acids (dark gray or black; ball and stick representation).

FIG. 32B provides a surface representation of the hFZD5 CRD chain A homodimer hydrophobic cavity (gray) mapped onto hFZD5 CRD (ribbon representation) in complex with two C16:ln-7 fatty acids each with 50% occupancy (black or gray; ball and stick representation).

FIG. 32C shows a zoomed-in view of FIG. 32B without ribbon representation. Residues proximal to the hydrophobic cavity are highlighted (dark gray, stick representation). C16: In-7 fatty acid carbons are numbered sequentially, starting from the carboxylic acid headgroup (C1) to the ω-carbon (C16). The base of the lipid-binding cavity is between C7 and C10.

FIG. 33A provides a crystal structure of human (h) FZD5 CRD dimer in complex with two molecules of n-octyl-β-D-glucoside (BOG) each with 50% occupancy (gray or black; ball and stick representation).

FIG. 33B shows a surface representation of hFZD5 CRD chain A's hydrophobic cavity mapped onto the structure of hFZD5 CRD chain A (white; ribbon representation) bound to n-octyl-β-D-glucoside (ball and stick representation) highlighting residues within ˜5 Å of n-octyl-β-D-glucoside (representation).

FIG. 33C shows a zoomed-in view of FIG. 33B without the carbon backbone displayed.

Hydrogen bonding interactions between hFZD5 CRD and the glycoside of n-octyl-β-D-glucoside are highlighted (dashed lines; black).

FIG. 33D shows the molecular structure of n-octyl-β-D-glucoside highlighting molecular components.

FIG. 34A shows a superimposition of C16: In-7 fatty acid (ribbon and stick representation) or n-octyl-β-D-glucoside (ribbon and stick representation) bound hFZD5 CRD (r.m.s deviation across 119 residues is 0.134 Å). Each C16:ln-7 fatty acid and n-octyl-β-D-glucoside within chain A have 50% occupancy due to symmetry mate averaging.

FIG. 34B shows a ribbon representation of hFZD5 CRD Chain B from FIG. 34A highlighting select residues near the C16:ln-7 fatty acid.

FIG. 34C shows hFZD5 CRD chain A from FIG. 34A highlighting residues that contact either C16:ln-7 fatty acid or n-octyl-β-D-glucoside (VDW overlap > to −0.4 Å). The zoomed-in view depicts hydrogen bonding interactions between FZD5 CRD and n-octyl-β-D-glucoside are shown (black dashed lines).

FIG. 35A shows the crystal structure of mFZD8 CRD dimer (PDB ID# MY; ribbon representation) with surface representation of the homodimer hydrophobic cavities. The dimer interface from the Examples and Dann, C. E. et al. “Insights into Wnt binding and signaling from the structures of two Frizzled cysteine-rich domains.” Nature 412, 86-90 (2001) are indicated.

FIG. 35B shows a superimposition of chain A and chain B homodimers with surface representations of their hydrophobic cavities.

FIG. 35C shows a zoomed-in view of FIG. 35B highlighting the side chains proximal to the hydrophobic cavity of mFZD8 CRD.

FIG. 35D shows a superimposition BOG-bound hFZD5 CRD (ribbon representation), C16:ln-7 fatty acid bound hFZD5 CRD (ribbon representation), apo-FZD7 CRD (ribbon representation), C24 fatty acid-bound hFZD7 CRD (ribbon representation) and apo-mFZD8 CRD (ribbon representation).

FIG. 35E shows a model of Wnt binding to the CRD of FZD receptors, utilizing the U-shaped hydrophobic cavity for cis-fatty acid selectivity. FZDs 5, 7 and 8 (possibly FZD1 and FZD2) receptors (CRDs, tan or blue ovals; 7-pass transmembrane domains, yellow ovals) are in their inactive state and may form a dimer configuration in which the hydrophobic cavities form a continuous and U-shaped cavity (red). Upon Wnt binding, the cis-49-unsaturated fatty acid occupies the lipid-binding cavity, utilizing the “kink” to traverse the dimer interface, and recruits ω-factors to stimulate downstream Wnt signaling.

FIG. 36A shows a table of dimer interaction interface potential energies of hFZD7 CRD, hFZD5 CRD and hFZD8 CRD.

FIG. 36B shows a table of FZD CRD dimer complementarity score (Sc).

FIG. 36C shows a visualization of the dimer complementarity score for the loop-loop interaction interface of hFZD7 CRD. According to the dimer complementarity score, the center of the interaction interface is hypothesized to has low complementarity and the helices at the periphery have high complementarity.

FIG. 36D shows a visualization of the dimer complementarity score for the loop-loop interaction interface of mFZD8 CRD. According to the dimer complementarity score, the center of the interaction interface has low complementarity and the helices at the periphery are have high complementarity.

FIG. 36E shows a visualization of the dimer complementarity score for the loop-loop interaction interface of hFZD5 CRD. According to the dimer complementarity score, the center of the interaction interface has low complementarity and the helices at the periphery have high complementarity.

FIG. 36F shows a visualization of the dimer complementarity score for the FZD7-like alpha-helical dimer interaction interface of hFZD5 CRD. According to the dimer complementarity score, the center of the interaction interface has low complementarity and the helices at the periphery have high complementarity.

FIG. 36G shows a visualization of the dimer complementarity score for the FZD7-like alpha-helical dimer interaction interface of hFZD7 CRD. According to the dimer complementarity score, the center of the interaction interface has low complementarity and the helices at the periphery high complementarity.

FIG. 36H shows a visualization of the dimer complementarity score for the FZD7-like alpha-helical dimer interaction interface of hFZD7 CRD. According to the dimer complementarity score, the center of the interaction interface has low complementarity and the helices at the periphery have high complementarity.

FIG. 37A shows a Clustal Omega sequence alignment of human FZD CRD family members.

Residues that form crystallographic FZD7-like dimer contacts between protomers are underlined (VDW overlap >−0.4 angstroms). Conservation of the FZD7-like dimer interface between FZD family members is denoted by “+” above the sequence alignment. Conserved cysteines are highlighted in blue. FZD7 (SEQ ID NO: 147), FZD2 (SEQ ID NO: 148), FZD1 (SEQ ID NO: 149), FZD5 (SEQ ID NO: 150), FZD8 (SEQ ID NO: 151), FZD4 (SEQ ID NO: 152), FZD9 (SEQ ID NO: 153), FZD10 (SEQ ID NO: 154), FZD6 (SEQ ID NO: 155), and FZD3 (SEQ ID NO: 156).

FIG. 37B shows the structure of apo-hFZD7 CRD (ribbon representation) is used as a surrogate to highlight the alpha-helical FZD7 dimer interface with 180° rotation. Inserts display zoomed-in views of the dimer interface and highlight residue-residue hydrogen bonding (black dashed lines).

FIG. 38A shows XWnt8 (light gray; ribbon representation) in complex with mFZD8 CRD (white; ribbon representation; PDB ID#4F0A) was superimposed onto the alpha-helical dimer of mFZD8 CRD (gray; ribbon representation; PDB ID#1IJY). The hydrophobic cavity surface of the indicated dimer (was mapped onto the FZD CRD structure with front surface transparency. The C14 fatty acid (ball and stick representation) is covalently bound at XWnt8S187. Water, crystallization cofactors and glycosylations are hidden for clarity.

FIG. 38B shows XWnt8 (light gray; ribbon representation) in complex with hFZD7 CRD (gray; ribbon representation) in complex with a C24 fatty acid (black; ball and stick representation). The hydrophobic cavity surface of the indicated dimer (was mapped onto the FZD CRD structure with front surface transparency. The C14 fatty acid (light gray; ball and stick representation) is covalently bound at XWnt8S187. Water, crystallization cofactors and glycosylations are hidden for clarity.

FIG. 38C shows XWnt8 (light gray; ribbon representation) in complex with hFZD5 CRD chain A homodimer (gray; ribbon representation) in complex with C16:ln-7 fatty acid (gray or black; ball and stick representation). The hydrophobic cavity surface of the indicated dimer was mapped onto the FZD CRD structure with front surface transparency. The C14 fatty acid (light gray; ball and stick representation) is covalently bound at XWnt8S187. Water, crystallization cofactors and glycosylations are hidden for clarity.

FIG. 39A provides a model of XWnt8a (PDB ID #4F0A) in complex with hFZD7 CRD (PDB ID #5URV) with an elongated fatty acyl moiety (C16:n-7) binding in the U-shaped hydrophobic cavity of hFZD7 CRD.

FIG. 39B provides an enlarged view of the portion of FIG. 39A in the black box.

FIG. 39C provides a proposed model for Wnt interaction with FZD7 CRD in the presence of peptide Fz7-21.

FIG. 39D provides an enlarged view of the portion of FIG. 39C in the black box.

FIG. 40 provides the results of fluorescence size-exclusion (FSEC) chromatography experiments that were performed to assess whether mutations at specific residues within the peptide-binding region on hFZD7 CRD reduced the binding of Fz7-21 compared to wild-type hFZD7 CRD.

FIG. 41A provides the results of experiments that were performed to determine whether treatment with dFz7-21 reduced the number of Lgr5-GFP stem cells in organoids derived from Lgr5-GFP mice.

FIG. 41B provides the results of experiments that were performed to determine whether treatment with dFz7-21 reduced the number of Lgr5-GFP stem cells in organoids derived from Lgr5-GFP mice.

FIG. 41C provides a quantification of the results shown in FIGS. 41A and 41B.

FIG. 42A provides the results of experiments that were performed to assess the effects of treatment with DMSO, Fz7-21 or Fz7-21S on Axin 2 mRNA levels in Lgr5-GFP organoids.

FIG. 42B provides the results of experiments that were performed to assess the effects of treatment with DMSO, Fz7-21 or Fz7-21S on Asc12 mRNA levels in Lgr5-GFP organoids.

FIG. 42C provides the results of experiments that were performed to assess the effects of treatment with DMSO, Fz7-21 or Fz7-21S on Axin2 mRNA levels in APC^{min} organoids.

FIG. 42D provides the results of experiments that were performed to assess the effects of treatment with DMSO, Fz7-21 or Fz7-21S on Asc12 mRNA levels in APC^{min} organoids.

FIG. 42E provides the results of experiments that were performed to assess the effects of treatment with DMSO, Fz7-21 or Fz7-21S on Lgr5 mRNA levels in APC^{min} organoids.

### DETAILED DESCRIPTION OF THE INVENTION

Provided are ligands comprising a non-naturally occurring peptide that binds or specifically binds the cysteine rich domain (CRD) of the Frizzled 7 (FZD7) receptor. In certain embodiments, these ligands further bind the cysteine rich domain (CRD) of a Frizzled (FZD) receptor selected from the group consisting of: Frizzled 1 (FZD1) and Frizzled 2 (FZD2). Such ligands demonstrate one or more of the following characteristics: inhibition of Wnt-mediated B-catenin signaling with and IC₅₀ less than about 100 nM; an EC50 value of less than 90 nM, binding to human FZD7 CRD and mouse FZD7 CRD, and/or binding to an binding region of human FZD7 CRD that comprises three or more of the following amino acids: Leu81, His84, Gln85, Tyr87, Pro88, Phe138, and Phe140.

Also provided are methods of using ligands comprising a non-naturally occurring peptide that binds or specifically binds the CRD FZD7 in treating cancer (such as colon cancer, pancreatic cancer, non-small cell lung cancer, cancer characterized by a mutation in RNF43, cancer characterized by USP6 overexpression, or cancer characterized by gene fusions involving R-spondin (RSPO) family members) in a subject. Also provided are methods of using ligands comprising a non-naturally occurring peptide that further binds the CRD of FZD1 and/or FZD2 in treating cancer (such as colon cancer, pancreatic cancer, non-small cell lung cancer, cancer characterized by a mutation in RNF43, cancer characterized by USP6 overexpression, or cancer characterized by gene fusions involving R-spondin (RSPO) family members) in a subject. Also provided are uses of ligands comprising a non-naturally occurring peptide that binds or specifically binds the CRD of FZD7 in the manufacture of a medicament for the treatment of ocular disease or disorders. Also provided are uses of such ligands comprising a non-naturally occurring peptide that further binds the CRD of FZD1 and/or FZD2 in the manufacture of a medicament for the treatment of ocular disease or disorders.

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N J, 2000; and the series “Methods in Enzymology,” Academic Press, San Diego, Calif.

### Definitions

As used herein “non-naturally occurring” means, e.g., a polypeptide comprising an amino acid sequence that is not found in nature, or, e.g., a nucleic acid comprising a nucleotide sequence that is not found in nature. A non-naturally occurring peptide provided herein can be produced by genetic engineering methods or by chemical synthesis methods. Thus, a non-naturally occurring peptide described herein may be recombinant, i.e., produced by a cell, or nucleic acid, or vector, that has been modified by the introduction of a heterologous nucleic acid or the alteration of a native nucleic acid to a form not native to that cell, or that the cell is derived from a cell so modified. Alternatively, a non-naturally occurring peptide described herein can be produced via chemical peptide synthesis.

As used herein, an “amino acid alteration” refers to the addition, deletion, or substitution of at least one amino acid in, e.g., a peptide sequence (such as in the sequence of a non-naturally occurring peptide that binds or specifically binds to the cysteine rich domain (CRD) of FZD7).

As used herein, “ligand” refers to a molecule comprising (such as consisting essentially of or consisting of) at least one non-naturally occurring peptide described herein that binds or specifically binds, e.g., the cysteine rich domain (CRD) of Frizzled 7 (FZD7). The binding of a ligand comprising (such as consisting essentially of or consisting of) at least one non-naturally occurring peptide described herein to the CRD is measurably different from a non-specific interaction, and can be detected by, e.g., binding assay to measure protein-ligand binding or an immunoassay.

A ligand of this invention which “binds” a receptor of interest is one that binds the receptor with sufficient affinity such that the ligand is useful as a diagnostic and/or therapeutic agent in targeting a protein or a cell or tissue expressing the receptor. With regard to the binding of a ligand to a target molecule or receptor, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess non-labeled target. In one particular embodiment, “specifically binds” refers to binding of a ligand to its specified target FZD7 CRD domain and not other specified FZD CRD domains. For example, the ligand specifically binds to the FZD7 CRD but does not specifically bind to FZD8 CRD.

In certain embodiments, the extent of binding of the ligand to a “non-target” FZD receptor (such as FZD3, FZD4, FZD5, FZD6, FZD8, FZD9, or FZD10) will be less than about 10% of the binding of the ligand to FZD1, FZD2, and/or FZD7, as determined by, e.g., fluorescence activated cell sorting (FACS) analysis or radioimmunoprecipitation (RIA). In certain embodiments, a ligand of the present disclosure specifically binds to FZD1, FZD2, and/or FZD7 with a dissociation constant (Kd) or IC₅₀ value equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; measured at a temperature of about 4° C., 25° C., 37° C., or 45° C. In certain embodiments, the extent of binding of the ligand to a “non-target” FZD receptor (such as FZD1, FZD2, FZD3, FZD4, FZD5, FZD6, FZD8, FZD9, or FZD10) will be less than about 10% of the binding of the ligand to FZD7, as determined by, e.g., fluorescence activated cell sorting (FACS) analysis or radioimmunoprecipitation (RIA). In certain embodiments, a ligand of the present disclosure specifically binds to FZD7 with a dissociation constant (Kd) or IC₅₀ value equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; measured at a temperature of about 4° C., 25° C., 37° C., or 45° C.

An “isolated” ligand is one which has been identified and separated and/or recovered from composition comprising the ligand and a contaminant or impurity. Contaminants or impurities are materials which would interfere with diagnostic or therapeutic uses of a ligand comprising (such as consisting essentially of or consisting of) a non-naturally occurring peptide that binds or specifically binds a cysteine-rich domain (CRD) of FZD7. Contaminants can include, e.g., host cell enzymes, hormones, and other proteinaceous or nonproteinaceous solutes, i.e., if the ligand is produced recombinantly, or, e.g., salts, reagents, truncated or degraded sequences, incompletely deprotected sequences, etc., i.e., if the ligand is produced via chemical synthesis. In preferred embodiments, a ligand provided herein will be purified (1) to greater than 95% by weight of ligand as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated ligands include the ligand in situ within recombinant cells. An isolated a ligand comprising (such as consisting essentially of or consisting of) a non-naturally occurring peptide that binds or specifically binds a cysteine-rich domain (CRD) of FZD7 will be prepared by at least one purification step.

“Percent (%) amino acid sequence identity” or “homology” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

As used herein the term “binding region” refers to a region capable of being specifically bound by a peptide (such as a non-naturally occurring peptide that specifically binds to the cysteine rich domain (CRD) of FZD7 provided herein). A binding region can comprise between about 3-10 amino acids in a spatial conformation, which is unique to the binding region. These amino acids can be linear within the protein (i.e., consecutive in the amino acid sequence) or they can be positioned in different parts of the protein (i.e., non-consecutive in the amino acid sequence). Methods of determining the spatial conformation of amino acids within a protein, or at the interface of two proteins, are known in the art, and include, for example, x-ray crystallography and 2-dimensional nuclear magnetic resonance.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

An “effective amount” of a ligand (or a composition comprising such a ligand) as disclosed herein is an amount sufficient to carry out a specifically stated purpose. An “effective amount” can be determined empirically and by known methods relating to the stated purpose.

The term “therapeutically effective amount” refers to an amount of a ligand or composition as disclosed herein, effective to “treat” a disease or disorder in a mammal (such as a human patient). In the case of cancer, the therapeutically effective amount of a ligand as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent that a ligand as disclosed herein can prevent growth and/or kill existing cancer cells, it can be cytostatic and/or cytotoxic. In one embodiment, the therapeutically effective amount is a growth inhibitory amount. In another embodiment, the therapeutically effective amount is an amount that extends the survival of a patient. In another embodiment, the therapeutically effective amount is an amount that improves progression free survival of a patient.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

A “disorder” is any condition that would benefit from treatment with a ligand comprising (such as consisting essentially of or consisting of) a non-naturally occurring peptide that binds a cysteine-rich domain (CRD) of FZD7, or with a ligand comprising (such as consisting essentially of or consisting of) a non-naturally occurring peptide that specifically binds a CRD of FZD7, described herein. For example, mammals who suffer from or need prophylaxis against abnormal Wnt expression or activity. This includes chronic and acute disorders or diseases including those pathological conditions which predispose the mammal to the disorder in question. Non-limiting examples of disorders to be treated herein include cancer and metastatic disease as described elsewhere herein. In certain embodiments, a ligand comprising (such as consisting essentially of or consisting of) a non-naturally occurring peptide that binds a cysteine-rich domain (FZD7, or with a ligand comprising (such as consisting essentially of or consisting of) a non-naturally occurring peptide that specifically binds a CRD of FZD7 can be used to promote tissue repair, wound healing, and bone growth.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “detecting” is intended to include determining the presence or absence of a substance or quantifying the amount of a substance (such as a FZD7 or a receptor thereof). The term thus refers to the use of the materials, compositions, and methods provided herein for qualitative and quantitative determinations. In general, the particular technique used for detection is not critical for practice of the invention.

For example, “detecting” according to the invention may include: observing the presence or absence FZD7; a change in the levels of FZD7; and/or a change in biological function/activity of FZD7. In certain embodiments, “detecting” may include detecting levels of a FZD7 (e.g., polypeptide levels of a human FZD1, a human FZ2, and/or a human FZD7). Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, or more e.g., 100-fold.

The word “label” when used herein refers to a detectable compound or composition which is conjugated directly or indirectly to the ligand comprising (such as consisting essentially of or consisting of) a non-naturally occurring peptide that specifically binds a cysteine rich domain (CRD) of a Frizzled 7 (FZD7). The label may itself be detectable by itself (e.g., radioisotope labels or fluorescent labels) or, in the case of an enzymatic label, may catalyze chemical alteration of a substrate compound or composition which is detectable.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the invention described herein include “comprising,” “consisting,” and “consisting essentially of” aspects and embodiments.

All references cited herein, including patent applications and publications, are hereby incorporated by reference in their entirety.

Ligands Comprising a Non-Naturally Occurring Peptide that Binds the Cysteine-Rich Domain of FZD7

Provided is a ligand comprising (such as consisting essentially of or consisting of) a non-naturally occurring peptide that binds or specifically binds a cysteine rich domain (CRD) of Frizzled 7 (FZD7). In certain embodiments, the ligand binds one or more Frizzled (FZD) receptors selected from the group consisting of: Frizzled 1 (FZD1), Frizzled 2 (FZD2), and Frizzled 7 (FZD7). In certain embodiments, the FZD1 is a human FZD1. In certain embodiments, the FZD1 is a mouse FZD1. In certain embodiments, the FZD2 is a human FZD2. In certain embodiments, the FZD2 is a mouse FZD2. In certain embodiments, the FZD7 is a human FZD7. In certain embodiments, the FZD7 is a mouse FZD7. In specific embodiments, the ligand comprises (such as consists essentially of or consists of) a non-naturally occurring peptide that specifically binds a cysteine rich domain (CRD) of Frizzled 7 (FZD7) (e.g., human FZD7 or mouse FZD7). In certain embodiments, the ligand comprises (such as consists essentially of or consists of) a non-naturally occurring peptide that does not bind the CRD of Frizzled 3 (FZD3), Frizzled 4 (FZD4), Frizzled 5 (FZD5), Frizzled 6 (FZD6), Frizzled 8 (FZD), Frizzled 9 (FZD9) or Frizzled 10 (FZD10). In certain embodiments, the ligand comprises (such as consists essentially of or consists of) a non-naturally occurring peptide that does not bind the CRD of Frizzled 1 (FZD1), Frizzled 2 (FZD2), Frizzled 3 (FZD3), Frizzled 4 (FZD4), Frizzled 5 (FZD5), Frizzled 6 (FZD6), Frizzled 8 (FZD), Frizzled 9 (FZD9) or Frizzled 10 (FZD10).

In certain embodiments, the ligand comprises (such as consists essentially of or consists of) a non-naturally occurring peptide that specifically binds or specifically binds the CRD of FZD7. In certain embodiments, the ligand comprises (such as consists essentially of or consists of) a non-naturally occurring peptide that binds or specifically binds the CRD of hFZD7. In certain embodiments, the ligand comprises (such as consists essentially of or consists of) a non-naturally occurring peptide that binds or specifically binds the CRD of mFZD7.

In certain embodiments, the ligand comprises (such as consists essentially of or consists of) a non-naturally occurring peptide that binds or specifically binds the CRD FZD7 and additionally binds the CRD of any one of 1) FZD1, 2) FZD2, or FZD1 and FZD2. In certain embodiments, the ligand comprises (such as consists essentially of or consists of) a non-naturally occurring peptide that binds the CRD of FZD1 and FZD7. In certain embodiments, the ligand comprises (such as consists essentially of or consists of) a non-naturally occurring peptide that binds the CRD of FZD2 and FZD7.

In certain embodiments, the ligand comprises a non-naturally occurring peptide that competes for binding to the binding region of FZD7 CRD bound by a peptide comprising the amino acid sequence LPSDDLEFWCHVMY (SEQ ID NO: 13). In certain embodiments, the ligand consists essentially of (such as consists of) a non-naturally occurring peptide that competes for binding to the binding region of FZD7 CRD bound by a peptide comprising the amino acid sequence LPSDDLEFWCHVMY (SEQ ID NO: 13). In certain embodiments, the ligand consists essentially of (such as consists of) a non-naturally occurring peptide that competes for binding to the binding region of FZD7 CRD bound by a peptide consisting of the amino acid sequence LPSDDLEFWCHVMY (SEQ ID NO: 13).

In certain embodiments, the ligand comprises a non-naturally occurring peptide that specifically binds the same binding region of the FZD7 CRD bound by a peptide comprising the amino acid sequence LPSDDLEFWCHVMY (SEQ ID NO: 13). In certain embodiments, the ligand consists essentially of (such as consists of) a non-naturally occurring peptide that specifically binds the same binding region of the FZD7 CRD bound by a peptide comprising the amino acid sequence LPSDDLEFWCHVMY (SEQ ID NO: 13). In certain embodiments, the ligand consists essentially of (such as consists of) a non-naturally occurring peptide that specifically binds the same binding region of the FZD7 CRD bound by a peptide consisting of the amino acid sequence LPSDDLEFWCHVMY (SEQ ID NO: 13).

In certain embodiments, the ligand comprises (such as consists essentially of or consists of) a peptide that specifically binds a binding region of FZD7 that comprises at least one, at least two, at least three, at least four, at least five, at least 6, or at least 7 amino acids selected from the group consisting of: Leu81, His84, Gln85, Tyr87, Pro88, Phe138, and Phe140.

In certain embodiments, the ligand comprises (such as consists essentially of or consists of) a non-naturally occurring peptide that specifically binds a binding region of FZD7 that comprises at least one, at least two, at least three, at least four, at least five, at least 6, or at least 7 amino acids selected from the group consisting of: Leu81, His84, Gln85, Tyr87, Pro88, Phe138, and Phe140. In certain embodiments, the ligand comprises (such as consists essentially of or consists of) a non-naturally occurring peptide that specifically binds a binding region of FZD7 that is within 4 Å of at least one, at least two, at least three, at least four, at least five, at least 6, or at least 7 amino acids selected from the group consisting of: Leu81, His84, Gln85, Tyr87, Pro88, Phe138, and Phe140.

In certain embodiments, the ligand is a chimeric molecule comprising a non-naturally occurring peptide described herein that binds or specifically binds the CRD of FZD1, FZD2, and/or FZD7 fused to one or more moieties. In certain embodiments, the ligand is a chimeric molecule comprising a non-naturally occurring peptide described herein that specifically binds the CRD of FZD7 fused to one or more moieties. In certain embodiments, the moiety is fused to the N-terminus of the peptide. In certain embodiments, the moiety is fused to the C-terminus of the peptide. In certain embodiments, a first moiety is fused to the N-terminus of the peptide, and a second moiety is fused to the N-terminus of a peptide. In certain embodiments, the one or more moieties are recombinantly fused to the peptide. In certain embodiments, the one or more moieties are linked to the peptide via linker (such as a cleavable linker). Exemplary moieties include, but are not limited to, peptides, polypeptides or fragments thereof, proteins or fragments thereof, fusion proteins.

In certain embodiments, the ligand comprises a non-naturally occurring peptide described herein that binds or specifically binds the CRD of FZD7 recombinantly fused to a heterologous polypeptide or amino acid sequence. In certain embodiments, the ligand comprises a non-naturally occurring peptide described herein that binds or specifically binds the CRD of FZD7 fused (e.g., at the N-terminus, C-terminus, or to both the N- and C-termini) to protein transduction domain which targets the ligand, e.g., for delivery to various tissues, or, e.g., across brain blood barrier, using, for example, the protein transduction domain of human immunodeficiency virus TAT protein (Schwarze et al., 1999, Science 285: 1569-72). In certain embodiments, the ligand comprises a non-naturally occurring peptide described herein that binds or specifically binds the CRD of FZD7 fused to a cell-permeable peptide such as primary amphipathic peptide MPG (GALFLGFLGAAGSTMGAWSQPKKKRKV SEQ ID NO: 104), Pep-1 (KETWWETWWTEWSQPKKKRKV SEQ ID NO 105), secondary amphipathic peptide CADY (Ac-GLWRALWRLLRSLWRLLWRA-Cya SEQ ID NO: 106) or octa-arginine (R(8) SEQ ID NO 107).

In certain embodiments, the ligand comprises a non-naturally occurring peptide described herein that binds or specifically binds the CRD FZD7 fused (e.g., at the N-terminus, C-terminus, or to both the N- and C-termini) to a domain or moiety that stabilizes the conformation (such as the alpha helical structure) of the non-naturally occurring peptide.

In certain embodiments, a ligand comprising (such as consisting essentially of or consisting of) a non-naturally occurring peptide described herein that binds specifically binds the CRD of FZD7 can be used as monospecific in monomeric form or as bi- or multi-specific (for different target ligands or different binding regions on the same target ligand) in multimer form. In certain embodiments, a ligand comprising (such as consisting essentially of or consisting of) a non-naturally occurring peptide described herein that binds or specifically binds the CRD of FZD7 is conjugated to LRP6. The attachments may be covalent or non-covalent. In certain embodiments, a dimeric bispecific ligand has one subunit with specificity for a first target or binding region and a second with specificity for a second target ligand or binding region. A ligand comprising a non-naturally occurring peptide described herein that binds or specifically binds the CRD of FZD7 can be joined in a variety of conformations that can increase the valency and thus the avidity of binding.

In certain embodiments a ligand provided herein comprises two or more (such as three, four, five, six, seven, eight, nine, ten, or more than ten) non-naturally occurring peptides provided herein that bind or specifically bind the CRD of FZD7. In certain embodiments, a nucleic acid can be engineered to encode two or more copies of a single non-naturally occurring peptides provided herein that bind or specifically bind the CRD of FZD7, which copies are transcribed and translated in tandem to produce a covalently linked multimer of identical subunits. In certain embodiments, the nucleic acid can be engineered to encode two or more different non-naturally occurring peptides provided herein that bind or specifically bind the CRD of FZD7, which copies are transcribed and translated in tandem to produce a covalently linked multimer of different subunits.

In another embodiment, a ligand comprises a non-naturally occurring peptide provided herein that binds or specifically binds the CRD of FZD7 fused with a tag polypeptide which provides an epitope to which an anti-tag antibody can selectively bind. The epitope tag is generally placed at the amino- or carboxyl-terminus of the ligand. The presence of such epitope-tagged forms of the ligand can be detected using an antibody against the tag polypeptide. Also, provision of the epitope tag enables the ligand comprising the non-naturally occurring peptide provided herein that binds or specifically binds the CRD of FZD7 to be readily purified by affinity purification using an anti-tag antibody or another type of affinity matrix that specifically binds to the epitope tag.

Various tag polypeptides and their respective antibodies are known in the art. Examples include poly-histidine (poly-His) or poly-histidine-glycine (poly-His-Gly) tags; the flu HA tag polypeptide and its antibody 12CA5 (Field et al. (1988) Mol. Cell. Biol. 8, 2159-2165); the c-myc tag and the 8F9, 3C7, 6E10, G4, B7 and 9E10 antibodies thereto (Evan et al. (1985) Mol. Cell. Biol. 5, 3610-3616]; and the Herpes Simplex virus glycoprotein D (gD) tag and its antibody (Paborsky et al. (1990) Protein Eng., 3, 547-553). Other tag polypeptides include the Flag-peptide (Hopp et al. (1988) BioTechnology, 6,1204-1210); the KT3 epitope peptide (Martin et al. (1992) Science, 255, 192-194]; an α-tubulin epitope peptide (Skinner et al. (1991) J Biol. Chem. 266, 15163-15166); and the T7 gene 10 protein peptide tag (Lutz-Freyermuth et al. (1990) Proc. Natl. Acad. Sci. USA 87, 6393-6397].

In certain embodiments, the ligand comprises a non-naturally occurring peptide provided herein that binds or specifically binds the CRD of FZD7 recombinantly fused to an immunoglobulin or a particular region of an immunoglobulin. For a bivalent form of the ligand (e.g., an “immunoadhesin”), such a fusion could be to the Fc region of an IgG molecule. Ig fusions provided herein include polypeptides that comprise approximately or only residues 94-243, residues 33-53 or residues 33-52 of human in place of at least one variable region within an Ig molecule. In a particularly preferred embodiment, the immunoglobulin fusion includes the hinge, CH2 and CH3, or the hinge, CHL CH2 and CH3 regions of an IgG1 molecule. For the production of immunoglobulin fusions see also, U.S. Pat. No. 5,428,130 issued Jun. 27, 1995. In certain embodiments, the ligand comprising a non-naturally occurring peptide provided herein that binds or specifically binds the CRD of FZD7 is fused, e.g., at the N or C terminus, to the constant region of an IgG (Fc). In certain embodiments, the ligand/Fc fusion molecule activates the complement component of the immune response. In certain embodiments, the ligand/Fc fusion protein increases the therapeutic value of the ligand comprising a non-naturally occurring peptide provided herein that binds or specifically binds the CRD of FZD7. In certain embodiments, the ligand comprising a non-naturally occurring peptide provided herein that binds or specifically binds the CRD of FZD7 is fused (such as recombinantly fused), e.g., at the N or C terminus, to a complement protein, such as CIq. Various publications describe methods for obtaining non-naturally occurring proteins whose half-lives are modified either by introducing an FcRn-binding polypeptide into the molecules (WO 1997/43316, U.S. Pat. Nos. 5,869,046, 5,747,035, WO 1996/32478, WO 1991/14438) or by fusing the proteins with antibodies whose FcRn-binding affinities are preserved but affinities for other Fc receptors have been greatly reduced (WO 1999/43713) or fusing with FcRn binding domains of antibodies (WO 2000/09560, U.S. Pat. No. 4,703,039). Specific techniques and methods of increasing half-life of physiologically active molecules (e.g., ligands comprising a non-naturally occurring peptide provided herein that binds or specifically binds the CRD of FZD7) can also be found in U.S. Pat. No. 7,083,784. In certain embodiments, the ligand comprising a non-naturally occurring peptide provided herein that binds or specifically binds the CRD of FZD7 is fused to an Fc region from an IgG that comprises amino acid residue mutations (as numbered by the EU index in Kabat): M252Y/S254T/T256E or H433K/N434F/Y436H.

In certain embodiments, the ligand comprises a non-naturally occurring peptide provided herein that binds or specifically binds the CRD of FZD7 fused with a molecule that increases or extends in vivo or serum half-life. In certain embodiments, the ligand comprises a non-naturally occurring peptide provided herein that binds or specifically binds the CRD of FZD7 fused with albumin, such as human serum albumin (HSA), polyethylene glycol (PEG), polysaccharides, immunoglobulin molecules (IgG), complement, hemoglobin, a binding peptide, lipoproteins or other factors to increase its half-life in the bloodstream and/or its tissue penetration.

Additional ligands comprising a non-naturally occurring peptide provided herein that binds or specifically binds the CRD of FZD7 may be generated through the techniques of gene-shuffling, motif-shuffling, exon-shuffling, and/or codon-shuffling (collectively referred to as “DNA shuffling”). DNA shuffling may be employed to alter the activities of the ligand comprising (such as consisting essentially of or consisting of) a non-naturally occurring peptide provided herein that binds or specifically binds the CRD of FZD7 (e.g., non-naturally occurring peptides with higher affinities and lower dissociation rates). See, generally, U.S. Pat. Nos. 5,605,793, 5,811,238, 5,830,721, 5,834,252, 5,837,458, Patten et al. (1997) Curr. Opinion Biotechnol. 8, 724-33; Harayama (1998) Trends Biotechnol. 16, 76-82; Hansson, et al., (1999) J Mol. Biol. 287, 265-76; and Lorenzo and Blasco, (1998) Biotechniques 24, 308-313

In certain embodiments, the ligand comprising (such as consisting essentially of or consisting of) a non-naturally occurring peptide provided herein that binds or specifically binds the CRD of FZD7 is altered by being subjected to random mutagenesis by error-prone PCR, random nucleotide insertion or other methods prior to recombination. In certain embodiments, one or more portions of a polynucleotide encoding a ligand that comprises (such as consists essentially of or consists of) a non-naturally occurring peptide provided herein that binds or specifically binds the CRD of FZD7 may be recombined with one or more components, motifs, sections, parts, domains, fragments, etc. of one or more heterologous molecules.

A ligand comprising (such as consisting essentially of or consisting of) a non-naturally occurring peptide provided herein that binds or specifically binds the CRD of FZD7 can be generated by standard techniques, for example, by expression of the fusion protein from a recombinant fusion gene constructed using publicly available gene sequences, or by chemical peptide synthesis.

In certain embodiments, a ligand provided herein comprises (such as consists essentially of or consists of) a non-naturally occurring peptide that is between 5-45 amino acids in length. In certain embodiments, a ligand provided herein comprises (such as consists essentially of or consists of) a non-naturally occurring peptide that is between 7-30 amino acids in length. In certain embodiments, a ligand provided herein comprises (such as consists essentially of or consists of) a non-naturally occurring peptide that is between 10-20 amino acids in length. In certain embodiments, a ligand provided herein comprises (such as consists essentially of or consists of) a non-naturally occurring peptide that is between 11-14 amino acids in length. In certain embodiments, a ligand provided herein comprises (such as consists essentially of or consists of) a non-naturally occurring peptide that is less than 45, 40, 35, 30, 25, 20, 15, 10, 9, 8, 7, 6, or 5 amino acids in length.

In certain embodiments, the ligand comprises a non-naturally occurring peptide comprising (such as consisting essentially of or consisting of) an amino acid sequence set forth in:

(SEQ ID NO: 100)
X₁X₂X₃DDLX₄X₅WCHVMY

wherein each of X₁-X₃ is no amino acid, any amino acid, or an unnatural amino acid, and wherein X₄-X₅ is any amino acid or an unnatural amino acid. In certain embodiments, X₁ is L, X₂ is P, X₃ is S, X₄ is E, and X₅ is F. In certain embodiments, the non-naturally occurring peptide is cyclized. In certain embodiments, the non-naturally occurring peptide is dimerized.

In certain embodiments, the ligand comprises a non-naturally occurring peptide comprising (such as consisting essentially of or consisting of) an amino acid sequence set forth in:

(SEQ ID NO: 101)
X₁X₂DDLX₃X₄WCHVMY

wherein each of X₁-X₂ is no amino acid, any amino acid, or an unnatural amino acid, and wherein X₃-X₄ is any amino acid or an unnatural amino acid. In certain embodiments, the non-naturally occurring peptide is cyclized. In certain embodiments, the non-naturally occurring peptide is dimerized.

In certain embodiments, the ligand comprises a non-naturally occurring peptide comprising (such as consisting essentially of or consisting of) an amino acid sequence set forth in:

(SEQ ID NO: 102)
X₁DDLX₂X₃WCHVMY

wherein X₁ is no amino acid, any amino acid, or an unnatural amino acid, and wherein X₂-X₃ is any amino acid or an unnatural amino acid. In certain embodiments, X₁ is S, X₂ is E, and X₃ is F. In certain embodiments, the non-naturally occurring peptide is cyclized. In certain embodiments, the non-naturally occurring peptide is dimerized.

In certain embodiments, the ligand comprises a non-naturally occurring peptide comprising (such as consisting essentially of or consisting of) an amino acid sequence set forth in:

(SEQ ID NO: 103)
DDLX₁X₂WCHVMY

wherein each of X₁ and X₂ is any amino acid or an unnatural amino acid. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 100)
X₁X₂X₃DDLX₄X₅WCHVMY

wherein each of X₁-X₃ is no amino acid, any amino acid, or an unnatural amino acid, and wherein X₄-X₅ is any amino acid or an unnatural amino acid. In certain embodiments, X₁ is L, X₂ is P, X₃ is S, X₄ is E, and X₅ is F. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 101)
X₁X₂DDLX₃X₄WCHVMY

wherein each of X₁-X₂ is no amino acid, any amino acid, or an unnatural amino acid, and wherein X₃-X₄ is any amino acid or an unnatural amino acid. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 102)
X₁DDLX₂X₃WCHVMY

wherein X₁ is no amino acid, any amino acid, or an unnatural amino acid, and wherein X₂-X₃ is any amino acid or an unnatural amino acid. In certain embodiments, X₁ is S, X₂ is E, and X₃ is F. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 103)
DDLX₁X₂WCHVMY

wherein each of X₁ and X₂ is any amino acid or an unnatural amino acid. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 114)
SDDLEFWCHVXY

wherein X is any amino acid, or an unnatural amino acid. In certain embodiments, X is 2-aminodecanoic acid. In certain embodiments, X is L-2-aminohexadecanoic acid. In certain embodiments, X is a derivative of lysine comprising an octanoic acid coupled to the lysine epsilon amino group. In certain embodiments, X is a derivative of lysine comprising a decanoic acid coupled to the lysine epsilon amino group. In certain embodiments, X is a derivative of lysine comprising a dodecanoic acid coupled to the lysine epsilon amino group. In certain embodiments, X is a derivative of lysine comprising a tetradecanoic acid coupled to the lysine epsilon amino group. In certain embodiments, X is a derivative of lysine comprising a hexadecanoic acid coupled to the lysine epsilon amino group. In certain embodiments, X is 2-amino-6-hydroxyhexanoic acid. In certain embodiments, X is oxohexanoic Acid t-butoxy. In certain embodiments, X is S-benzyl-L-homocysteine (i.e., homocysteine coupled via thioether bond to a benzyl group). In certain embodiments, X is the unnatural amino acid represented by CAS#374899-60-2. In certain embodiments, X is 2-amino-3-decyloxy-propionic acid. In certain embodiments, X is L-homophenylalanine. In certain embodiments, X is 2-aminophenylpentanoic acid. In certain embodiments, X is L-alpha-aminoadipic acid delta-tert-butyl ester. In certain embodiments, X is the unnatural amino acid represented by CAS#:159751-47-0. In certain embodiments, X is butyryl lysine. In certain embodiments, X is pentyl lysine. In certain embodiments, X is amino-8-(benzyloxy)-8-oxooctanoic acid. In certain embodiments, X is the unnatural amino acid represented by CAS#:182059-59-2. In certain embodiments, X is L-2-aminoheptanoic acid. In certain embodiments, X is L-3-styryl alanine. In certain embodiments, X is 6-hydroxy-L-norleucine. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 115)
SDDXEFWCHVMY

wherein X is any amino acid, or an unnatural amino acid. In certain embodiments, X is a derivative of lysine comprising an octanoic acid coupled to the lysine epsilon amino group. In certain embodiments, X is a derivative of lysine comprising a decanoic acid coupled to the lysine epsilon amino group. In certain embodiments, X is a derivative of lysine comprising a dodecanoic acid coupled to the lysine epsilon amino group. In certain embodiments, X is homophenylalanine. In certain embodiments, X is L-homoleucine. In certain embodiments, X is the unnatural amino acid represented by CAS#180414-94-2 In certain embodiments, X is t-butyl alanine. In certain embodiments, X is cyclobutylalanine. In certain embodiments, X is cyclopentyl L alanine. In certain embodiments, X is 3-styryl phenylalanine. In certain embodiments, X is biphenyl. In certain embodiments, X is L-2-aminoheptanoic acid. In certain embodiments, X is L-2-aminooctanoic acid. In certain embodiments, X is L-2-aminodecanoic acid. In certain embodiments, X is 3-quinolyl-L-alanine. In certain embodiments, X is 4-quinolyl-L-alanine. In certain embodiments, X is trifluoromethyl-L-leucine. In certain embodiments, X is cyclohexyl-L-alanine. In certain embodiments, X is F (phenylalanine). In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 116)
SDDX₁EFWCHVX₂Y

wherein each of X₁ and X₂ is any amino acid or an unnatural amino acid. In certain embodiments, X₁ is L-homophenylalanine, and X₂ is a derivative of lysine comprising a tetradecanoic acid coupled to the lysine epsilon amino group. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 117)
SDDLEFWCHXMY

wherein X is any amino acid, or an unnatural amino acid. In certain embodiments, X is L. In certain embodiments, X is I. In certain embodiments, X is T. In certain embodiments, X is 3-amino-L-alanine. In certain embodiments, X is the unnatural amino acid represented by CAS#:181954-34-7. In certain embodiments, X is beta hydroxy norvaline. In certain embodiments, X is t-butyl alanine. In certain embodiments, X is t-butyl-L-alanine. In certain embodiments, X is cyclobutyl-L-glycine. In certain embodiments, X is cyclopropyl-L-alanine. In certain embodiments, X is cyclopentyl-L-alanine. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 118)
LPSDDLEFWCHVMX

wherein X is any amino acid, or an unnatural amino acid. In certain embodiments, X is 4-(trifluoromethyl)-L-phenylalanine. In certain embodiments, X is 4-chloro-L-phenylalanine. In certain embodiments, X is 4-methyl-L-phenylalanine. In certain embodiments, X is 3-(3-quinolinyl)-L-alanine. In certain embodiments, X is 3-(2-quinolinyl)-L-alanine. In certain embodiments, X is 3-(2-quinoxalinyl)-L-alanine. In certain embodiments, X is 3-[3,4-bis(trifluoromethyl)phenyl]-L-alanine. In certain embodiments, X is 3,4-difluoro-L-phenylalanine. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 119)
SDXLEFWCHVMY

wherein X is any amino acid, or an unnatural amino acid. In certain embodiments, X is E (glutamic acid.) In certain embodiments, X is L-alpha-aminoadipic acid. In certain embodiments, X is the unnatural amino acid represented by CAS#250384-77-1. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 120)
LPSDXLEFWCHVMY

wherein X is any amino acid, or an unnatural amino acid. In certain embodiments, X is Q (glutamine). In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 121)
SDDLEXWCHVMY

wherein X is any amino acid, or an unnatural amino acid. In certain embodiments, X is 4-chloro-L-phenylalanine. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 122)
XDDLEFWCHVMY

wherein X is any amino acid, or an unnatural amino acid. In certain embodiments, X is T (threonine). In certain embodiments, X is beta hydroxyl norvaline. In certain embodiments, the non-naturally occurring peptide is cyclized. In certain embodiments, the non-naturally occurring peptide is dimerized.

In certain embodiments, the ligand consists essentially of (such as consists of) a non-naturally occurring peptide comprising (such as consisting essentially of or consisting of) an amino acid sequence set forth in:

(SEQ ID NO: 123)
LPSDDLEFWXHVMY

wherein X is any amino acid, or an unnatural amino acid. In certain embodiments, X is A (alanine). In certain embodiments, the non-naturally occurring peptide is cyclized. In certain embodiments, the non-naturally occurring peptide is dimerized.

In certain embodiments, a ligand provided herein comprises a non-naturally occurring peptide comprising the amino acid sequence LPSDDLEFWCHVMY (SEQ ID NO: 13). In certain embodiments, a ligand provided herein comprises a peptide consisting of the amino acid sequence LPSDDLEFWCHVMY (SEQ ID NO: 13). In certain embodiments, a ligand provided herein consists of a non-naturally occurring peptide comprising the amino acid sequence LPSDDLEFWCHVMY (SEQ ID NO: 13). In certain embodiments, a ligand provided herein consists of a non-naturally occurring peptide consisting of the amino acid sequence LPSDDLEFWCHVMY (SEQ ID NO: 13). In certain embodiments, the peptide is modified in such a way as to nucleate or stabilize its alpha helical structure, as described in, e.g., Mahon et al. (2012) Drug Discovery Today: Tech. 9: e57-e62; Forood et al. (1993) Proc Natl Acad Sci U.S.A 90: 838-842; Klein (2014) Med Chem Lett. 5: 838-839; and references cited therein. In certain embodiments, the peptide is a stapled peptide. In certain embodiments, the peptide is cyclized. In certain embodiments, the peptide is dimerized.

In certain embodiments, a ligand provided herein comprises a non-naturally occurring peptide comprising the amino acid sequence SDDLEFWCHVMY (SEQ ID NO: 99). In certain embodiments, a ligand provided herein comprises a non-naturally occurring peptide consisting of the amino acid sequence SDDLEFWCHVMY (SEQ ID NO: 99). In certain embodiments, a ligand provided herein is a non-naturally occurring peptide comprising the amino acid sequence SDDLEFWCHVMY (SEQ ID NO: 99). In certain embodiments, a ligand provided herein is a non-naturally occurring peptide consisting of the amino acid sequence SDDLEFWCHVMY (SEQ ID NO: 99). In certain embodiments, the peptide is modified in such a way as to nucleate or stabilize its alpha helical structure, as described in, e.g., Mahon et al. (2012) Drug Discovery Today: Tech. 9: e57-e62; Forood et al. (1993) Proc Natl Acad Sci U.S.A 90: 838-842; Klein (2014) Med Chem Lett. 5: 838-839; and references cited therein. In certain embodiments, the peptide is a stapled peptide. In certain embodiments, the peptide is cyclized. In certain embodiments, the peptide is dimerized.

In certain embodiments, the ligand comprises (such as consists essentially of or consists of) a non-naturally occurring peptide that is a variant of LPSDDLEFWCHVMY (SEQ ID NO: 13). In certain embodiments, such peptide variant comprises at least 1, at least 2, at least 3, at least 4, or at least 5, amino acid substitutions in SEQ ID NO: 13. In certain embodiments, the amino acid(s) at position(s) 1, 2, 3, 7, and/or 8 in SEQ ID NO: 13 are substituted.

In certain embodiments, the ligand comprises (such as consists essentially of or consists of) a non-naturally occurring peptide that is a variant of SDDLEFWCHVMY (SEQ ID NO: 99). In certain embodiments, such a variant comprises at least 1, at least 2, or at least 3 amino acid substitutions in SEQ ID NO: 99. In certain embodiments, the amino acid(s) at position(s) 1, 5, and/or 6 in SEQ ID NO: 99 are substituted.

In certain embodiments, the amino acid substitution(s) are conservative amino acid substitution(s). In certain embodiments, the amino acid substitution(s) are substitution(s) with unnatural amino acid(s). In certain embodiments, the amino acid substitutions do not substantially reduce the ability of the non-naturally occurring peptide to bind the CRD of FZD7. For example, conservative alterations (e.g., conservative substitutions as described herein) that do not substantially reduce FZD7 CRD binding affinity may be made. The binding affinity of a ligand comprising (such as consisting essentially of or consisting of) a variant of SEQ ID NO: 13 or SEQ ID NO: 99 can be assessed using a method described in the Examples below.

Conservative substitutions are shown in Table 1 below under the heading of “conservative substitutions.” More substantial changes are provided in Table 1 under the heading of “exemplary substitutions,” and as further described below in reference to amino acid side chain classes. Amino acid substitutions may be introduced into a variant of SEQ ID NO: 13 or SEQ ID NO: 99, and the products screened for a desired activity, e.g., retained/improved binding to the CRD of FZD7, using methods described in the Examples.

TABLE 1
Conservative Substitutions

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Non-conservative substitutions will entail exchanging a member of one of these classes for another class. An exemplary substitutional variant of SEQ ID NO: 13 or SEQ ID NO: 99 is an affinity matured peptide, which may be conveniently generated, e.g., using phage display based affinity maturation techniques such as those described in the Examples. Briefly, one or more residues in a peptide described herein is altered (i.e., added, deleted, or substituted) and the variant peptide is displayed on phage and screened for FZD7 CRD binding affinity. In certain embodiments of affinity maturation, diversity is introduced into the variable peptides chosen for maturation by any of a variety of methods (e.g., error-prone PCR or oligonucleotide-directed mutagenesis). A secondary library is then created. The library is then screened to identify any peptide variants with the desired affinity for FZD7 CRD. In certain embodiments, introducing diversity involves randomizing one or more residues in a peptide described herein. In certain embodiments, the residues in a peptide described herein that are involved in binding to the CRD of FZ7 may be identified, e.g., using alanine scanning mutagenesis, serine scanning mutagenesis, valine scanning mutagenesis, aspartic acid scanning mutagenesis or modeling.

In some embodiments, the ligand comprises (such as consists essentially of or consists of) a non-naturally occurring peptide comprising (such as consisting essentially of or consisting of) an amino acid sequence set forth in LPSDDAEFWCHVMY (SEQ ID NO: 109), LPSDDLEFACHVMY (SEQ ID NO: 110), LPSDDLEFWCHVAY (SEQ ID NO: 111), LPSDDLEFWCHVMA (SEQ ID NO: 112), LPSDQLEFWCHVMY (SEQ ID NO: 131), or LPSDDLEFWAHVMY (SEQ ID NO: 133). n certain embodiments, the C-terminal carboxyl group of the peptide is amidated. In certain embodiments, the N-terminal amine of the peptide is acetylated. In certain embodiments, the C-terminal carboxyl group of the peptide is amidated, and the N-terminal amine of the peptide is acetylated. In certain embodiments, the non-naturally occurring peptide is cyclized. In certain embodiments, the non-naturally occurring peptide is dimerized. In certain embodiments, the peptides in the dimer are linked by way of disulfide bond between, e.g., C10 in the peptide. In certain embodiments, the peptides in the dimer are linked by way of chemical linker, such as a chemical linker described elsewhere herein. In certain embodiments, the peptides in the dimer are fused to a spacer peptide (e.g., an intervening peptide).

In some embodiments, the ligand comprises (such as consists of or consists essentially of) a non-naturally occurring peptide comprising (such as consisting essentially of or consisting of) an amino acid sequence set forth in SDDLEFWCHVEY (SEQ ID NO: 125), SDDLEFWCHLMY (SEQ ID NO: 126), SDDLEFWCHIMY (SEQ ID NO: 127), SDDLEFWCHTMY (SEQ ID NO: 128), SDDFEFWCHVMY (SEQ ID NO: 129), SDELEFWCHVMY (SEQ ID NO: 130), or TDDLEFWCHVMY (SEQ ID NO: 132). In certain embodiments, the C-terminal carboxyl group of the peptide is amidated. In certain embodiments, the N-terminal amine of the peptide is acetylated. In certain embodiments, the C-terminal carboxyl group of the peptide is amidated, and the N-terminal amine of the peptide is acetylated. In certain embodiments, the non-naturally occurring peptide is cyclized. In certain embodiments, the non-naturally occurring peptide is dimerized. In certain embodiments, the peptides in the dimer are linked by way of disulfide bond between, e.g., C8 in the peptide. In certain embodiments, the peptides in the dimer are linked by way of chemical linker, such as a chemical linker described elsewhere herein. In certain embodiments, the peptides in the dimer are fused to a spacer peptide (e.g., an intervening peptide).

Additionally or alternatively, in certain embodiments, the ligand comprises (such as consists essentially of or consists of) a peptide that is a variant of LPSDDLEFWCHVMY (SEQ ID NO: 13) or a variant of SDDLEFWCHVMY (SEQ ID NO: 99) in which one or more amino acids are substituted with unnatural amino acids. Additionally or alternatively, in certain embodiments, the ligand comprises (such as consists essentially of or consists of) a peptide that is a variant of LPSDDLEFWCHVMY (SEQ ID NO: 13) or a variant of SDDLEFWCHVMY (SEQ ID NO: 99) in which one or more unnatural amino acid residues are added (e.g., at the N-terminus, at the C-terminus, or at both the N- and C-termini) In certain embodiments, the unnatural amino acid is an unnatural amino acid described elsewhere herein.

In certain embodiments, the ligand comprises (such as consists essentially of or consists of) a peptide that is a variant of LPSDDLEFWCHVMY (SEQ ID NO: 13) or a variant of SDDLEFWCHVMY (SEQ ID NO: 99), wherein the C-terminal carboxyl group of the peptide is amidated. In certain embodiments, the ligand comprises (such as consists essentially of or consists of) a peptide that is a variant of LPSDDLEFWCHVMY (SEQ ID NO: 13) or a variant of SDDLEFWCHVMY (SEQ ID NO: 99), wherein the N-terminal amine of the peptide is acetylated. In certain embodiments, the ligand comprises (such as consists essentially of or consists of) a peptide that is a variant of LPSDDLEFWCHVMY (SEQ ID NO: 13) or a variant of SDDLEFWCHVMY (SEQ ID NO: 99), wherein the C-terminal carboxyl group of the peptide is amidated and wherein the N-terminal amine of the peptide is acetylated.

In certain embodiments, a ligand provided herein comprises (such as consists essentially of or consists of) a dimerized non-naturally occurring peptide that specifically binds the CRD of FZD7. In certain embodiments, each peptide in the dimer comprises (such as consists essentially of or consists of) the amino acid set forth in SEQ ID NO: 13. In certain embodiments, the peptides in the dimer are linked by way of disulfide bond between, e.g., C10 of SEQ ID NO: 13. In certain embodiments, each peptide in the dimer comprises (such as consists essentially of or consists of) the amino acid set forth in SEQ ID NO: 99. In certain embodiments, the peptides in the dimer are linked by way of disulfide bond between, e.g., C8 of SEQ ID NO: 99. In certain embodiments, the peptides in the dimer are linked by way of chemical linker, such as a chemical linker described elsewhere herein. In certain embodiments, the peptides in the dimer are fused to a spacer peptide (e.g., an intervening peptide).

In certain embodiments, the ligand comprises (such as consists essentially of or consists of) two non-naturally occurring peptides. In certain embodiments, the first non-naturally occurring peptide comprises (such as consists essentially of or consists of) an amino acid sequence set forth in SDDLEFWCHVMYX (SEQ ID NO: 134), wherein X is L-homopropargylglycine, and the second non-naturally occurring peptide comprising (such as consisting essentially of or consisting of) an amino acid sequence set forth in SDDLXFWCHVMY (SEQ ID NO: 135), wherein X is 5-azido-L-ornithine or S-acetaminomethyl-L-cysteine. In certain embodiments, the first and second non-naturally occurring peptides are covalently linked by reacting the unnatural amino acids via click chemistry. In certain embodiments, the first non-naturally occurring peptide comprises (such as consists essentially of or consists of) an amino acid sequence set forth in SDDLEFWCHVMYX (SEQ ID NO: 134), wherein X is L-bishomopropargylglycine, and the second non-naturally occurring peptide comprising (such as consisting essentially of or consisting of) an amino acid sequence set forth in SDDLXFWCHVMY (SEQ ID NO: 135), wherein X is azido-homoalanine or S-acetaminomethyl-L-cysteine. In certain embodiments, the first and second non-naturally occurring peptides are covalently linked by reacting the unnatural amino acids via click chemistry.

In certain embodiments, the ligand comprises (such as consists essentially of or consists of) a non-naturally occurring peptide comprising (such as consisting essentially of or consisting of) an amino acid sequence set forth in SDDLEFWXHVMY (SEQ ID NO: 124), wherein X is L-selenocysteine. In certain embodiments, the peptide is cyclized and/or dimerized via the L-selenocysteine.

In certain embodiments, a ligand provided herein comprises a non-naturally occurring peptide (such as a linear peptide) comprising an amino acid sequence set forth in any one of SEQ ID NOs: 1-12, 14-31, and 39-98. In certain embodiments, a ligand provided herein comprises a non-naturally occurring peptide (such as a linear peptide) consisting of an amino acid sequence set forth in any one of SEQ ID NOs: 1-12, 14-31, and 39-98. In certain embodiments, a ligand provided herein is a non-naturally occurring peptide (such as a linear peptide) comprising an amino acid sequence set forth in any one of SEQ ID NOs: 1-12, 14-31, and 39-98. In certain embodiments, a ligand provided herein is a non-naturally occurring peptide (such as a linear peptide) consisting of an amino acid sequence set forth in any one of SEQ ID NOs: 1-12, 14-31, and 39-98.

In certain embodiments, a ligand provided herein comprises a non-naturally occurring (such as a cyclic peptide) comprising an amino acid sequence set forth in any one of SEQ ID NOs: 32-38. In certain embodiments, a ligand provided herein comprises a non-naturally occurring peptide (such as a cyclic peptide) consisting of an amino acid sequence set forth in any one of SEQ ID NOs: 32-38. In certain embodiments, a ligand provided herein is a non-naturally occurring peptide (such as a cyclic peptide) comprising an amino acid sequence set forth in any one of SEQ ID NOs: 32-38. In certain embodiments, a ligand provided herein is a non-naturally occurring peptide (such as a cyclic peptide) consisting of an amino acid sequence set forth in any one of SEQ ID NOs: 32-38.

The amino acid sequences of SEQ ID NOs: 1-12 and 14-98 are provided in Table 2 below:

TABLE 2

| SEQ ID | | SEQ ID | | SEQ ID | |
|---|---|---|---|---|---|
| NO: | | NO: | | NO: | |
| 1 | YEHLHDLMDLIRPW | 36 | FDFCTVMPHFIYCPGD | 70 | AASDDLEAWCHVMY |
| 2 | TYFDDICNLILPWANP | 37 | FDFCSVMPHFIYCPGD | 71 | APSDDLASWCHVMY |
| 3 | PQDLLDWCHYMIVSSD | 38 | HLSDVFCSDWCDLVFW | 72 | APADDLEFWCHVMY |
| 4 | ACSYVIDLWNQCLT | 39 | VAADDLAAWCHVMY | 73 | APSDDLAAWCHVMY |
| 5 | PCSVICLPDWSSLLFI | 40 | AASDDLEFWCHVMY | 74 | APADDLAFWCHVVY |
| 6 | DTDLHQWCLWFT | 41 | AASDDLEFWCHVMY | 75 | APSDDLEAWCDVMY |
| 7 | FWMLLQEGFAFWFP | 42 | APSDDVAFWCHVMY | 76 | VPSDDLEAWCDVMY |
| 8 | FELLLDLGDLIRLW | 43 | APADDVEFWCHVMY | 77 | AASDDLAFWCHVMY |
| 9 | ACSYVIDLWNLCLR | 44 | APSDDLEFWCHVMY | 78 | VPADDLASWCDVMY |
| 10 | ASELHDWCRMMFPW | 45 | APADDLEAWCHVMY | 79 | LPSADLESWCHVMY |
| 11 | ISLIEAMIALDRVF | 46 | APSDDLEFWCHAMY | 80 | AAADDLAFWCHVMY |
| 12 | PPNVHEGCWSMFPW | 47 | VASDDLEAWCHVMY | 81 | LASDDLEFWCHVMY |
| 14 | DTDLLQWCLWFT | 48 | AAADDLEFWCHVMY | 82 | LPAADLAAWCHVMY |
| 15 | FWMQLQDGFAIWFP | 49 | AASDDLAAWCHVMY | 83 | VPSADLETWCHVMY |
| 16 | PCSVICLPDWSSLLFI | 50 | AASDDLESWCHVMY | 84 | LPADDLAAWCHVMY |
| 17 | GDFWPGSLLWEILV | 51 | APADDLAFWCHVMY | 85 | PPADDLAFWCDVMY |
| 18 | ILTFEYFWILGLIL | 52 | LPADDLAVWCDVMY | 86 | VASDDLASWCHVVY |
| 19 | LPLFFLSYVL | 53 | LPSDDLESWCHVMY | 87 | AAADDVASWCHVMY |
| 20 | FLPDQHSHLFLPWGEP | 54 | AAADDLEVWCHVMY | 88 | VAADDLAFWCDVMY |
| 21 | SCQMWSNLRVLFLSYW | 55 | VAADALEFWCHVMY | 89 | APADDLEFWCHAMY |
| 22 | VFVPFSELTSLC | 56 | APSDDLAAWCHVVY | 90 | APADDLAFWCDVMY |
| 23 | IWFKGRFVEFSSLV | 57 | AAADDLAAWCDVMY | 91 | APSDDLAFWCDVMY |
| 24 | NAFWRDQCLEWFIICL | 58 | VASDDLEFWCHVMY | 92 | LPADDLAFWCDVMY |
| 25 | EHDLLLRAMNSFVLIF | 59 | AAADDLEAWCAVMY | 93 | AAADDLAFWCDVMY |
| 26 | FCENPYIICW | 60 | LAADDLESWCHVMY | 94 | LPADDLEFWCHVMY |
| 27 | NPPPECFLSK | 61 | APADDLASWCHVMY | 95 | VPSDDLEFWCAVMY |
| 28 | VFFYHSLFFIKLILDP | 62 | VASDDLASWCHAMY | 96 | APADDLESWCHVMY |
| 29 | ERRVCYPWFEVSQP | 63 | VPADDLASWCHVMY | 97 | APSDDLAFWCHVVY |
| 30 | LSSGKKVSSYWFNFWF | 64 | APADDLEFWCHVVY | 98 | AAADDLAAWCHVVY |
| 31 | FWFDFWFG | 65 | VPSDDLAFWCHVMY | | |
| 32 | SSDFSGCLSWCDLIFG | 66 | VPADALAVWCDVMY | | |
| 33 | FDFCSVMPQFIYCPGD | 67 | VPADDLAFWCHVMY | | |
| 34 | HLSDVCCSDWCDLVFW | 68 | VPSDDLASWCHVMY | | |
| 35 | TSDFSWCLSWCDLIFW | 69 | VPSADLESWCHVMY | | |

In certain embodiments a ligand provided herein comprises (such as consists essentially of or consists of) a non-naturally occurring peptide described herein that binds or specifically binds the CRD of FZD7 conjugated to a lipid. In certain embodiments, the lipid is a long chain fatty acid (i.e., LCFA), comprising between 12-20 carbon atoms. In certain embodiments, the lipid is a short chain fatty acid (i.e., SCFA), comprising 6 or fewer carbon atoms. In certain embodiments, the lipid is a saturated fatty acid. In certain embodiments, the lipid is an unsaturated fatty acid. In certain embodiments, the lipid comprises an aromatic tail. In certain embodiments, the lipid is octanoic acid. In certain embodiments, the lipid is decanoic acid. In certain embodiments, the lipid is dodecanoic acid. In certain embodiments, the lipid is tetradecanoic acid. In certain embodiments, the lipid is hexadecanoic acid. In certain embodiments, the lipid is amino-6-hydroxyhexanoic acid. In certain embodiments, the lipid is amino-6-hydroxyhexanoic acid. In certain embodiments, the lipid is aminophenylpentanoic acid. In certain embodiments, the lipid is L-alpha-aminoadipic acid delta-tert-butyl ester. In certain embodiments, the lipid is amino-8-(benzyloxy)-8-oxooctanoic acid. In certain embodiments, the lipid is 2-aminoheptanoic acid. In certain embodiments, the lipid is L-2-aminodecanoic acid. In certain embodiments, the lipid is 2-aminooctanoic acid. Methods of coupling lipids to peptides are well-known in the art and typically entail reacting the carboxylic acid group of the lipid with the epsilon amine of a lysine side chain in the peptide under standard amide coupling conditions. Additional methods are reviewed in Gerauer, M., Koch, S., Brunsveld, L. and Waldmann, H. 2008. Lipidated peptide synthesis. Wiley Encyclopedia of Chemical Biology. 1-11. In certain embodiments, lipid-coupled amino acids are incorporated into the peptide using standard peptide synthesis techniques, as described elsewhere herein.

In certain embodiments a ligand provided herein comprises (such as consists essentially of or consists of) a non-naturally occurring peptide described herein, wherein the C-terminal carboxyl group of the peptide is amidated. In certain embodiments a ligand provided herein comprises (such as consists essentially of or consists of) a non-naturally occurring peptide described herein, wherein the N-terminal amine of the peptide is acetylated. In certain embodiments a ligand provided herein comprises (such as consists essentially of or consists of) a non-naturally occurring peptide described herein, wherein the C-terminal carboxyl group of the peptide is amidated and wherein the N-terminal amine of the peptide is acetylated.

In certain embodiments a ligand provided herein comprises (such as consists essentially of or consists of) a non-naturally occurring peptide described herein that binds or specifically binds the CRD of FZD7 conjugated (such as covalently or non-covalently) to a nucleic acid molecule, a small molecule, a mimetic agent, a synthetic drug, an inorganic molecule, and organic molecules. In certain embodiments a ligand provided herein comprises (such as consists essentially of or consists of) a non-naturally occurring peptide described herein that binds or specifically binds the CRD of FZD7 conjugated (such as covalently or non-covalently) to a heterologous protein or polypeptide (or fragment thereof, to a polypeptide of at least 10, at least 20, at least 30, at least 40, at least 50, at least 60, at least 70, at least 80, at least 90 or at least 100 amino acids).

In certain embodiments a ligand provided herein comprises (such as consists essentially of or consists of), a non-naturally occurring peptide described herein that binds or specifically binds the CRD of FZD7 conjugated a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes. Other toxins include maytansine and maytansinoids, calicheamicin and other cytotoxic agents. A variety of radionuclides are available for the production of radioconjugated ligands comprising (such as consisting essentially of or consisting of) a non-naturally occurring peptide provided herein that specifically binds the CRD of FZD1, FZD2, and/or FZD7. Examples include ²¹²Bi, ¹³¹I, ¹³¹In, ⁹⁰Y, and ¹⁸⁶Re.

Conjugates of a ligand comprising (such as consisting essentially of or consisting of) a non-naturally occurring peptide provided herein that binds or specifically binds the CRD of FZD7 and, e.g., cytotoxic agent, are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionuclide to a ligand comprising (such as consisting essentially of or consisting of) a non-naturally occurring peptide provided herein that binds or specifically binds the CRD of FZD7. See, WO94/11026.

In certain embodiments, a ligand comprising (such as consisting essentially of or consisting of) a non-naturally occurring peptide provided herein that binds or specifically binds the CRD of FZD7 is engineered to provide reactive groups for conjugation. In certain embodiments, the N-terminus and/or C-terminus may also serve to provide reactive groups for conjugation. In certain embodiments, the N-terminus is conjugated to one moiety (such as, but not limited to PEG) while the C-terminus is conjugated to another moiety (such as, but not limited to biotin), or vice versa.

In certain embodiments, a ligand comprising (such as consisting essentially of or consisting of) a non-naturally occurring peptide provided herein that binds or specifically binds the CRD of FZD7 may be conjugated to a diagnostic or detectable agent. Such ligand conjugates can be useful for monitoring or prognosing the development or progression of a disease as part of a clinical testing procedure, such as determining the efficacy of a particular therapy. Such diagnosis and detection can be accomplished by coupling the ligand comprising (such as consisting essentially of or consisting of) a non-naturally occurring peptide provided herein that binds or specifically binds the CRD of FZD7 to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I),I carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tn; positron emitting metals using various positron emission tomographies, nonradioactive paramagnetic metal ions, and molecules that are radiolabeled or conjugated to specific radioisotopes.

Also provided is a ligand comprising (such as consisting essentially of or consisting of) a non-naturally occurring peptide provided herein that binds or specifically binds the CRD of FZD7 conjugated to a therapeutic moiety. In certain embodiments, the a ligand comprising (such as consisting essentially of or consisting of) a non-naturally occurring peptide provided herein that binds or specifically binds the CRD of FZD7 may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

In certain embodiments, a ligand comprising (such as consisting essentially of or consisting of) a non-naturally occurring peptide provided herein that binds or specifically binds the CRD of FZD7 is conjugated to a therapeutic moiety such as a radioactive metal ion, such as alpha-emitters such as ²¹³Bi or macrocyclic chelators useful for conjugating radiometal ions, including but not limited to, ¹³¹In, ¹³¹Lu, ¹³¹Y, ¹³¹Ho, ¹³¹Sm, to polypeptides. In certain embodiments, the macrocyclic chelator is 1, 4, 7, 10-tetraazacyclododecane-N,N′,N″,N′″-tetra-acetic acid (DOTA) which can be attached to the a ligand comprising (such as consisting essentially of or consisting of) a non-naturally occurring peptide provided herein that binds or specifically binds the CRD of FZD7 via a linker molecule. Such linker molecules are commonly known in the art and described in, e.g., Denardo et al. (1998) Clin Cancer Res. 4, 2483-90; Peterson et al. (1999) Bioconjug. Chem. 10, 553-557; and Zimmerman et al. (1999) Nucl. Med. Biol. 26, 943-50.

Techniques for conjugating therapeutic moieties to antibodies are well known and can be applied to a ligand comprising (such as consisting essentially of or consisting of) a non-naturally occurring peptide provided herein that binds or specifically binds the CRD FZD7 (see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radio labeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

The therapeutic moiety or drug conjugated to a ligand comprising (such as consisting essentially of or consisting of) a non-naturally occurring peptide provided herein that binds or specifically binds the CRD FZD7 should be chosen to achieve the desired prophylactic or therapeutic effect(s) for a particular disorder in a subject. A clinician or other medical personnel should consider the following when deciding on which therapeutic moiety or drug to conjugate to the ligand: the nature of the disease, the severity of the disease, and the condition of the subject.

In certain embodiments, ligands comprising (such as consisting essentially of or consisting of) a non-naturally occurring peptide provided herein that binds or specifically binds the CRD of FZD7 can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Nucleic acid molecules encoding a ligand comprising (such as consisting essentially of or consisting of) a non-naturally occurring peptide provided herein that binds or specifically binds the CRD of FZD7, expression vectors comprising nucleic acid molecules encoding the ligand, and cells comprising the nucleic acid molecules are also contemplated. Also provided herein are methods of producing a ligand comprising (such as consisting essentially of or consisting of) a non-naturally occurring peptide provided herein that binds or specifically binds the CRD of FZD7 by culturing such cells, expressing the ligand, and recovering the ligand from the cell culture.

In certain embodiments, a ligand comprising (such as consisting essentially of or consisting of) a non-naturally occurring peptide provided herein that binds or specifically binds the CRD of FZD7 is produced via in vitro translation, as described elsewhere herein.

In certain embodiments, a ligand comprising (such as consisting essentially of or consisting of) a non-naturally occurring peptide provided herein that binds or specifically binds the CRD of FZD7 is generated via chemical peptide synthesis, e.g., by grafting a non-naturally occurring peptide described herein that has been chemically synthesized to one or more moieties, or by chemically synthesizing the entire ligand.

In certain embodiments, a ligand comprising (such as consisting essentially of or consisting of) a non-naturally occurring peptide provided herein that binds or specifically binds the CRD of FZD7 is used as a therapeutic agent in the treatment of diseases or conditions wherein aberrant Wnt signaling is involved.

In certain embodiments, provided is a method of killing a cancer cell (e.g., a colon cancer cell, a pancreatic cancer cell, a non-small cell lung cancer cell, a cancer cell comprising a RNF43 mutation, a cancer characterized by USP6 overexpression, or a cancer cell characterized by gene fusions involving R-spondin (RSPO) family members) comprising contacting the cancer cell with a ligand comprising (such as consisting essentially of or consisting of) a non-naturally occurring peptide provided herein that binds or specifically binds the CRD domain of FZD7.

In certain embodiments, provided is a method of killing a cancer stem cell (e.g., a colon cancer stem cell, a pancreatic cancer stem cell, a non-small cell lung cancer stem cell, a cancer stem cell comprising a RNF43 mutation, a cancer stem cell characterized by USP6 overexpression, or a cancer stem cell characterized by gene fusions involving R-spondin (RSPO) family members) comprising contacting the cancer cell with a ligand comprising (such as consisting essentially of or consisting of) a non-naturally occurring peptide provided herein that binds or specifically binds the CRD domain of FZD7.

In certain embodiments, provided is a method of inhibiting Wnt-mediated β-catenin signaling in a cell (such as a colon cancer cell, a pancreatic cancer cell, a non-small cell lung cancer cell, a cancer cell comprising a RNF43 mutation, a cancer cell characterized by USP6 overexpression, or a cancer cell characterized by gene fusions involving R-spondin (RSPO) family members) comprising contacting the cancer cell with a ligand comprising (such as consisting essentially of or consisting of) a non-naturally occurring peptide provided herein that binds or specifically binds the CRD domain of FZD7.

### Functional Characteristics

In certain embodiments, a ligand comprising (such as consisting essentially of or consisting of) a non-naturally occurring peptide provided herein that binds or specifically binds the CRD of FZD7 has a binding affinity (Kd) value of no more than about 1×10⁻⁷ M, preferably no more than about 1×10⁻⁸ and most preferably no more than about 1×10⁻⁹ M) but has a binding affinity for the CRD of FZD3, FZD4, FZD5, FZD6, FZD8, FZD9, and/or FZD10 which is at least about 50-fold, or at least about 500-fold, or at least about 1000-fold, weaker than its binding affinity the CRD FZD7. In certain embodiments, a ligand comprising (such as consisting essentially of or consisting of) a non-naturally occurring peptide provided herein that binds or specifically binds the CRD of FZD7 has a binding affinity (Kd) value of no more than about 1×10⁻⁷ M, preferably no more than about 1×10⁻⁸ and most preferably no more than about 1×10⁻⁹ M) but has a binding affinity for the CRD of FZD1, FZD2, FZD3, FZD4, FZD5, FZD6, FZD8, FZD9, and/or FZD10 which is at least about 50-fold, or at least about 500-fold, or at least about 1000-fold, weaker than its binding affinity the CRD FZD7.

In certain embodiments, the extent of binding of a ligand comprising (such as consisting essentially of or consisting of) a non-naturally occurring peptide provided herein to, e.g., the CRD of a non-target FZD receptor (e.g., FZD1, FZD2, FZD3, FZD4, FZD5, FZD6, FZD8, FZD9, and/or FZD10) is less than about 10% of the binding of the ligand comprising (such as consisting essentially of or consisting of) a non-naturally occurring peptide provided herein to the CRD of FZD7 as determined by methods known in the art, such as ELISA, fluorescence activated cell sorting (FACS) analysis, or radioimmunoprecipitation (RIA). In certain embodiments, the extent of binding of a ligand comprising (such as consisting essentially of or consisting of) a non-naturally occurring peptide provided herein to proteins that are structurally related to FZD proteins, (such as secreted Frizzled Related Proteins, e.g., sFRP1, sFRP2, sFRP3, sFRP4, and/or sFRP5) is less than about 10% of the binding of the ligand comprising (such as consisting essentially of or consisting of) a non-naturally occurring peptide provided herein to the CRD of FZD7 as determined by methods known in the art, such as ELISA, fluorescence activated cell sorting (FACS) analysis, or radioimmunoprecipitation (RIA).

Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. Other methods of assessing the binding of a ligand comprising (such as consisting essentially of or consisting of) a non-naturally occurring peptide provided herein that “specifically binds” the CRD of FZD7 are described in the Examples.

The term “specific binding” or “specifically binds to” or is “specific for” a particular polypeptide or a binding region on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or binding region on a particular polypeptide without substantially binding to any other polypeptide or binding region.

In certain embodiments, a ligand comprising (such as consisting essentially of or consisting of) a non-naturally occurring peptide provided herein binds the CRD of FZD7 with a Kd between about 1 pM to about 500 nM. In certain embodiments, the ligand comprising (such as consisting essentially of or consisting of) a non-naturally occurring peptide provided herein binds the CRD of FZD7 with a Kd between about 1 pM to about 50 pM, between about 50 pM to about 250 pM, between about 250 pM to about 500 pM, between about 500 pM to 750 pM, between about 750 pM to about 1 nM, between about 1 nM to about 25 nM, between about 25 nM to about 50 nM, between 50 nM to about 100 nM, between about 100 nM to about 250 nM, or between about 250 nM to about 500 nM. In certain embodiments the, the Kd is determined via surface plasmon resonance.

In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value of less than about any one of 300 nM, 275 nM, 250 nM, 200 nM, 175 nM, 150 nM, 140 nM, 130 nM, 120 nM, 110 nM, 100 nM, 90 nM, 80 nM, 70 nM, 60 nM, 50 nM, 40 nM, 30 nM, 20 nM, 10 nM, 5 nM, 1 nM, 0.9 nM, 0.8 nM, 0.7 nM, 0.6 nM, 0.5 nM, 0.4 nM, 0.3 nM, 0.2 nM, or 0.1 nM, including any range in between these values.

In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 10 nM and 200 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 20 nM and 200 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 30 nM and 200 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 40 nM and 200 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 200 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 180 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 160 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 140 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 120 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 50 nM and 100 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 40 nM and 100 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 30 nM and 100 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 20 nM and 100 nM. In certain embodiments, a ligand comprising (such as consisting essentially of or consisting of) a non-naturally occurring peptide provided herein that specifically binds the CRD of FZD7 inhibits Wnt-mediated β-catenin signaling with an IC₅₀ value between 10 nM and 100 nM. In certain embodiments, IC₅₀ is determined as a measure of luciferase activity in a dual-luciferase assay, as described in further detail in the Examples.

In certain embodiments, a ligand comprising (such as consisting essentially of or consisting of) a non-naturally occurring peptide provided herein that specifically binds the CRD of FZD7 has an EC₅₀ value of less than about any one of 300 nM, 275 nM, 250 nM, 200 nM, 175 nM, 150 nM, 100 nM, 90 nM, 80 nM, 70 nM, 60 nM, 50 nM, 40 nM, 30 nM, 20 nM, 10 nM, 5 nM, 1 nM, 0.9 nM, 0.8 nM, 0.7 nM, 0.6 nM, 0.5 nM, 0.4 nM, 0.3 nM, 0.2 nM, or 0.1 nM, including any range in between these values. In certain embodiments, the EC50 is determined via FSEC using a 5FAM-labeled peptide, as described in further detail in the Examples.

In certain embodiments, a ligand comprising (such as consisting essentially of or consisting of) a non-naturally occurring peptide provided herein that specifically binds the CRD of FZD7 enhances the recruitment and binding of a Wnt to the CRD of FZD7. In certain embodiments, the recruitment and binding of a Wnt to the CRD of FZD7 is enhanced when the peptide is present at a concentration of less than about 10 μM, 5 μM, 1 μM, 0.9 μM, 0.8 μM, 0.7 μM, 0.6 μM, 0.5 μM, 0.4 μM, 0.3 μM, 0.2 μM 0.1 μM, 0.005 μM, 0.001 μM, or 0.0005 μM, including any range in between these values. In certain embodiments, the Wnt is Wnt5a. In certain embodiments, the Wnt is Wnt 3a. In certain embodiments, a ligand comprising (such as consisting essentially of or consisting of) a non-naturally occurring peptide provided herein that specifically binds the CRD of FZD7 does not enhance the recruitment and binding of a Wnt to the CRD of FZD3, FZD4, FZD5, FZD6, FZD8, FZD9, and/or FZD10 or to the CRD of FZD1, FZD2, FZD3, FZD4, FZD5, FZD6, FZD8, FZD9, and/or FZD10. In certain embodiments, the Wnt is Wnt5a. In certain embodiments, the Wnt is Wnt 3a.

Methods of Identifying Non-Naturally Occurring Peptides that Specifically Binds the Cysteine-Rich Domain of FZD7

In certain embodiments, provided herein is a method of obtaining a non-naturally occurring peptide that binds or specifically binds the CRD of FZD7. In certain embodiments, the method comprises a) contacting the CRD of FZD7 with a library of non-naturally peptides (such as linear or cyclic peptides) under conditions that allow a non-naturally occurring peptide: CRD complex to form, (b) detecting the formation of the complex, and (c) obtaining from the complex the non-naturally occurring peptide that specifically binds the CRD of FZD7. In certain embodiments, the method further comprises (d) determining the nucleic acid sequence of the non-naturally occurring peptide that specifically binds the CRD of FZD7. In certain embodiments, provided is a complex comprising a non-naturally occurring peptide and the CRD of FZD7.

In certain embodiments, a non-naturally occurring peptide that binds or specifically binds the CRD of FZD7 is subject to affinity maturation. In this process, a peptide that has been found to bind the CRD of FZD7 is subject to a scheme that selects for increased affinity for FZD7 CRD (see Wu et al. (1998) Proc Natl Acad Sci USA. 95, 6037-42). In certain embodiments, a non-naturally occurring peptide that specifically binds the CRD of FZD7 is further randomized after identification from a library screen. For example, in certain embodiments, the method of obtaining a non-naturally occurring peptide that specifically binds the CRD of FZD7 further comprises (e) randomizing at least 1, at least 2, at least 3, at least 4, at least 5, at least 6, at least 7, at least 8, at least 9, at least 10, at least 11, at least 12, at least 13, at least 14, at least 15, at least 16, or more than 16 amino acids of the non-naturally occurring peptide obtained from the peptide: CRD identified previously to generate further randomized non-naturally occurring peptides, (f) contacting the CRD of FZD7 with the further randomized non-naturally occurring peptides, (g) detecting the formation of the further randomized peptide: CRD complex, and (h) obtaining from the complex the further randomized non-naturally occurring peptide that specifically binds the CRD of FZD7. In certain embodiments, the method further comprises (i) determining the nucleic acid sequence of the non-naturally peptides that specifically binds the CRD of FZD7.

In certain embodiments, the method is used to identify a peptide that specifically binds the CRD of FZD1, FZD2, or FZD7. In certain embodiments, the method is used to identify a peptide that specifically binds the CRD of FZD1, FZD2, and FZD7. In certain embodiments, the method is used to identify a peptide that specifically binds the CRD of FZD1 or FZD7. In certain embodiments, the method is used to identify a peptide that specifically binds the CRD of FZD1 and FZD7. In certain embodiments, the method is used to identify a peptide that specifically binds the CRD of FZD2 or FZD7. In certain embodiments, the method is used to identify a peptide that specifically binds the CRD of FZD2 and FZD7. In certain embodiments, the method is used to identify a peptide that specifically binds the CRD of FZD1 or FZD2. In certain embodiments, the method is used to identify a peptide that specifically binds the CRD of FZD1 and FZD2. In certain embodiments, the method is used to identify a peptide that specifically binds the CRD of FZD1. In certain embodiments, the method is used to identify a peptide that specifically binds the CRD of FZD2. In certain embodiments, the method is used to identify a peptide that specifically binds the CRD of FZD7.

In a specific embodiment, the method is used to identify a peptide that binds or specifically binds the CRD of FZD7.

Multiple rounds of randomization, screening and selection can be performed until non-naturally occurring peptides having sufficient affinity for the target CRD(s) are obtained. Thus, in certain embodiments, steps (e)-(h) or steps (e)-(i) are repeated one, two, three, four, five, six, seven, eight, nine, ten, or more than ten times in order to identify the non-naturally occurring peptide that specifically binds the CRD of FZD7.

In certain embodiments, the peptide that has undergone at least two, three, four, five, six, seven, eight, nine, ten, or more than ten rounds of randomization, screening and selection binds the CRD of FZD7 with an affinity that is at least as high as that of the peptide that has undergone one round of randomization, screening, and selection. In certain embodiments, the non-naturally occurring peptide that has undergone at least two, three, four, five, six, seven, eight, nine, ten, or more than ten rounds of randomization, screening and selection binds the CRD of FZD7 with an affinity that is higher than that of the non-naturally peptide that has undergone one round of randomization, screening, and selection.

A library of non-naturally occurring peptides described herein may be screened by any technique known in the art for evolving new or improved peptides that binds or specifically bind the CRD FZD7. In certain embodiments, the CRD FZD7 is immobilized on a solid support (such as a column resin or microtiter plate well), and the CRD FZD7 is contacted with a library of candidate non-naturally occurring peptides. Selection techniques can be, for example, phage display (Smith (1985) Science 228, 1315-1317), mRNA display (Wilson et al. (2001) Proc Natl Acad Sci USA 98: 3750-3755) bacterial display (Georgiou, et al. (1997) Nat Biotechnol 15:29-34), yeast display (Boder and Wittrup (1997) Nat. Biotechnol. 15:553-5577) or ribosome display (Hanes and Pluckthun (1997) Proc Natl Acad Sci USA 94:4937-4942 and WO2008/068637).

In certain embodiments, provided is a phage particle displaying a non-naturally occurring peptide described herein that binds or specifically binds the CRD of FZD7.

Phage display is a technique by which a plurality non-naturally occurring peptide variants are displayed as fusion proteins to the coat protein on the surface of bacteriophage particles (Smith, G. P. (1985) Science, 228:1315-7; Scott, J. K. and Smith, G. P. (1990) Science 249: 386; Sergeeva, A., et al. (2006) Adv. Drug Deliv. Rev. 58:1622-54). The utility of phage display lies in the fact that large libraries of selectively randomized protein variants (or randomly cloned cDNAs) can be rapidly and efficiently sorted for those sequences that bind to a target ligand with high affinity.

Display of peptides (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352:624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668; Wu et al. (1998) Proc Natl Acad Sci USA. May 95, 6037-42). Polyvalent phage display methods have been used for displaying small random peptides and small proteins through fusions to either gene III or gene VIII of filamentous phage. (Wells and Lowman, Curr. Opin. Struct. Biol., 3:355-362 (1992), and references cited therein.) In a monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. (Lowman and Wells, Methods: A companion to Methods in Enzymology, 3:205-0216 (1991).)

Sorting phage libraries of non-naturally occurring peptides that bind the CRD of FZD7 entails the construction and propagation of a large number of variants, a procedure for affinity purification using the target ligand, and a means of evaluating the results of binding enrichments (see for example, U.S. Pat. Nos. 5,223,409, 5,403,484, 5,571,689, and 5,663,143).

Most phage display methods use filamentous phage (such as M13 phage). Lambdoid phage display systems (see WO1995/34683, U.S. Pat. No. 5,627,024), T4 phage display systems (Ren et al. (1998) Gene 215:439; Zhu et al. (1998) Cancer Research, 58:3209-3214; Jiang et al., (1997) Infection & Immunity, 65:4770-4777; Ren et al. (1997) Gene, 195:303-311; Ren (1996) Protein Sci., 5:1833; Efimov et al. (1995) Virus Genes, 10:173) and T7 phage display systems (Smith and Scott (1993) Methods in Enzymology, 217:228-257; U.S. Pat. No. 5,766,905) are also known.

Many other improvements and variations of the basic phage display concept have now been developed. These improvements enhance the ability of display systems to screen peptide libraries for binding to selected target ligands and to display functional proteins with the potential of screening these proteins for desired properties. Combinatorial reaction devices for phage display reactions have been developed (WO 1998/14277) and phage display libraries have been used to analyze and control bimolecular interactions (WO 1998/20169; WO 1998/20159) and properties of constrained helical peptides (WO 1998/20036). WO 1997/35196 describes a method of isolating an affinity ligand in which a phage display library is contacted with one solution in which the ligand will bind to a target ligand and a second solution in which the affinity ligand will not bind to the target ligand, to selectively isolate binding ligands. WO 1997/46251 describes a method of biopanning a random phage display library with an affinity purified antibody and then isolating binding phage, followed by a micropanning process using microplate wells to isolate high affinity binding phage. Such method can be applied to the non-naturally occurring peptides disclosed herein that bind the CRD of FZD1, FZD2, and/or FZD7. The use of Staphylococcus aureus protein A as an affinity tag has also been reported (Li et al. (1998) Mol Biotech. 9:187). WO 1997/47314 describes the use of substrate subtraction libraries to distinguish enzyme specificities using a combinatorial library which may be a phage display library. Additional methods of selecting specific binding proteins are described in U.S. Pat. Nos. 5,498,538, 5,432,018, and WO 1998/15833. Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

Methods of Producing a Ligand Comprising a Non-Naturally Occurring Peptide that Specifically Binds the CRD of FZD1, FZD2, and/or FZD7

In certain embodiments, a ligand comprising (such as consisting essentially of or consisting of) a non-naturally occurring peptide provided herein that binds or specifically binds the CRD of FZD7 is generated via genetic engineering. A variety of methods for mutagenesis have been previously described (along with appropriate methods for screening or selection). Such mutagenesis methods include, but are not limited to, e.g., error-prone PCR, loop shuffling, or oligonucleotide-directed mutagenesis, random nucleotide insertion or other methods prior to recombination. Further details regarding these methods are described in, e.g., Abou-Nadler et al. (2010) Bioengineered Bugs 1, 337-340; Firth et al. (2005) Bioinformatics 21, 3314-3315; Cirino et al. (2003) Methods Mol Biol 231, 3-9; Pirakitikulr (2010) Protein Sci 19, 2336-2346; Steffens et al. (2007) J. Biomol Tech 18, 147-149; and others. Accordingly, in certain embodiments, provided is a ligand comprising (such as consisting essentially of or consisting of) a non-naturally occurring peptide provided herein that specifically binds the CRD of FZD7 generated via genetic engineering techniques.

In certain embodiments, a ligand comprising (such as consisting essentially of or consisting of) a non-naturally occurring peptide provided herein that binds or specifically binds the CRD of FZD7 is generated via in vitro translation. Briefly, in vitro translation entails cloning the protein-coding sequence(s) into a vector containing a promoter, producing mRNA by transcribing the cloned sequence(s) with an RNA polymerase, and synthesizing the protein by translation of this mRNA in vitro, e.g., using a cell-free extract. A desired variant protein can be generated simply by altering the cloned protein-coding sequence. Many mRNAs can be translated efficiently in wheat germ extracts or in rabbit reticulocyte lysates. Further details regarding in vitro translation are described in, e.g., Hope et al. (1985) Cell 43, 177-188; Hope et al. (1986) Cell 46, 885-894; Hope et al. (1987) EMBO J. 6, 2781-2784; Hope et al. (1988) Nature 333, 635-640; and Melton et al. (1984) Nucl. Acids Res. 12, 7057-7070.

Accordingly, provided are nucleic acid molecules encoding a ligand comprising (such as consisting essentially of or consisting of) a non-naturally occurring peptide provided herein that binds or specifically binds the CRD of FZD7. An expression vector operably linked to a nucleic acid molecule encoding such ligand is also provided. Host cells (including, e.g., prokaryotic host cells such as E. coli, eukaryotic host cells such as yeast cells, mammalian cells, CHO cells, etc.) comprising a nucleic acid encoding such ligand are also provided.

In certain embodiments, a ligand comprising (such as consisting essentially of or consisting of) a non-naturally occurring peptide provided herein that binds or specifically binds the CRD of FZD7 is generated via in vitro translation. Briefly, in vitro translation entails cloning the protein-coding sequence(s) into a vector containing a promoter, producing mRNA by transcribing the cloned sequence(s) with an RNA polymerase, and synthesizing the protein by translation of this mRNA in vitro, e.g., using a cell-free extract. A desired mutant protein can be generated simply by altering the cloned protein-coding sequence. Many mRNAs can be translated efficiently in wheat germ extracts or in rabbit reticulocyte lysates. Further details regarding in vitro translation are described in, e.g., Hope et al. (1985) Cell 43, 177-188; Hope et al. (1986) Cell 46, 885-894; Hope et al. (1987) EMBO J. 6, 2781-2784; Hope et al. (1988) Nature 333, 635-640; and Melton et al. (1984) Nucl. Acids Res. 12, 7057-7070.

In certain embodiments, a ligand comprising (such as consisting essentially of or consisting of) a non-naturally occurring peptide provided herein that binds or specifically binds the CRD of FZD7 is generated via chemical synthesis. In certain embodiments, a non-naturally occurring peptide provided herein that binds or specifically binds the CRD of FZD7 is chemically synthesized and grafted (such as covalently linked) to one or more moieties, as described elsewhere herein.

Methods of solid phase and liquid phase peptide synthesis are well known in the art and described in detail in, e.g., Methods of Molecular Biology, 35, Peptide Synthesis Protocols, (M. W. Pennington and B. M. Dunn Eds), Springer, 1994; Welsch et al. (2010) Curr Opin Chem Biol 14, 1-15; Methods of Enzymology, 289, Solid Phase Peptide Synthesis, (G. B. Fields Ed.), Academic Press, 1997; Chemical Approaches to the Synthesis of Peptides and Proteins, (P. Lloyd-Williams, F. Albericio, and E. Giralt Eds), CRC Press, 1997; Fmoc Solid Phase Peptide Synthesis, A Practical Approach, (W. C. Chan, P. D. White Eds), Oxford University Press, 2000; Solid Phase Synthesis, A Practical Guide, (S. F. Kates, F Albericio Eds), Marcel Dekker, 2000; P. Seneci, Solid-Phase Synthesis and Combinatorial Technologies, John Wiley & Sons, 2000; Synthesis of Peptides and Peptidomimetics (M. Goodman, Editor-in-chief, A. Felix, L. Moroder, C. Tmiolo Eds), Thieme, 2002; N. L. Benoiton, Chemistry of Peptide Synthesis, CRC Press, 2005; Methods in Molecular Biology, 298, Peptide Synthesis and Applications, (J. Howl Ed) Humana Press, 2005; and Amino Acids, Peptides and Proteins in Organic Chemistry, Volume 3, Building Blocks, Catalysts and Coupling Chemistry, (A. B. Hughs, Ed.) Wiley-VCH, 2011.

### Covalent Modifications

Covalent modifications of a ligand comprising (such as consisting essentially of or consisting of) a non-naturally occurring peptide provided herein that binds or specifically binds the CRD of FZD7 are also contemplated. One type of covalent modification includes reacting targeted amino acid residues of a ligand comprising (such as consisting essentially of or consisting of) a non-naturally occurring peptide provided herein that binds or specifically binds the CRD FZD7 with an organic derivatizing agent that is capable of reacting with selected side chains or the N- or C-terminal residues of the ligand. Derivatization with bifunctional agents is useful, for instance, for crosslinking the ligand to a water-insoluble support matrix or surface for use in the method for purifying FZD7, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)-dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification of a ligand comprising (such as consisting essentially of or consisting of) a non-naturally occurring peptide provided herein that binds or specifically binds the CRD FZD7 comprises linking the ligand to one of a variety of nonproteinaceous polymers, e.g., polyethylene glycol (PEG), polypropylene glycol, or polyoxyalkylenes, in the manner set forth in U.S. Pat. Nos. 4,640,835, 4,496,689, 4,301,144, 4,670,417, 4,791,192 or U.S. Pat. No. 4,179,337.

The term “polyethylene glycol” or “PEG” means a polyethylene glycol compound or a derivative thereof, with or without coupling agents, coupling or activating moieties (e.g., with thiol, triflate, tresylate, azirdine, oxirane, N-hydroxysuccinimide or a maleimide moiety). The term “PEG” is intended to indicate polyethylene glycol of a molecular weight between 500 and 150,000 Da, including analogues thereof, wherein for instance the terminal OR-group has been replaced by a methoxy group (referred to as mPEG).

In certain embodiments, a ligand comprising (such as consisting essentially of or consisting of) a non-naturally occurring peptide provided herein that binds or specifically binds the CRD of FZD7 is derivatized with polyethylene glycol (PEG). PEG is a linear, water-soluble polymer of ethylene oxide repeating units with two terminal hydroxyl groups. PEGs are classified by their molecular weights which typically range from about 500 daltons to about 40,000 daltons. In a presently preferred embodiment, the PEGs employed have molecular weights ranging from 5,000 daltons to about 20,000 daltons. PEGs coupled to the ligand comprising (such as consisting essentially of or consisting of) a non-naturally occurring peptide provided herein binds the CRD of FZD7 can be either branched or unbranched. See for example, Monfardini, C. et al. 1995 Bioconjugate Chem 6:62-69). PEGs are commercially available from Nektar Inc., Sigma Chemical Co. and other companies. Such PEGs include, but are not limited to, monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, the hydrophilic polymer which is employed, for example, PEG, is capped at one end by an unreactive group such as a methoxy or ethoxy group. Thereafter, the polymer is activated at the other end by reaction with a suitable activating agent, such as cyanuric halides (for example, cyanuric chloride, bromide or fluoride), diimadozle, an anhydride reagent (for example, a dihalosuccinic anhydride, such as dibromosuccinic anhydride), acyl azide, p-diazoiumbenzyl ether, 3-(p-diazoniumphenoxy)-2-hydroxypropylether) and the like. The activated polymer is then reacted with a ligand comprising (such as consisting essentially of or consisting of) a non-naturally occurring peptide provided herein that binds or specifically binds the CRD of FZD7 to produce a ligand derivatized with a polymer. Alternatively, a functional group in the a ligand comprising (such as consisting essentially of or consisting of) a non-naturally occurring peptide provided herein that binds or specifically binds the CRD of FZD7 can be activated for reaction with the polymer, or the two groups can be joined in a concerted coupling reaction using known coupling methods. It will be readily appreciated that the a ligand comprising (such as consisting essentially of or consisting of) a non-naturally occurring peptide provided herein that binds or specifically binds the CRD of FZD7 can be derivatized with PEG using a myriad of other reaction schemes known to and used by those of skill in the art.

### Liposomes

Ligands comprising (such as consisting essentially of or consisting of) a non-naturally occurring peptide provided herein that binds or specifically binds the CRD of FZD7 can also be formulated as liposomes. Such liposomes can be prepared by methods known in the art, such as described in Epstein et al., Proc Natl Acad Sci USA, 82: 3688 (1985); Hwang et al., Proc Natl Acad Sci USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. A second therapeutic agent is optionally also contained within the liposome. See, Gabizon et al., J. National Cancer Inst., 81(19): 1484 (1989).

### Pharmaceutical Compositions and Formulations

In certain embodiments, provided herein is a pharmaceutical composition comprising a ligand comprising (such as consisting essentially of or consisting of) a non-naturally occurring peptide provided herein that binds or specifically binds the CRD of FZD7 and a pharmaceutically acceptable excipient. In certain embodiments the composition may also contain, buffers, carriers, stabilizers, preservatives and/or bulking agents, to render the composition suitable for ocular administration to a patient to achieve a desired effect or result.

Ligands comprising (such as consisting essentially of or consisting of) a non-naturally occurring peptide provided herein that binds or specifically binds the CRD of FZD7 can be formulated with suitable carriers or excipients so that they are suitable for administration. Suitable formulations of the ligands disclosed herein are obtained by mixing ligands disclosed herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary antibody formulations, which can be applied to the ligands comprising (such as consisting essentially of or consisting of) a non-naturally occurring peptide provided herein that specifically binds the CRD of FZD1, FZD2, and/or FZD7, or to the ligands comprising (such as consisting essentially of or consisting of) a non-naturally occurring peptide provided herein that specifically binds the CRD of FZD7 are described in WO 98/56418, expressly incorporated herein by reference. Lyophilized formulations adapted for subcutaneous administration are described in WO 97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent. Such molecules are suitably present in combination in amounts that are effective for the purpose intended. The effective amount of such other agents depends on the amount of in the formulation, the type of disease or disorder or treatment, and other factors discussed above. These are generally used in the same dosages and with administration routes as described herein or about from 1 to 99% of the heretofore employed dosages. The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared. Suitable examples of sustained release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and. ethyl-L-glutamate, non-degradable ethylene-vinyl, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Lipofectins or liposomes can be used to deliver a ligand comprising (such as consisting essentially of or consisting of) a non-naturally occurring peptide provided herein that binds or specifically binds the CRD of FZD7 provided herein into cells.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's PHARMACEUTICAL SCIENCES, supra.

Sustained-release preparations can be prepared. Suitable examples of sustained-release preparations include semipermeable matrices of solid hydrophobic polymers containing a ligand comprising (such as consisting essentially of or consisting of) a non-naturally occurring peptide provided herein that binds or specifically binds the CRD of FZD7 which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated ligands comprising (such as consisting essentially of or consisting of) a non-naturally occurring peptide provided herein that binds or specifically binds the CRD remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

The formulations to be used for in vivo administration must be sterile. This is readily accomplished by, e.g., filtration through sterile filtration membranes.

Methods of Using Ligands Comprising a Non-Naturally Occurring Peptide that Bind or Specifically Binds the Cysteine-Rich Domain of FZD7

Stem cells are required for the continuous tissue maintenance within diverse organs. Wnt signaling has been identified as regulating stem cells in several organs (including, e.g., the gastrointestinal tract, breast, skin, kidney, and ovary). See Clevers et al. (2014) Science doi: 10.1126/science.1248012. Stem cells are able to self-renew and proliferate autonomously, if they are located in their niche environment within a tissue, and thus already possess some characteristics of cancer cells (Phesse et al. (2009) Br. J Cancer 100, 221-227). Consequently, stem cells have been identified as the cells of origin for several different cancers including the intestine, stomach, prostate and lung (Visvader et al. (2011) Nature 469, 314-322). Wnt signaling has been shown to regulate stem cells in several organs and as such, deregulated Wnt signaling in stem cells is able to induce tumorigenesis in these organs and tissues (Barker et al. (2009) Nature. 457, 608-611). Fzd7 has recently been demonstrated to be the predominant receptor transmitting critical Wnt signals to Lgr5+ intestinal stem cells (Flanagan et al. (2015) Stem Cell Reports, 4, 759-767). Loss-of-function (LOF) mutations to the E3 family ligases ZNRF3 and RNF43, which serve to negatively regulate Fzd receptor turnover, are commonly observed in human colon tumor biopsies (TCGA. Comprehensive molecular characterization of human colon and rectal cancer. Nature 2012, 487, 330-337). Inactivating mutations of RNF43 also confer Wnt dependency in pancreatic ductal adenocarcinoma. (Jiang et al. (2013) Proc Natl Acad Sci USA 110, 12649-12654). R-spondin (RSPO) fusion products have been shown to active Wnt signaling in colon cancer (Seshagiri et al. (2012) “Recurrent R-spondin fusions in colon cancer.” Nature 488, 660-664). In addition, USP6 oncogene promotes Wnt signaling by deubiquitylating Frizzleds (Madan et al. (2016) Proc Natl Acad Sci USA 113, E2945-2954 (2016) Such tumors are predicted to be hypersensitive to Wnt signaling.

Thus, in certain embodiments, provided is a method of inhibiting stem cell proliferation, comprising contacting a stem cell with a ligand comprising (such as consisting essentially of or consisting of) a non-naturally occurring peptide provided herein that binds or specifically binds the CRD domain of FZD7. In certain embodiments, provided is a method of inhibiting stem cell proliferation, comprising contacting a stem cell with a ligand comprising (such as consisting essentially of or consisting of) a non-naturally occurring peptide provided herein that specifically binds the CRD domain of FZD7. In certain embodiments, the stem cell is an intestinal stem cell.

In certain embodiments, provided is a method of treating cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members) in a subject comprising administering an effective amount of a ligand comprising (such as consisting essentially of or consisting of) a non-naturally occurring peptide provided herein that binds or specifically binds the CRD of FZD7. In certain embodiments, provided is a method of treating cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members) in a subject comprising administering an effective amount of a ligand comprising (such as consisting essentially of or consisting of) a non-naturally occurring peptide provided herein that specifically binds the CRD of FZD7.

In certain embodiments, provided is a ligand comprising (such as consisting essentially of or consisting of) a non-naturally occurring peptide provided herein that binds or specifically binds the CRD of FZD7 for use in the manufacture of a medicament for the treatment of cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members). In certain embodiments, provided is a ligand comprising (such as consisting essentially of or consisting of) a non-naturally occurring peptide provided herein that specifically binds the CRD of FZD7 for use in the manufacture of a medicament for the treatment of cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members). In certain embodiments, provided is a ligand comprising (such as consisting essentially of or consisting of) a non-naturally occurring peptide provided herein that binds or specifically binds the CRD of FZD7 for use in treating cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members) in a subject. In certain embodiments, provided is a ligand comprising (such as consisting essentially of or consisting of) a non-naturally occurring peptide provided herein that specifically binds the CRD of FZD7 for use in treating cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members) in a subject. In certain embodiments, provided is a composition (such as a pharmaceutical composition) comprising a ligand comprising (such as consisting essentially of or consisting of) a non-naturally occurring peptide provided herein that binds or specifically binds the CRD of FZD7 for use in treating cancer e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members, in a subject. In certain embodiments, provided is a composition (such as a pharmaceutical composition) comprising a ligand comprising (such as consisting essentially of or consisting of) a non-naturally occurring peptide provided herein that specifically binds the CRD of FZD7 for use in treating cancer e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members, in a subject. In certain embodiments, the ligand comprises (such as consisting essentially of or consists of) a non-naturally occurring peptide comprising an amino acid sequence set forth in SEQ ID NO: 13 or SEQ ID NO: 99. In certain embodiments, the subject to be treated is a mammal (e.g., human, non-human primate, rat, mouse, cow, horse, pig, sheep, goat, dog, cat, etc.). In certain embodiments, the subject is a human. In certain embodiments, the subject is a clinical patient, a clinical trial volunteer, an experimental animal, etc. In certain embodiments, the subject is suspected of having or at risk for having cancer (e.g., colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members).

### Administration and Dosing

Administration of a ligand comprising (such as consisting essentially of or consisting of) a non-naturally occurring peptide provided herein that binds or specifically binds the CRD of FZD7 can be by any suitable route including, e.g., intravenous, intramuscular, or subcutaneous. In certain embodiments, a ligand comprising (such as consisting essentially of or consisting of) a non-naturally occurring peptide provided herein that specifically binds the CRD of that binds or specifically binds the CRD of FZD7 is administered in combination with a second, third, or fourth agent (including, e.g., an antineoplastic agent, a growth inhibitory agent, a cytotoxic agent, or a chemotherapeutic agent) to treat the diseases or disorders involving, e.g., aberrant Wnt activity. In certain embodiments, a ligand comprising (such as consisting essentially of or consisting of) a non-naturally occurring peptide provided herein that binds or specifically binds the CRD of FZD7 is conjugated to the additional agent. Such agents include, e.g., chemotherapeutic agents. In certain embodiments, the ligand comprising (such as consisting essentially of or consisting of) a non-naturally occurring peptide provided herein that binds or specifically binds the CRD of FZD7 is conjugated to the additional agent.

A ligand comprising (such as consisting essentially of or consisting of) a non-naturally occurring peptide provided herein that binds or specifically binds the CRD of FZD7 can be administered to an individual via any route, including, but not limited to, intravenous (e.g., by infusion pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracistemal, intraventricular, rectal (i.e., via suppository), vaginal (i.e., via pessary), intracranial, intraurethral, intrahepatic, and intratumoral. In some embodiments, a ligand comprising (such as consisting essentially of or consisting of) a non-naturally occurring peptide provided herein that binds or specifically binds the CRD of FZD7 is administered systemically (for example by intravenous injection). In some embodiments, a ligand comprising (such as consisting essentially of or consisting of) a non-naturally occurring peptide provided herein that binds or specifically binds the CRD of FZD7 is administered locally (for example by intraarterial or intraocular injection).

Depending on the indication to be treated and factors relevant to the dosing that a physician of skill in the field would be familiar with, a ligand comprising (such as consisting essentially of or consisting of) a non-naturally occurring peptide provided herein that binds or specifically binds the CRD of FZD7 will be administered at a dosage that is efficacious for the treatment of that indication while minimizing toxicity and side effects. A typical dose can be, for example, in the rage of 0.001 to 1000 n; however, doses below or above this exemplary range are within the scope of the invention. The daily dose can be about 0.1 μg/kg to about 100 mg/kg of total body weight (e.g., about 5 μg/kg, about 10 μg/kg, about 100 μg/kg, about 500 μg/kg, about 1 mg/kg, about 50 mg/kg, or a range defined by any two of the foregoing values), preferably from about 0.3 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 1 mg/kg of total body weight (e.g., about 3 μg/kg, about 15 μg/kg, about 75 μg/kg, about 300 μg/kg, about 900 μg/kg, or a range defined by any two of the foregoing values), and even more preferably from about 0.5 to 10 mg/kg body weight per day (e.g., about 2 mg/kg, about 4 mg/kg, about 7 mg/kg, about 9 mg/kg, or a range defined by any two of the foregoing values). As noted above, therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

A pharmaceutical composition comprising a ligand comprising (such as consisting essentially of or consisting of) a non-naturally occurring peptide provided herein that binds or specifically binds the CRD of FZD7 can be administered one, two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection

A ligand comprising (such as consisting essentially of or consisting of) a non-naturally occurring peptide provided herein that binds or specifically binds the CRD of FZD7 may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgically implanted in various locations.

### Articles of Manufacture and Kits

In certain embodiments, provided is an article of manufacture containing a ligand comprising (such as consisting essentially of or consisting of) a non-naturally occurring peptide provided herein that binds or specifically binds the CRD of FZD7 herein and/or a pharmaceutical composition comprising such a ligand, as well as materials useful for the treatment of cancer (such as colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members). The article of manufacture can comprise a container and a label or package insert on or associated with the container. Suitable containers include, for example, bottles, vials, syringes, etc. The containers may be formed from a variety of materials such as glass or plastic. In certain embodiments, the container holds sterile unit-dose packages. The label or package insert indicates that the composition is used for treating cancer (such as colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members). The label or package insert will further comprise instructions for administering ligand comprising (such as consisting essentially of or consisting of) a non-naturally occurring peptide provided herein that binds or specifically binds the CRD of FZD7 to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In certain embodiments, the package insert indicates that the ligand comprising (such as consisting essentially of or consisting of) a non-naturally occurring peptide provided herein that binds or specifically binds the CRD of FZD7 (or the pharmaceutical composition comprising such ligand) is used for treating cancer (such as colon cancer, pancreatic cancer, non-small cell lung cancer, a cancer characterized by a mutation in RNF43, a cancer characterized by USP6 overexpression, or a cancer characterized by gene fusions involving R-spondin (RSPO) family members). Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for isolation or detection of FZD7 in patients, optionally in combination with the articles of manufacture. For isolation and purification of FZD7, the kit can contain a ligand comprising (such as consisting essentially of or consisting of) a non-naturally occurring peptide provided herein that binds or specifically binds the CRD of FZD7 coupled to beads (e.g., sepharose beads). For isolation and purification of FZD7, the kit can contain a ligand comprising (such as consisting essentially of or consisting of) a non-naturally occurring peptide provided herein that specifically binds the CRD of FZD7 coupled to beads (e.g., sepharose beads). Kits can be provided which contain the ligand comprising (such as consisting essentially of or consisting of) a non-naturally occurring peptide provided herein that binds or specifically binds the CRD of FZD7 for detection and quantitation of FZD7 in vitro, e.g. in an ELISA or blot. As with the article of manufacture, the kit comprises a container and a label or package insert on or associated with the container. For example, the container holds a composition comprising at least one ligand comprising (such as consisting essentially of or consisting of) a non-naturally occurring peptide provided herein that binds or specifically binds the CRD of FZD7 described herein. Additional containers may be included that contain, e.g., diluents and buffers, control antibodies, etc. The label or package insert may provide a description of the composition as well as instructions for the intended in vitro or diagnostic use.

### EXAMPLES

### Example 1: Materials and Methods for Examples 2-5

### Reagents, Plasmids, Antibodies and Recombinant Proteins.

Mouse Wnt3a (cat. no. 1324-WN/CF) proteins were purchased from R & D systems. FZD CRD-Fc and sFRP proteins (R&D systems) were dissolved in PBS to 10 μM and include: hFZD1 CRD-Fc (cat no. 5988-FZ), mFZD2 CRD-Fc (cat no. 1307-FC), hFZD4 CRD-Fc (cat no. 5847-FZ), hFZD5 CRD-Fc (cat no. 1617-FZ), hFZD7 CRD-Fc (cat no. 6178-FZ), mFZD7 CRD-Fc (cat no. 198-FZ), hFZD8 CRD-Fc (cat no. 6129-FZ), mFZD9 CRD-Fc (cat no. 2440-FZ), hFZD10 CRD-Fc (cat no. 3459-FZ), hsFRP1 (cat. no. 5396-SF), hsFRP2 (cat. no. 6838-FR), hsFRP-3 (cat. no. 192-SF), hsFRP-4 (cat. no. 1827-SF), and hsFRP-5 (cat. no. 6266-SF). Biotinylated Wnt3a (Bio-Wnt3a) and Wnt5a (Bio-Wnt5a) were generated by R&D systems (not currently a catalogue item). In brief, Wnt3a or Wnt5a was dialyzed into PBS containing 0.5% CHAPS, pH 7.4 and biotinylated according to the manufacturer's recommendation (Pierce, cat no. 21338). pcDNA3.2 Wnt1 and Wnt3a constructs were obtained from the Open Source Wnt project (Najdi et al. (2012) Differentiation 84, 203-213). Anti-Lrp6 and anti-ragweed antibodies were generated as previously described (Tian et al. (2015) Cell Rep 11, 33-42. Peptides were synthesized by standard Fmoc chemistry. Other reagents included: biotinylated peroxidase (cat. no. 432040; Invitrogen), goat anti-human IgG Fc-HRP (cat. no. A18817; ThermoFisher Scientific), DMSO (cat. no. D2650; Sigma), and FuGENE-HD (cat. no. E2311; Promega).

hFZD7 CRD-His was expressed as a secreted protein in Trichoplusia ni cells expressing EndoH, treated with Kifunensine, and then was purified by standard Ni-NTA affinity chromatography followed by size-exclusion chromatography as described elsewhere (Bourhis et al. (2010) J. Biol. Chem. 285, 9172-9179) Affinity beads were washed with 150 mM NaCl, 50 mM Tris-HCl, pH 7.5. The major peak corresponding to the size of a monomer was purified with multiple rounds of size-exclusion chromatography using a Superdex75 column at 0.5 mL/min in the same buffer. hFZD7 CRD fused to Fz7-21 was expressed as a secreted protein in Trichoplusia ni cells and purified by Ni-NTA affinity chromatography. Beads were washed with 150 mM NaCl, 50 mM Tris-HCl, pH 7.5. The major dimer peak was purified with multiple rounds of size exclusion chromatography using an SRT-10 21.2×300 mm SEC300 column at 7 mL/min in the same buffer. Multiple construct designs for FZD3 and FZD6 proteins were tested in Trichoplusia ni or SF9 insect cells as secreted proteins and their expression was monitored by SDS-PAGE in either reducing or non-reducing conditions. No conditions tested provided protein of sufficient quality for downstream analysis.

Sequence alignments were done using the following Uniprot IDs: FZD1, Q9UP38; FZD2, Q14332; FZD3, Q9NPG1; FZD4, Q9ULV1; FZD5, Q13467; FZD6, 060353; FZD7, 075084; FZD8, Q9H461; FZD9, 000144; FZD10, Q9ULW2; hsFRP1, Q8N474; hsFRP2, Q96HF1; hsFRP3, Q92765; hsFRP4, Q6FHJ7; and hsFRP5, Q5T4F7.

Phage Sorting Against hFZD7 CRD-Fc.

Phage pools of linear peptide libraries (Stanger, K. et al. Allosteric peptides bind a caspase zymogen and mediate caspase tetramerization. Nat Chem Biol 8, 655-660 (2012)) were cycled through rounds of binding selections with hFZD7 CRD-Fc following standard protocols (Tonikian et al. (2007) Nat Protoc 2, 1368-1386). Herceptin (10 μM) was included in sorting solution to block potential Fc binders during all rounds of sorting. After four rounds of binding selection, individual phage clones were analyzed in a high-throughput spot phage ELISA using plate-immobilized FZD7 CRD-Fc as target or off-target for specificity test (hFZD8 CRD-Fc and Herceptin). To measure off-target affinity, duplicate phage particle were assayed against BSA to measure non-specific binding. Clones with a phage-binding signal >0.5 and signal-to-noise ratio >10 were considered to be positive clones and were subjected to DNA sequence analysis. General data handling was done using Microsoft Excel 2010 unless otherwise noted.

### Peptide Synthesis.

All peptides were synthesized using standard 9-fluorenylmethoxycarbonyl protocols as described elsewhere (Zhang et al. (2009) Nat Chem Biol 5, 217-219), with the N termini protected as acetamides and C termini protected as carboxamides, and purified by RP-HPLC. Peptide quality (>90% purity) was checked by LC-MS, and peptide identity was confirmed by MS.

### Cell Culture and Transfection.

HEK293 cells stably integrated with a firefly luciferase Wnt reporter (TOPbrite, TB) (Zhang et al. (2009) Nat Chem Biol 5, 217-219) and pRL-SV40 Renilla luciferase (cat. no. E2231; Promega) were grown in DMEM:F12 (50:50) supplemented with 10% FBS, 2 mM Glutamax™ (cat. no. 35050-061; Gibco) and 40 μg/ml hygromycin (cat. no. 30-240-CR; Cellgro). Cells were incubated in a 5% CO₂ humidified incubator at 37° C. for 24 h before any experiments. For the luciferase reporter assay, 50 μl HEK293-TB cells (20,000 cells/well) were seeded in each well of clear bottom white polystyrene 96-well plates (cat. no. 353377; Falcon). After 24 h, cells were transfected with the indicated Wnt constructs and Fugene HD (cat. no. E2312; Promega; cDNA and Fugene were mixed in 10 μl OptiMEM [cat. no. 31985-070; Gibco]) for 24 h, then treated with the indicated peptide for 6 h, and processed with 50 μL of Dual-Glo Luciferase Assay system (cat. no. E2940; Promega). For treatment with peptides, all peptides were diluted in DMSO, and added to cells at a final DMSO concentration of 1%. For treatment with recombinant Wnt proteins, all Wnts were diluted in PBS, simultaneously added with peptides to cells and then processed as described above. The firefly and renilla luminescence were measured on a Perkin Elmer EnVision® multilabel reader. The ratios of firefly luminescence over renilla luminescence were calculated, background subtracted, and normalized to the ratio in control cells treated with Wnt3a only (50 ng/mL). Cell lines were obtained from the Genentech gCell laboratory and were tested for mycoplasma contamination and authenticated by SNP analysis.

### FACS Analysis.

Cell lines stably expressing gD-FZD were grown to ˜80% confluence in DMEM:F12 (50:50) media supplemented with 10% FBS (cat. no. 1500-100; Seradigm), 1× GlutMAX (cat. no. 35050-061; Gibco) and G418 (200 μg/mL; cat. no. A1720; Sigma), released with 0.05% trypsin/EDTA in PBS (cat. no. 15400-054; Gibco), collected on ice and buffer exchanged into ice cold FACS buffer (0.5% BSA and 0.05% NaAz in PBS). 100,000 cells/well were plated into an ice cold U-bottomed plate (cat. no. 3799; Costar) and cells pelleted by centrifugation at 1200 rpm for 3 min. Cells were treated with of 5FAM-Fz7-21 (12.5 μM) or DMSO (cat. no. D2650; Sigma) supplemented with mouse anti-gD antibody (Genentech; gD:952; 500 ng/mL) in FACS buffer for 1 h in the dark on ice. Cells treated with 5FAM-Fz7-21S displayed significant non-specific binding and were excluded from analysis. Cells were washed three times with ice cold FACS Wash buffer (0.5% BSA and 0.1% NaAz in PBS) on ice. Cells were then treated with Alexa 647 conjugated donkey anti-mouse IgG (H+L) secondary antibody (500 ng/mL; cat. no. A-31571; Invitrogen) in the dark for 1 h on ice. Cells were washed three times with ice cold FACS wash buffer on ice then incubated for 15 min with SYTOX (cat. no. 534857; Invitrogen) prior to analysis on a BD Fortessa instrument. Data was collected with FACSDiva (BD; version 8.0.1) and analyzed using FLowJo (FlowJo, LLC; v10.1).

### ELISA.

384-well Maxisorp plates (cat. no. 464718; Thermo Scientific) were incubated with 30 μL of Neutravidin (cat. no. 31000, Thermo Scientific; 2 μg/mL in PBS) overnight at 4° C. Plates were then washed with PBST and blocked with PBS/1% BSA for 1 hour at RT and then washed with PBST prior to further processing. The binding assay was conducted in 96 well plates (cat no. 83007-374; VWR) which were blocked with PBS/1% BSA overnight at 4° C. hFZD4 CRD-Fc or hFZD7 CRD-Fc (63 ng/mL) were incubated with either Bio-Wnt3a (25 ng/mL) or Bio-Wnt5a (50 ng/mL) in the presence of 4-fold serial dilutions of the indicated peptide in PBS overnight at 4° C. All assays included hFZD9 CRD-mIgG2A protein as a negative control. hFZD7 CRD-His was suspended in 150 mM NaCl, 50 mM Tris-HCl at pH 7.5 and when used in assays an equal volume of the same buffer control was used. The binding assay mixtures were transferred to the Neutravidin coated plates and incubated for ˜1 h at RT. The wells were then washed with PBST, incubated with anti-hIgG1-HRP (1:10,000 suspended in PBS supplemented with 1% BSA; cat. no. A18817, Invitrogen), and washed again with PBST. The signal was developed with the addition of TMB reagent (following the manufacturer's recommendation; KPL; 50-76-00) for 15 min prior to the addition of an equal volume of 1 M phosphoric acid for 10 min Absorbance was measured at 450 nm using a Tekan M1000 plate reader. Values were collected and plotted using Prism Graphpad software. All assay conditions using peptides were at 1% DMSO final concentration.

### Fluorescence Size-Exclusion Chromatography.

Fluorescence size-exclusion chromatography (FSEC) experiments were run using an AKTA FPLC system (General Electric Company) with UNICORN software package (version 5.31). Samples were resolved using a SEC3000 or SEC2000 (Phenomenex; Torrence, Calif.) column at 0.5 mL/min in phosphate buffer. Fluorescence was monitored using a FP-2020 Plus fluorescence detector (Jasco Analytical Instruments, Easton, Md.) in normal mode (excitation/emission, 550/494 nm; gain=100; STD=32). All samples were prepared with a final DMSO concentration of 1% and incubated overnight at 4° C. prior to sample separation. FZD CRD-Fc samples were kept at ˜250 nM in the presence of excess 5FAM labeled peptide (1 μM). Human secreted Frizzled-related proteins were maintained at ˜250 nM in the presence of excess 5FAM-labeled peptide (10 μM).

Size-Exclusion Chromatography Coupled with Multi-Angle Light Scattering.

hFZD7 CRD (15.8 μM) was incubated with Fz7-21, Fz7-21S or DMSO at the indicated ratio of protein to peptide for 2 h prior to analysis by size-exclusion chromatography (SEC) and detection with multi-angle static light scattering (MALS). Samples were resolved by a Superdex S200 3.2/300 column (General Electric Healthcare Life Science, Pittsburgh, Pa.; cat. no. 28990946) at 0.15 mL/min in 150 mM NaCl, 50 mM Tris-HCl pH 7.5 buffer with 1% DMSO final concentration. Runs were performed on a 1260 infinity HPLC (Agilent Technology, Santa Clara, Calif.) connected to a Dawn Heleos-II multi-angle static Light Scattering (MALS) detector and a Optilab T-rEX differential Refractive Index (dRI) detector (Wyatt Technologies, Santa Barbara, Calif.).

### X-Ray Crystallography.

hFZD7 CRD-His was purified, buffer exchanged into 150 mM NaCl, 50 mM Tris-HCl, pH 7.5 and then concentrated to ˜25 mg/mL by centrifugation (cat. no. UFC900325; Millipore Amicon Ultra 15). The protein was diluted (1:1) with 25% PEG 2000 MME (w/v) with MES pH 6.5 (cat. no. 134312; Qiagen) and grown at 19° C. by vapor diffusion. Crystals of sufficient quality grew within 10 days, were cryo-protected in the mother liquor supplemented with 30% PEG2000 MME, and flash frozen in liquid nitrogen. Data sets were collected at the Stanford Synchrotron Radiation Lightsource (SSRL) 12-2 and solved by molecular replacement at 2.00 Å resolution. (Nile, et al. (2017) “Unsaturated fatty acyl recognition by Frizzled receptors mediates dimerization.”Proc. Natl. Acad. Sci. USA 114, 4147-4152 LID-4110.1073/pnas.1618293114 [doi].) hFZD7 CRD [Gln33-Gly168] fused to Fz7-21 was expressed as a secreted protein in Trichoplusia ni cells expressing EndoH and treated with Kifunensin. hFZD7 CRD was inserted into pACGP67-A vector (BD Biosciences-Pharmingen) as a secreted protein. hFZD7 CRD fused to Fz7-21 was passed over Ni-NTA beads (Qiagen; 1018401), washed with 300 mM NaCl, 50 mM Tris, HCl pH 7.5, then eluted with the same buffer containing 300 mM imidazole. The eluent was collected, concentrated and buffer exchanged into 150 mM NaCl, 50 mM Tris HCl, pH 7.5 and the dimer pool was collected by gel filtration (Superdex 200; GE Healthcare Life Sciences) in 150 mM NaCl, 50 mM Tris-HCl, pH 8.0. The dimer fraction was concentrated to ˜20 mg/mL by centrifugation and protein was diluted to a 1:1 mix with 1-propanol 14% (v/v; cat. no. 09158; Fluka), 9% PEG5000 MME (cat. no. HR-2-615; Hampton Research), and 0.1 M MES at pH 6.9 (cat. no. HR2-243; Hampton Research) and grown at 4° C. by vapor diffusion. X-ray diffraction data were collected at the Advanced Photon Source (Argonne National Laboratory) beam line 17ID with a pixel-array detector (Pilatus, Dectris AG, Switzerland). X-ray wavelength was set at 1.00000 Å.

A complete data set was collected with a single crystal under cryogenic temperature (−180° C.). The diffraction data were integrated using program XDS (Kabsch, W. Integration, scaling, space-group assignment and post-refinement. Acta Crystallographica Section D: Biological Crystallography 66, 133-144 (2010)) and scaled using program aimless (Blessing, R. H. An empirical correction for absorption anisotropy. Acta Crystallogr A 51 (Pt 1), 33-38 (1995)) of CCP4 package Murshudov, G. N., Vagin, A. A. & Dodson, E. J. Refinement of macromolecular structures by the maximum-likelihood method. Acta Crystallogr D Biol Crystallogr 53, 240-255 (1997)). (The structure was phased by molecular replacement (MR) method using program Phaser (McCoy, A. J. et al. Phaser crystallographic software. Journal of Applied Crystallography 40, 658-674 (2007)). hFZD7 CRD structure was used as the MR search model (PDB ID# 5URV) Nile, A. H., Mukund, S., Stanger, K., Wang, W. & Hannoush, R. N. Unsaturated fatty acyl recognition by Frizzled receptors mediates dimerization. Proc. Natl. Acad. Sci. USA 114, 4147-4152 LID-4110.1073/pnas.1618293114 [doi] (2017)). The structure was subsequently subjected to iterative model building with graphics program COOT (Emsley, P., Lohkamp, B., Scott, W. G. & Cowtan, K. Features and development of Coot. Acta Crystallographica Section D: Biological Crystallography 66, 486-501 (2010)) and maximum-likelihood least-square refinement protocols encoded in program PHENIX (Adams, P. D. et al. PHENIX: a comprehensive Python-based system for macromolecular structure solution. Acta Crystallographica Section D 66, 213-221 (2010)). The final structure was refined to 2.88 Å resolution, with 88.3% of residues falling in favored regions in the Ramachandran plot, and 11.1% in allowed regions, 0.3% in generally allowed regions and 0.2% in disallowed regions. Random electron densities were observed within the FZD7 CRD hydrophobic cavity; however, a confident assignment could not be made. More detailed diffraction data processing and structure refinement statistics are available in Table 10. X-ray diffraction data were collected at the Advanced Photon Source (Argonne National Laboratory) beam line. The structure of FZD7 CRD in complex with Fz7-21 was deposited to the PDB database, with code 5WBS.

### Structural Analysis.

To calculate and display the conservation of FZD molecules, the surface of human (h) FZD8 was used as a surrogate to render the conservation between the ten hFZD CRDs (hFZD1 through hFZD10; cyan, low conservation; maroon, high conservation). hFZD CRD sequences were acquired from Uniprot, aggregated with UGENE46 (v1.14.1) (Okonechnikov, K., Golosova, O., Fursov, M. & team, t.U. Unipro UGENE: a unified bioinformatics toolkit. Bioinformatics 28, 1166-1167 (2012)), and aligned with Clustal Omega33. Sequences were trimmed, guided by iterative alignments using hFZD7 CRD (from Y35 to D167) as a guide relying heavily on the absolutely conserved cysteine residues as reference for CRD boundaries. Alignment files were exported to UCSF Chimera (v1.10.2) (Pettersen, E. F. et al. UCSF Chimera—a visualization system for exploratory research and analysis. J Comput Chem 25, 1605-1612 (2004)) and sequence conservation was calculated using the AL2CO algorithm (Pei, J. & Grishin, N. V. AL2CO: calculation of positional conservation in a protein sequence alignment. Bioinformatics 17, 700-712 (2001)) using frequency estimation by independent counts, measuring conservation through an entropy-based approach and an average window of 1 with a gap fraction of 0.5. Conservation values calculated from AL2CO represent the standard deviations from the mean and range from +1.678 (most conserved; maroon) to −1.539 (least conserved; cyan) over a color gradient and the gradient was displayed as a molecular surface in mFZD8 CRD (PDB ID #4F0A).

To calculate the intramolecular interactions between dFz7-21, the find clash/contact utility within UCSF Chimera47 was used to identify the overlap between two atoms defined as the sum of their VDW radii minus the distance between them and minus an allowance for potentially hydrogen-bonded pairs [overlapij=rVDWi+rVDWj−dij−allowanceij]. This calculation identifies all direct interactions including polar and nonpolar, favorable and unfavorable (including clashes) where the VDW overlap is >−0.4 Å with no overlap subtracted for potential hydrogen bonding pairs. The solvent accessible surface (SAS) molecular surface of an isolated dFz7-21 molecule (chain A and chain B) was calculated using the MSMS package (Sanner, M. F., Olson, A. J. & Spehner, J. C. Reduced surface: an efficient way to compute molecular surfaces. Biopolymers 38, 305-320 (1996)) (probe radius of 1.4 Å) within the UCSF chimera package.

hFZD7 CRD Hydrophobic Cavity Representation.

To visualize the hydrophobic cavity within hFZD7 CRD in complex with Fz7-21, two XWnt8 molecules in complex with mFZD8 CRD (PDB ID# F40A) were superimposed onto hFZD7 CRD structure using the MatchMaker utility in UCSF Chimera, employing the Needleman-Wunsch alignment algorithm and the BLOSUM-62 matrix. The continuous hydrophobic surface was identified within ˜5 Å of the fatty acyl moieties. In the superimposition of apo hFZD7 CRD with F40A, the fatty acids were truncated from their w-carbons to eliminate clashes between the fatty acids within the hydrophobic cavity, and then hydrophobic cavity search was performed as described above to identify hydrophobic surfaces within ˜5 Å of the fatty acyl group.

### In Silico Analysis.

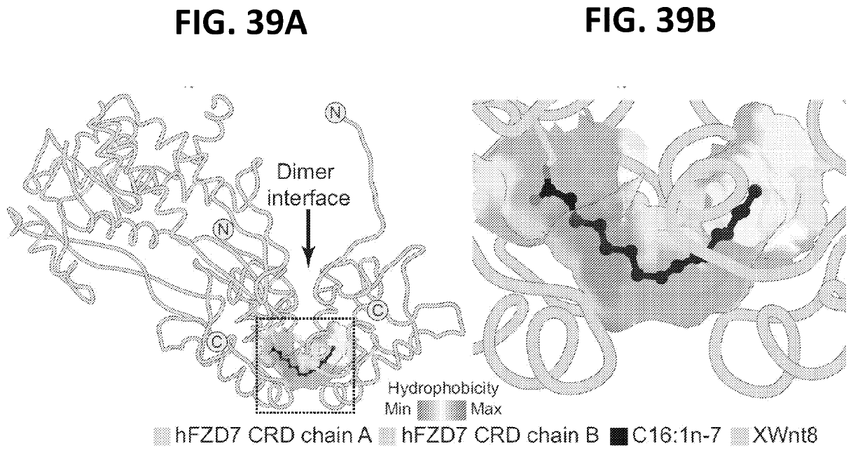

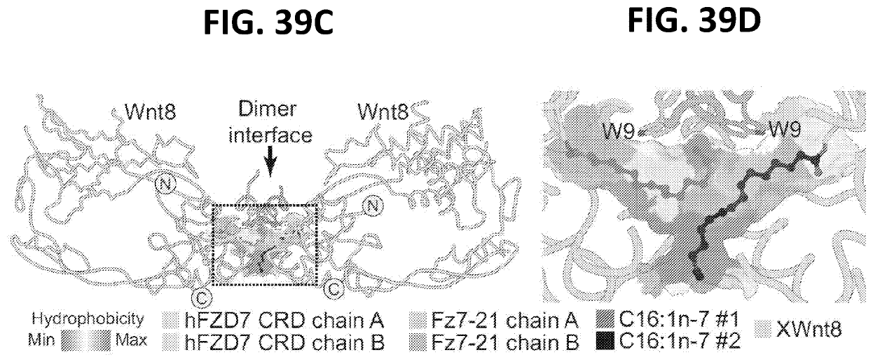

The model presented in FIGS. 39A and 39B of XWnt8a (PDB ID #4F0A) in complex with hFZD7 CRD (PDB ID #5URV) with an elongated fatty acyl moiety (C16:n-7) binding in the U-shaped hydrophobic cavity of hFZD7 CRD was constructed in MOE (Chemical Computing Group; version 2017.05). XWnt8a was superimposed onto the C24 bound hFZD7 CRD structure (PDB ID #5URV) using the mFZD8 CRD (PDB ID #4F0A) as guide. Protonate 3D and structure preparation utilities were applied using the default settings to optimize hydrogen placement. The C14 fatty acid covalently linked at XWnt8a Ser187 was pruned and elongated to follow the contour of the C24 fatty acid within the hFZD7 CRD hydrophobic cavity. Then the fatty acid and residues in close proximity were energy minimized using the MMFF94x forcefield (eps=r; Cutoff [8,10]) with rigid water and a 0.1 RMS kcal/mol/A² gradient. The model found in FIGS. 39C and 39D of two molecules of XWnt8a (PDB ID #4F0A) in complex with hFZD7 CRD bound to Fz7-21 with an elongated fatty acyl moiety (C16:n-7) binding in the hydrophobic cavity of hFZD7 CRD was constructed in MOE (Chemical Computing Group; version 2017.05) as described above. For each structural representation, the respective ligands were used to define the hydrophobic cavity. The continuous hydrophobic surface was identified within ˜5 Å of the bound ligand and visualized according to the hydrophobicity of the cavity. The molecular surface by amino acid hydrophobicity is scaled based on the Kyte-Doolittle scoring metric (Kyte, J. & Doolittle, R. F. A simple method for displaying the hydropathic character of a protein. Journal of Molecular Biology 157, 105-132 (1982)). Coordinates were exported into UCSF Chimera (version 1.11) for figure generation.

### NMR Spectroscopy.

All NMR experiments were performed on a Bruker Ultrashield plus 600 MHz spectrometer equipped with a cryoprobe. The peptides were resuspended at 1 mM in 10 mM phosphate buffer (pH 7.3) and 10% (v/v) acentonitrile-d₃ (ISOTEC, Inc.; cat. no. T82-05013-ML) in H₂O for determination of NMR structure in solution. Total Correlation Spectroscopy (TOCSY) and Nuclear Overhauser Effect Spectroscopy (NOESY) of the peptide were recorded with mixing time of 70 and 350 ms, respectively, at 27° C. Topspin (Bruker Biospin) and CCPN analysis were used for spectral processing, visualization and peak picking (Vranken et al. (2005) Proteins 59, 687-696). Based on homonuclear TOCSY and NOESY spectra, the residues of the peptide were assigned. Nearly complete resonance assignments of the protons were obtained based on spin-system identification and sequential assignments (Wüthrich, K. NMR of Proteins and Nucleic Acids. (Wiley Interscience, New York, 1986). Interproton distance restraints were obtained from the NOESY spectra. Three-dimensional structure of dFz7-21 was initially calculated using the CYANA 3.97 package (Guntert & Buchner (2015) Journal of biomolecular NMR 62, 453-471; Guntert et al. (1997) J Mol Biol 273, 283-298). Because only a single set of resonances was observed, dFz7-21 was treated as a symmetrical dimer in CYANA calculation with duplicated sequences and symmetric distance restraints. See Table 16.

The structure was then refined with CNS (Brunger, A. T. et al. Crystallography & NMR system: A new software suite for macromolecular structure determination. Acta Crystallogr D Biol Crystallogr 54, 905-921 (1998); Brunger, A. T. Version 1.2 of the Crystallography and NMR system. Nat Protoc 2, 2728-2733 (2007)). A total of 50 structures were generated and 20 structures with the lowest energy were selected. Ramachandran analysis of ordered residues (residue 6-14) by Procheck shows 63.1% residues are in most favored regions, 27.2% residues are in additionally allowed regions and 9.7% residues are in generously allowed regions with 0% residues in disallowed regions. The peptide of interest is not isotopically labelled, so assignments of backbone nuclei were not obtained (Ca, Cb, Ha, CO). With no backbone assignments, dihedral angle restraints were not included in the structure calculation. The chemical shift assignments and NOE restraints used in structure calculations were deposited into BMRB with code 30311. The structure was deposited to the PDB database, with code 5W96.

### Organoid Culture.

Mouse organoids were established from isolated crypts collected from the entire length of the small intestine and maintained as previously described (Sato et al. (2009) Nature 459, 262-265 (2009). All mouse derived tissue was performed according to the animal use guidelines of Genentech, a Member of the Roche Group, and the Institutional Animal Care and Use Committee, conforming to California State legal and ethical practices. Mouse sex was not selected for. Organoids were passaged at least twice per week and grown using IntestiCult Organoid growth media (cat. no. 06005; StemCell Technologies). Peptides were suspended in DMSO and dissolved in media to 1% DMSO final concentration, then added to organoids to initiate drug treatment. Antibodies were added to 10 μg/ml concentration. Organoids were imaged using a Nikon Eclipse Ti scope with a Nikon Plan Fluor 10×Ph1 DLL objective using an Andor Neo camera (1×1 binning; 200 ms exposure) and acquired using NIS Elements (v 4.50 64-bit; Nikon). APC^{min} organoids were generated as previously described (Melo, F.d.S.e. et al. A distinct role for Lgr5+ stem cells in primary and metastatic colon cancer. Nature 543, 676-680 (2017)). For organoids treated with CHIR99021 (5 μM; Stemcell Technologies) organoids were split and treated with DMSO or CHIR99021 (5 μM) for 24 hr. prior to the addition of peptide or DMSO for 24 hr.

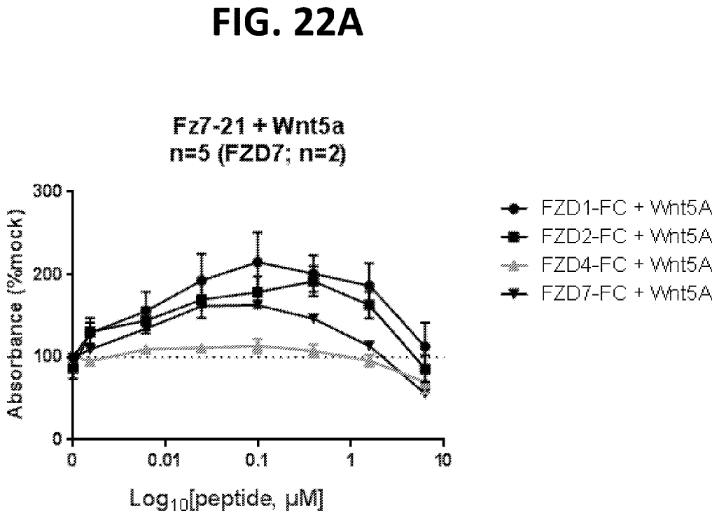

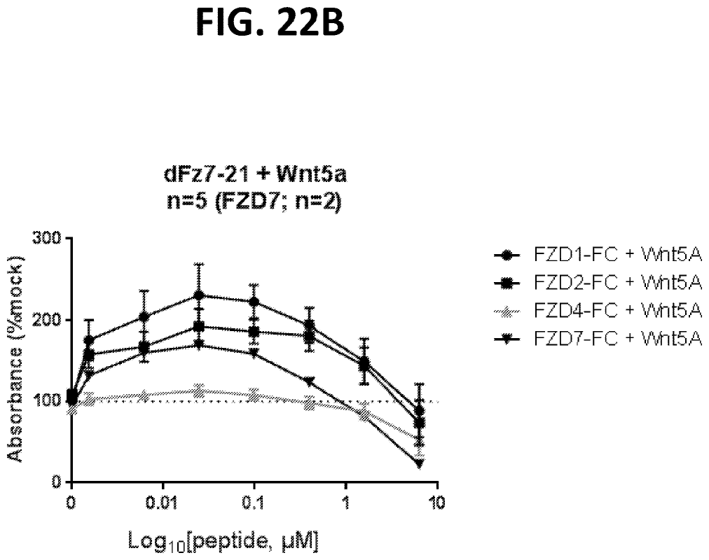

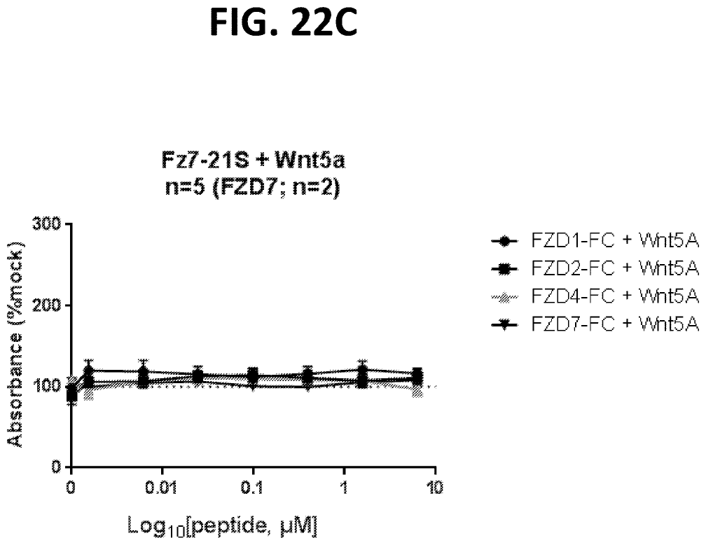

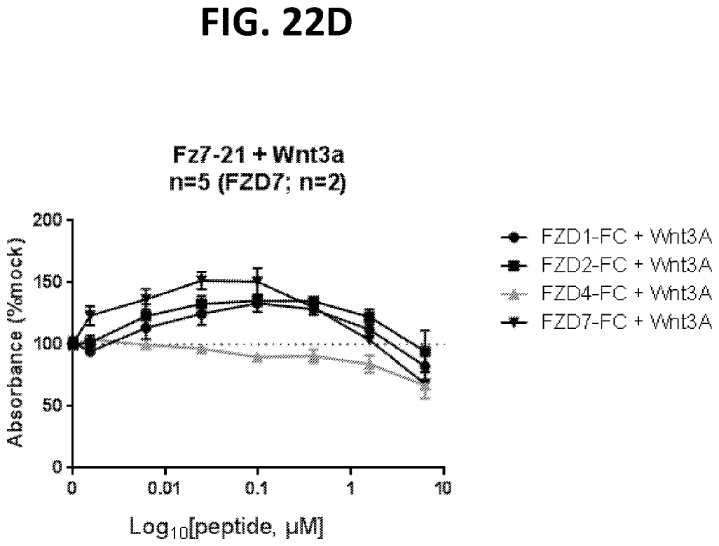

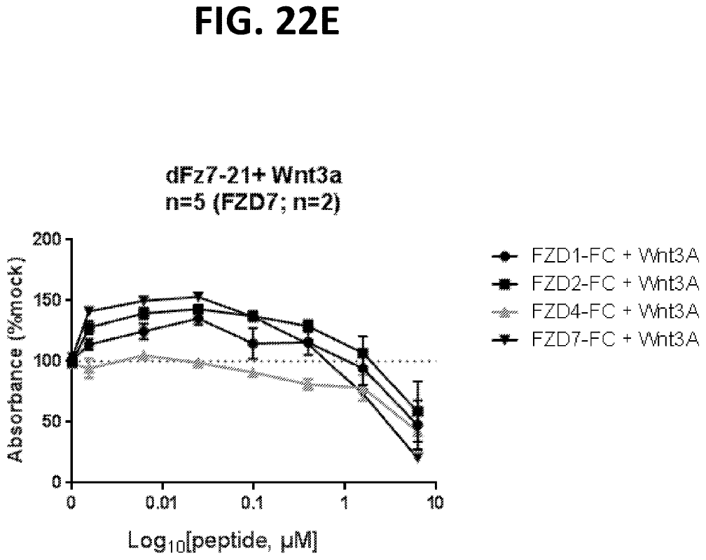

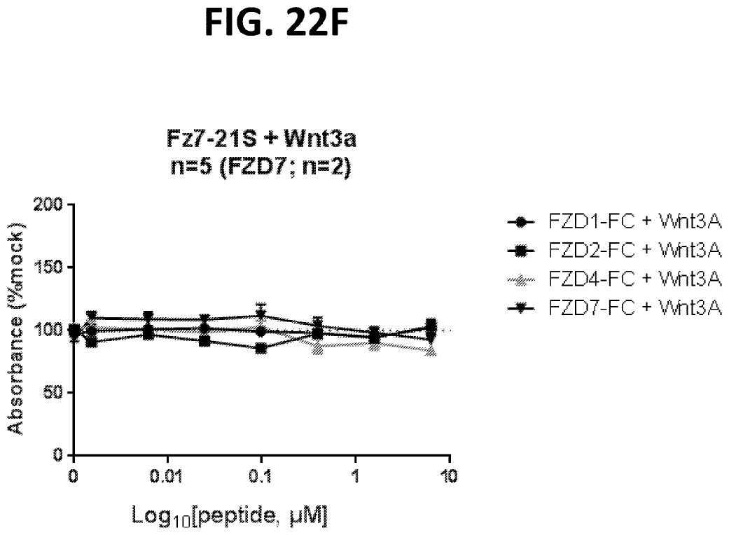

For confocal images found in Supplementary FIG. 22 a LEICA SP5 laser scanning confocal microscope was used to collect images with a 40× oil-immersion objective (HCX PL APO CS UV, 1.25NA) in the blue (DAPI) and green (GFP) channels. All images were acquired under identical conditions in sequential mode as follows: sequence 1 (blue channel): UV laser (405 nm): 85% power of excitation, PMT settings: 425-470 nm emission, active gain: 900, offset: −15; sequence 2 (green channel): Argon laser (488 nm), 20% output, 55% power of excitation, PMT settings: 500-550 nm emission, active gain 700, offset: −5. Images of 2048×2048 pixel resolution (1.5× zoom) were collected at 400 Hz unidirectional scanning speed, with a 1 Airy unit pinhole. A stack of 10-20 optical sections (0.968 micrometer thickness) were collected and images of the maximum intensity projections were used in the figures.

### RNA Extraction and RT-PCR.

RNA from organoid samples was isolated using the RNeasy Micro kit (cat. no. 74004; Qiagen) according to the manufacturer's instructions. Mouse small intestine RNA was collected using the RNeasy Mini Kit (cat. no. 74104; Qiagen). qRT-PCR was performed in 10 μL reactions with 50 ng total RNA using One-step Real-time RT-PCR mastermix (cat. no. 4392938; Life Technologies) according to the manufacturer's instructions. Taqman probes from Life Technologies were used: Actb (Mm01205647_g1); Lgr5 (Mm01251801_m1); Axin2 (Mm00443610_m1); Ascl2 (Mm01268891_g1); Olfm4 (Mm01320260_m1); Muc2 (Mm01276696_m1); ACTB(Hs99999903_m1); ACTB(Mm00607939_s1); GAPDH (Mm99999915_g1); and GAPDH(Hs03929097_g1). RT-PCR reactions were run using a 7900HT Fast Real-Time PCR system (ABI) at the following thermal cycling conditions: Holding step of 30 min at 48° C., followed by a holding step of 10 min at 95° C., and 40 cycles of 10 sec at 95° C. and 1 min at 60° C. Values were normalized to actin transcript levels and then normalized to control as described in the figure legend.

### RNA Sequencing.

RNA-Seq libraries were prepared using TruSeq RNA Sample Preparation kit (Illumina, CA). The libraries were sequenced on Illumina HiSeq 2500 sequencers to obtain on average 34 million single-end reads (50 bp) per sample. RNAseq reads were first aligned to ribosomal RNA sequences to remove ribosomal reads. The remaining reads were aligned to the mouse reference genome (NCBI Build 38) using GSNAP (version 2013-10-10) (Wu, T. D. & Nacu, S. Fast and SNP-tolerant detection of complex variants and splicing in short reads. Bioinformatics 26, 873-881 (2010)) allowing a maximum of two mismatches per 50 base sequence (parameters: ‘-M 2-n 10-B 2-i 1-N 1-w 200000-E 1-pairmax-rna=200000-clip-overlap’). Transcript annotation was based on the RefSeq database (NCBI Annotation Release 104). To quantify gene expression levels, the number of reads mapped to the exons of each RefSeq gene was calculated. Read counts were scaled by library size, quantile normalized and precision weights calculated using the “voom” R package (Law, C. W., Chen, Y., Shi, W. & Smyth, G. K. voom: precision weights unlock linear model analysis tools for RNA-seq read counts. Genome Biology 15, R29 (2014)). Subsequently, differential expression analysis on the normalized count data was performed using the “limma” R package (Ritchie, M. E. et al. limma powers differential expression analyses for RNA-sequencing and microarray studies. Nucleic Acids Research 43, e47-e47 (2015)) by contrasting dFz7-21 treated samples with either DMSO or Fz7-21S treated samples at 6 h and 24 h, respectively. In addition, gene expression was obtained in form of normalized Reads Per Kilobase gene model per Million total reads (nRPKM) as described earlier (Srinivasan, K. et al. Untangling the brain's neuroinflammatory and neurodegenerative transcriptional responses. Nat Commun 7, 11295 (2016)). The collected RNAseq data is available through NCBI's Gene Expression Omnibus under accession GSE94159.

### Gene Set Analysis.

We performed Quantitative Set Analysis for Gene Expression (QuSAGE) (Yaari, G., Bolen, C. R., Thakar, J. & Kleinstein, S. H. Quantitative set analysis for gene expression: a method to quantify gene set differential expression including gene-gene correlations. Nucleic Acids Research 41, e170-e170 (2013)) to identify relevant biological processes associated with Wnt inhibition. For that purpose dFz7-21 treated samples were contrasted with DMSO samples at either 6 h or 24 h. For each contrast the gene set activity (i.e. the mean difference in log 2 expression of the individual genes that compose the set) for selected sets was calculated as defined in Kim et al. Single-Cell Transcript Profiles Reveal Multilineage Priming in Early Progenitors Derived from Lgr5+ Intestinal Stem Cells. Cell Reports 16, 2053-2060 (2016) that are related to intestinal biology.

### Animal Studies.

C57BL/6 female mice (8 weeks old) were subjected to intraperitoneal injection once per hour (˜150-200 μL injection volume for five injections) until the indicated peptide dose was reached. Control anti-Lrp6 or anti-Ragweed antibodies (15 mg/kg) were both administered intraperitoneally at experiment onset and the small intestine was harvested after 6 h. dFz7-21 peptide was suspended to 20 mg/mL (for 160 mg/kg injections) or 10 mg/mL (for 80 mg/kg injections) in 90.9 mM ammonium bicarbonate, 18.2 mM histidine acetate, 218.2 mM sucrose, and 0.018% polysorbate-20 and pH between 7.5 and 8. Antibodies were resuspended in 20 mM histidine acetate, 240 mM sucrose, 0.02% polysorbate-20, pH 5.5. After 6 h, mice were sacrificed, and the small intestine was collected for mRNA extraction. All studies involving animals were approved by Genentech's Institutional Animal Care and Use Committee and adhere to the NRC Guidelines for the Care and Use of Laboratory Animals.

### Example 2: Identification of Peptides that Selectively Bind FZD7 CRD

FZD7 plays a critical role in broad stem cell processes as it is upregulated in multiple tissue-specific stem cells (Vincan & Barker (2008) Clinical &experimental metastasis 25, 657-663; Phesse, et al. (2016) Cancers 8). Among the ten mammalian Frizzleds, FZDs 1, 2 and 7 (which belong to the FZD7 sub-class) are enriched at the base of the mammalian adult intestinal crypts, where multipotent stem cells are known to exist (Mariadason et al. (20051) Gastroenterology 128, 1081-1088; Gregorieff et al. (2005) Gastroenterology 129, 626-638). It was shown recently that FZD7 is enriched in Lgr5+ intestinal stem cells (ISCs), and is required for stem cell-mediated regeneration of the intestinal epithelium after gamma irradiation, implicating it as the critical FZD receptor responsible for mediating Wnt activity in intestinal stem cells (Flanagan et al. (2015) Stem cell reports 4, 759-767). FZD7 genetic knockdown experiments established that FZD7 is also essential for maintaining human embryonic stem cells in their undifferentiated state (Fernandez, et al. (2014) Proceedings of the National Academy of Sciences 111, 1409-1414, doi:10.1073/pnas.1323697111). Moreover, recent data demonstrated that FZD7 is upregulated in subsets of colon, pancreatic and gastric tumors (Vincan et al. (2008) Clinical & experimental metastasis 25, 657-663; Phesse (2016) Cancers 8). Additionally, FZD7 has been implicated in tumor initiation and metastatic growth of melanomas (as well as their drug resistance to BRAF inhibitors (Tiwary & Xu (2016) PLoS One 11, e0147638); Anastas et al. (2014) The Journal of clinical investigation 124, 2877-2890). These findings highlight FZD7 receptor as a critical regulator of stem cells and an attractive pharmacological target for diseases associated with stem cell dysfunction.

Wnt signaling is initiated at the cell surface upon interaction of secreted Wnt glycoproteins with FZD receptors. The covalently-linked cis-unsaturated fatty acyl group (palmitoleate) present on Wnt proteins binds to the lipid-binding groove within the extracellular N-terminal CRD of FZD receptors (Janda et al. (2012) “Structural basis of Wnt recognition by Frizzled.” Science 337, 59-64; Nile and Hannoush (2016) “Fatty acylation of Wnt proteins.”Nature Chemical Biology 12, 60-69), leading stabilization of nuclear β-catenin and initiation of downstream Wnt signaling. In colorectal cancer, the majority of Wnt pathway mutations occur at the level of β-catenin (Polakis, P. (2012) Cold Spring Harb Perspect Biol 4). However, aberrant activation of the Wnt pathway could also occur at the level of the receptors, as has been observed in cholangiocarcinomas (Boulter et al. (2015) J. Clin. Invest. 125, 1269-1285 LID-1210.1172/JCI76452 [doi] LID-76452 [pii]), and is driven by upstream mutations in the pathway. For instance, inactivating mutations in the tumor suppressor E3 ubiquitin ligases ZNRF3 and RNF43, which are frequently mutated genes in colorectal and endometrial cancers Giannakis et al. (2014) Nat Genet 46, 1264-1266), lead to stabilization and higher levels of frizzled receptors at the cell surface (Jiang et al. (2015) Molecular Cell 58, 522-533). Treatment with C59, a small molecule inhibitor of global Wnt palmitoylation and secretion, prevents the growth Rnf43^{-/-} (deficient in RING finger protein 43) and Znf3^{-/-} (deficient in zinc/RING finger protein 3) intestinal neoplasia in mice, highlighting a role for upstream Wnt signaling in these tumors (Koo et al. (2015) Proceedings of the National Academy of Sciences 112, 7548-7550). It is noteworthy that no significant adverse effects on adjacent intestinal crypts were observed upon treatment with C59. While these findings suggest that upstream inhibition of the Wnt/FZD interaction may be tolerated in a highly proliferating stem cell compartment such as the gut, it remains important to evaluate in detail the general toxic side effects of Wnt pathway inhibition in future studies. In sum, the above findings highlight the biological role of FZDs in cancer and indicate that pharmacological targeting of the FZD receptors could be an attractive approach for inhibiting the Wnt pathway upstream at the cell surface.

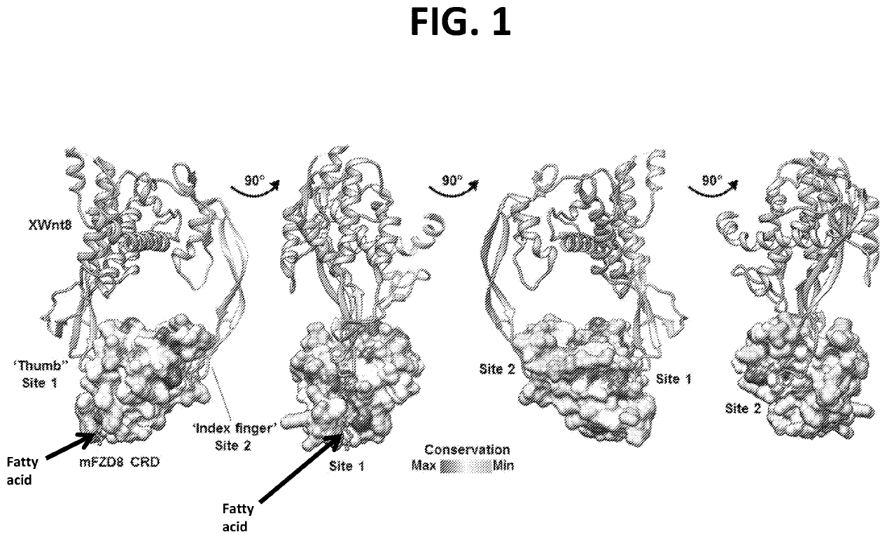

Molecular understanding of the FZD receptor protein family has been limited to the X-ray crystal structures of human (h) FZD4, 5 and 7 CRDs as well as mouse (m) FZD8 CRD. Although the first X-ray ω-crystal structure of Xenopus Wnt8 in complex with mFZD8 CRD was reported, the structural basis for the specificity of Wnt-FZD interactions (Dijksterhuis et al. (2015) J Biol Chem 290, 6789-6798) remains poorly understood. Despite a good structural understanding of Wnt-FZD interactions, it has been challenging to develop high-affinity small ligands that bind at the lipid-binding groove and disrupt FZD-dependent signaling. Selective targeting of specific FZD receptors is also a challenge, due to a high degree of sequence similarity. See FIG. 1; Lee et al. (2015) J Biol Chem 290, 30596-30606; Gurney et al. (2012) Proc Natl Acad Sci USA 109, 11717-11722. FIG. 1 shows the crystal structure of Xenopus (X) Wnt8 (in ribbon representation; fatty acid pointed out with arrow) in complex with mouse (m) FZD8 CRD (surface representation). XWnt8 interacts with mFZD8 CRD at two distinct sites. Site one (the “thumb”) is primarily a lipid-protein interface between the Wnt fatty acyl group and the hydrophobic groove on mFZD8 CRD, whereas site two (the “index finger”) is composed of a protein-protein interaction interface. mFZD8 CRD crystallized as a monomer in complex with XWnt8. Clustal Omega (Sievers, F. et al. Fast, scalable generation of high-quality protein multiple sequence alignments using Clustal Omega. Mol. Sys. Biol. 7, 539 (2011)) sequence alignment of all human FZD CRDs were scored for their percent conservation, which was applied as a color gradient on the surface of mFZD8 CRD as a surrogate structure (dark gray, high conservation; light gray, low conservation). Glycosylation groups are hidden for clarity.

In order to investigate the role of FZD7 in intestinal stem cell function, experiments were performed to identify ligands that selectively bind FZD7 CRD. A naïve linear peptide library (4-16 amino acids in length) and a cyclic peptide library were screened via phage display against FZD7 CRD-Fc. Peptides found to bind FZD7 CRD-Fc were then screened via spot phage ELISA against FZD7 CRD-Fc, FZD8 CRD-Fc, and Herceptin to identify candidates that bind FZD7 CRD but not FZD8 CRD or herceptin. The amino acid sequences of candidate FZD7 CRD-binding linear peptides are shown in Table 3 below, and the amino acid sequences of candidate FZD7 CRD-binding cyclic peptides are shown in Table 4.

TABLE 3

| SEQ ID | PEPTIDE | PEPTIDE | | SIGNAL: NOISE RATIO^{B} | | |
|---|---|---|---|---|---|---|
| NO | SEQUENCE | ID | N^{A} | FZD7 CRD | HERCEPTIN | FZD8 CRD |
| 1 | YEHLHDLMDLIRPW | Fz7-07 | 20 | 23.2 | 1.2 | 1.6 |
| 2 | TYFDDICNLILPWANP | | 1 | 27.5 | 1.2 | 1.4 |
| 3 | PQDLLDWCHYMIVSSD | | 1 | 29.1 | 1.2 | 1.3 |
| 4 | ACSYVIDLWNQCLT | | 4 | 26.8 | 1.1 | 3.1 |
| 5 | PCSVICLPDWSSLLFI | | 1 | 22.7 | 1.0 | 1.5 |
| 6 | DTDLHQWCLWFT | | 21 | 30.2 | 1.1 | 9.3 |
| 7 | FWMLLQEGFAFWFP | | 1 | 30.5 | 1.3 | 6.4 |
| 8 | FELLLDLGDLIRLW | | 1 | 26.9 | 1.8 | 2.0 |
| 9 | ACSYVIDLWNLCLR | | 1 | 27.6 | 1.3 | 1.5 |
| 10 | ASELHDWCRMMFPW | | 3 | 28.9 | 1.2 | 1.3 |
| 11 | ISLIEAMIALDRVF | Fz7-06 | 7 | 27.0 | 1.1 | 25.8 |
| 12 | PPNVHEGCWSMFPW | | 1 | 28.1 | 1.1 | 1.3 |
| 13 | LPSDDLEFWCHVMY | Fz7-21 | 3 | 24.5 | 1.0 | 1.1 |
| 14 | DTDLLQWCLWFT | | 1 | 31.1 | 1.2 | 6.0 |
| 15 | FWMQLQDGFAIWFP | | 27 | 27.8 | 1.7 | 2.9 |
| 16 | PCSVICLPDWSSLLFI | | 2 | 5.0 | 1.1 | 1.1 |
| 17 | GDFWPGSLLWEILV | | 3 | 4.4 | 1.2 | 1.0 |
| 18 | ILTFEYFWILGLIL | | 5 | 4.5 | 1.1 | 1.1 |
| 19 | LPLFFLSYVL | | 5 | 6.3 | 1.1 | 1.1 |
| 20 | FLPDQHSHLFLPWGEP | | 1 | 5.3 | 1.1 | 1.0 |
| 21 | SCQMWSNLRVLFLSYW | | 1 | 1.0 | 0.7 | 0.7 |
| 22 | VFVPFSELTSLC | | 1 | 7.8 | 1.1 | 1.0 |
| 23 | IWFKGRFVEFSSLV | Fz7-17 | 4 | 7.7 | 1.1 | 1.0 |
| 24 | NAFWRDQCLEWFIICL | | 4 | 5.5 | 1.2 | 1.1 |
| 25 | EHDLLLRAMNSFVLIF | | 1 | 7.6 | 1.4 | 1.0 |
| 26 | FCENPYIICW | | 1 | 8.6 | 1.1 | 0.8 |
| 27 | NPPPECFLSK | | 1 | 5.2 | 1.2 | 1.1 |
| 28 | VFFYHSLFFIKLILDP | | 1 | 4.7 | 1.3 | 1.3 |
| 29 | ERRVCYPWFEVSQP | | 1 | 2.6 | 1.3 | 1.2 |
| 30 | LSSGKKVSSYWFNFWF | | 1 | 3.5 | 3.8 | 1.0 |
| 31 | FWFDFWFG | | 1 | 4.2 | 4.1 | 0.8 |

^{A}“N” refers to the occurrence of each peptide.
^{B}For “SIGNAL: NOISE RATIO.” “Signal” is the spot phage ELISA signal detected against FZD7 CRD, Herceptin, or FZD 8 CRD. “Noise” is the ELISA signal against BSA.

TABLE 4

| SEQ ID | PEPTIDE | PEPTIDE | | SIGNAL: NOISE RATIO^{C} | | |
|---|---|---|---|---|---|---|
| NO | SEQUENCE^{A} | ID | N^{B} | FZD7 CRD | HERCEPTIN | FZD8 CRD |
| 32 | SSDFSG [symbol] LSW [symbol] DLIFG | | 1 | 28.4 | 1.22 | 1.39 |
| 33 | FDF [symbol] SVMPQFIY [symbol] PGD | Fz7-20 | 11 | 26.0 | 1.14 | 2.10 |
| 34 | HLSDV [symbol] SDW [symbol] DLVFW | | 1 | 16.1 | 0.99 | 1.09 |
| 35 | TSDFSW [symbol] LSW [symbol] DLIFW | | 79 | 28.4 | 1.19 | 1.27 |
| 36 | FDF [symbol] TVMPHFIY [symbol] PGD | | 1 | 27.4 | 1.13 | 2.01 |
| 37 | FDF [symbol] SVMPHFIY [symbol] PGD | | 1 | 27.5 | 1.10 | 1.12 |
| 38 | HLSDVF [symbol] SDW [symbol] DLVFW | | 1 | 17.5 | 1.14 | 1.28 |

^{A}Underlined cysteine residues may form disulfide bonds.
^{B}“N” refers to the occurrence of each peptide.
^{C}For “SIGNAL: NOISE RATIO,” “signal” is the spot phage ELISA signal detected against FZD7 CRD, Herceptin, or FZD 8 CRD, and “noise” is the ELISA signal against BSA.

Five peptides, Fz7-06, Fz7-07, Fz7-17, Fz7-20, and Fz7-21, were selected for chemical synthesis and further characterization.

Shotgun alanine scanning was performed on Fz7-21 to identify the amino acid residues in the peptide that are critical for binding to FZD7 CRD. Briefly, sixty Fz7-21-derived peptides (see Table 5), each containing at least one random alanine and/or valine/and/or aspartic acid and/or serine substitution, were generated and screened via spot phage ELISA (i.e., as described above) against FZD7 CRD-Fc and Herceptin. As shown in Table 5, most of the alanine-, aspartic acid-, serine- or valine-substituted Fz7-21-derived peptides demonstrated FZD7 CRD binding activity.

TABLE 5

| SEQ ID | PEPTIDE | PEPTIDE | | | | | |
|---|---|---|---|---|---|---|---|
| NO | ID | SEQUENCE | FZD7 CRD-Fc | | HERCEPTIN | | |
| 13 | Fz7-21 | LPSDDLEFWCHVMY | S/W^{A} | S/N^{B} | S/W^{A} | S/N^{B} | N^{C} |
| 39 | | VAADDLAAWCHVMY | 3.4 | 60.7 | 0.0 | 1.4 | 1 |
| 40 | | AASDDLEFWCHVMY | 3.2 | 60.5 | 0.0 | 1.4 | 5 |
| 41 | | AASDDLEFWCHVMY | 3.0 | 59.6 | 0.0 | 1.3 | 1 |
| 42 | | APSDDVAFWCHVMY | 3.1 | 58.6 | 0.0 | 1.6 | 1 |
| 43 | | APADDVEFWCHVMY | 3.1 | 58.6 | 0.0 | 1.8 | 1 |
| 44 | | APSDDLEFWCHVMY | 3.1 | 58.3 | 0.0 | 1.3 | 2 |
| 45 | | APADDLEAWCHVMY | 3.1 | 58.1 | 0.0 | 1.2 | 1 |
| 46 | | APSDDLEFWCHAMY | 3.1 | 57.2 | 0.0 | 1.3 | 1 |
| 47 | | VASDDLEAWCHVMY | 3.1 | 57.0 | 0.0 | 1.4 | 1 |
| 48 | | AAADDLEFWCHVMY | 3.1 | 56.1 | 0.0 | 1.2 | 2 |
| 49 | | AASDDLAAWCHVMY | 3.0 | 55.7 | 0.0 | 1.8 | 1 |
| 50 | | AASDDLESWCHVMY | 2.9 | 55.6 | 0.0 | 1.2 | 1 |
| 51 | | APADDLAFWCHVMY | 3.0 | 55.1 | 0.0 | 1.3 | 2 |
| 52 | | LPADDLAVWCDVMY | 3.1 | 54.9 | 0.0 | 1.5 | 1 |
| 53 | | LPSDDLESWCHVMY | 3.0 | 54.5 | 0.0 | 1.2 | 1 |
| 54 | | AAADDLEVWCHVMY | 3.1 | 54.4 | 0.0 | 1.4 | 1 |
| 55 | | VAADALEFWCHVMY | 3.0 | 54.2 | 0.0 | 1.3 | 1 |
| 56 | | APSDDLAAWCHVVY | 2.9 | 54.0 | 0.0 | 1.2 | 1 |
| 57 | | AAADDLAAWCDVMY | 3.1 | 54.0 | 0.0 | 1.4 | 1 |
| 58 | | VASDDLEFWCHVMY | 3.2 | 54.0 | 0.0 | 1.3 | 2 |
| 59 | | AAADDLEAWCAVMY | 3.1 | 53.6 | 0.0 | 1.2 | 1 |
| 60 | | LAADDLESWCHVMY | 3.0 | 53.5 | 0.0 | 1.2 | 1 |
| 61 | | APADDLASWCHVMY | 3.2 | 53.5 | 0.0 | 1.3 | 1 |
| 62 | | VASDDLASWCHAMY | 3.0 | 53.5 | 0.0 | 1.3 | 1 |
| 63 | | VPADDLASWCHVMY | 3.1 | 53.4 | 0.0 | 1.2 | 1 |
| 64 | | APADDLEFWCHVVY | 3.1 | 53.2 | 0.0 | 1.4 | 1 |
| 65 | | VPSDDLAFWCHVMY | 3.0 | 53.2 | 0.0 | 1.3 | 2 |
| 66 | | VPADALAVWCDVMY | 2.9 | 53.0 | 0.0 | 1.3 | 1 |
| 67 | | VPADDLAFWCHVMY | 3.1 | 52.7 | 0.0 | 1.3 | 3 |
| 68 | | VPSDDLASWCHVMY | 3.1 | 52.2 | 0.0 | 1.4 | 1 |
| 69 | | VPSADLESWCHVMY | 2.9 | 52.2 | 0.0 | 1.6 | 1 |
| 70 | | AASDDLEAWCHVMY | 3.1 | 52.1 | 0.0 | 1.4 | 1 |
| 71 | | APSDDLASWCHVMY | 3.3 | 52.0 | 0.0 | 1.1 | 1 |
| 72 | | APADDLEFWCHVMY | 3.0 | 51.6 | 0.0 | 1.2 | 2 |
| 73 | | APSDDLAAWCHVMY | 3.0 | 51.3 | 0.0 | 1.3 | 1 |
| 74 | | APADDLAFWCHVVY | 3.2 | 50.6 | 0.0 | 1.3 | 1 |
| 75 | | APSDDLEAWCDVMY | 2.7 | 50.2 | 0.0 | 1.3 | 1 |
| 76 | | VPSDDLEAWCDVMY | 2.7 | 49.8 | 0.0 | 1.3 | 1 |
| 77 | | AASDDLAFWCHVMY | 3.1 | 49.7 | 0.0 | 1.1 | 5 |
| 78 | | VPADDLASWCDVMY | 2.9 | 49.2 | 0.1 | 1.9 | 1 |
| 79 | | LPSADLESWCHVMY | 2.9 | 48.6 | 0.0 | 1.4 | 1 |
| 80 | | AAADDLAFWCHVMY | 3.1 | 48.2 | 0.0 | 1.4 | 3 |
| 81 | | LASDDLEFWCHVMY | 3.0 | 48.1 | 0.0 | 1.2 | 2 |
| 82 | | LPAADLAAWCHVMY | 3.0 | 48.0 | 0.0 | 1.2 | 1 |
| 83 | | VPSADLETWCHVMY | 2.8 | 47.3 | 0.0 | 1.4 | 1 |
| 84 | | LPADDLAAWCHVMY | 3.1 | 46.7 | 0.0 | 1.4 | 1 |
| 85 | | PPADDLAFWCDVMY | 2.9 | 44.9 | 0.0 | 1.1 | 1 |
| 86 | | VASDDLASWCHVVY | 2.5 | 44.8 | 0.0 | 1.2 | 1 |
| 87 | | AAADDVASWCHVMY | 2.3 | 44.5 | 0.0 | 1.3 | 1 |
| 88 | | VAADDLAFWCDVMY | 3.0 | 40.7 | 0.0 | 1.2 | 1 |
| 89 | | APADDLEFWCHAMY | 1.9 | 36.3 | 0.0 | 1.4 | 1 |
| 90 | | APADDLAFWCDVMY | 3.0 | 33.8 | 0.0 | 0.9 | 1 |
| 91 | | APSDDLAFWCDVMY | 3.1 | 27.4 | 0.0 | 0.7 | 1 |
| 92 | | LPADDLAFWCDVMY | 2.8 | 23.8 | 0.0 | 0.7 | 1 |
| 93 | | AAADDLAFWCDVMY | 2.7 | 8.5 | -0.2 | 0.4 | 1 |
| 94 | | LPADDLEFWCHVMY | 0.1 | 2.8 | 0.0 | 1.2 | 1 |
| 95 | | VPSDDLEFWCAVMY | 0.1 | 2.1 | 0.0 | 1.2 | 1 |
| 96 | | APADDLESWCHVMY | 2.9 | 53.9 | 0.1 | 2.1 | 1 |
| 97 | | APSDDLAFWCHVVY | 2.5 | 45.0 | 0.1 | 2.1 | 1 |
| 98 | | AAADDLAAWCHVVY | 2.7 | 50.8 | 0.1 | 2.2 | 1 |

^{A}“S/W” value is the signal minus the noise, where “signal” refers to the spot phage ELISA signal detected against FZD8 CRD-FC or Herceptin immobilized on the 384-well Maxisorp plate, and where “noise” refers to the ELISA
signal against BSA on the same plate.
^{B}“S/N” value is the signal to noise ratio
^{C}“N” refers the occurrence of each peptide.

Next, a “W/A” value was determined for each amino acid position in peptide Fz7-21. “W/A” was calculated by dividing the number of Fz7-21-derived peptides having a wild-type (WT) residue at a particular acid position by the number of Fz7-21-derived peptides having a substitution at that position. (If no substitutions were found at a certain amino acid position, then A=1.) Data were collected from 60 peptide variants. The “W/A” value represents the peptide binding-dependence of each wild-type Fz7-21 residue for interaction with FZD7 CRD. Residues that play a critical role in the binding of Fz7-21 to FZ7 CRD were defined as having a W/A≥25 and categorized as “class 2” residues. Residues that play an important role in the binding of Fz7-21 to FZ7 CRD were defined as having a W/A value between 6 and 19 (i.e., 5≤W/A<25) and categorized as “class 1” residues. Dispensable residues were defined as having a W/A<5 and were categorized as “class 0” residues. As shown in Table 6 below, D5, L6, W9, C10, M13, and Y14 in Fz7-21 were determined to be critical for binding to FZ7 CRD; D4, H11, and V12 in Fz7-21 were determined to be important for binding to FZ7 CRD; and L1, P2, S3, E7, and F8 in Fz7-21 were found to be dispensable.

TABLE 6

| Position | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Fz7-21 Sequence | L | P | S | D | D | L | E | F | W | C | H | V | M | Y |
| W/A | 0.3 | 1.7 | 0.9 | 14 | 29 | 57 | 0.8 | 2.0 | 60 | 60 | 23 | 14 | 54 | 60 |
| Residue Class | 0 | 0 | 0 | 1 | 2 | 2 | 0 | 0 | 2 | 2 | 1 | 1 | 2 | 2 |

A derivative of the Fz7-21 peptide containing a D5N substitution, i.e., Fz7-21N, was synthesized to probe the contribution of the charged aspartic acid residue at amino acid position 5 in Fz7-21 on FZD7 CRD binding.

The functional activities of the peptide were characterized in a series of cell-based assays. In one set of assays, the effects of Fz7-06, Fz7-07, Fz7-17, Fz7-20, Fz7-21, and Fz7-21N on Wnt-signaling was tested in HEK293-TB cells. Briefly, HEK293-TB cells (i.e., cells that had been stably transfected with a TCF/LEF-responsive firefly luciferase reporter construct and a constitutively expressed Renilla luciferase construct) were stimulated with recombinant mWnt3a (50 ng/mL) in 3-fold serial dilutions of Fz7-06, Fz7-07, Fz7-17, Fz7-20, Fz7-21, or Fz7-21N for 6 hours. DMSO controls were performed in parallel. β-catenin signaling was measured as the ratio of Firefly luciferase to Renilla luciferase normalized to the DMSO control. Results are shown in Table 7. The values in Table 7 are averages of at least 3 independent replicates, ±standard deviation. The most potent peptide, Fz7-21 (SEQ ID NO: 13), impaired Wnt3a-mediated β-catenin signaling in HEK293 cells with an IC₅₀ between about 90-100 nM (see Table 7). Fz7-21N impaired Wnt3a-mediated β-catenin signaling in HEK293 cells stimulated with exogenous Wnt3a (IC₅₀=100 nM.

In a complementary set of experiments, the effects of Fz7-06, Fz7-07, Fz7-17, Fz7-20, Fz7-21, and Fz7-21N on Wnt-mediated stabilization of β-catenin protein in mouse L-cells (as described in Hannoush (2008) PLoS One 3, e3498). The L cells were treated with Wnt3a (2.5 nM) and in 3-fold serial dilutions of Fz7-06, Fz7-07, Fz7-17, Fz7-20, Fz7-21, or Fz7-21N for 5 hours. DMSO controls were performed in parallel. Following treatment, the L-cells were fixed, permeabilized, probed with fluorescently labeled anti-β-catenin antibodies, and observed via in-cell western assay (i.e., as described in Hannoush, R. N. (2008) PLoS One 3, e3498) to assess β-catenin protein levels. Results are shown in Table 7. The values in Table 7 are averages of at least 3 independent replicates, ±standard deviation. Fz7-21 blocked Wnt3a-mediated stabilization of β-catenin in mouse L cells, indicating that this peptide inhibits Wnt signaling. Fz7-21N blocked Wnt3a-mediated stabilization of β-catenin in mouse L cells, indicating that this peptide inhibits Wnt signaling.

TABLE 7

[symbol]

| | HEK293S-TB^{A,D} | | L CELLS^{B,E} | | PEPTIDE |
|---|---|---|---|---|---|
| | | % Remaining | | % Remaining | SEQUENCE |
| PEPTIDE | IC50 (μM) | Activity^{C} | IC50 (μM) | Activity^{C} | (SEQ ID NO) |
| Fz7-06 | 10.45 ± 0.56 | 4.70 ± 2.07 | n. i. | 174.95 ± | ISLIEAMIALDRVF |
| | | | | 16.83 | (SEQ ID NO: 11) |
| Fz7-07 | >100 | 46.04 ± 6.94 | >100 | 67.20 ± 10.22 | YEHLHDLMDLIRPW |
| | | | | | (SEQ ID NO: 1) |
| Fz7-17 | 34.77 ± 0.34 | 4.59 ± 1.30 | ~35 | 38.20 ± 0.06 | IWFKGRFVEFSSLV |
| | | | | | (SEQ ID NO: 23) |
| Fz7-20 | 20.81 ± 1.61 | 9.78 ± 0.53 | 15.22 ± 10.83 | 23.68 ± 4.40 | FDF [symbol] SVMPQFIY [symbol] PGD |
| | | | | | (SEQ ID NO: 33) |
| Fz7-21 | 0.10 ± 0.05 | 5.15 ± 2.88 | 0.05 ± 0.03^{F} | 50.98^{G} ± 3.76 | LPSDDLEFWCHVMY |
| | | | | | (SEQ ID NO: 13) |
| Fz7-21N | 0.41 ± 0.16 | 5.51 ± 0.13 | 0.18 ± 0.12^{F} | 59.77^{G} ± 6.12 | LPSDNLEFWCHVMY |
| | | | | | (SEQ ID NO: 108) |

[symbol]

Values represent the mean ± standard deviation.
>100 μM indicates that the peptide showed partial inhibition at the highest concentration of peptide tested (100 μM)
^{A}Measured by readout from a TOPbrite reporter containing the β-catenin region of TCF
^{B}Measured by amount of β-catenin detected via immunofluorescence
^{C}Measured at 100 μM peptide
^{D}Value is an average of at least 2 replicates; ± standard deviation
^{E}Value is an average of at least 3 replicates; ± standard deviation
^{F}β-catenin activity reaches a plateau of ~50% inhibition at 11.11 μM of peptide
^{G}Measured at 11.11 μM of peptide

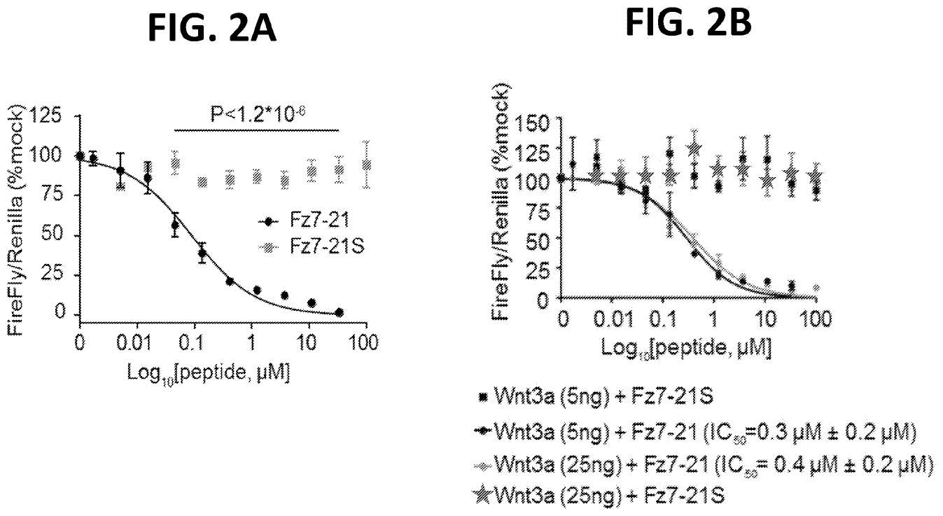

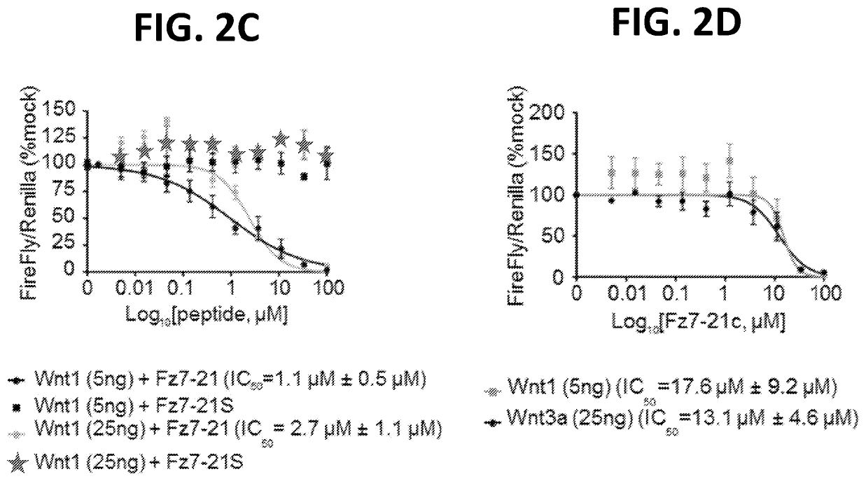

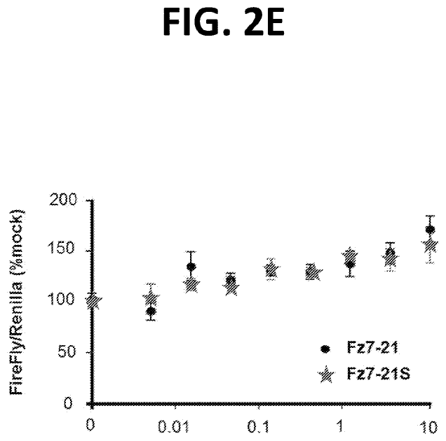

Without being bound by theory, the observed difference in maximum Wnt pathway inhibition between HEK293 and mouse L cells, as measured by TOPbrite luciferase reporter and β-catenin imaging assays, respectively, could likely be due to differences in cell surface expression levels of various FZD receptors between the two cell lines (Zhou et al. (2014) Dev. Cell 31, 248-256 LID-210.1016/j.devce1.2014.1008.1018 [doi] LID-S1534-5807(1014)00548-00546 [pii]). Fz7-21S, i.e., a derivative of Fz7-21 containing a C10S substitution, was synthesized to probe the contribution of the cysteine residue at amino acid position 10 in Fz7-21 on FZD7 CRD binding. The amino acid sequence of Fz7-21S is LPSDDLEFWSHVMY (SEQ ID NO: 113). In a first assay HEK293-TB cells were stimulated with recombinant mWnt3a (50 ng/mL) in 3-fold serial dilutions of Fz7-21, Fz7-21S, or a DMSO control for 6 hrs. As shown in FIG. 2A, Fz7-21 inhibited Wnt3a-stimulated β-catenin signaling with an IC₅₀ of 93.7 nM±27.9 nM. Fz7-21S was not found to inhibit Wnt3a signaling.

In a subsequent assay, HEK293-TB cells were transfected with 5 ng pCDNA3.2-Wnt3a or 25 ng pCDNA3.2-Wnt3a. After 24 hours, the transfected cells were treated with 3 fold serial dilutions of Fz7-21, Fz7-21S, or DMSO for 6 hours. As shown in FIG. 2B, Fz7-21 inhibited Wnt3a-stimulated β-catenin signaling with an IC₅₀ of 329.8 nM±157 nM in cells transfected with 5 ng pCDNA3.2-Wnt3a and an IC50 of 419.3 nM±168.6 nM in cells transfected with 25 ng pCDNA3.2-Wnt3a. Fz7-21S was not found to inhibit Wnt3a signaling.

In a further assay, HEK293-TB cells were transfected with 5 ng pCDNA3.2-Wnt1 or 25 ng pCDNA3.2-Wnt1. After 24 hours, the transfected cells were treated with 3 fold serial dilutions of Fz7-21, Fz7-21s, or DMSO for 6 hours. As shown in FIG. 2C, Fz7-21 inhibited Wnt1-stimulated β-catenin signaling with an IC₅₀ of 1.0864 μM±0.5077 μM in cells transfected with 5 ng pCDNA3.2-Wnt3a and an IC₅₀ of 2.661 μM±1.124 μM in cells transfected with 25 ng pCDNA3.2-Wnt3a. Fz7-21S was not found to inhibit Wnt1 signaling.

Next, HEK293-TB cells were transfected with 5 ng pCDNA3.2-Wnt1 or 25 ng pCDNA3.2-Wnt3a. After 24 hours, the transfected cells were treated with 3-fold serial dilutions of Fz7-21C, i.e., an Fz7-21-derived peptide containing a D-cysteine stereoisomer at position 10, for 6 hours. As shown in FIG. 2D, the substitution of an L-cysteine with a D-cysteine at position 10 of Fz7-21 reduced the potency of Wnt1 inhibition by 16-fold, and reduced the potency of Wnt3a inhibition by 31-fold.

Taken together, the results shown in FIGS. 2A-2D highlight the functional contribution of the Cys10 residue on FZD7 CRD binding.

An epistasis study was carried out in HEK293-TB cells treated with 6-BIO (6-bromoindirubin-3′-oxime), an inhibitor of GSK-3α/β that stabilizes β-catenin and activates downstream Wnt signaling independent of receptor activation (Meijer et al. (2003) Chemistry & Biology 10, 1255-1266). HEK293-TB cells were stimulated with 10 μM 6-BIO in the presence of 3-fold serial dilutions of Fz7-21, Fz7-21S, or DMSO for 6 hr. Neither Fz7-21 nor Fz7-21S inhibited Wnt signaling in 6-BIO-treated cells (see FIG. 2E). Such result indicates that Fz7-21 acts upstream of GSK3α and β-catenin, likely at the level of the FZD receptor.

In FIGS. 2A-2E, β-catenin signaling was measured as the ratio of Firefly to Renilla normalized to the DMSO control. The calculated values were background subtracted (cells not stimulated by Wnt3a), normalized to DMSO-treated samples, and represent the mean±s.e.m of at least three independent experiments with technical triplicates. Inhibition curves were generated using Graphpad Prism (v6.05) using the log(inhibitor) vs. normalized response [variable slope equation (Y=100/(1+10\hat{ }((Log IC50−X)*HillSlope))]. IC₅₀s represent the mean±95% confidence interval. All samples maintained 1% final DMSO concentration. Statistical significance was determined using the Holm-Sidak method, with alpha=5% where P*<1.17*10⁻⁶. Significance assumes that all samples are from populations with the same scatter.

Next, a 5-caboxyfluorescien-labeled version of the Fz7-21 peptide, 5FAM-Fz7-21, was generated and tested for binding to hFZD1 CRD-Fc, mFZD2 CRD-Fc, hFZD4 CRD-Fc, hFZD5 CRD-Fc, hFZD7 CRD-Fc, mFZD7 CRD-Fc, hFZD8 CRD-Fc, mFZD9 CRD-Fc, and hFZD10 CRD-Fc. Control experiments using 5FAM-Fz7-21S were performed in parallel. 5FAM-Fz7-21 or 5FAM-Fz721S was incubated with each of the aforementioned FZD CRD-Fcs overnight at 4° C. in PBS and resolved via SEC. 5FAM-Fz7-21 showed selective and preferential binding to hFZD1 CRD-Fc, hFZD2 CRD-Fc, hFZD7 CRD-Fc, and mFZD7 CRD-Fc, with affinities in the low nM range (39-116 nM).

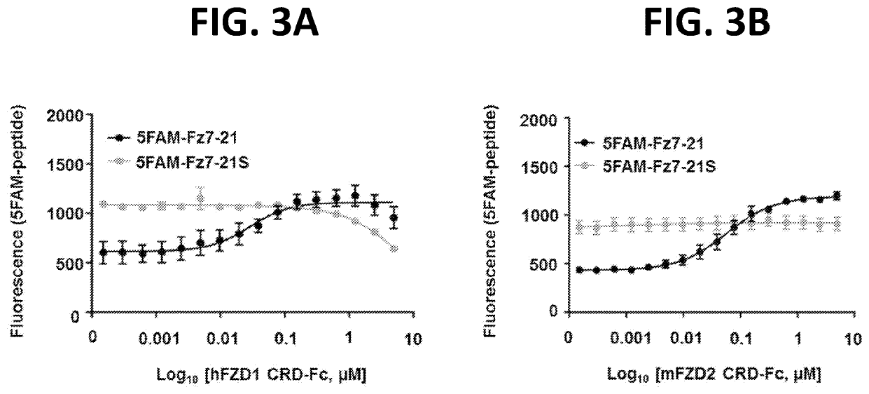

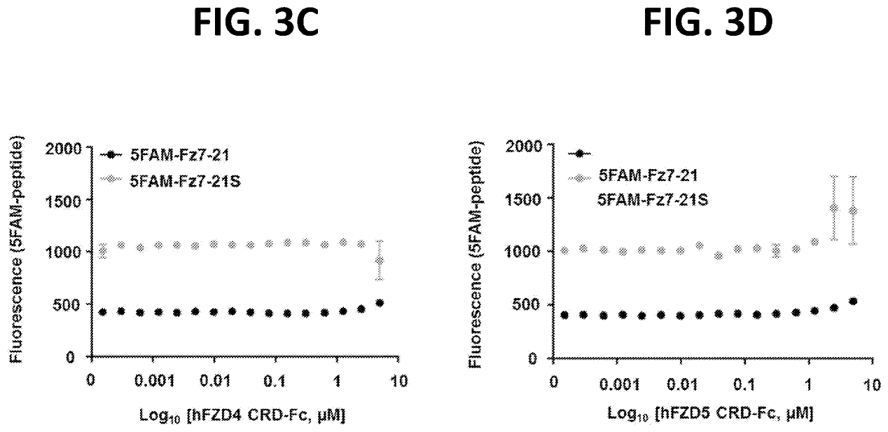

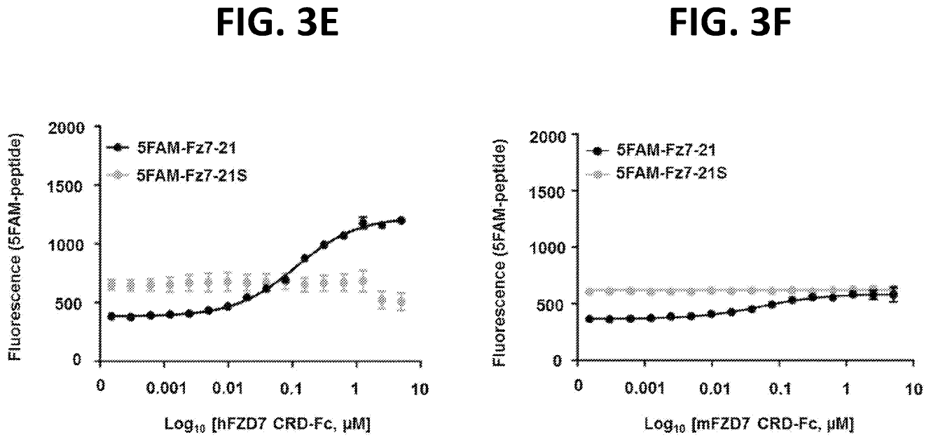

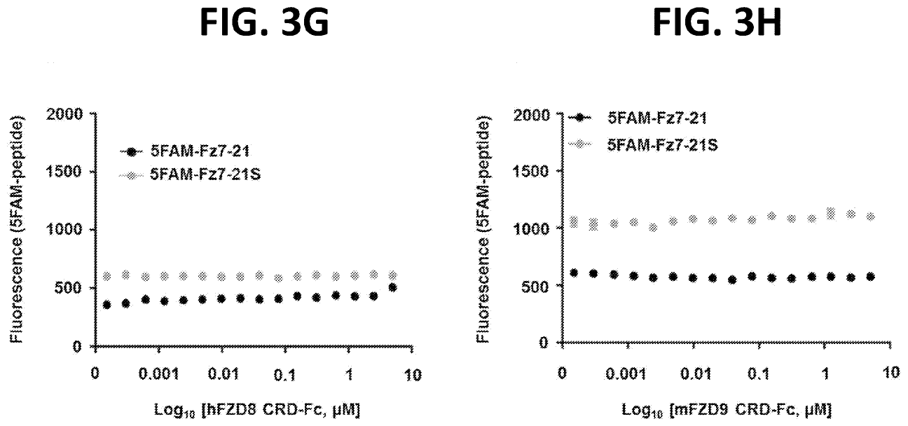

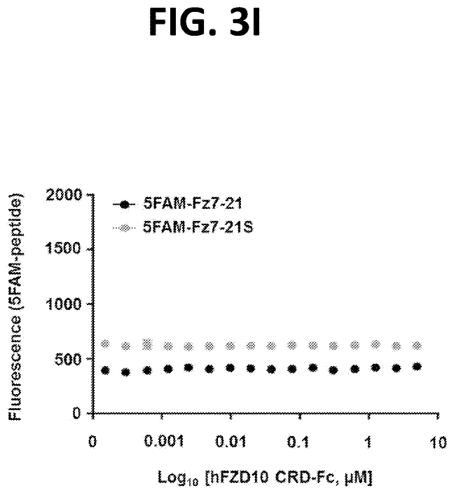

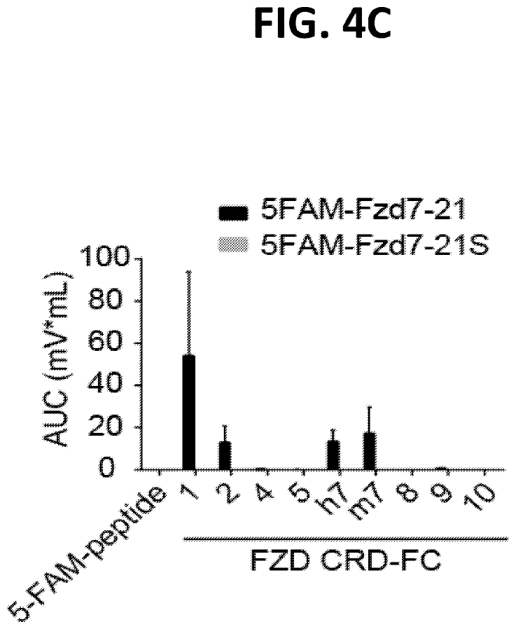

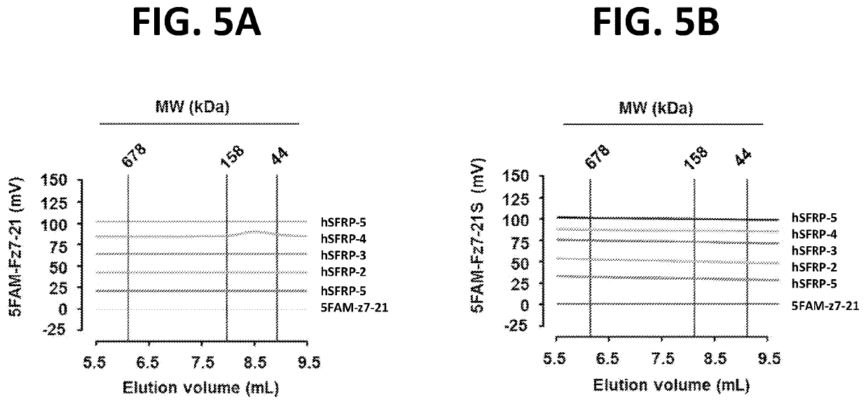

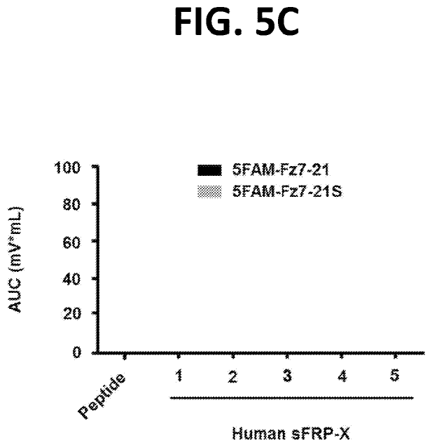

See Table 8 and FIGS. 3A-3I. In FIGS. 3A-3I, 5FAM-Fz7-21 or 5FAM-Fz7-21S (50 nM in PBS) was incubated with increasing concentration of FZD CRD-Fc (2-fold serial dilutions), and fluorescence intensity was measured using either a Monolith NT.115 or NT.115Pico instrument (NanoTemper Technologies). EC50 values represent the 95% confidence interval of at least three independent experiments, excepting hFZD1 CRD-Fc+5FAM-Fz7-21S, which was done twice. EC₅₀ ovalues were calculated with Prism Graphpad using the Log(agonist) vs. response [variable slope (four parameters) function: Y=Bottom+(Top-Bottom)/(1+10\hat{ }((Log EC−X)*HillSlope))]. Plotted values represent the mean±s.e.m.

TABLE 8

| FZD CRD- | 5FAM-Fz7-21 | 5FAM-Fz7-21S |
|---|---|---|
| FC | EC₅₀ (nM) | EC₅₀ |
| hFzd1 | 39 ± 26 | >10 μM |
| mFzd2 | 60 ± 18 | NC^{A} |
| hFzd4 | NC^{A} | NC^{A} |
| hFzd5 | NC^{A} | NC^{A} |
| hFzd7 | 116 ± 20 | NC^{A} |
| mFzd7 | 67 ± 39 | NC^{A} |
| hFzd8 | NC^{A} | NC^{A} |
| mFzd9 | NC^{A} | NC^{A} |
| hFzd10 | NC^{A} | NC^{A} |

^{A}NC = no change

Consistent with the hypothesis that Fz7-21 specifically binds to CRDs of the FZD7 subclass, little to no binding of 5FAM-Fz7-21 to FZD5, FZD8, FZD9 and FZD10 CRDs was observed. The control peptide 5FAM-Fz7-21S showed no detectable binding to any members of the FZD CRD family. See FIGS. 3A-3I and Table 8.

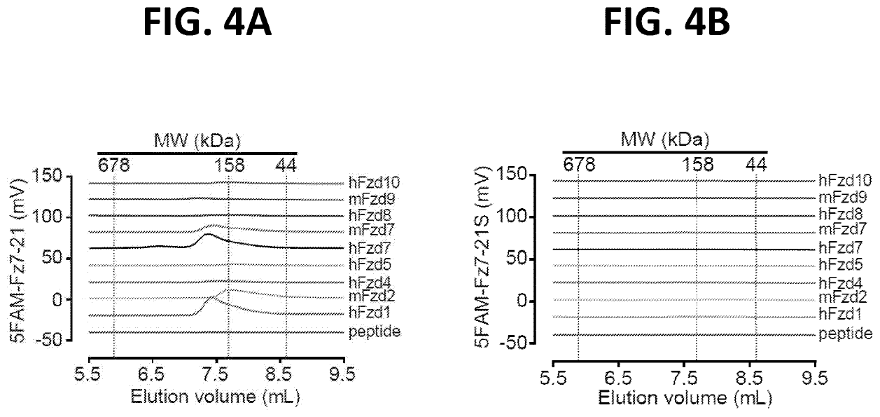

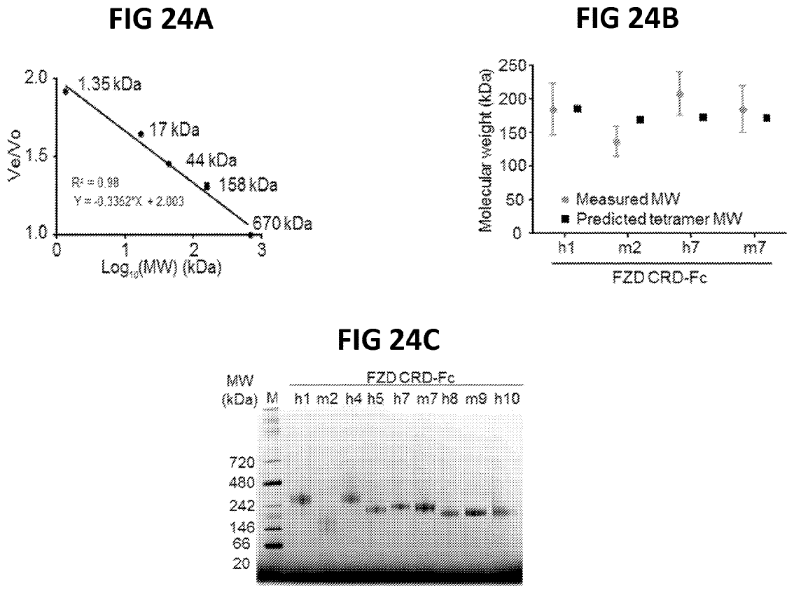

Selective targeting of the FZD7 receptor subclass was also observed chromatographically. Complexes of 5FAM-Fz7-21 with FZD1 CRD-Fc, FZD2 CRD-Fc, and FZD7 CRD-Fc could be resolved by fluorescence size-exclusion chromatography (FSEC). By contrast, no complex was observed with 5FAM-Fz7-21S. See FIGS. 4A and 4B, which provide representative fluorescence traces of 1 μM 5FAM-Fz7-21 and 1 μM 5FAM-Fz7-21S, respectively, incubated with various FZD CRD-Fc proteins (250 nM) overnight at 4° C. prior to analysis by FSEC. Vertical dotted lines represent the elution volume of molecular weight standards. Molar stoichiometry of FZD7 CRD to peptide is indicated. Quantification of fluorescence intensity (area under curve, AUC) for FIGS. 4A and 4B are provided in FIG. 4C. The results in FIGS. 4A-4C indicate that 5FAM-Fz7-21 preferentially binds to FZD7-class CRDS, whereas 5FAM-Fz7-21S does not bind any FZD CRD-Fc. In these experiments, the FZD CRD Fc-fusion constructs showed a retention profile consistent with the formation of tetramers comprising two FZD CRD dimers held together by two Fc fragments. In FIG. 24A, molecular weight (MW) standards analyzed by UV absorption were plotted as a function of elution volume (Ve) over void volume (Vo). Values represent the mean±s.e.m. of three independent experiments. FIG. 24B shows the observed molecular weights of FZD CRD-Fc proteins bound to 5FAM-Fz7-21 (gray circles) vs. the predicted FZD CRD-Fc tetrameric MW (black squares). Measured values represent the mean±standard deviation (SD) of three independent experiments. FIG. 24C shows a native PAGE (4-16%) of different FZD CRD-Fc proteins used (˜2 μg). NativMark was used as the molecular weight standard. Protein samples were diluted with native PAGE sample buffer, separated using dark blue cathode running buffer, resolved, and fixed according to the manufacture's recommendation. Binding to recombinant FZD3 and 6 CRDs could not be assessed due to challenges with the expression of these proteins. However, by flow cytometry analysis, 5FAM-Fz7-21 showed predominant binding to FZD7 with minimal binding to FZD4, 5 or 6 receptors that were stably expressed in HEK293 cells, consistent with the chromatographic experiments discussed above.

Further FSEC experiments were performed to determine whether 5FAM-Fz7-21 or 5FAM-Fz7-21S bind human sFRP1, sFRP2, sFRP3, sFRP4, or sFRP5 (i.e., secreted Frizzled-Related Proteins, a family of soluble proteins that are structurally related to FZD proteins). 5FAM-Fz7-21 or 5FAM-Fz7-21S were not found to bind any of the sFRPS tested. See, e.g., FIGS. 5A-5C. Vertical dotted lines represent the elution volume of molecular weight standards.

For FIGS. 4C and 5C, values represent the mean±s.e.m. of three independent experiments. The areas under the curve (AUC) for FIGS. 4C and 5C were integrated using UNICORN (v5.31; General Electric Bio-Sciences) and represent the mean±s.e.m. from three independent experiments. Samples were resolved using a SEC3000 column (Phenomenex; Torrence, Calif.) at 0.5 mL/min in phosphate buffer saline (PBS) and fluorescence was monitored with an FP-2020 Plus fluorescence detector (Jasco Analytical Instruments, Easton, Md.) using normal mode (excitation/emission 550/494 nm); gain=100; STD=32. All solutions maintained 1% final DMSO. Line traces were adjusted upward by n+20-units to enable differentiation between traces. Molecular weights were determined by standards (BioRad catalogue number, 151-1901) run prior to sample runs.

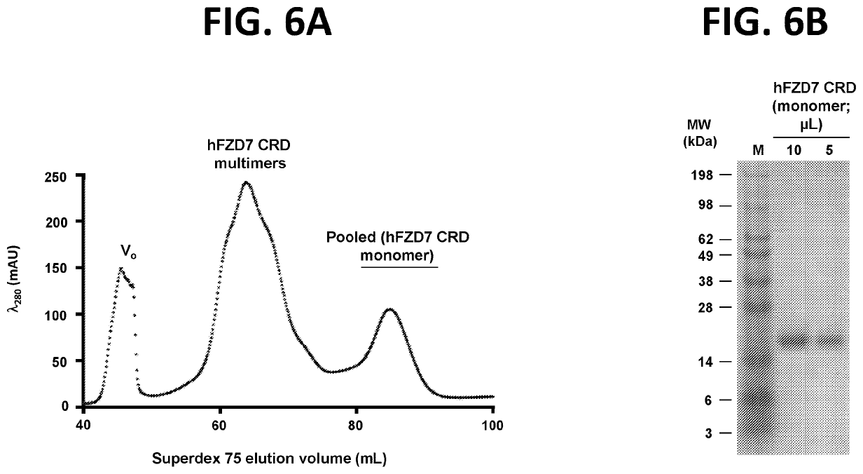

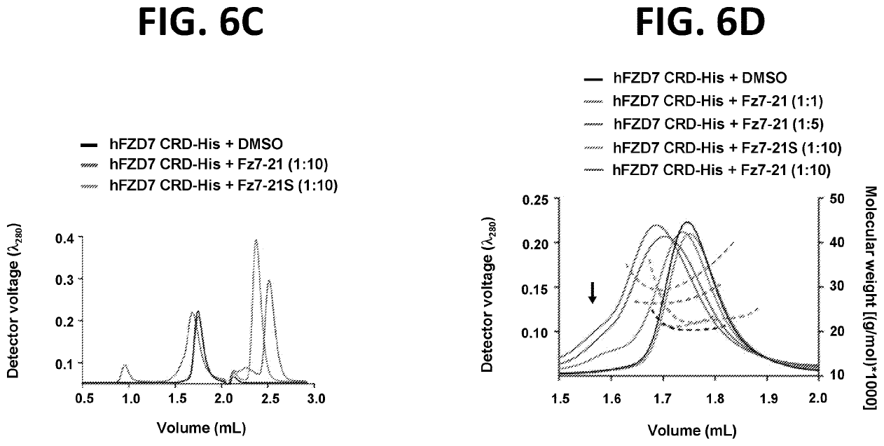

Further experiments were performed to examine peptide binding to a soluble monomeric form of hFZD7 CRD. Size exclusion chromatography (SEC) was performed using an EndoH- and Kifunensine-treated recombinant hFZD7 CRD-His generated from baculovirus that had been captured with Ni-NTA agarose resin, concentrated, and resolved using a GE Healthcare HiLoad 16/60 Superdex 75 PG at 0.5 mL/min with 150 mM NaCl, 50 mM Tris-HCl, pH 7.5. As shown in FIG. 6A, hFZD7 CRD elutes as two major peaks, a monomer at ˜16 kDa and as higher molecular weight multimers. “Vo”=void volume. FIG. 6B shows the SDS-PAGE of pooled monomer from FIG. 6A. Samples were treated with sample buffer supplemented with 1 mM DTT and heated to 98° C. for 10 min prior to analysis by SDS-PAGE. M, marker (10 μL, See blue Plus2 pre-stained protein standard).

To further understand the mechanism of action of Fz7-21, hFZD7 CRD-His (15.8 μM) was incubated with (a) Fz7-21 at a ratio of 1:1, 1:5, or 1:10, (b) with Fz7-21S at a ratio of 1:10, or (c) DMSO for 2 hours prior to resolution via size exclusion chromatography (SEC) and detection via multi-angle light scattering (MALS) and absorption at λ₂₈₀. Samples were resolved by a Superdex S200 3.2/300 column (General Electric Healthcare Life Science, Pittsburgh, Pa.; 28990946) at a 0.15 mL/min in 150 mM NaCl, 50 mM Tris-HCl pH 7.5 buffer with 1% DMSO final concentration. Column was connected to a 1260 infinity HPLC (Agilent Technology, Santa Clara, Calif.) connected to a Dawn Heleos-II Multi-Angle static Light Scattering (MALS) detector and the Optilab T-rEX differential Refractive Index (dRI) detectors (Wyatt Technologies, Santa Barbara, Calif.). The results of SEC-MALS analysis are shown in FIG. 6C. FIG. 6D shows a zoom-in view (1.5 mL to 2.0 mL range) of FIG. 6C with additional peptide concentrations tested. Quantification of MALS signal and the change in molecular weight (AMW) relative to the DMSO control are shown in Table 9. Incubation with Fz7-21 promotes the formation of multimer(s) near Ve=1.6 mL. See FIG. 6D.

TABLE 9

| | | | Uncertainty | |
|---|---|---|---|---|
| Protein | Treatment | MW (kDa) | (%) | ΔMW (kDa) |
| BSA | DMSO | 70.1 | 0.3 | N/A |
| hFZD7 CRD | DMSO | 20.9 | 1.4 | 0 |
| hFZD7 CRD | Fz7-21 (1:1) | 21.9 | 1.6 | 1 |
| hFZD7 CRD | Fz7-21 (1:5) | 27.3 | 1 | 6.4 |
| hFZD7 CRD | Fz7-21 (1:10) | 32 | 1.4 | 11.1 |
| hFZD7 CRD | Fz7-21S | 23.5 | 1.3 | 2.6 |
| | (1:10) | | | |

Such results demonstrate that peptide Fz7-21 binds to monomeric hFZD7 CDR-His. See Table 9. Unexpectedly, Fz7-21 induced homodimerization or oligomerization of hFZD7 CRD-His in a concentration-dependent manner. See Table 9. Collectively, such findings demonstrate that peptide Fz7-21 displays binding selectivity to the FZD7 receptor subclass. Such results also indicate that Fz7-21 induces dimerization of monomeric FZD7 CRD, and that Fz7-21 binds to the preformed tetrameric FZD7 CRD-Fc. (See FIGS. 6A-6D.)

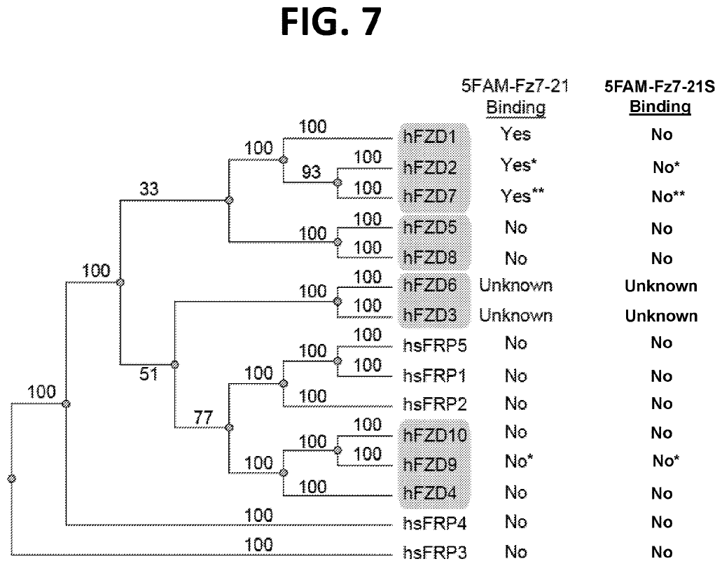

The results discussed above demonstrate that Fz7-21 selectively binds to the FZD7 subclass of evolutionarily conserved proteins, namely FZD1, FZD2, and FZD7. See FIG. 7. Such binding selectivity is unexpected in view of the high degree of sequence similarity between FZD proteins.

In FIG. 7, protein CRDs were manually trimmed and aligned with Clustal Omega. The cladogram in FIG. 7 was generated using PHYLIP Neighbor Joining with Jones-Taylor-Thorton distance matrix and 100 bootstrapped iteration and a transition/transversion ratio of 2.0. To the right of the cladogram in FIG. 7 is a summary of the binding preference of 5FAM-Fz7-21 or 5FAM-Fz7-21S to each protein. “*” indicates that the mouse protein was tested. “**” indicates that both mouse and human proteins were tested. “sFRP” refers to “secreted frizzled-related protein.” The following accession numbers were used as the source sequences: hFZD1 (Q9UP38); hFZD2 (Q14332); hFZD3 (Q9NPG1); hFZD4 (Q9ULV1); hFZD5 (Q13467); hFZD6 (060353); hFZD7 (075084); hFZD8 (Q9H461); hFZD9 (000144); hFZD10 (Q9ULW2).

### Example 3: Structural Characterization of the Interaction Between FZD7 CRD and Fz7-21

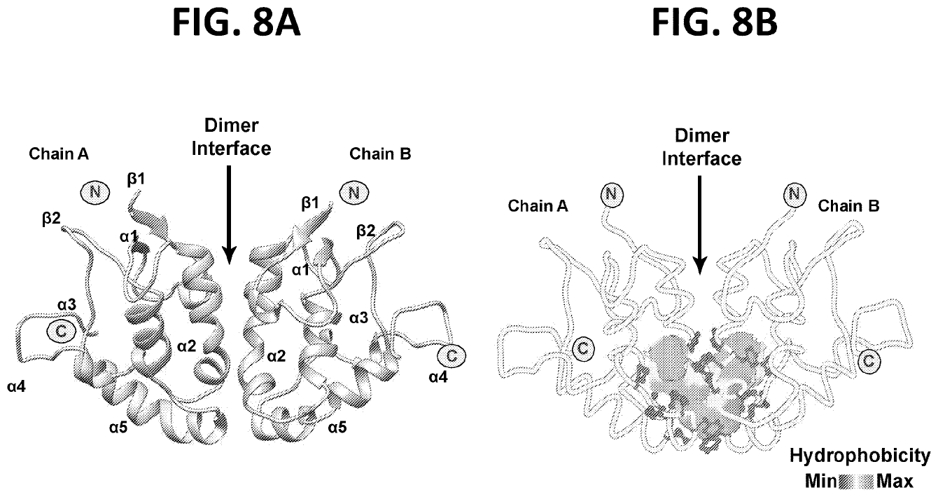

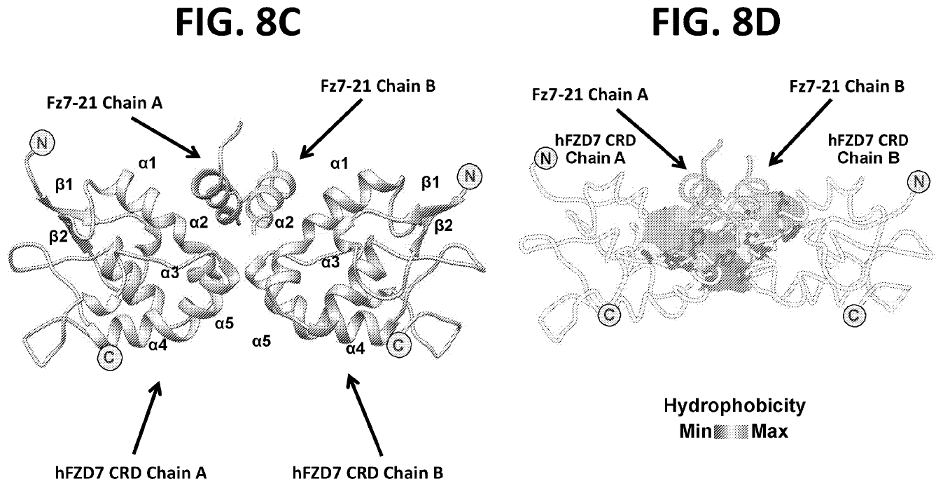

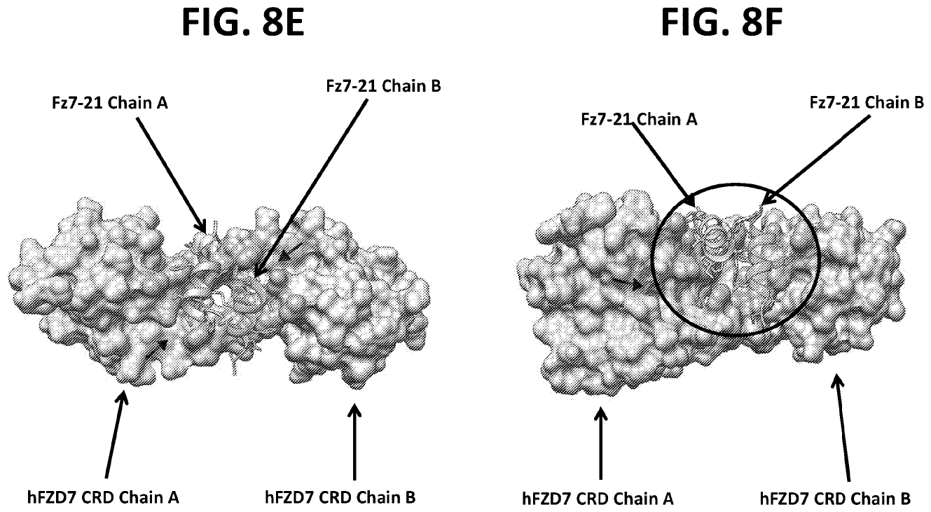

The X-ray crystal structures of hFZD7 CRD in its apo form or in complex with a C24 fatty acid was recently reported (Nile et al. (2017) Proc. Natl. Acad. Sci. USA 114, 4147-4152 LID-4110.1073/pnas.1618293114 [doi]). In both structures, hFZD7 CRD comprised a dimer with an α-helical dimer interface and a U-shaped lipid-binding cavity that bridges the dimer interface Nile et al. (2017) Proc. Natl. Acad. Sci. USA 114, 4147-4152 LID-4110.1073/pnas.1618293114 [doi]). In order to gain insight into the mechanisms of peptide-FZD CRD selectivity, further experiments were performed to characterize the peptide-protein interaction at a molecular level. Extensive efforts aimed at obtaining the co-crystal structure of hFZD7 CRD in complex with Fz7-21, including co-crystallization and soaking techniques, were unsuccessful. A construct in which the N terminus of Fz7-21 was fused to the C terminus of hFZD7 CRD, flanked with a linker, was used. The X-ray crystal structures of FZD7 CRD (apo form) and hFZD7 CRD bound to peptide Fz7-21 were determined at 2.00 Å and 2.88 Å resolution, respectively (see FIGS. 8A-8F and Table 10 below). The structure of apo FZD7 CRD revealed a dimer with a unique architecture. As shown in FIGS. 8A-8F, the structure of the lipid binding cavity of FZD7 CRD is surprisingly different from the reported structures of lipid binding cavities of other FZD CRD family members. Two lipid binding grooves exist in the FZD7 CRD dimer. The tails of each FZD7 CRD monomer face each other at the dimer interface. See FIGS. 8A-8B. This creates a contiguous and bent (U-shaped) lipid-binding cavity that bridges the dimer interface. See FIG. 8B. Residues proximal to the hydrophobic cavity are displayed in ball-and-stick form in FIG. 8B. FIG. 8C shows the crystal structure of hFZD7 CRD bound to Fz7-21. FIG. 8D shows a surface representation of the hydrophobic cavity mapped onto the structure of hFZD7 CRD (ribbon representation) bound to Fz7-21 (ribbon representation). Residues that form the hydrophobic cavity are shown in ball and stick representation). FIG. 8E shows a top view surface representation of the crystal structure of hFZD7 CRD bound to Fz7-21 (ribbon representation; disulfide shown). FIG. 8F shows a side view surface representation of the crystal structure of hFZD7 CRD bound to Fz7-21 (ribbon representation; disulfide shown). The binding epitope of Fz7-21 (defined within a 4 Å distance) is within the circle. The lipid binding cavity is indicated by arrows. For all structures, the glycan moieties are hidden for clarity.

TABLE 10
X-ray crystallography data processing and structural refinement statistics
for apo hFZD7 CRD and Fz7-21 bound hFZD7 CRD

| | hFZD7 CRD | hFZD7 CDR-Fz721 |
|---|---|---|
| PDB Code | TBD | TBD |
| X-ray source | 2015_01_21_SSRL_122 | 2015_11_14_APS_17ID |
| | (CRY18650) | (CRY20775) |
| Space group | P2₁2₁2₁ | P2₁ |
| Unit cell | a = 39.1 Å, b = 62.8 Å, c = 103.2 Å, | a = 62.6 Å, b = 164.7 Å, c = 97.8 Å, α = |
| | α = β = γ = 90° | 90°, β = 108.3°, γ = 90° |
| Resolution | 2.00 Å | 2.88 Å |
| Total number of reflections | 17922 (103)¹ | 42391 (426)¹ |
| Completeness (%) | 99.5 (97.1) | 99.7 (99.6) |
| Redundancy | 6.4 (6.2) | 3.0 (2.9) |
| I/σ | 10.3 (2.6) | 7.4 (1.2) |
| Rsym² | 0.084 (0.430) | 0.080 (0.600) |
| Resolution range | 50-2.00 Å | 50-2.88 Å |
| Rcryst³/Rfree⁴ | 0.199/0.252 | 0.196/0.243 |
| Non-hydrogen atoms | 1926 | 8368 |
| Water molecules | 98 | 1 |
| Average B, overall | 40.09 | 89.12 |
| r.m.s.d. bond lengths | 0.006 Å | 0.007 Å |
| r.m.s.d. bond lengths | 0.994° | 1.159° |
| Ramachandran | 0.944/0.056/0/0 | 0.883/0.111/0.003/0.002 |
| (C/A/G/D)⁵ | | |

¹Values in parentheses are of the highest resolution shell.
²Rsym = Σ|Ihi − Ih|/ΣIhi, where Ihi is the scaled intensity of the ith symmetry-related observation of reflection h and Ih is the mean value.
³Rcryst = Σh|Foh − Fch|/ΣhFoh, where Foh and Fch are the observed and calculated structure factor amplitudes for reflection h.
⁴Value of Rfree is calculated for 5% randomly chosen reflections not included in the refinement.
⁵C—core; A—additionally allowed; G—generally allowed; D—disallowed.

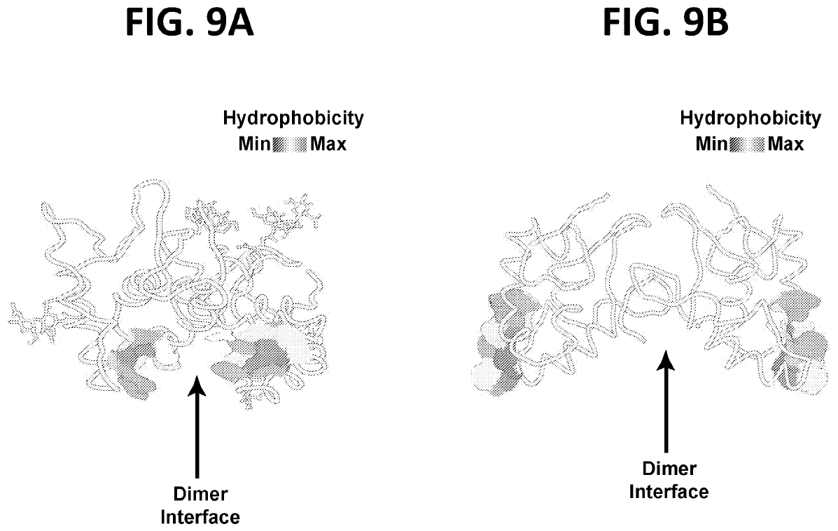

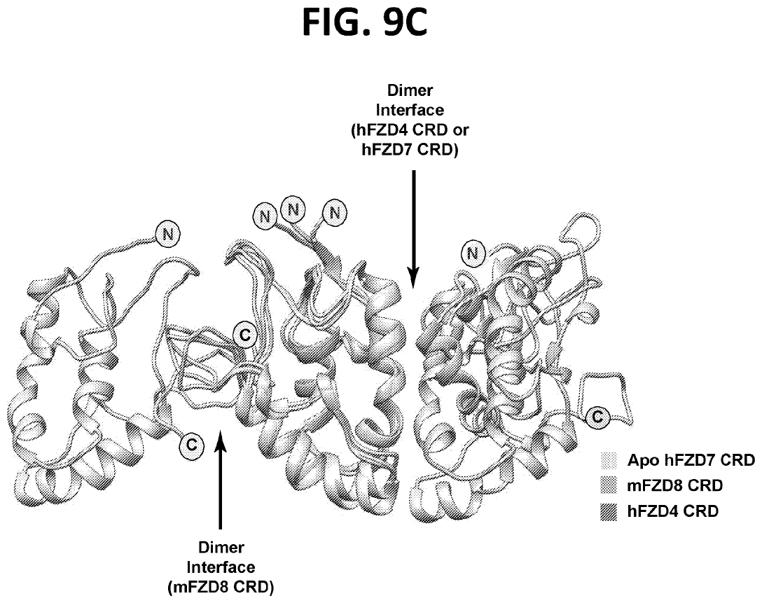

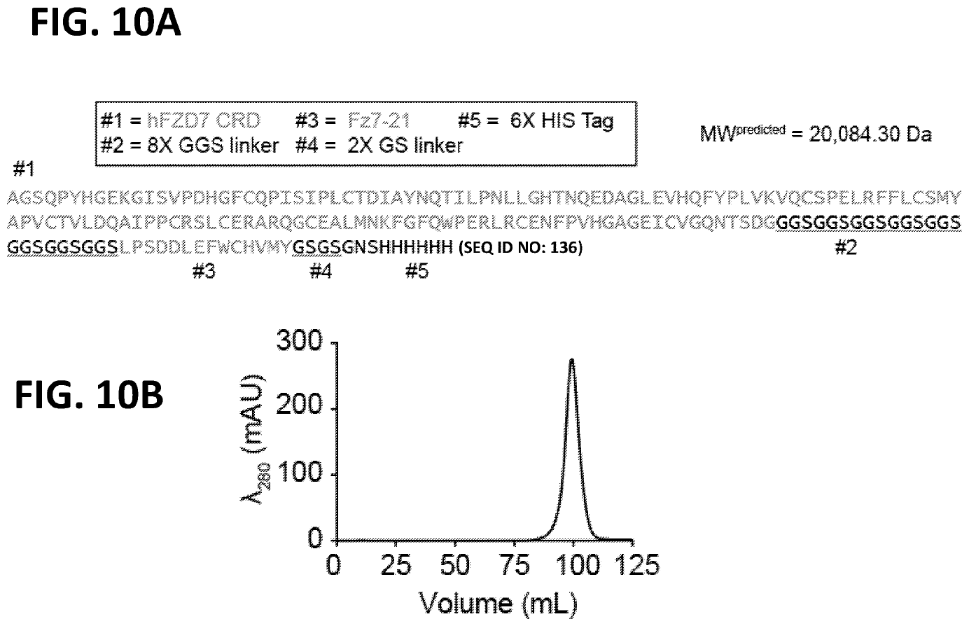

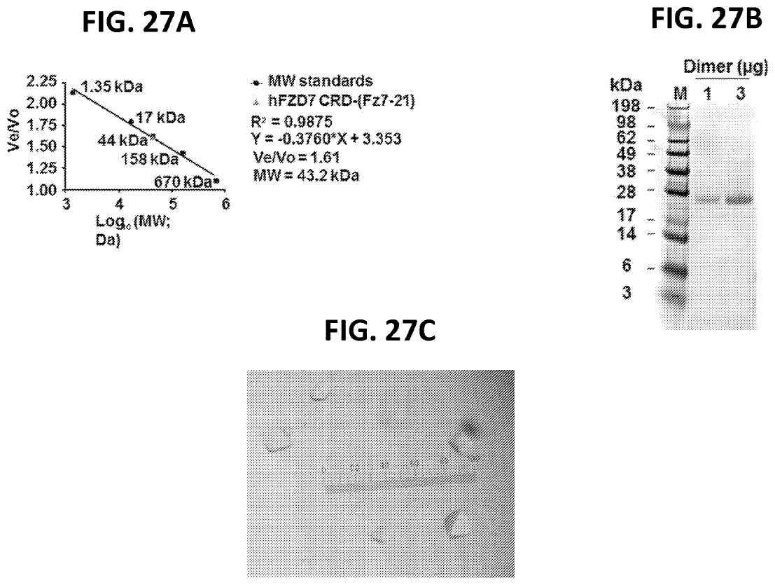

This arrangement is in stark contrast to the positioning of the lipid-binding grooves on other FZD CRDs such as those of hFZD4 (see FIG. 9A) and mFZD8 (see FIG. 9B), which are located on opposite sides away from the dimer interface, and are thereby separated from each other (see, e.g., Janda et al. (2012) Science 337, 59-64; Dann et al. (2001) Nature 412, 86-90; and Shen et al. (2015) Cell Res. 25, 1078-1081). The lipid binding cavities of hFZD4 (see FIG. 9A) and mFZD8 (see FIG. 9B) are shaded according to amino acid hydrophobicity over a gradient (dark gray=hydrophilic; light gray=hydrophobic). Furthermore, the lipid-binding grooves of hFZD4 CRD and mFZD8 CRD also differ from that of hFZD7 CRD in that they are solvent-exposed and display an ‘extended’ conformation. FIG. 9C provides a superimposition of apo hFZD7 CRD with mFZD8 CRD and hFZD4 CRD, highlighting the different dimer interfaces. Extensive efforts aimed at obtaining the ω-crystal structure of hFZD7 CRD in complex with Fz7-21, including ω-crystallization and soaking techniques, were unsuccessful. A construct was designed in which the N-terminus of Fz7-21 was fused to the C-terminus of hFZD7 CRD, flanked with a linker and is referred to as hFZD7 CRD-GS (FIGS. 10A and 10B and Table 10). The fusion construct was expressed in insect cells, and purified to near homogeneity by size-exclusion chromatography (SEC). FIG. 10B shows the SEC profile of purified hFZD7 CRD-GS with a determined molecular weight (MW) of 42.7 kDa, corresponding to a dimer. FIG. 27A shows the associated MW standards used to determine the MW of hFZD7 CRD-GS. FIG. 27B shows an SDS-PAGE of the fusion protein in FIG. 10A under reducing conditions, corresponding to a monomer. FIG. 27C shows a bright field image of crystals obtained from the fusion protein in FIG. 10A.

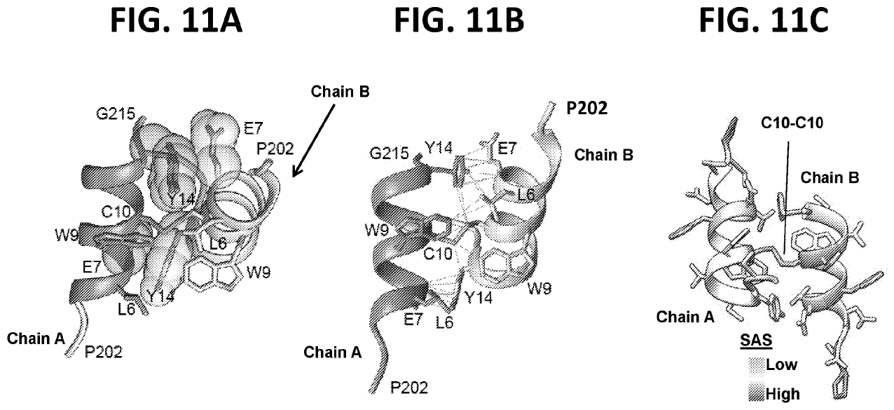

The crystal structure of the fusion construct revealed that peptide Fz7-21 bound as a dimer proximal to the lipid-binding groove, making contacts with residues that line the lipid-binding cavity at the dimer interface of hFZD7 CRD (FIGS. 8C-8F and Tables 11 and 12). The peptide dimer comprised two anti-parallel alpha helices oriented at a ˜45° angle relative to each other and are held together by one disulfide bond at Cys10 and numerous backbone and side chain interactions, forming a solvent-protected core surrounding the disulfide bond (FIGS. 8C-8F and FIGS. 11A-11C). The asymmetric unit comprised four structurally similar CRD dimer pairs, each bridged by Fz7-21 dimer peptide. The linker region could not be resolved due to lack of electron density.

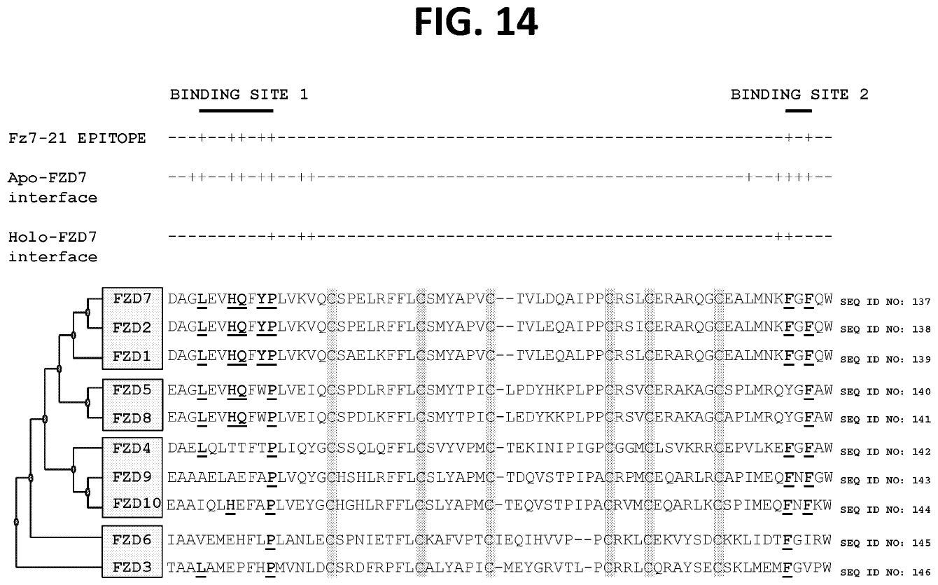

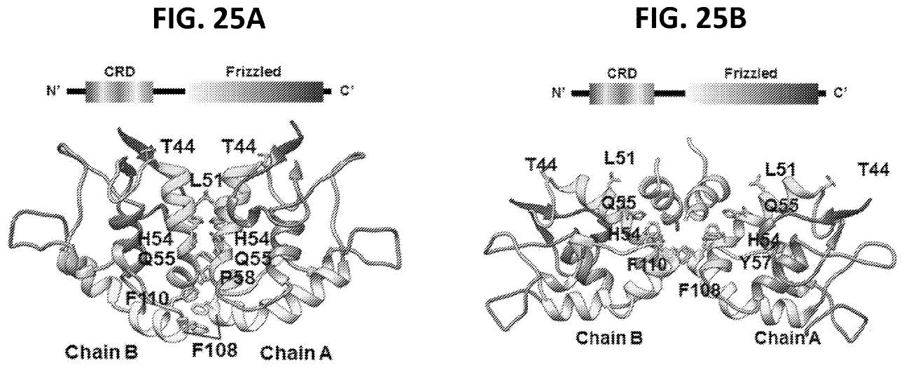

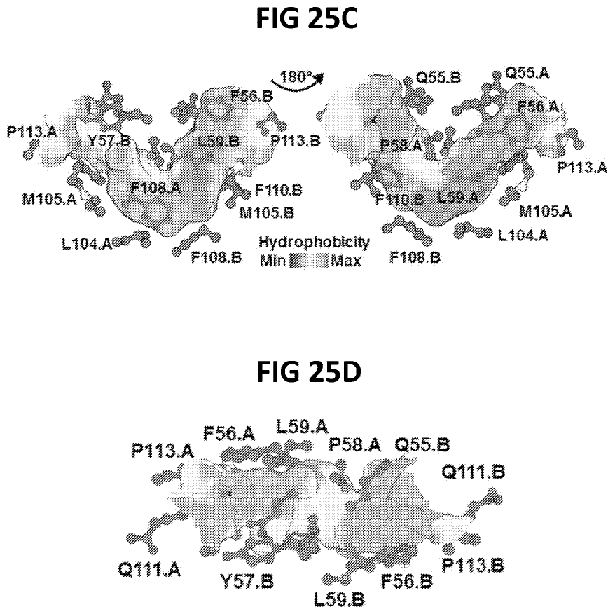

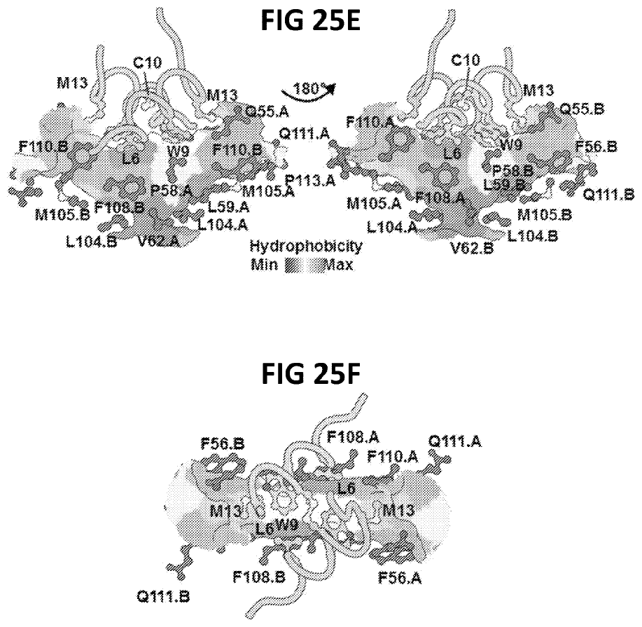

The architecture of the peptide-bound hFZD7 CRD showed a unique conformation. The geometry of the lipid-binding cavity from is altered from a bent form (i.e., in the apo structure), showing a ˜16° angle at the dimer interface, to an extended form (i.e., in the peptide-bound form), showing a 90° at the dimer interface. Thus, binding of Fz7-21 dimer to the lipid binding cavity of hFZD7 CRD displaces the dimer interface by ˜75° (FIGS. 8A-8D, FIGS. 12A and 12B and FIGS. 25A-25F). FIG. 25A shows a ribbon representation of apo hFZD7 CRD crystal structure (rainbow coloration; N-terminal, blue; C-terminal, red), with schematic of full length FZD7 illustrating the CRD placement within FZD7, and FIG. 25B shows a ribbon representation of the structure of hFZD7 CRD (rainbow coloration) bound to Fz7-21. In both apo and bound structures, select paired residues at the dimer interface are shown in FIGS. 25A and 25B. FIG. 25C provides a zoomed-in side view of the hydrophobic cavity in apo hFZD7 CRD, and FIG. 25D provides a top view from of FIG. 25C. FIG. 25E provides a zoomed-in side view of hFZD7 CRD bound to Fz7-21 with the hydrophobic cavity highlighted and protein backbone hidden for clarity with 180° rotation, and FIG. 25F shows the top view of FIG. 25E. The hydrophobic cavity in FIGS. 25C-25E is depicted as dark gray, hydrophobic; white, neutral; blue, light gray. In total, the number of residues constituting the FZD7 CRD α-helical dimer interface decreased from 13 (P55, E77, G80, L81, H84, Q85, Y87, P88, K91, V92, L134, F138 (which is alternatively identified as F130 throughout the specification), and F140) per monomer in the apo structure to 5 (P88, K91, V92, K137 and F138 (which is alternatively identified as F130 throughout the specification)) per monomer in the peptide-bound structure. (See FIG. 14.)

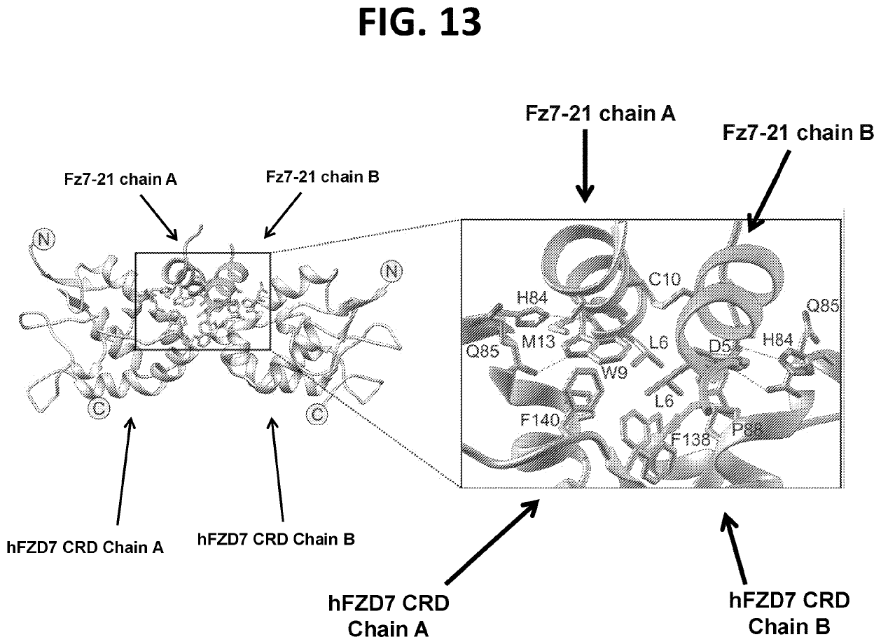

In this binding configuration, the peptide α-helical dimer forms a “lid” on top of the extended lipid-binding groove, and the binding residues from each helix make contacts with residues on both hFZD7 CRD monomers (FIG. 8C-8F and Tables 11 and 12 below). As shown in FIG. 13, Leu6 on Fz7-21 (Chain B) makes key hydrophobic contacts with Phe138 (which is alternatively identified as Phe130 throughout the specification) and Phe140 (Chain A) of hFZD7 CRD, whereas the backbone carbonyl of Asp5 on Fz7-21 (Chain B) forms a hydrogen bond with the side chain of His84 (Chain B) of hFZD7 CRD. In FIG. 13, select Fz7-21-hFZD7 CRD interactions are highlighted within the crystal structure of hFZD7 CRD bound to Fz7-21 (ribbon and stick representation). Dotted lines represent hydrogen bonding interactions. Moreover, the indole nitrogen of Trp9 on Fz7-21 forms a hydrogen bond with the backbone carbonyl of Gln85 of hFZD7 CRD. See FIG. 13. In sum, there are sixteen residues on Fz7-21 (eight residues per monomer) which are involved in the interaction with hFZD7 CRD, primarily at the α-helical dimer interface lining the surface of the lipid-binding groove (see Table 12).

TABLE 11
FZD7 CRD dimer residues that are within 4 Å of Fz7-21
hFZD7 CRD residues within 4 Å of Fz7-21

| (Dimer Chain A) | (Dimer Chain B) |
|---|---|
| Leu81 | Leu81 |
| His84 | His84 |
| Gln85 | Gln85 |
| Ty87 | Ty87 |
| Pro88 | Pro88 |
| Phe138* | Phe138* |
| Phe140 | Phe140 |

*Phe138 is alternatively identified as Phe130 throughout the specification

TABLE 12
Fz7-21 residues that are within 4 Å of the hFZD7 CRD dimer
Fz7-21 residues within 4 Å of hFZD7 CRD

| Fz7-21 (Chain A) | Fz7-21(Chain B) |
|---|---|
| Ser3 | Ser3 |
| Asp5 | Asp5 |
| Leu6 | Leu6 |
| Phe8 | Phe8 |
| Trp9 | Trp9 |
| Val12 | Val12 |
| Met13 | Met13 |
| Tyr14 | Tyr14 |

These interactions provide a rationale for the peptide selectivity towards the FZD7 receptor sub-class, based in part on sequence conservation of the peptide binding epitope within the FZD CRD family. See FIG. 14, which provides an alignment of the amino acid sequences of the CRDs of hFZD7, hFZD1, hFZD2, mFZD8, hFZD5, hFZD4, hFZD9, hFZD10, hFZD3, and hFZD6. In FIG. 14, the Fz7-21 binding epitope, and residues in the alpha-helical dimer interface of hFZD7 CRD (chain A vs. chain B) in its apo form or in complex with Fz7-21 (holo-FZD7 interface; chain A vs. chain B) are denoted by “+” above the sequence alignment. Conserved cysteines are highlighted in gray and conserved epitope residues are in underlined. Additionally, the dimer interface and lipid-binding groove geometry within hFZD7 CRD contributes to the peptide binding footprint and hence may influence peptide selectivity. The backbone and side chain interactions that help to stabilize the peptide helix interdimer are shown in Table 13.

TABLE 13
Summary of Fz7-21 Intramolecular Interactions

| Residue 1 | Residue 2 | Comments |
|---|---|---|
| Cys10 | Cys10 | Disulfide |
| Tyr14 | Glu7 | Van der Waals interactions |
| | | stabilizing dimer geometry |
| Tyr14 | Glu7 | Van der Waals interactions |
| | | with peptide backbone |
| Tyr14 | Leu6 | Hydrophobic interactions |
| Tyr14 | Cys10 | Hydrophobic, promotes solvent |
| | | excluded area surrounding |
| | | Cys10-Cys10 disulfide |
| Trp9 | Trp9 | Hydrophobic interactions |
| Trp9 | Cys10 | Hydrophobic, promotes solvent |
| | | excluded area surrounding |
| | | Cys10-Cys10 disulfide |
| Trp9 | Leu6 | Hydrophobic interactions |
| | | stabilizes peptide geometry |
| Tyr14 | Cys10 | Hydrophobic, promotes solvent |
| | | excluded area surrounding |
| | | Cys10-Cys10 disulfide |
| Phe8 | Leu12 | Hydrophobic interactions |
| (side chain) | (side chain) | |
| Trp9 | M13 | Stacking/hydrophobic interactions |
| Pro2.A (carbonyl) | Asp4.A | Main chain H-bond forms α-helix |
| | (amino) | |
| Asp4.A (carbonyl) | Phe8.A | Main chain H-bond forms α-helix |
| | (amino) | |
| Asp5.A (carbonyl) | Trp9.A (amino) | Main chain H-bond forms α-helix |
| Leu6.A (carbonyl) | Cys10.A | Main chain H-bond forms α-helix |
| | (amino) | |
| Glu7.A (carbonyl) | Cys10.A | Main chain H-bond forms α-helix |
| | (amino) | |
| Glu7.A (carbonyl) | His11.A | Main chain H-bond forms α-helix |
| | (amino) | |
| Phe8.A (carbonyl) | His11.A | Main chain H-bond forms α-helix |
| | (amino) | |
| Phe8.A (carbonyl) | Val12.A | Main chain H-bond forms α-helix |
| | (amino) | |
| Cys10.A | Tyr14.A | Main chain H-bond forms α-helix |
| (carbonyl) | (amino) | |
| His11.A | Gly15.A | Main chain H-bond forms α-helix |
| (carbonyl) | (amino) | |
| Asp4.B (carbonyl) | Phe8.B (amino) | Main chain H-bond forms α-helix |
| Asp5.B (carbonyl) | Trp9.B (amino) | Main chain H-bond forms α-helix |
| Leu6.B (carbonyl) | Cys10 (amino) | Main chain H-bond forms α-helix |
| Phe8.B (carbonyl) | Val12.B | Main chain H-bond forms α-helix |
| | (amino) | |
| Trp9.B (carbonyl) | Met13.B | Main chain H-bond forms α-helix |
| | (amino) | |
| Cys10.B | Tyr14.B | Main chain H-bond forms α-helix |
| (carbonyl) | (amino) | |

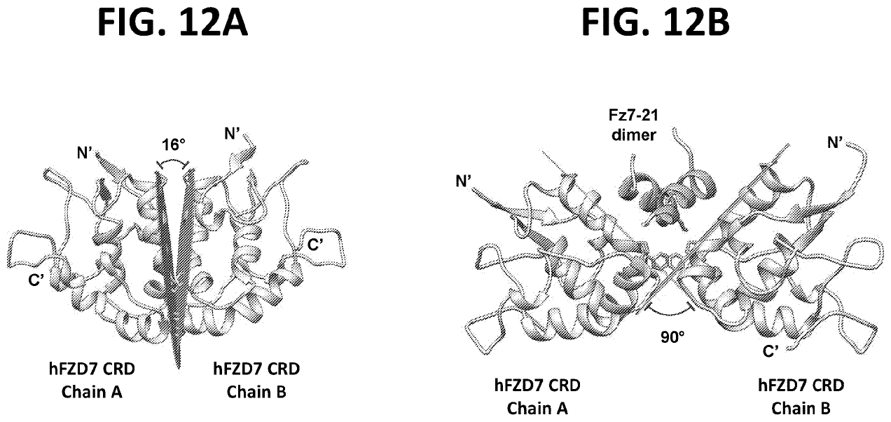

The crystal structure of apo FZD7 CRD reveals an unexpected molecular picture about the unique geometry of the lipid binding cavity within this FZD subclass, which may offer an explanation as to why cis-unsaturated fatty acids on Wnt proteins may have preferentially evolved to bind to the lipid binding cavity of hFZD CRDs. Notably, the architecture of the peptide-bound FZD7 CRD showed a unique conformation, with altered geometry of the lipid binding cavity from a bent U shape (in the apo structure, shown in FIG. 12A) to an extended form, coupled with a substantial displacement of the dimer interface by 75° to form a ˜90° angle. The distance (A) between amino acid residues at the dimer interface in the apo form and Fz7-21-bound form of FZD7 CRD are provided in Table 14 below.

TABLE 14
Inter-residue Distances (Å)

| | Apo | Fz7-21-bound | |
|---|---|---|---|
| Residue | FZD7 CRD | FZD7-CRD | Δ |
| T74 | 23.5 | 53.9 | 30.4 |
| L81 | 6.4 | 29.2 | 22.8 |
| H84 | 4.6 | 23.0 | 18.4 |
| Q85 | 7.2 | 21.0 | 13.8 |
| Y87 | 8.3 | 17.3 | 9.0 |
| P88 | 5.1 | 8.3 | 3.2 |
| F138 | 9.7 | 14.8 | 5.1 |
| F140 | 16.9 | 21.5 | 4.6 |

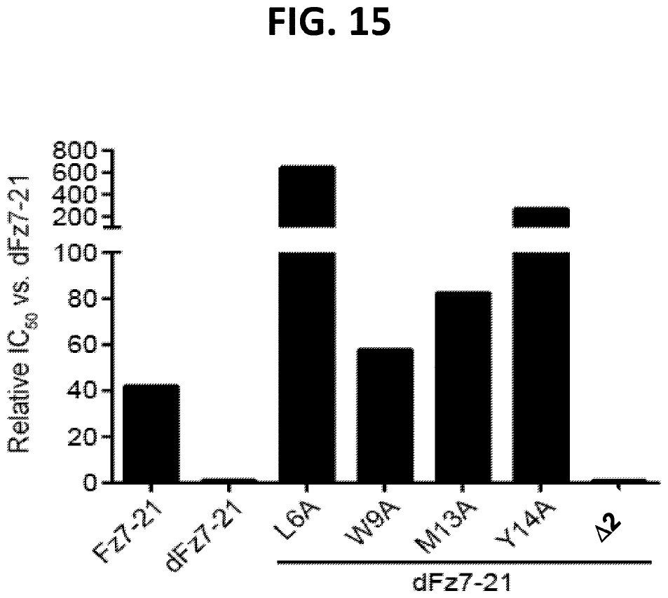

The observed interactions from the crystallographic model are supported by several of functional analyses described above. First, shotgun alanine scanning identified several residues on Fz7-21, i.e., Asp5, Leu6, Trp9, Cys10, Met13 and Tyr14, that are important for interaction with hFZD7 CRD-Fc (see Table 6 above), in line with the crystal structure (see FIG. 13). Second, observations from the crystal structure suggested that the dimeric form of Fz7-21 would exhibit improved activity. dFz7-21 (i.e., a dimeric form of Fz7 obtained synthetically dimerizing Fz7-21 via disulfide bond at Cys10) demonstrated ˜40-fold improvement compared to monomeric Fz7-21 in inhibiting Wnt3a signaling in HEK293 cells (FIG. 15), as measured by TOPbrite reporter assay in HEK293-TB cells stimulated with recombinant mWnt3a (50 ng/mL). (The values in FIG. 15 represent the fold-change in IC₅₀ of the indicated peptide relative to dFz7-21. To account for expression levels, firefly luminescence signal was normalized to renilla luciferase luminescence. The calculated values were background subtracted, normalized to mock treated samples, and represent the mean of at least three independent experiments, each with technical triplicates.) Additionally, alanine point mutations at various positions within dFz7-21 that were predicted to disrupt its interaction with hFZD7 CRD (e.g., L6A), led to a reduction in cellular potency by ˜800-fold (see FIG. 15 and Table 15). By contrast, truncations at the N-terminus did not interfere with dFz7-21 activity (e.g. peptide dFz7-21-Δ2, lacking the 2 N-terminal residues), consistent with the crystal structure and shotgun alanine scanning data (see Table 6, Table 15, and FIG. 15). Fz7-21S was tested in parallel as a negative control.

TABLE 15

| | | IC50 (nM) | Reduction | |
|---|---|---|---|---|
| | | (95% | in fold | PEPTIDE |
| | IC50 | Confidence | potency | SEQUENCE |
| PEPTIDE | (nM) | interval) | vs. dimer | (SEQ ID NO.) |
| Fz7-21 | 464.8 | 296.6 to 633.3 | 42 | LPSDDLEFWCHVMY |
| | | | | (SEQ ID NO: 13) |
| dFz7-21 | 11.1 | 8.3 to 13.9 | 1 | [symbol] |
| dFz7-21-L6A | 7,120.7 | 4,905.7 to 9,335.7 | 642 | LPSDDAEFWCHVMY |
| | | | | (SEQ ID NO: 109) |
| dFz7-21-W9A | 639.7 | 440.5 to 838.9 | 58 | LPSDDLEFACHVMY |
| | | | | (SEQ ID NO: 110) |
| dFz7-21-M13A | 913.1 | 692.9 to 1,133.3 | 82 | LPSDDLEFWCHVAY |
| | | | | (SEQ ID NO: 111) |
| dFz7-21-Y14A | 2,964.2 | 1,985.9 to 3,942.4 | 267 | LPSDDLEFWCHVMA |
| | | | | (SEQ ID NO: 112) |
| dFz7-21-Δ2 | 8.8 | 6.6 to 10.9 | 1 | SDDLEFWCHVMY |
| | | | | (SEQ ID NO: 99) |
| Fz7-21-C10S | Beyond | Beyond | >9,000 | LPSDDLEFWSHVMY |
| | solubility | Solubility | | (SEQ ID NO: 113) |

[symbol]

dFz7-21 is a dimeric form of Fz7 obtained synthetically dimerizing Fz7-21 via disulfide bond at Cys10

To obtain the data in FIG. 15 and Table 15, Wnt signaling was measured by TOPbrite reporter assay in HEK293-TB cells stimulated with recombinant mWnt3a (50 ng/mL). The values in FIG. 15 and Table 15 represent the fold-change in IC₅₀ of the indicated peptide relative to dFz7-21. To account for expression levels, firefly luminescence signal was normalized to renilla luciferase luminescence. The calculated values were background subtracted, normalized to mock treated samples, and represent the mean of at least three independent experiments, each with technical triplicates.

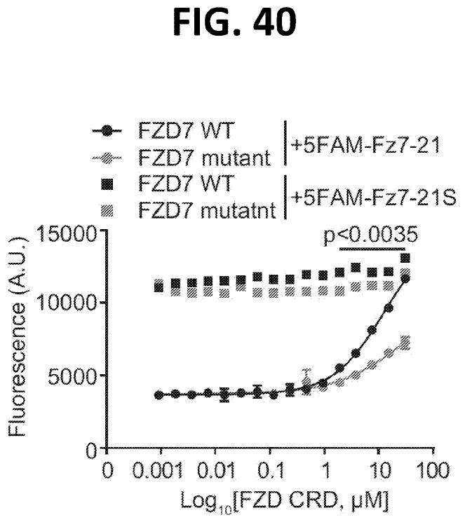

Further, mutations introduced at specific residues within the peptide-binding region on hFZD7 CRD (H84A, Y87A, F138A (which is alternatively identified as F130A throughout the specification), F140A) reduced the binding of Fz7-21 compared to wild-type hFZD7 CRD (see FIG. 40), consistent with structural predictions. Moreover, Fz7-21 promoted monomeric to dimeric transition of hFZD7 CRD in solution (data not shown), in line with the structural model depicting 2:2 stoichiometry for peptide-CRD binding. Fifth, FZD7 CRD-Fz7-21 fusion construct was predominantly dimeric in solution (data not shown), compared to a fusion construct containing Cys10 to Ser mutation within the peptide sequence, which showed a mixed population of monomers and oligomers with less pronounced dimeric species (data not shown). Additional Ala mutations of Fz7-21 residues that were predicted to be important for peptide-peptide or peptide-FZD7 interactions (i.e., L6A; W9A; Y14A; L6A and W9A; L6A and Y14A; and L6A, W9A, and Y14A) led to reduced dimerization of the FZD7 CRD-Fz7-21 fusion construct (data not shown). These findings are consistent with the above SEC-MALS data and the crystallographic observations, further supporting the notion that the active form of Fz7-21 is a dimer which enhances dimer formation of FZD7 CRD.

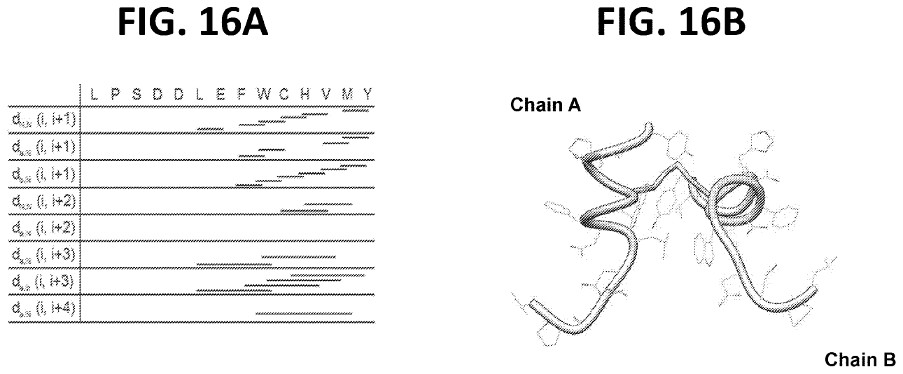

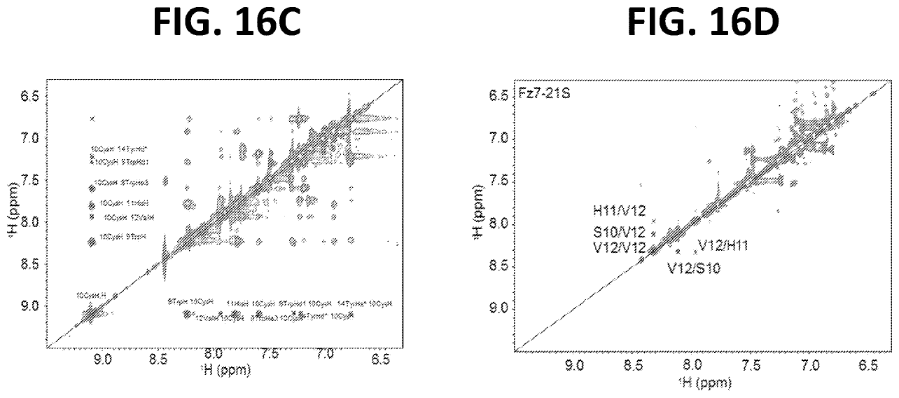

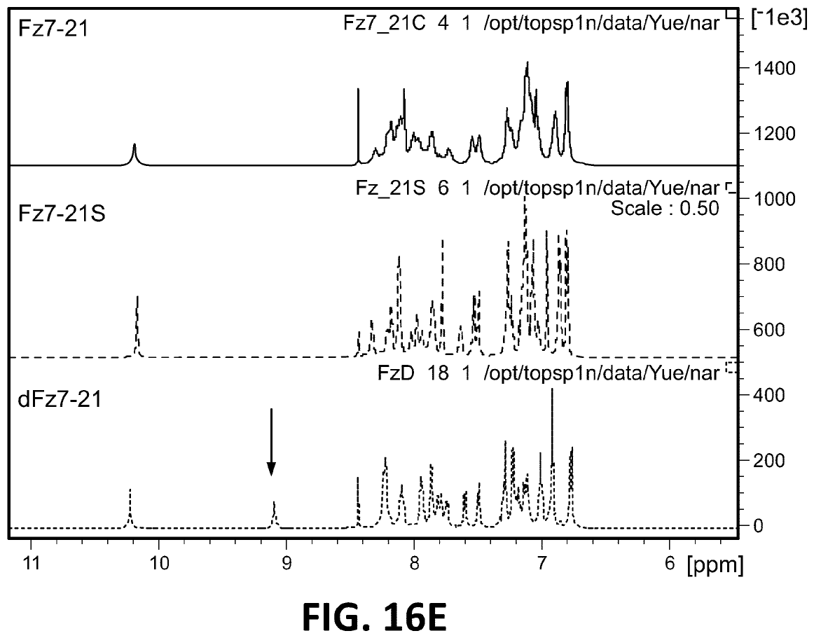

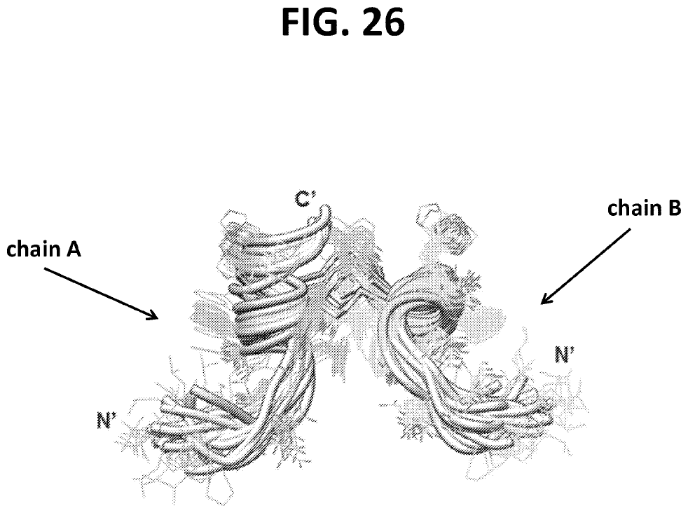

The NMR solution structure of dFz7-21 revealed that the peptide dimer was structured, exhibiting intramolecular interactions and an α-helical character (see FIGS. 16A-16E and Table 16), similar to observations from the crystal structure. FIG. 16A provides a NOESY connectivity plot of dFz7-21. FIG. 26 shows a superimposition of the 20 lowest energy NMR structures of dFz7-21 (chain A and Chain B, ribbon representation; side chains, line representation). FIG. 16B shows a representative NMR solution structure of dFz7-21 based on superimposition of the 20 lowest energy NMR structures of dFz7-21 (amino acid side chains are shown as lines). In the NMR solution structure, the two peptide chains were oriented at a 90° angle with respect to one another and the N-terminal region appeared disordered, indicating that interaction with FZD7 CRD may induce a conformational change and help promote secondary structure formation within the peptide. FIG. 16C shows a 2D NOESY plot for dFz7-21 displaying α-helical characteristics. By contrast, the 2D NOESY plot for Fz7-21S shown in FIG. 16D indicates that Fz7-21S has limited secondary structure. FIG. 16E shows 1D NMR spectra of Fz7-21, Fz7-21S and dFz7-21 peptides. In FIG. 16E, Fz7-21 shows peak broadening relative to Fz7-21S and dFz7-21 in the tested buffer conditions making assignment of its secondary structure difficult. The arrow in FIG. 16E indicates the proton peak of the amide group from the disulfide cysteine. Together, the presented structure-activity relationship and NMR data provide further biochemical support to the structural model.

TABLE 16
NMR Statistics of Fz7-21 dimer

| Distance restraints | |
|---|---|
| Total | 414 |
| Intra-residue (i − j = 0) | 94 |
| Inter-residue | |
| Sequential (\|i−j\| = 1) | 124 |
| Medium-range (2 ≤ \|i−j\| ≤ 4) | 108 |
| Long-range (\|i − j\| ≥ 5) | 88 |
| Intermolecular | 0 |
| Total dihedral angle restraints | |
| φ | 0 |
| ψ | 0 |
| Average number of violations | |
| Distance constraints | 0.00 ± 0.00 |
| Max. distance constraint violation (Å) | 0.01 |
| Deviations from idealized geometry | |
| Bond lengths (Å) | 0.0006 |
| Bond angles (°) | 0.41 |
| Impropers (°) | 0.079 |
| Ramachandran plot (%) | |
| Residues in most favored regions | 64.3 |
| Residues in additionally allowed regions | 26.6 |
| Residues in generously allowed regions | 9.1 |
| Residues in disallowed regions | 0 |
| Average pairwise r.m.s. deviation (Å) | |
| Residue 5-13 (105-113), Backbone | 0.4 |
| Residue 5-13 (105-113), Heavy atom | 0.8 |

As discussed above, the sequences of hFZD7, hFZD1, hFZD2, mFZD8, hFZD5, hFZD4, hFZD9, hFZD10, hFZD3, and hFZD6 CRDs were aligned in order to gain further insight into the selectivity of Fz7-21 for FZD7 class CRDs. See FIG. 14. The alignment was generated using Clustal Omega followed by manual alignment using disulfide linked cysteines as guide (cysteines are highlighted in gray). Residues on hFZD7 that come into direct contact with the Fz7-21 dimer and identical residues on other FZD CRDs are depicted in bold and underlined text. As Fz7-21 only binds to FZD7-class CRDs (i.e., FZD7 CRD, FZD1 CRD, and FZD2 CRD), the combination of both Binding Site 1 (i.e., LXXHQXYP) and Binding Site 2 (i.e., FGF) shown in FIG. 14 is likely required for specific and selective FZD7-class binding.

The arrangement of hFZD7 CRD dimer is similar to other related frizzled family members such as hFZD5 and mFZD8 CRDs but is in stark contrast to more distantly-related FZD CRD family members, such as hFZD4 CRD (Janda et al. (2012) Science 337, 59-64; Nile et al. (2017) Proc. Natl. Acad. Sci. USA 114, 4147-4152 LID-4110.1073/pnas.1618293114 [doi]; Dann et al. (2001) Nature 412, 86-90; Shen et al. (2015) Cell Res. 25, 1078-1081), in which the lipid-binding grooves appear rather separated from each other and are located on opposite sides away from the dimer interface (FIG. 9B). However, a recently reported structure of hFZD4 CRD in complex with C16: In-7 fatty acid revealed a dimeric CRD with a configuration that is intermediate between the CRD found in hFZD7 CRD/C16:ln-7 complex and that of hFZD7 CRD/Fz7-21 complex. The different architecture of the dimer interface in hFZD4 CRD compared to that of hFZD7 CRDs, in addition to differences in the amino acid sequence within the peptide binding region (FIG. 14), may explain in part the lack of peptide binding to hFZD4 CRD.

Although FZD5, 7 and 8 CRDs share an α-helical dimer architecture (Nile et al. (2017) Proc. Natl. Acad. Sci. USA 114, 4147-4152 LID-4110.1073/pnas.1618293114 [doi]), there are subtle changes in the amino acid residues comprising the dimer interface. In particular, Tyr87 and Phe138 (which is alternatively identified as F130 throughout the specification) in FZD7 CRD are changed to Trp and Tyr in FZD5/8 CRDs, respectively. These two residues comprise part of the Fz7-21 peptide binding region on hFZD7 CRD, and are the only residues (out of seven total CRD residues) within the peptide epitope that are not conserved between FZD7- and FZD8-class CRDs. Although, the residue differences do not present major amino acid changes based on in silico predictions, F138Y may pose steric incompatibility with Fz7-21 at Leu6 and with the FZD7 CRD dimer partner at Val92 and Lys91, potentially disrupting the ‘extended’ lipid-binding groove form of hFZD7 CRD. (F138Y is alternatively identified as F130Y throughout the specification.) These interactions may provide a rationale for the peptide selectivity towards the FZD7 receptor sub-class, based in part on sequence conservation of the peptide binding epitope within the FZD CRD family. Additionally, the dimer interface and lipid-binding groove geometry within hFZD7 CRD contributes to the peptide binding footprint and hence may influence peptide selectivity, not to mention CRD protein dynamics

### Example 4: In Vivo Characterization of Fzd7 Function

To address whether Fz7-21 interferes with FZD7-mediated signaling by disrupting the interaction of Wnts with FZD7, an enzyme-linked immunosorbent assay (ELISA) was developed to measure the binding of biotinylated Wnt (bio-Wnt) to different FZD CRDs in the presence or absence of Fz7-21, dFz7-21 or Fz7-21S. Treatment with Fz7-21 or dFz7-21 enhanced the binding of bio-Wnt3a or bio-Wnt5a to FZD7 class proteins by ˜1.5-2 fold; however, it did not show any effect on the binding of the same Wnt proteins to FZD4 which does not interact with Fz7-21. Moreover, the control peptide Fz7-21S showed no effect on the binding of the different Wnts to FZD CRDs as expected. Without being bound be theory, the substantial conformational change induced by dFz7-21 on hFZD7 CRD, as exemplified by the altered geometry of the lipid binding cavity, may facilitate the accommodation of two Wnt molecules simultaneously onto the hFZD7 CRD dimer (FIG. 4e-h). Molecular modeling suggests that the extended hydrophobic cavity in the peptide-bound hFZD7 CRD structure could potentially accommodate two Wnt fatty acyl moieties which otherwise would be sterically incompatible in the apo hFZD7 CRD structure, providing a plausible explanation for the observed 2-fold increase in ELISA signal. Together the experimental and modeling data suggest that dFz7-21 could enhance the recruitment of Wnt3a and Wnt5a towards FZD7-class CRDs but not FZD4 CRD. The drastic conformational changes observed in the Fz7-21-FZD7 CRD complex may render the receptor incompetent for proper signaling, despite enhanced Wnt binding, as demonstrated by the peptide's inhibitory effects on Wnt signaling.

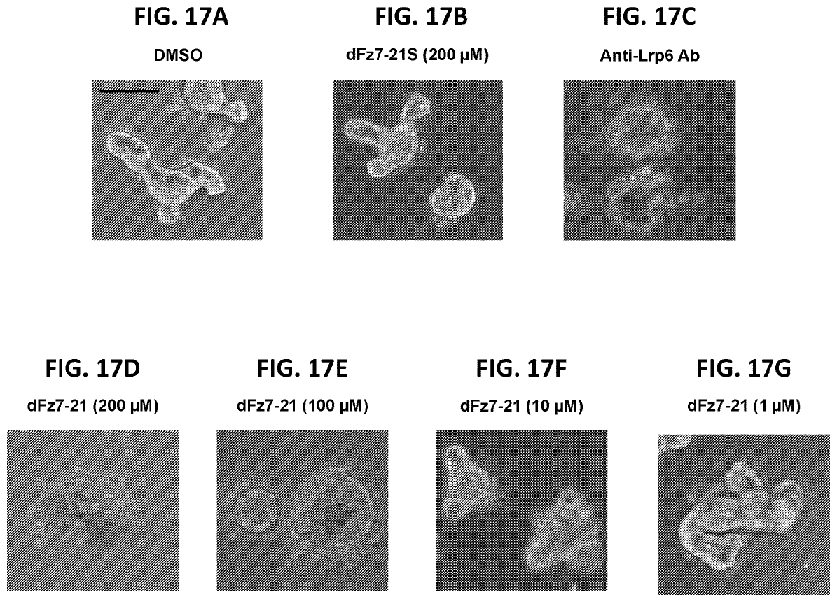

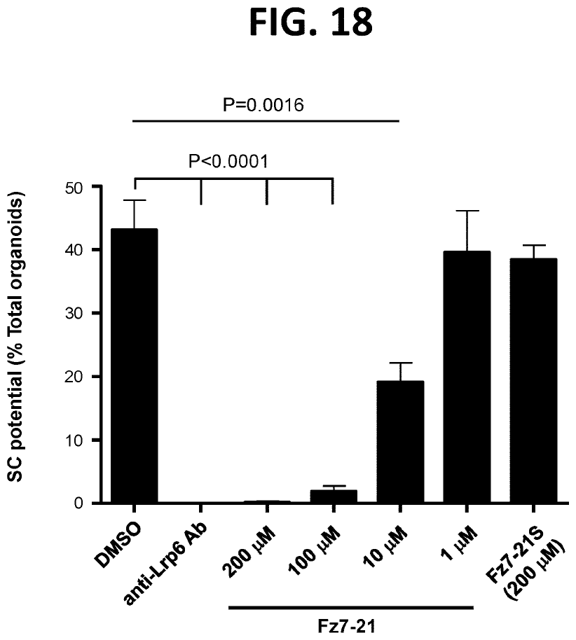

To pharmacologically address the role of FZD7 in stem cell function, the effect of Fz7-21 treatment on organoid cultures established from adult mice intestinal epithelium was assessed (Fatehullah et al (2016) “Organoids as an in vitro model of human development and disease.” Nat Cell Biol 18, 246-254; Barker, N. et al. (2007) “Identification of stem cells in small intestine and colon by marker gene Lgr5.” Nature 449, 1003-1007). The organoid culture system faithfully recapitulates the intestinal epithelium including the presence of crypt-villus morphology. Notably, the crypt region undergoes continuous budding, which is a direct measure of the presence of functional intestinal stem cells (ISCs). Therefore, stem cell function of the organoid population (a Wnt-dependent process) was quantitatively assessed by scoring the number of formed buds per organoid (Grabinger et al. (2014) “Ex vivo culture of intestinal crypt organoids as a model system for assessing cell death induction in intestinal epithelial cells and enteropathy.” Cell Death & Disease 5, e1228). Intestinal mouse organoids were grown in matrigel in the presence of growth factors (noggin, EGF, and R-spondin) and (a) DMSO, (b) 200 μM Fz7-21S (i.e., negative control), (c) anti-Lrp6 blocking antibody (i.e., positive control), (d) 200 μM dimerized dFz7-21, (e) 100 μM dimerized dFz7-21, (f) 10 μM dimerized dFz7-21, or (g) 1 μM dimerized dFz7-21 for 48 h. Morphologies of representative mouse intestinal organoids after 48 h treatment are shown in FIGS. 17A-G. Next, organoid stem cell (SC) potential after peptide treatment was quantified. Organoid SC potential indicates the % of organoids with ≥one bud per organoid. Organoids were treated as above and collected 48 h post-treatment. As shown in FIG. 18, treatment with dFz7-21 dramatically reduced budding events in a concentration-dependent manner, whereas the negative control peptide Fz7-21S did not show any major effects. (The values in FIG. 18 represent the mean±s.e.m from at least three biological replicates. Total number of organoids scored in each condition: n=554 (DMSO); n=547 (dFz7-21, 200 μM); n=627 (dFz7-21, 100 μM); n=648 (dFz7-21, 10 μM); n=287 (dFz7-21, 1 μM); n=528 (Fz7-21S, 200 μM); n=715 (anti-LRP6 Ab, 10 μg/ml).)

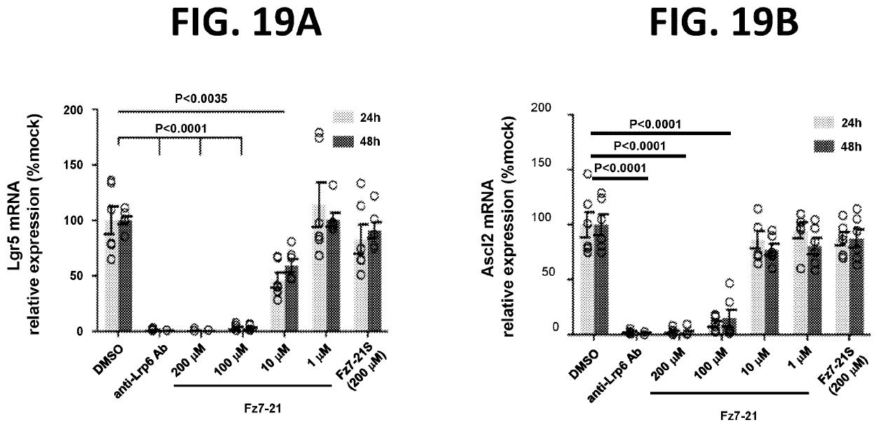

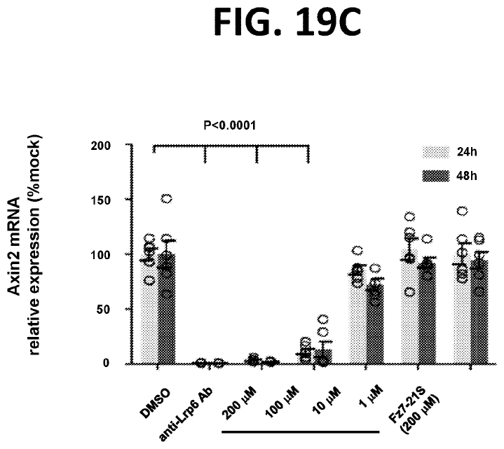

Consistent with disruption of ISC potential, well-characterized ISC markers (Clevers, H. (2012) “The intestinal crypt, a prototype stem cell compartment.” Cell 154, 274-284) such as Lgr5 (FIG. 19A) and Ascl2 (FIG. 19B) were significantly down regulated after 24 and 48 h treatment with dFz7-21. Treatment with Fz7-21S had no effect. See FIGS. 19A and 19B. Similarly, Axin2, a well-established Wnt target gene, was significantly reduced upon dFz7-21 treatment, but not upon Fz7-21S treatment. See FIG. 19C. (The values in FIGS. 19A-19C represent the mean±s.e.m of six biological replicates, each with two technical replicates. Peptide-treated samples were normalized to the DMSO control. Statistics were performed with parametric unpaired t test assuming that both populations have the same SD.) By contrast, Muc2 was upregulated after 24 and 48 h treatment with dFz7-21 but not Fz7-21S. The effects of dFz7-21 on both ISC potential and stem cell transcripts were similar to a control anti-Lrp6 antibody (i.e., a general inhibitor of Wnt signaling).

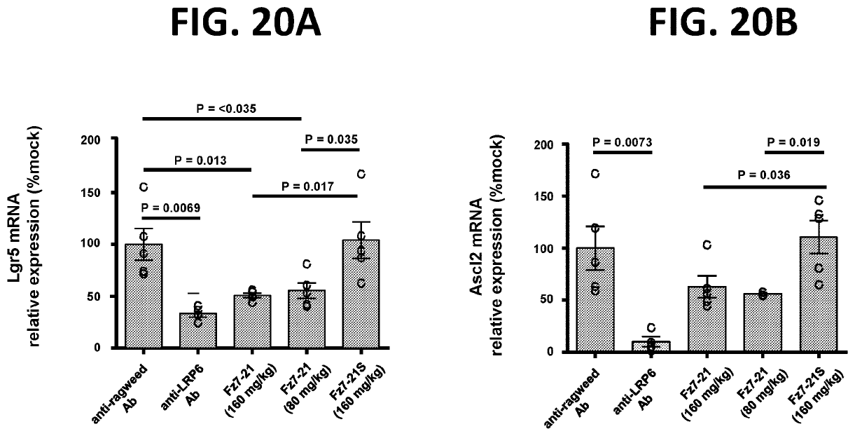

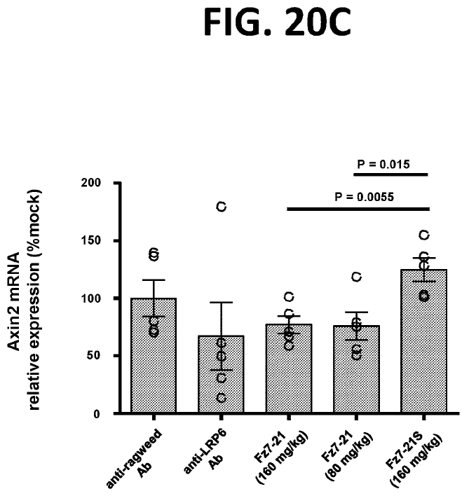

The pharmacological effect of dFz7-21 was further investigated in vivo. Briefly, intestinal epithelia were collected from C57BL/6 mice treated (i.e., via intraperitoneal administration) with dFz7-21, Fz7-21S, anti-ragweed antibody (negative control), or anti-Lrp6 antibody (positive control for inhibition of Wnt signaling) for 6 h. Following treatment, RT-PCR was performed to quantify transcript levels. Various intestinal stem cell (ISC) and Wnt transcripts such as Lgr5 (FIG. 20A), Ascl2 (FIG. 20B) and Axin2 (FIG. 20C) were significantly down regulated after dFz7-21 treatment (The values in FIGS. 20A-20C represent the mean±s.e.m. of five mice with technical duplicates. Statistics were performed with parametric unpaired t test assuming that both populations have the same SD. One value was removed after using the ROUT outlier elimination method where Q=1%. All values were normalized to the anti-ragweed negative control.) This effect was also observed in the sample treated with the anti-Lrp6 antibody. By contrast, treatment with Fz7-21S did not affect Lgr5, Ascl2 and Axin2 transcript levels. See FIGS. 20A-20C. These results are consistent with the data in FIGS. 17A-17G, FIG. 18, and FIGS. 19A-19C.

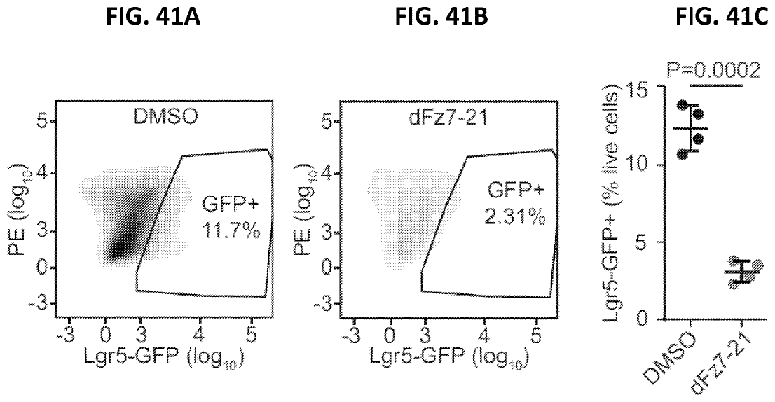

The pharmacological effect of dFz7-21 was further investigated by conducting RNA sequencing on intestinal organoid samples that were treated with dFz7-21 for 6 and 24 h. Unbiased analysis revealed a substantial down-regulation of markers known to be expressed in Lgr5+ ISCs and reserve ISCs upon treatment with dFZ7-21 in a time-dependent manner. Concurrently, there was also an up-regulation of enterocyte markers (pro-differentiation) in the dFz7-21 treated organoids. Among the most down regulated genes upon dFz7-21 treatment were Lgr5, Olfm4 and Asc12. Little or no change in gene expression was observed in the samples treated with mock peptide Fz7-21S or DMSO. Finally, to assess the effect of dFz7-21 directly on stem cells, the presence of GFP+ stem cells in organoids derived from Lgr5-GFP mice was monitored as described in Tian, H. et al. “A reserve stem cell population in small intestine renders Lgr5-positive cells dispensable.” Nature 478, 255-259 (2011). Treatment with dFz7-21 reduced the number of Lgr5-GFP stem cells compared to control DMSO as assayed by flow cytometry (see FIGS. 41A-41C) and confocal microscopy (data not shown). In FIGS. 41A and 41B, Lgr5-GFP organoids were dissociated, treated with SYTOX and analyzed by flow cytometry. FIG. 41A shows a representative plot of live cells expressing GFP+ treated with DMSO, and FIG. 41B shows a representative plot of live cells expressing GFP+ treated with dFz7-21. Quantification of FIGS. 41A and 41B is shown in FIG. 41C.

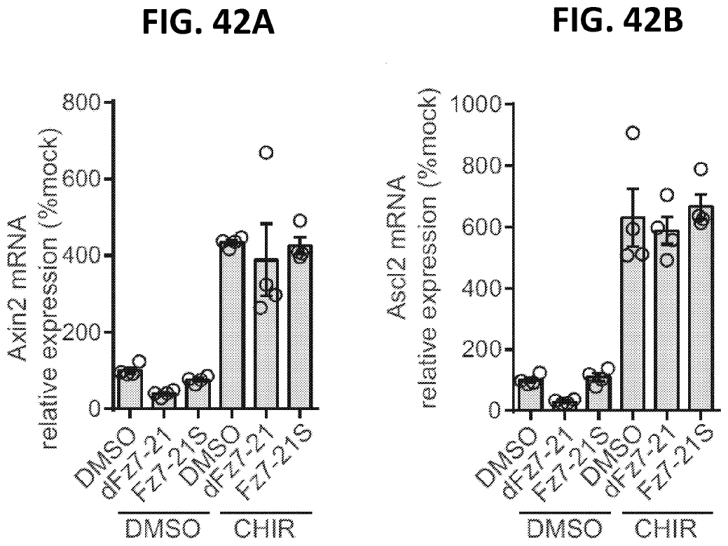

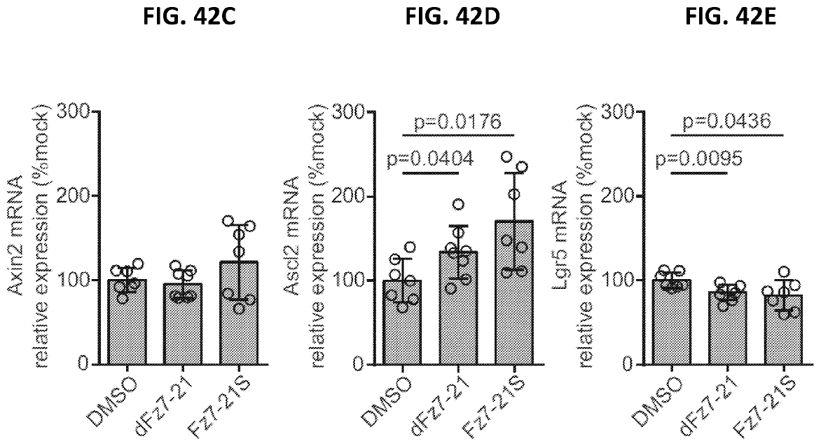

To validate the mechanism of inhibition of dFz7-21 and its specificity, the effect of peptide treatment on organoids that exhibited active downstream Wnt signaling was tested. Briefly, Lgr5-GFP organoids were pre-treated with DMSO or the Gsk3β inhibitor CHIR99021 (5 μM) for 24 h, then treated with DMSO, dFz7-21 (100 μM), or Fz7-21S (100 μM)±CHIR99021. Transcripts were quantified by RT-PCR for Axin2 (FIG. 42A), and Asc12 (FIG. 42B). In a second set of experiments, APC^{min} organoids were cultured and treated with DMSO, dFz7-21 (100 μM), Fz7-21S (100 μM) for 24 h, and transcripts were quantified by RT-PCR for Axin2 (FIG. 42C), Asc12 (FIG. 42D), and Lgr5 (FIG. 42C). (Apc^{Min} (Multiple Intestinal Neoplasia) mice carry a single mutant Apc allele and develop 50-100 benign adenomas in the small intestine by 4-6 months of age, invariably associated with loss of the remaining wild-type gene.) Little or no effect was observed on stem cell (Lgr5, Ascl2) and Wnt signaling (Axin2) markers in intestinal organoids that were pre-treated with CHIR99021 (a Gsk3(3 inhibitor) or had an Apc mutant background. These findings indicate that dFz7-21 acts upstream of β-catenin and APC at the level of the receptor, consistent with the 2D cell culture data (FIG. 2E).

Overall, the representative results discussed in the Examples demonstrate that selective targeting of FZD7 CRD sub-class by dFz7-21 at the lipid binding groove is sufficient to impair Wnt signaling and stem cell function. The drastic conformational changes observed in the Fz7-21-FZD7 CRD complex likely render the receptor incompetent for proper signaling, as demonstrated by the peptide's inhibitory effects in vitro and in vivo, thereby providing a mechanism for the peptide's mode of action.

A potent and selective small peptide antagonist (i.e., Fz7-21 and it derivatives) has been described. This peptide is differentiated, through its high potency, selectivity and pharmacological mode of action, from previously reported antibody- or small molecule-based antagonists, which target multiple sub-classes of the FZD receptor family. See, e.g., Lee et al. (2015) “Structure-based Discovery of Novel Small Molecule Wnt Signaling Inhibitors by Targeting the Cysteine-rich Domain of Frizzled.” J Biol Chem 290, 30596-30606 and Gurney et al. (2012) “Wnt pathway inhibition via the targeting of Frizzled receptors results in decreased growth and tumorigenicity of human tumors.” Proc Natl Acad Sci U.S.A 109, 11717-11722.

The crystal structure of hFZD7 CRD in complex with Fz7-21 reveals a novel CRD conformation. Moreover, the data provided herein define a lipid groove-binding mechanism as the basis for isoform selective FZD inhibition. Peptide Fz7-21 alters the dimer configuration of FZD7 CRD and its lipid-binding cavity, providing the first example of an open-state FZD CRD with an open dimer interface and an ‘extended’ lipid-binding groove relative to apo FZD7 CRD (see FIGS. 12A and 12B). The peptide does not compete with the high affinity Wnt ligands for FZD7 CRD binding, which could be beneficial especially in an in vivo context. While there is additional recruitment of Wnt ligands to FZD CRD in the presence of Fz7-21 in vitro, the drastic conformational changes observed in the Fz7-21-FZD7 CRD complex likely render the receptor incompetent for proper signaling, as demonstrated by the peptide's inhibitory effects in cells and intestinal organoids, thereby offering a plausible explanation for the peptide's mode of action. Moreover, without being bound by theory, it is possible that the peptide will bind to FZD1 and FZD2 CRDs in a similar fashion to FZD7 CRD, given the high degree of sequence conservation among these proteins and the similar findings from ELISA.

The data discussed about indicate that selective targeting of FZD7 CRD sub-class at the lipid binding groove is sufficient to impair Wnt and stem cell function. Fz7-21 serves as a selective pharmacological tool to further probe the role of FZD7 in stem cell and cancer biology. Such findings suggest that there is a critical role for the FZD7 CRD lipid-binding groove geometry in mediating the Wnt signal to regulate Lgr5+ intestinal stem cells. The apparent lack of functional redundancy of FZD7-class receptors at the bottom of the intestinal crypt, coupled with data that show that selective targeting of the FZD7 receptor sub-class, suggests a possible path to pharmacologically target FZD7 with small ligands in tumors dependent on active upstream Wnt signaling (Seshagiri (2012) “Recurrent R-spondin fusions in colon cancer.” Nature 488, 660-664; Jiang et al. (2013) “Inactivating mutations of RNF43 confer Wnt dependency in pancreatic ductal adenocarcinoma. Proc Natl Acad Sci U S. A. 110, 12649-12654; Madan et al. (2016) “USP6 oncogene promotes Wnt signaling by deubiquitylating Frizzleds.” Proc Natl Acad Sci U S. A. 113, E2945-2954).

### Example 5: Fz7-21 Peptide Derivatives

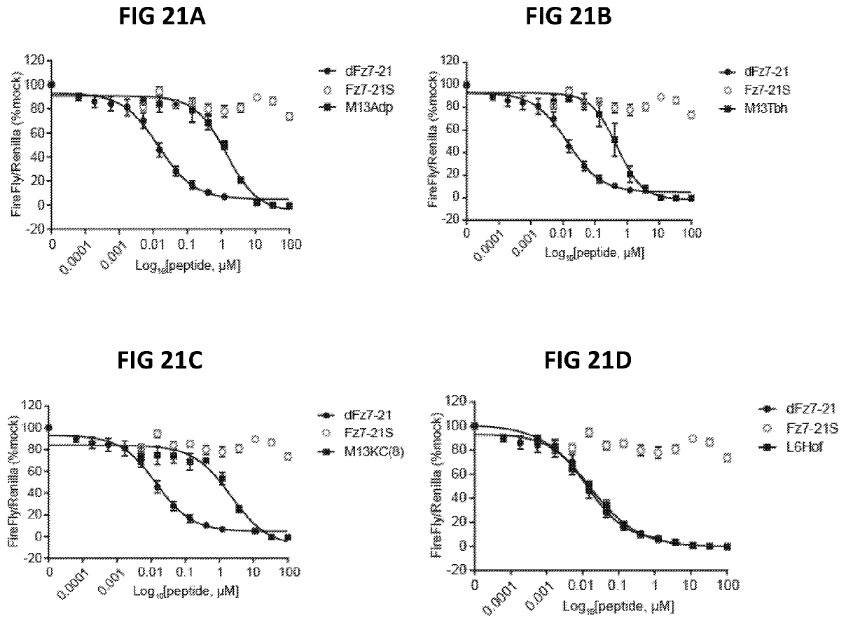

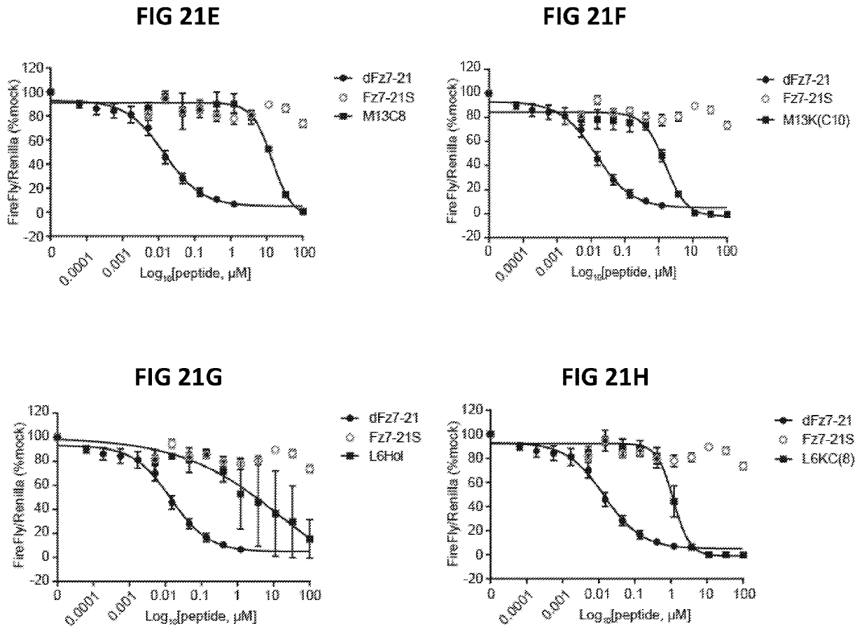

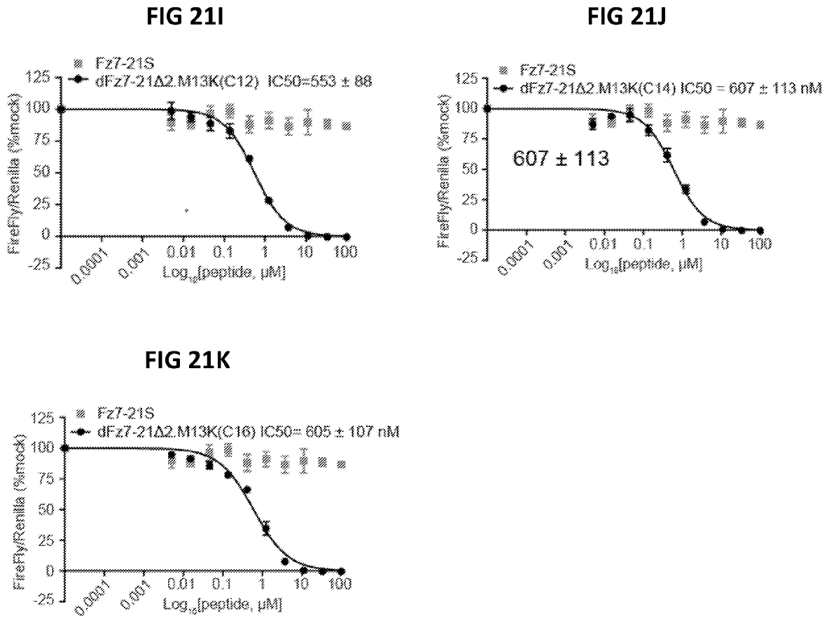

M13 and L6 of Fz7-21 face the open lipid-binding groove on FZD7 CRD. See FIGS. 13, 25A, and 25B. Further experiments were performed to assess whether conjugating a lipid to Fz7-21 at these positions would enhance the affinity of the peptide to FZD7 CRD, interfere with Wnt binding to FZD7 CRD, or both. Lipid-containing derivatives of Fz7-21 (listed in Table 17) were generated and assessed for their abilities to inhibit Wnt3a-mediated β-catenin signaling, i.e., as described above. Briefly, HEK293-TB cells that had been stably transfected with a TCF/LEF-responsive firefly luciferase reporter construct and a constitutively expressed Renilla luciferase construct were stimulated with recombinant mWnt3a (50 ng/mL) in 3-fold serial dilutions of dimerized peptides listed in Table 17 below for 6 hours. DMSO controls were performed in parallel. β-catenin signaling was measured as the ratio of Firefly luciferase to Renilla luciferase normalized to the DMSO control. As shown in Table 17 and FIGS. 21A-21K, dFz7-d21Δ2.L6H and Fz7-21Δ2 impaired Wnt3a-mediated β-catenin signaling in HEK293 cells stimulated with exogenous Wnt3a, with IC50 values comparable to that of dFz7-21. (The values in Table 17 and FIGS. 21A-21K are averages of at least 3 independent replicates (except where otherwise stated), ±standard deviation.

TABLE 17

| | | | SEQUENCE OF | |
|---|---|---|---|---|
| | Best Fit | 95% | MONOMERIC | UNNATURAL |
| | IC50 (nM) | Confidence | PEPTIDE | AMINO |
| PEPTIDE | (n = 3) | Interval | (SEQ ID NO) | ACID |
| dFz7-21** | 14.1 | 8.8 to 22.5 | LPSDDLEFWCHVMY | N/A |
| | | | (SEQ ID NO: 13) | |
| Fz7-21S | Not | Not | LPSDDLEFWSHVMY | N/A |
| | inhibited | inhibited | (SEQ ID NO: 113) | |
| dFz7-21Δ2.M13Adp* | 1,409.7 | 940.0 to | SDDLEFWCHVXY | X = 2-amino-3- |
| | | 2,114.3 | (SEQ ID NO: 114) | decyloxy- |
| | | | | propionic acid |
| dFz7-21Δ2.M13Tbh* | 476.6 | 308.4 to | SDDLEFWCHVXY | X = 6-hydroxy-L- |
| | | 736.7 | (SEQ ID NO: 114) | norleucine |
| dFz7-21Δ2.M13K (C8)* | 2,127.6 | 981.9 to | SDDLEFWCHVXY | X = lysine with |
| | | 4,610.2 | (SEQ ID NO: 114) | octanoic acid |
| | | | | coupled at |
| | | | | epsilon amino |
| | | | | group |
| dFz7-21Δ2.L6Hof* | 14.9 | 11.0 to 20.1 | SDDXEFWCHVMY | X = L- |
| | | | (SEQ ID NO: 115) | homophenylalanine |
| dFz7-21Δ2.M13C8* | 14,0000.0 | 8,498.6 to | SDDLEFWCHVXY | X = 2- |
| | | 23,060.0 | (SEQ ID NO: 114) | aminodecanoic |
| | | | | acid |
| dFz7-21Δ2.M13K | 1505.00 | 1,032.6 to | SDDLEFWCHVXY | X = lysine; |
| (C10)* | | 2,193.6 | (SEQ ID NO: 114) | decaonic acid |
| | | | | coupled at |
| | | | | epsilon amino |
| | | | | group |
| dFz7-21Δ2 (Q519)* | 4.3 | 0.13 to 146.3 | SDDLEFWCHVMY | N-terminal amine |
| | | | (SEQ ID NO: 99) | of peptide is |
| | | | | acetylated and C- |
| | | | | terminal carboxyl |
| | | | | group of peptide |
| | | | | is amidated. |
| dFz7-21Δ2.L6Hol | 16,570.0 | 0.0003 to | SDDXEFWCHVMY | X = L-homoleucine |
| (n = 2)*^{§} | | 9.0x 10¹¹ | (SEQ ID NO: 115) | |
| dFz7-21Δ2.L6KC (8)* | 1,165.7 | 886.0 to | SDDXEFWCHVMY | X = lysine, with |
| | | 1,533.8 | (SEQ ID NO: 115) | octanoic acid |
| | | | | coupled at |
| | | | | epsilon amino |
| | | | | group |
| dFz7-21Δ2.M13K | 553 | | SDDLEFWCHVXY | X = lysine; |
| (C12)* | | | (SEQ ID NO: 114) | dodecaonic acid |
| | | | | coupled at |
| | | | | epsilon amino |
| | | | | group |
| dFz7-21Δ2.M13K | 607 | | SDDLEFWCHVXY | X = lysine; |
| (C14)* | | | (SEQ ID NO: 114) | tetradecaonic |
| | | | | acid coupled at |
| | | | | epsilon amino |
| | | | | group |
| dFz7-21Δ2.M13K | 607 | | SDDLEFWCHVXY | X = lysine; |
| (C16)* | | | (SEQ ID NO: 114) | hexadecaonic acid |
| | | | | coupled at |
| | | | | epsilon amino |
| | | | | group |

**peptide dimerized via disulfide bond at Cys10.
*peptide dimerized via disulfide bond at Cys8.
^{§}peptide was tested in duplicate experiments.

In a subsequent set of experiments, ELISA assays were performed to assess the effects of low concentrations of Fz7-21, dFz7-21, and Fz7-21S (e.g., concentrations that would be within same range as the cellular IC50 of ˜100 nM) the on the binding of Wnt to FZD CRD. Briefly, the assays were performed using (a) FZD1 CRD-Fc, (b) FZD2 CRD-Fc, (c) FZD4 CRD-Fc, or (d) FZD7 CRD-Fc as the capture reagent and biotinylated-Wnt5a as the detection reagent. Peptide (1) dFz7-21, (2) Fz7-21, (3) Fz7-21S or (4) DMSO control was added at incremental concentrations between 0-10 μM. Streptavidin-HRP was used to detect biotinylated Wnt5a, and HRP activity was measured as a function of chemiluminescence detection at 428 nm. Notably, binding of Wnt5a to FZD1 CRD, FZD2 CRD, and FZD7 CRD increased in the presence of Fz7-21 at concentrations below about 0.5 μM (FIG. 22A), indicating that Fz7-21 enhances recruitment of Wnt5a to FZD1-CRD, FZD2 CRD, and FZD7 CRD in a concentration-dependent manner. The most binding was seen in the presence of 0.1 μM Fz7-21. By contrast, Fz7-21 had no such effect on the binding of Wnt5a to the CRD on FZD4, i.e., a FZD that shares fewer sequence similarities with FZD1, FZD2, and FZD7 than FZD1, FZD2, and FZD7 share with each other. Binding of Wnt5a to FZD1-CRD, FZD2 CRD, and FZD7 CRD also increased in the presence of dFz7-21 at concentrations below about 0.5 μM (FIG. 22B), with the most binding seen in the presence of 0.05 μM dFz7-21. dFz7-21 had no such effect on the binding of Wnt5a to the CRD on FZD4. Fz7-21S had no effect binding of Wnt5a to any of the FZD CRDs tested (FIG. 22C).

Similar results were observed using Wnt3a. See FIGS. 22D-22F. Binding of Wnt3a to FZD1 CRD, FZD2 CRD, and FZD7 CRD increased in the presence of Fz7-21 at concentrations below about 0.5 μM (FIG. 22D), with the most binding seen in the presence of 0.05 μM-0.1 μM dFz7-21. Binding of Wnt3a to FZD1 CRD, FZD2 CRD, and FZD7 CRD increased in the presence of dFz7-21 at concentrations below about 0.5 μM (FIG. 22E), with the most binding seen in the presence of 0.01 μM-0.05 μM dFz7-21. Neither Fz7-21 nor dFz7-21 had any effect on Wnt3a binding to FZD4 CRD. Fz7-21S had no effect on Binding of Wnt3a to any of the FZD CRDs tested (FIG. 22F).

The values in FIGS. 22A-22F were normalized to the DMSO control. All assays were performed at 1% DMSO final concentration

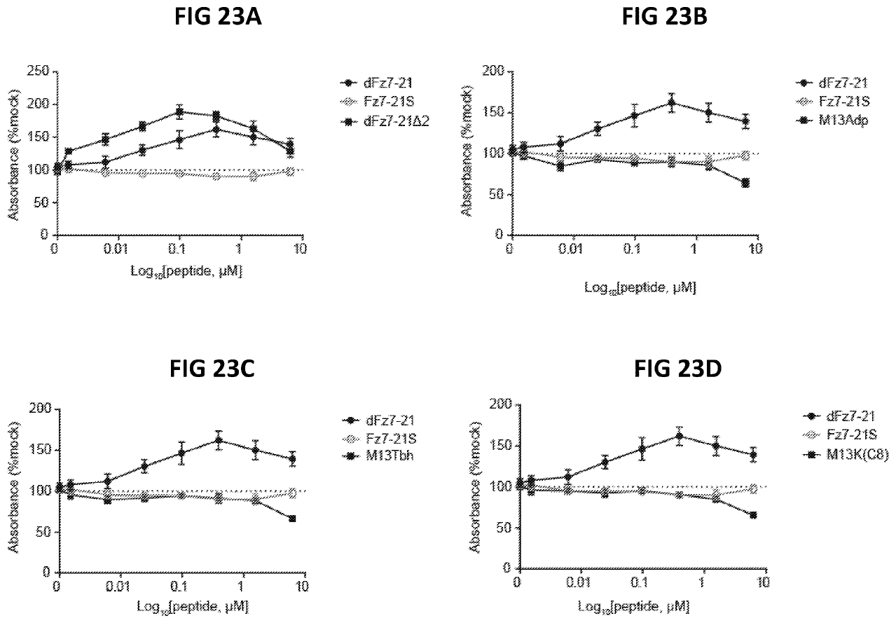

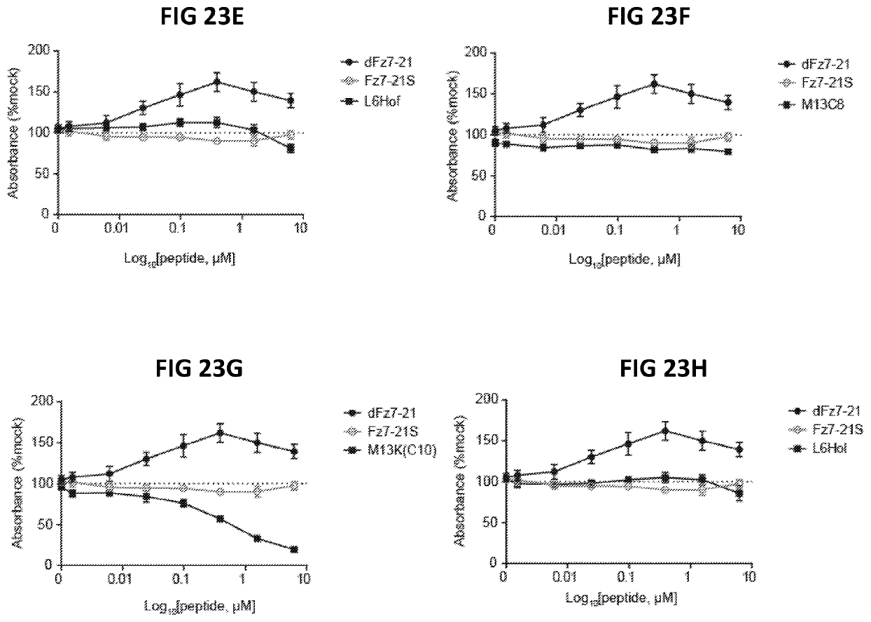

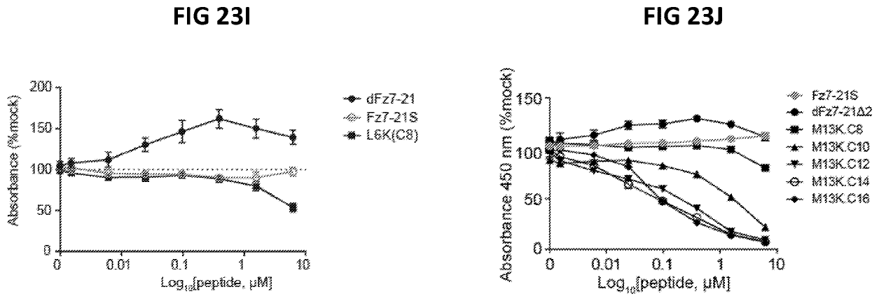

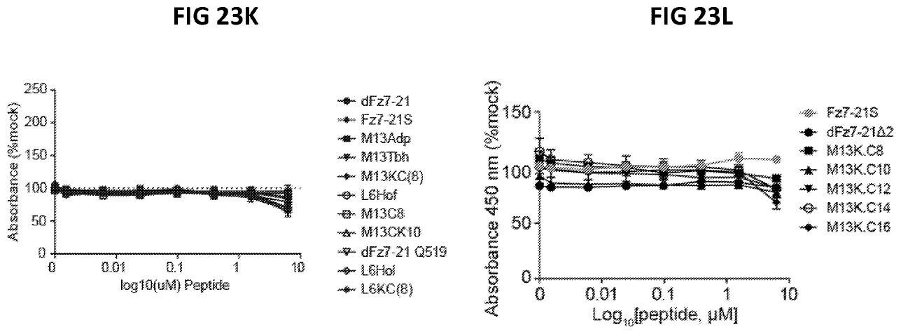

In view of the surprising effect of Fz7-21 and dFz7-21 on the binding of Wnt to FZD1 CRD, FZD2 CRD, and FZD7 CRD, the assays described above were repeated to test the effects of the Fz7-21-derived peptide dimers listed in Table 17 on Wnt5a binding to FZD4 CRD and FZD7 CRD. All Fz7-21-derived peptides dimers tested inhibited recruitment of Wnt5a to FZD7 CRD. FIGS. 23A-23J. The Fz7-21-derived peptide dimers had no effect on Wnt5a binding to FZD4 CRD. See FIGS. 23K and 23L. Fz7-21S had no effect on Wnt5a binding to FZD CRD tested. See FIGS. 23A-23L. In summary, Applicants have surprisingly shown that Fz7-21-derived peptide dimers, which each differ from dFz7-21 by the presence of a hydrophobic unnatural amino acid, inhibit Wnt5a recruitment to FZD7 CRD. Such results suggest that the binding of the Fz7-21-derived peptide dimers listed Table 17 to FZD7 CRD alters lipid-binding cavity of FZD7 CRD from the bent form in the apo structure to an extended form (see FIGS. 12A and 12B), which allows the hydrophobic portion of the unnatural amino acid present in each Fz7-21-derived peptide dimer to infiltrate the elongated lipid binding groove, thus blocking Wnt5a binding to FZD7 CRD.

### Example 6: Materials and Methods of Example 7

### Reagents and Recombinant Proteins

hFZD7 CRD-His [Gln33-Gly168] was expressed as a secreted protein in Trichoplusia ni cells expressing EndoH and treated with Kifunensin. It was then was purified by standard Ni-NTA affinity chromatography followed by size-exclusion chromatography as described earlier²⁰. hFZD5 CRD-His [Ala27-Ala155] was expressed as a secreted protein in Trichoplusia ni cells and was then purified by standard Ni-NTA affinity chromatography followed by size-exclusion chromatography as described in Bourhis, E. et al. Reconstitution of a frizzled8.Wnt3a.LRP6 signaling complex reveals multiple Wnt and Dkk1 binding sites on LRP6. J Biol. Chem. 285, 9172-9 (2010).

### X-Ray Crystallography

C24 bound hFZD7 CRD-His was purified, buffer exchanged into 150 mM NaCl, 50 mM Tris-HCl, pH 7.5 and then concentrated to 25 mg/mL by centrifugation (cat. no. UFC900325; Millipore Amicon Ultra 15). The protein was mixed (1:1) with reservoir solution containing 2.2 M ammonium sulfate, 100 mM Bis-Tris pH 6.5 and incubated at 19° C. by vapor diffusion. Diffraction data were collected at the Stanford Synchrotron Radiation Lightsource (SSRL) 12-2 and solved by molecular replacement at 2.20 Å resolution. hFZD5 CRD-His was purified and buffer exchanged into 150 mM NaCl, 50 mM Tris-HCl, pH 8.0, and then concentrated to 8 mg/mL by centrifugation. Protein was mixed 1:1 with reservoir solution containing 0.1 M sodium citrate tribasic dehydrate pH 5.5, 22% polyethylene glycol 3350 and incubated at 19° C. by vapor diffusion. 0.1% n-Octyl-β-D-glucoside (BOG) was included in the crystallization medium for the FZD5 BOG ω-crystals. For FZD5 CRD ω-crystallization with palmitoleic acid, the latter was prepared as a stock solution (10 mg/ml) in 150 mM NaCl, 50 mM Tris-HCl, pH 8.0 buffer containing 50% DMSO, and it was then mixed with FZD5 CRD (1:1). Co-crystallization was set-up by mixing the protein—fatty acid complex with reservoir solution (1:1) as described above. The reservoir solution also contained 0.1% palmitoleic acid. Diffraction data for hFZD5 CRD:C16:ln-7 and hFZD5 CRD:BOG crystals were collected at Advanced Light Source (ALS) beam line 5.0.1 and solved with molecular replacement at 2.10 Å and 2.20 Å resolution, respectively. Statistics are available in Supplementary Table 1.

hFZD7 CRD Hydrophobic Cavity Representation

To visualize the hydrophobic cavity within hFZD8 CRD, two XWnt8 molecules in complex with mFZD8 CRD (PDB ID# F40A) were superimposed onto each hFZD8 CRD dimer pair using the MatchMaker utility in UCSF Chimera, employing the Needleman-Wunsch alignment algorithm and the BLOSUM-62 matrix. For hFZD5 CRD bound to BOG or C161n-7 and hFZD7 CRD bound to C24 fatty acid, their respective ligands were used to define the hydrophobic cavity. The continuous hydrophobic surface was identified within 5 Å of the bound ligand and visualized according to the hydrophobicity of the cavity.

### In Silico FZD CRD Dimer Interface Analysis

Figures were generated using UCSF Chimera visualization suite (version 1.11). Dimer interface energetics were computed in MOE (Chemical Computing Group; version 2017.05) using the potential energy utility with Amber 10:EHT force field and Born solvation. The energy values from the force field are reported in kcal/mol and are not scaled to reflect actual binding free energies. The complementarity between the Connolly surfaces was ranked using an implementation of the Sc algorithm, which ignores surfaces within 1.5 Å of the edge of the surface (world wide web-ccp4.ac.uldnewsletters/newsletter39/02_sc.html). Prior to calculations, crystallographic models were protonated using Protonate3D to optimize hydrogen placement (MOE version 2017.05). The root-mean-square deviations (RMSDs) were calculated using the alpha carbon of each aligned and superimposed residue of the indicated structures using the MatchMaker utility within the UCSF Chimera suite (version 1.11).

### Example 7: Fz7-21 Peptide Derivatives

FZD receptors mediate Wnt signaling in diverse processes ranging from bone growth to stem cell activity. Yet, the molecular basis for recognition of Wnt cis-unsaturated fatty acyl groups by the CRD of FZD receptors remained elusive until the crystal structures reported herein were invented. This example shows the first crystal structure of human FZD5 CRD bound to C16:1 cis-49-unsaturated fatty acid. Unexpectedly, the crystal structure of human FZD7 CRD bound to a C24 fatty acid was also obtained. Both structures share a conserved novel dimeric arrangement of the CRD. The lipid-binding groove spans both monomers and adopts a U-shaped geometry that accommodates the fatty acid. The mouse FZD8 CRD structure reveals that it also shares the same architecture as the FZD5 and FZD7 CRDs. This example shows a common mechanism for recognition of the Wnt cis-unsaturated fatty acyl group by multiple FZD receptors, and aids in the development of specific FZD receptor inhibitors.

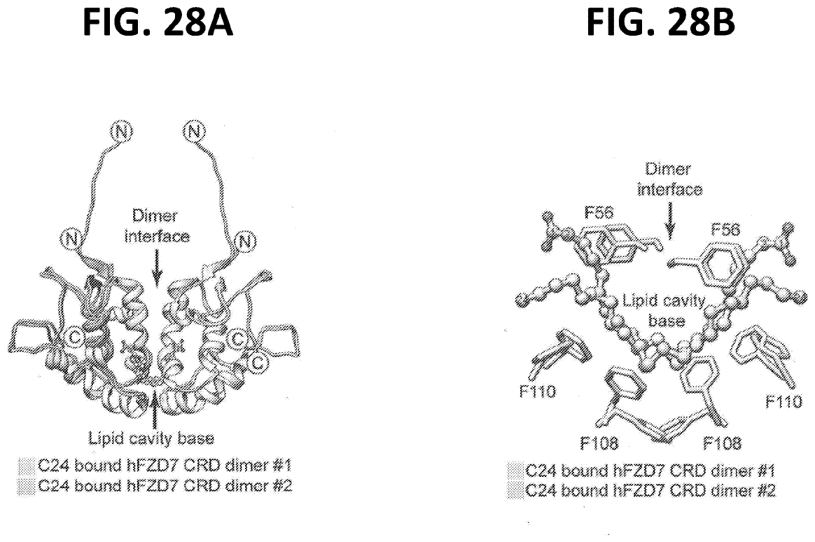

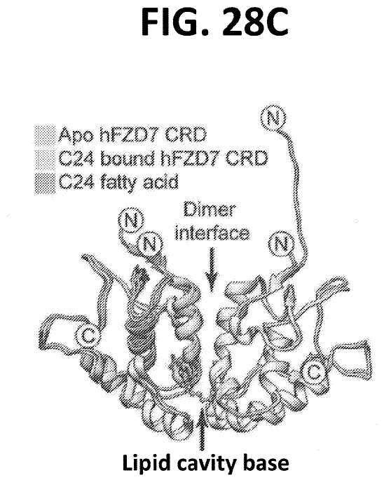

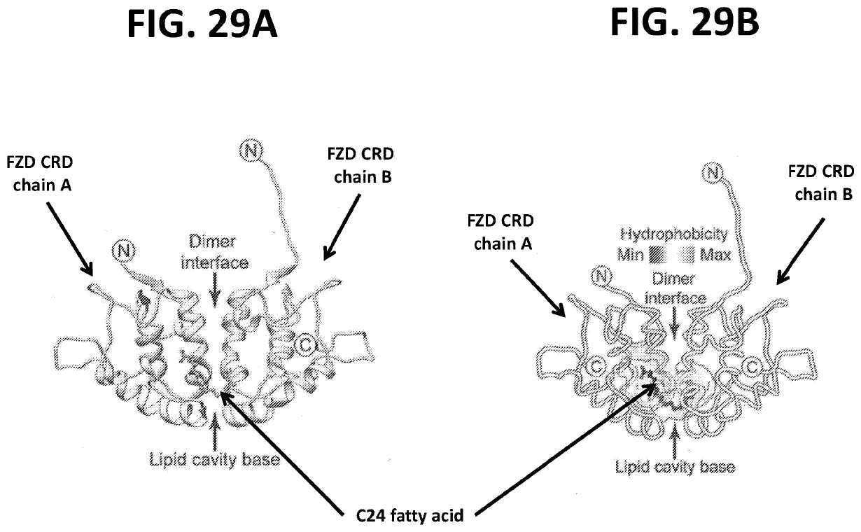

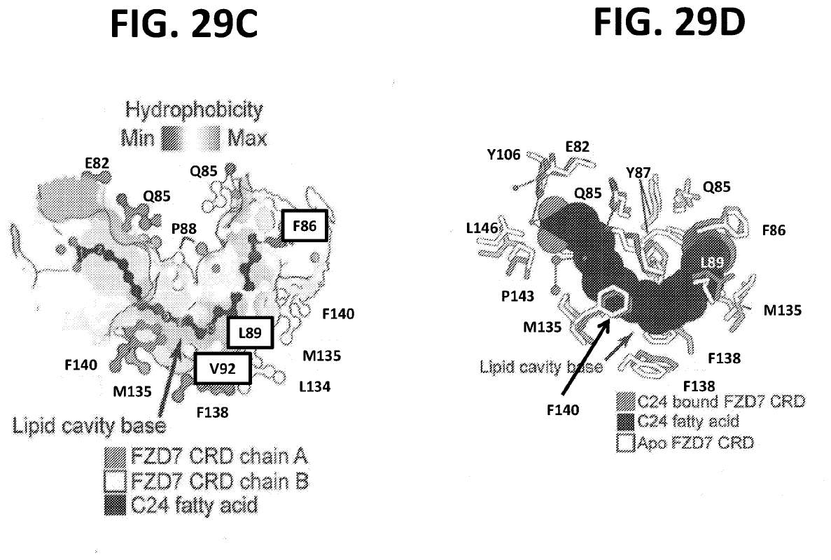

The initial goal of the experiments of this example was to determine the crystal structure of apo hFZD7 CRD. The hFZD7 CRD (residues Gln33-Gly168) was expressed and purified as a soluble secreted protein in insect cells. The X-ray crystal structure of hFZD7 CRD was solved by molecular replacement and refined to a resolution of 2.20 Å (see Table 18). hFZD7 CRD adopted a homo-dimer arrangement, with an alpha-helical dimer interface between two protomers (chains A and B) that comprise the crystallographic asymmetric unit. Unexpectedly, an extra electron density in the lipid-binding cavity was observed, showing an elongated shape that resembled features of a free fatty acid molecule (C24). Such a fatty acid presumably originated in the insect cell expression host. The observed electron density indicated that the carboxylate group of the fatty acid was present in one monomer (chain A), whereas the methyl end of the hydrocarbon chain was present in the other monomer (chain B) (FIGS. 29A, 29B, 29C, and 29D). Each monomer in the hFZD7 CRD dimer contained a lipophilic groove. The two lipid-binding grooves meet at the dimer interface, forming a contiguous and bent (U-shaped) cavity (FIGS. 29B and 29C). Despite the nearly strict non-crystallographic symmetry (NCS) 2-fold arrangement of the C24-bound hFZD7 CRD dimer, there are important differences in the interior structure of the lipophilic grooves in the CRD dimer, likely induced by the inherent asymmetry of the C24 fatty acid (chain A vs chain B r.m.s deviation of 0.847 Å across all 118 atom-pairs; see FIGS. 28A and 28B). Notably, Phe138 and Phe140, two key residues that line the inner surface of the lipid-binding groove, adopt different side chain rotamers between chains A and B, thereby presenting an asymmetric tunnel for binding the asymmetric fatty acid (see FIG. 28B). Additionally, the fatty acid appeared to be predominantly buried in the U-shaped lipid-binding cavity, with C9-C13 positioned at the base of the hydrophobic cavity (FIGS. 29C and 28B).

TABLE 18

| | hFZD5 CRD + | hFZD5 CRD + | hFZD7 CRD + | |
|---|---|---|---|---|
| | BOG | C16:1n-7 | C24 fatty acid | Apo hFZD7 CRD |
| PDB code | TBD | TBD | TBD | TBD |
| X-ray source | 2012_09_19_ALS_502 | 2012_09_19_ALS_502 | 2015_01_21_SSRL_122 | 2015_01_21_SSRL_122 |
| | (CRY11859) | (CRY11964) | (CRYI8651) | (CRY18650) |
| Space group | P3₁21 | P3₁21 | P2₁2₁2 | P2₁2₁2₁ |
| Unit cell | a = b = 123.1 Å, c = 46.9 Å | a = b = 123.4 Å, c = 46.9 Å | a = 97.6 Å, b = 104.9 Å, | a = 39.1 Å, b = 62.8 Å, |
| | α = β = 90°, γ = 120° | α = β = 90°, γ = 120° | c = 41.4 Å | c = 103.2 Å |
| | | | α = β = γ = 90° | α = β = γ = 90° |
| Resolution | 2.20 Å | 2.10 Å | 2.20 Å | 2.00 Å |
| Total number | 20901 (2055)¹ | 24306 (2480)¹ | 22293 (233)¹ | 17922 (103)¹ |
| of reflections | | | | |
| Completeness | 99.9 (99.8) | 100 (100) | 99.9 (99.9) | 99.5 (97.1) |
| (%) | | | | |
| Redundancy | 10.8 (10.0) | 10.9 (10.9) | 6.6 (6.8) | 6.4 (6.2) |
| I/σ | 25.4 (3.2) | 28.1 (3.9) | 14.4 (3.7) | 10.3 (2.6) |
| Rsym² | 0.104 (0.771) | 0.085 (0.716) | 0.134 (0.645) | 0.084 (0.430) |
| Resolution | 50-2.20 Å | 50-2.10 Å | 50-2.20 Å | 50-2.00 Å |
| range | | | | |
| Rcryst³/Rfree⁴ | 0.180/0.224 | 0.171/0.205 | 0.163/0.210 | 0.199/0.252 |
| Non-hydrogen | 2071 | 2152 | 2308 | 1926 |
| atoms | | | | |
| Water | 97 | 139 | 239 | 98 |
| molecules | | | | |
| Average B, | 58.1 | 47.9 | 31.64 | 40.09 |
| Overall | | | | |
| r.m.s.d. bond | 0.011 Å | 0.009 Å | 0.011 Å | 0.006 Å |
| lengths | | | | |
| r.m.s.d. angles | 1.215° | 1.194° | 1.325° | 0.994° |
| Ramachandran | 0.924/0.071/0.005/0 | 0.915/0.085/0/0 | 0.937/0.063/0/0 | 0.944/0.056/0/0 |
| (C/A/G/D) | | | | |

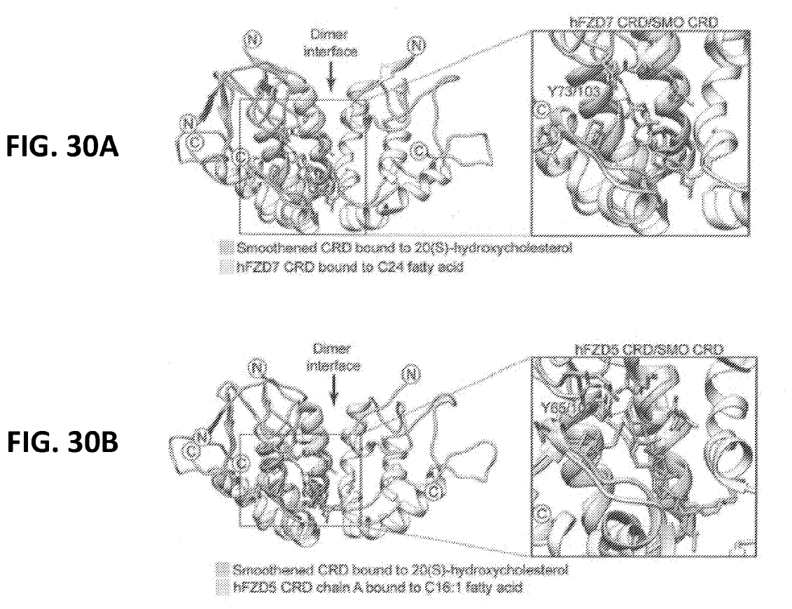

Under different crystallization conditions, the crystal structure of apo hFZD7 CRD was also obtained, which showed a similar dimer configuration and lipid-binding groove geometry as that observed in the structure of C24-bound hFZD7 CRD. Intriguingly, hFZD7 CRD crystallized this time in the absence of any endogenous fatty acid originating from the expression host. Even though both apo and lipid-bound structures superimposed well (r.m.s deviation of 0.979 Å across all 117 atom-pairs; see FIG. 28C), there were some key conformational changes which were obvious in the lipid-bound structure. For instance, the side chain orientations of residues lining the hydrophobic cavity such as Tyr87 and Phe138 (FIG. 29D) were altered, suggesting that the lipid-binding cavity is flexible and may accommodate fatty acids of variable chain length. These findings are consistent with and explain earlier biochemical data demonstrating that Wnt proteins incorporate 13-16 carbon chain fatty acyl groups (Gao, X. & Hannoush, R. N. Single-cell imaging of Wnt palmitoylation by the acyltransferase porcupine. Nat Chem Biol 10, 61-68 (2014)). Finally, it is noteworthy that the carboxylate group of C24 was anchored by a hydrogen bond, which was mediated by a water molecule, to Tyr76 (FIG. 29D). Given the high degree of sequence conservation of Tyr76 across the FZD family members, this interaction highlights a potential molecular mechanism for recognition of free fatty acids by FZD receptors. Of note is that Tyr76 is also conserved within smoothened (SMO), a hedgehog pathway receptor which contains an extracellular FZD-like CRD (Sharpe, H. J., Wang, W., Hannoush, R. N. & de Sauvage, F. J. Regulation of the oncoprotein Smoothened by small molecules. Nat Chem Biol 11, 246-55 (2015)), and forms a polar contact with the hydroxyl group of 20(S)-hydroxycholesterol (Huang, P. et al. Cellular Cholesterol Directly Activates Smoothened in Hedgehog Signaling. Cell 166, 1176-1187.e14 (2016)). Remarkably, the latter molecule was bound in the hydrophobic groove of SMO at the same position where the free fatty acid could bind in the FZD7 CRD structure (FIG. 30A).

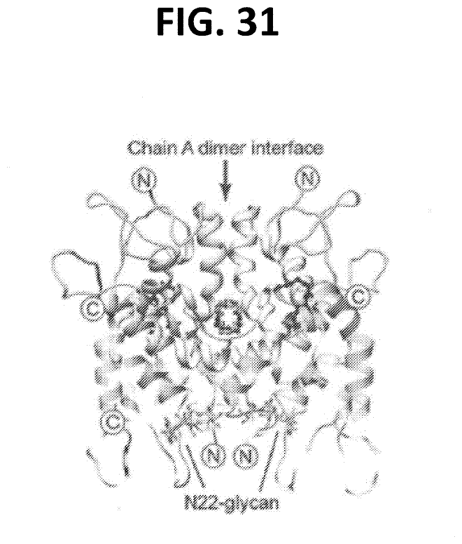

The above unexpected observations in hFZD7 CRD:C24 complex structure, in particular the U-shaped geometry of the lipid-binding groove around the dimer interface and its asymmetric recognition of the fatty acid, prompted the further examination of how other FZD CRDs bind to a C16:1 cis-49-unsaturated fatty acid (C16: In-7), which is the physiologically relevant lipid present on Wnt proteins. There are no reported structures of FZD5 CRD, or any other FZD CRD in complex with unsaturated fatty acids. FZD5 CRD exhibits evolutionary proximity to FZD7 and FZD8 CRDs (FIG. 7). Therefore, hFZD5 CRD (residues Ala27-Ala155) was expressed as a soluble secreted protein in insect cells and purified it to near homogeneity. hFZD5 CRD was ω-crystallized in complex with C16: In-7 fatty acid. The obtained X-ray crystal structure of the complex was solved by molecular replacement and refined to a resolution of 2.10 Å (see Table 18). The crystallographic asymmetric unit consisted of two monomers of hFZD5 CRD (FIG. 31).

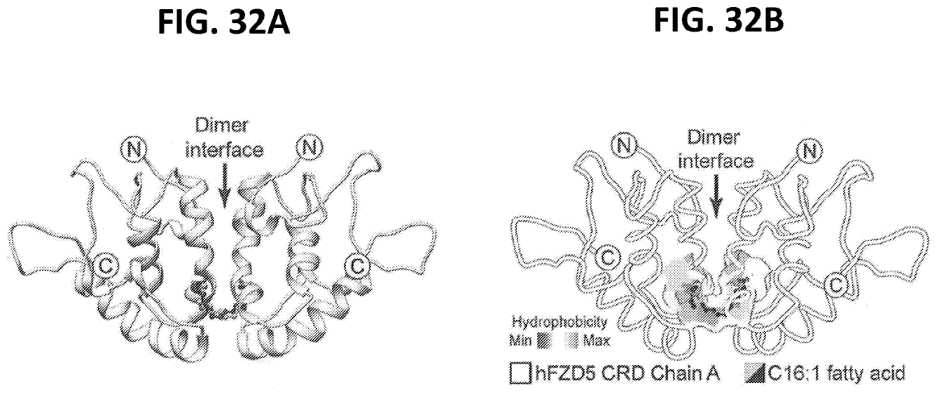

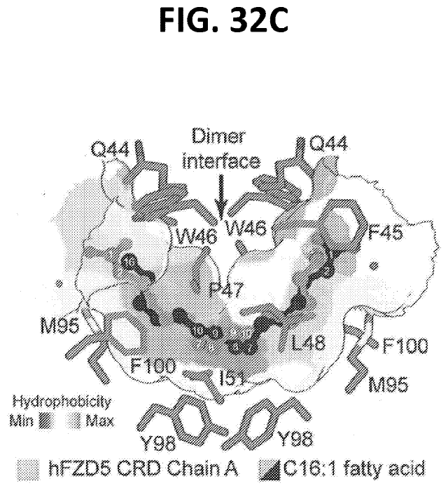

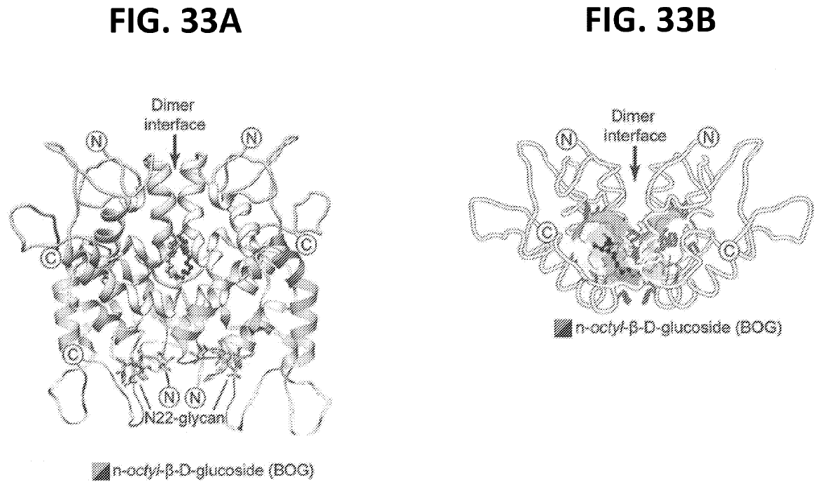

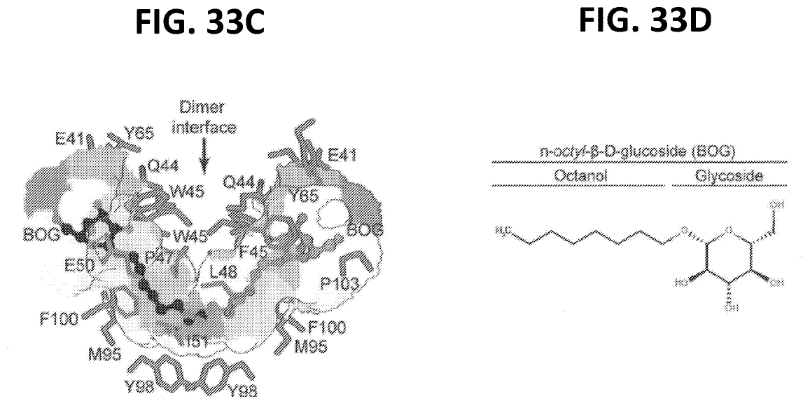

Inspection of hFZD5 CRD structure and crystallography symmetry mates revealed an additional protein-protein interface (chain A homodimer) that is similar to that observed in the structure of apo hFZD7 CRD and hFZD7 CRD bound to C24 (FIGS. 32A and 32B) and FIG. 31). There were two lipid-binding grooves, each originating from one monomer, which formed a contiguous U-shaped cavity, similar to what was observed in hFZD7 CRD dimer structure. The bound C16:ln-7 fatty acid was refined with a 50% occupancy in the structure due to its special location on the two-fold axis, which is equivalent to 100% occupancy within the hydrophobic cavity. The fatty acid can bind in either direction within the lipid-binding groove, with the carboxylate head group positioned proximal to Trp46 and Gln44 residues (FIG. 32C). Remarkably, the “kinked” cis-Δ9-unsaturation site (C9-C10) within the hydrocarbon chain was located at the base of the U-shaped lipid-binding cavity near residues Ile51 and Try98 (Val92 and Phe138 in hFZD7 CRD, respectively) (FIG. 32C). Thus, the crystal structure of hFZD5 CRD in complex with C16: In-7 as disclosed herein reveals an unprecedented atomic resolution view of the geometry of the lipid-binding cavity and how it accommodates a free unsaturated fatty acid, potentially explaining the preferential binding of FZD receptor CRDs to cis-unsaturated fatty acyls on Wnt proteins.

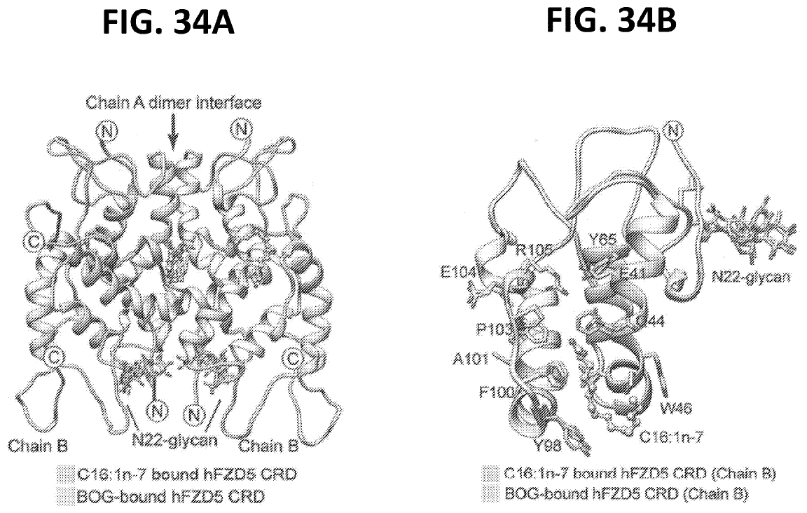

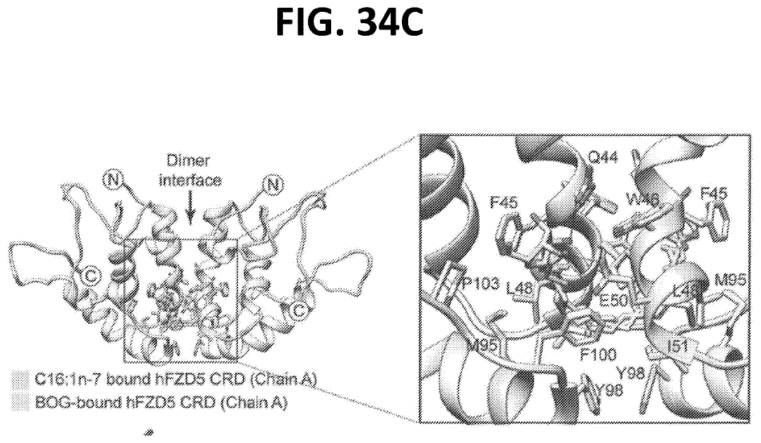

Finally, experiments were performed to determine the crystal structure of apo hFZD5 CRD. However, in this case, hFZD5 CRD crystallized (2.2 Å resolution) in the presence of n-octyl-β-D-glucoside (BOG), which was present in the optimal crystallization buffer. Interestingly, the BOG ligand bound in the hydrophobic cavity of hFZD5 CRD, with 50% occupancy (FIGS. 34A, 34B, 34C, and 34D and Table 18). Overall, the BOG-bound hFZD5 CRD structure was very similar, in terms of the helix-helix dimer interface, the U-shaped lipid-binding cavity and the positioning of the ligand within the hydrophobic cavity, to FZD5 CRD in complex with C16: In-7 (r.m.s deviation of 0.134 over 119 residues; FIGS. 34A, 34B, 34C, and 34D) and FZD7 CRD in complex with C24.

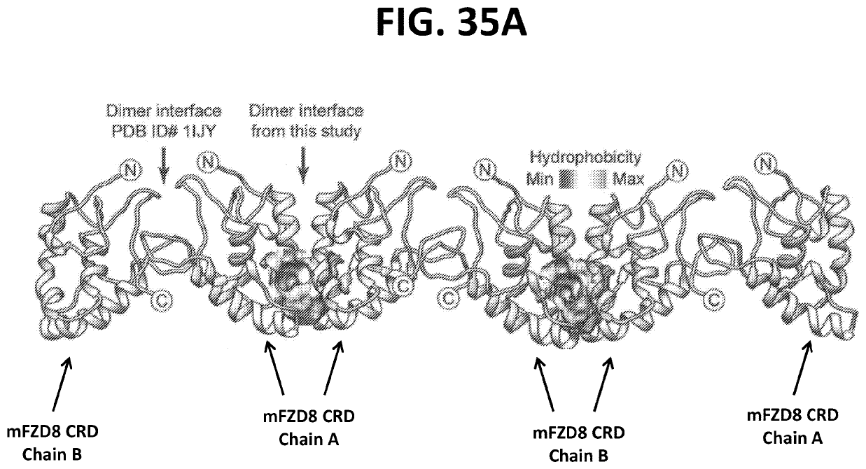

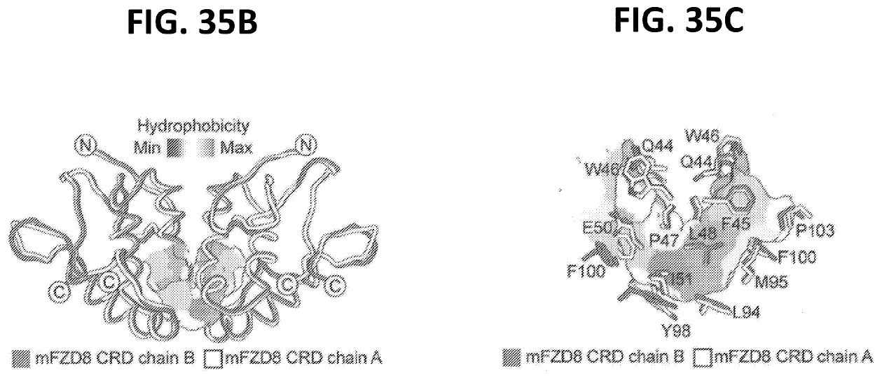

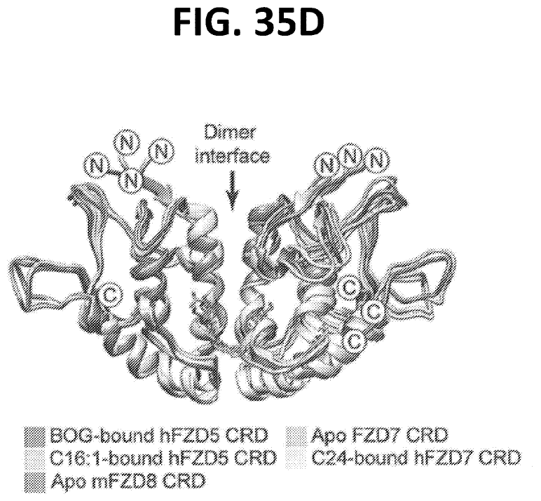

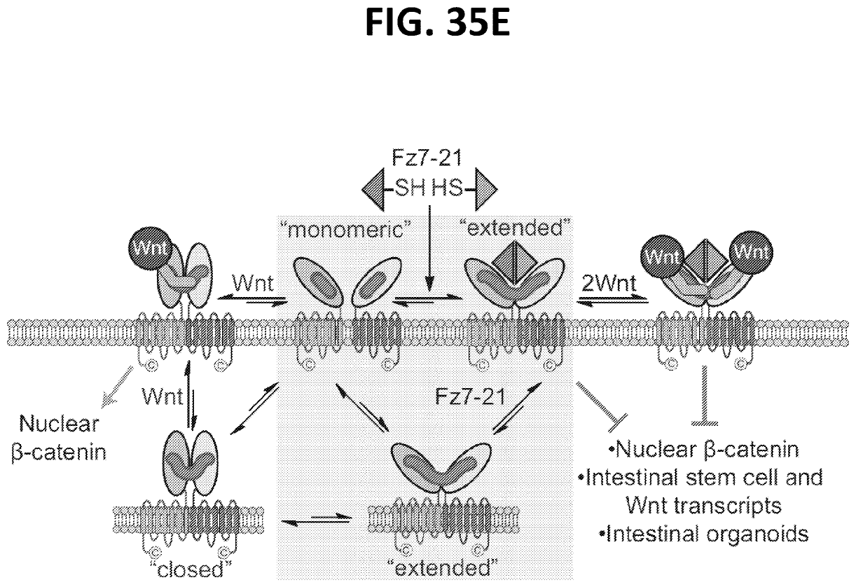

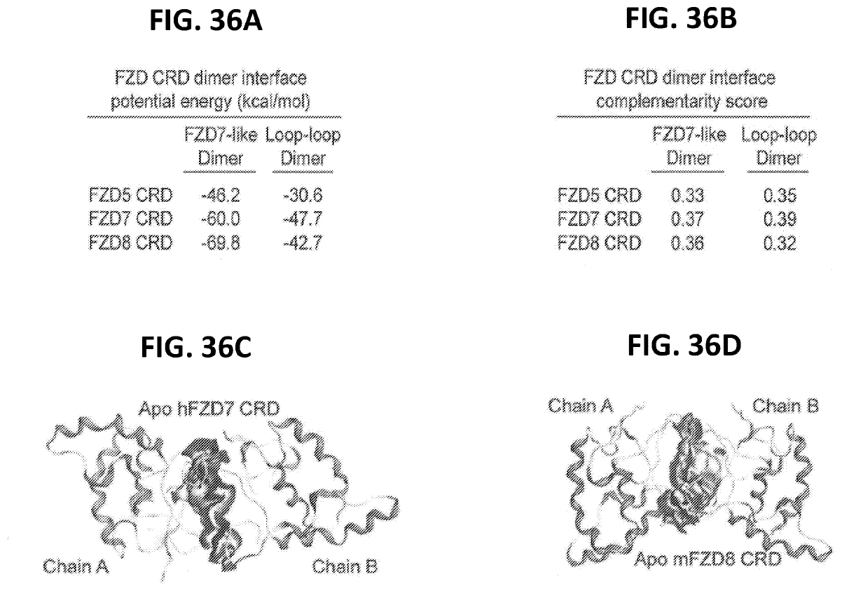

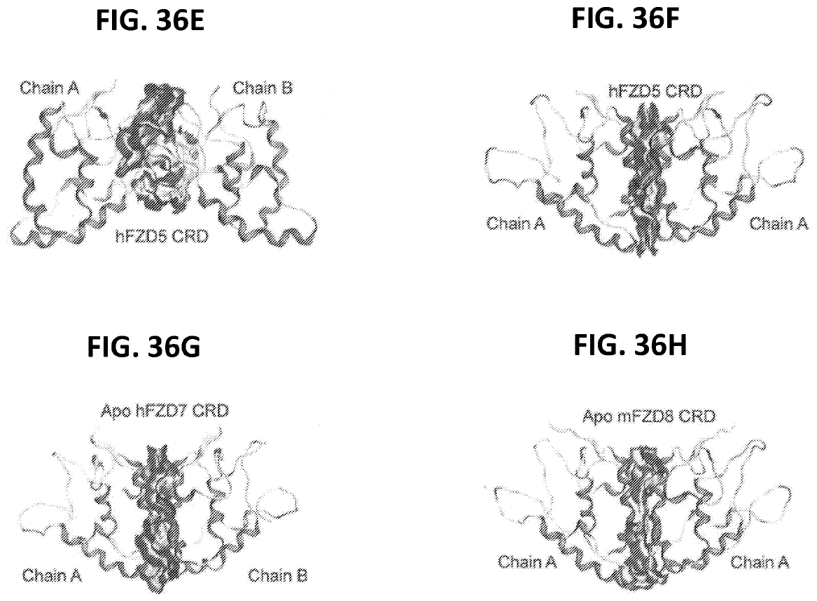

Based on the structural similarity between apo hFZD7, hFZD7 CRD:C24, hFZD5 CRD:C16:ln-7 and hFZD5 CRD:BOG, in particular the dimer configuration and the lipid-binding groove architecture, the published crystallographic data of mFZD8 CRD, the most evolutionarily conserved FZD CRD was reexamined (PDB ID#1IJY)(Dann, C. E. et al. Insights into Wnt binding and signalling from the structures of two Frizzled cysteine-rich domains. Nature 412, 86-90 (2001)) (FIG. 7). Symmetry mate analysis displayed identical dimer configuration as both FZD7 and FZD5 CRDs (FIGS. 35A, 35B, 35C, and 35D). Dann et al. assigned the asymmetric unit based on its similarity to the secreted frizzled-related protein 3 (sFRP3) and its favorable complementarity scores between the loop-loop regions which mediated the dimer interface (Dann, C. E. et al. Insights into Wnt binding and signalling from the structures of two Frizzled cysteine-rich domains. Nature 412, 86-90 (2001)). At the time of publication, neither the cis-unsaturated fatty acylation status of Wnts nor its fatty acid-mediated binding to FZD CRD was known (Janda, C. Y., Waghray, D., Levin, A. M., Thomas, C. & Garcia, K. C. Structural Basis of Wnt Recognition by Frizzled. Science 337, 59-64 (2012); Takada, R. et al. Monounsaturated Fatty Acid Modification of Wnt Protein: Its Role in Wnt Secretion. Developmental Cell 11, 791-801 (2006)), providing no clues about the U-shaped lipid-binding cavity. The in silico analysis disclosed herein, based on energy and complementarity scores (Lawrence, M. C. & Colman, P. M. Shape complementarity at protein/protein interfaces. J Mol. Biol. 234, 946-950 (1993)), supports a helix-helix (FZD7-like) dimer interface as the potential biological interface within the mFZD8 CRD dimer (see FIGS. 36A, 36B, 36C, 36D, 36E, 36F, 36G, and 36H). Computationally, both loop-loop and helix-helix dimer interfaces are similar in their complementarity score; however, the helix-helix dimer interface is energetically more favorable compared to the loop-loop interface for both hFZD7 and mFZD8 CRDs (FIGS. 36A, 36B, 36C, 36D, 36E, 36F, 36G, and 36H).

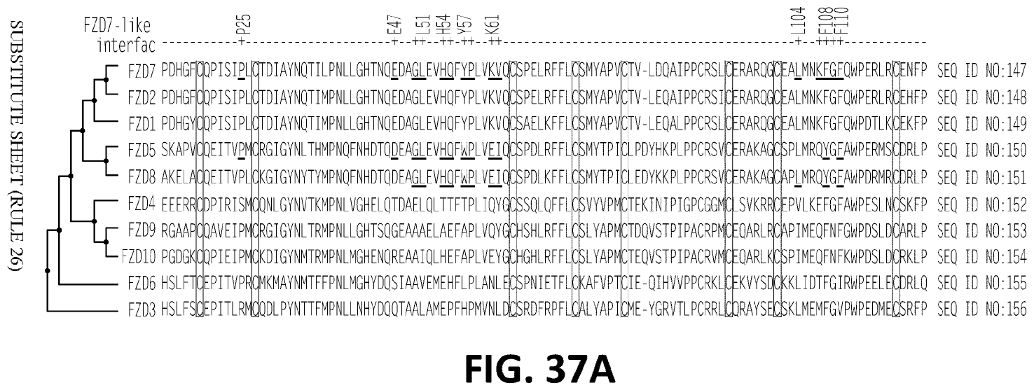

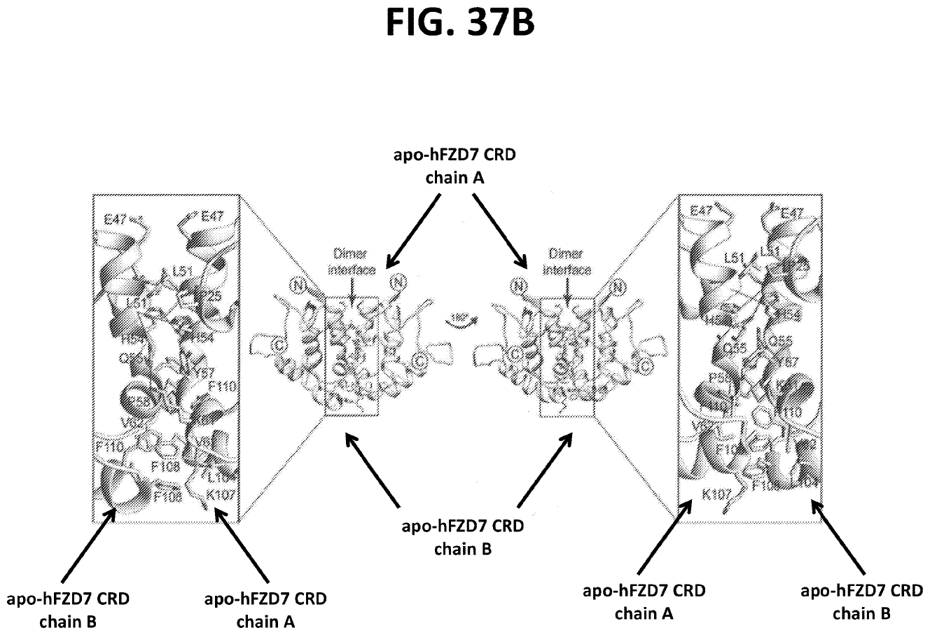

Several lines of evidence support the notion that the helix-helix dimer interface configuration with a U-shaped lipid-binding groove is likely the biological unit, based on the following observations: (a) The mode of binding of the fatty acid is consistent between two different FZD CRD structures (FZD5 and 7), which were independently crystallized under different conditions, revealing that the hydrocarbon fatty acyl chain spans the two lipid-binding grooves simultaneously on both FZD CRD monomers, with the carboxylic acid end on one monomer and the methyl end of the hydrocarbon chain on the other monomer; (b) the conservation of the helix-helix dimer interface across FZD7 and 8 family members (FIGS. 37A and 37B) and (c), the structural similarity and conservation of the “kinked” hydrophobic cavity across multiple FZD family members as shown in this study (FIG. 35D). Additional studies are required to explore the functional relevance of the observed dimer geometry and its regulation of the lipid-binding cavity both in vitro and in vivo. Nonetheless, the structural studies disclosed herein provide compelling support for a contiguous hydrophobic lipid-binding cavity that is occupied by a single fatty acid.

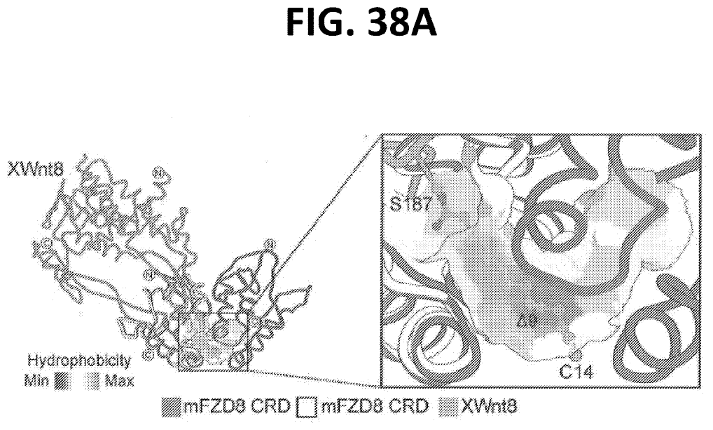

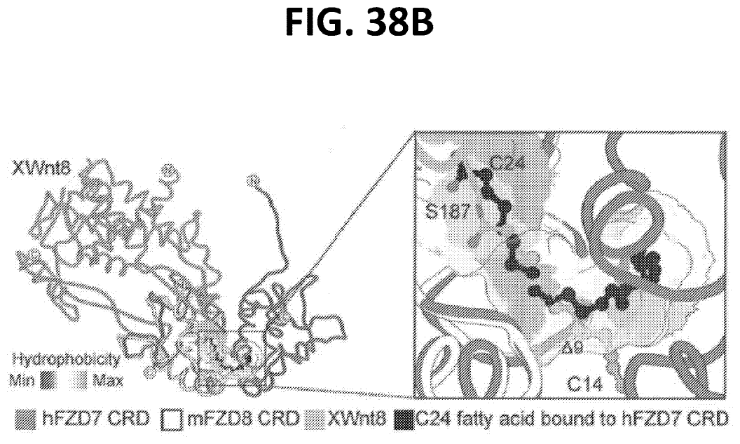

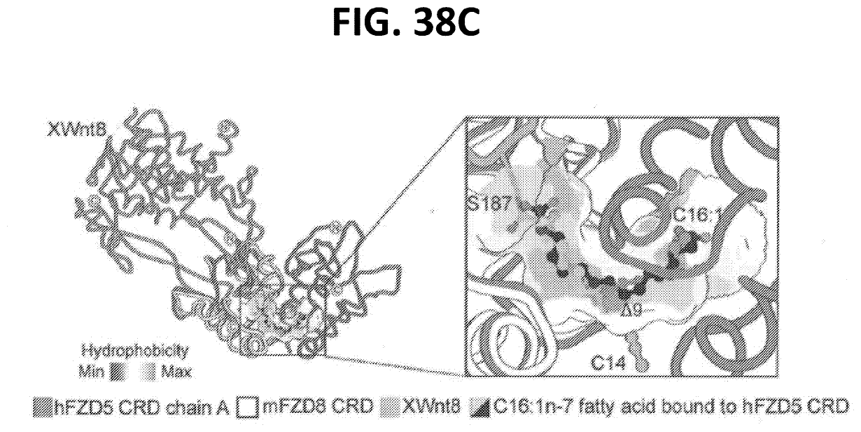

Based on the structural data presented in this study, a molecular model for Wnt interaction with FZD CRDs is proposed. The Wnt fatty acyl group occupies the U-shaped lipid-binding cavity of FZD CRD, with the ‘kinked’ cis-Δ9-unsaturation site positioned at the base of the cavity (FIG. 35E and FIGS. 38A, 38B, and 38C). Superimposition of hFZD5 CRD reported in this study with the published mFZD8 CRD structure bound to XWnt8 (Janda, C. Y., Waghray, D., Levin, A. M., Thomas, C. & Garcia, K. C. Structural Basis of Wnt Recognition by Frizzled. Science 337, 59-64 (2012)) shows that a cis-unsaturated fatty acyl chain originating from a single XWnt8 could occupy both lipid-binding cavities simultaneously, thereby bridging the hFZD5 CRD dimer interface (FIGS. 38A, 38B, and 38C). The same is true for FZD7 and FZD8 CRD dimers, thereby indicating a 1:2 stoichiometry for Wnt-FZD CRD interaction. This is quite different from the 1:1 stoichiometry depicted in the published mFZD8 CRD structure bound to XWnt8 (Janda, C.Y., Waghray, D., Levin, A. M., Thomas, C. & Garcia, K. C. Structural Basis of Wnt Recognition by Frizzled. Science 337, 59-64 (2012)). In this latter case, the lipid-binding groove is in a solvent exposed orientation, which is unfavorable, and does not fully blurry the 16-carbon fatty acyl chain (Janda, C. Y., Waghray, D., Levin, A. M., Thomas, C. & Garcia, K. C. Structural Basis of Wnt Recognition by Frizzled. Science 337, 59-64 (2012)) (FIGS. 38A, 38B, and 38C). It is conceivable that the XWnt8-mFZD8 CRD fusion construct used in the study, which contained an Fc fragment, may not have favored FZD CRD dimer formation, thereby providing an explanation for the observed discrepancy. It is also interesting to note that the authors could not unambiguously determine whether the lipid present was palmitoleic acid or saturated palmitic acid. Indeed, the fatty acyl chain modeled in the XWnt8-mFZD8 CRD structure is surprisingly straight (FIG. 38A), rather than kinked as one would expect from a monounsaturated fatty acid, although the resolution is low; if this was the case, the presence of such a lipid would preclude FZD CRD dimerization. Nonetheless, the Wnt-FZD CRD 1:2 stoichiometry proposed here suggests that the Wnt protein, through its unsaturated fatty acyl group, may help promote FZD CRD dimerization, thereby enhancing FZD receptor clustering at the cell surface and facilitating subsequent downstream signalosome assembly (Bienz, M. Signalosome assembly by domains undergoing dynamic head-to-tail polymerization. Trends Biochem Sci 39, 487-95 (2014)).

In conclusion, reported herein is the first crystal structures for two FZD CRDs (FZD5 and FZD7) in complex with free fatty acids, providing a long sought-after structural rationale for how unsaturated fatty acid may interact with FZD CRDs. These studies reveal that the lipid-binding groove is flexible, accommodating a single fatty acid that binds to both CRD monomers simultaneously. Upon reexamining the earlier published structures (Dann, C. E. et al. Insights into Wnt binding and signalling from the structures of two Frizzled cysteine-rich domains. Nature 412, 86-90 (2001)), it was found that mFZD8 CRD also shares similar structural features as hFZD7 and hFZD5 CRDs reported here, including an alpha-helical dimer interface and a contiguous U-shaped lipid-binding cavity, resulting in the revisiting of the interpretation of the dimer in that structure. Together the experimental data provided in this example suggests a common mechanism among multiple FZD CRDs for recognition of cis-unsaturated fatty acids, and provide a molecular picture into how Wnts could interact with the frizzled receptor and promote its dimerization via the cis-unsaturated fatty acyl group. Finally, the findings herein may facilitate the development of pharmacological strategies to target specific FZD CRD receptor sub-classes by taking advantage of the newly discovered dimer interface, and the lipid-binding groove configuration and its flexibility.

Regeneration of the adult intestinal epithelium is mediated by a pool of cycling stem cells, located at the base of the crypt, that express the leucine-rich repeat-containing G-protein coupled receptor 5 (Lgr5). Out of the ten mammalian frizzled (FZD) receptors, FZD7 is enriched in Lgr5+ intestinal stem cells and plays a critical role in their self-renewal. Recent studies suggest that FZD7 could be a potential pharmacological target for diseases associated with stem cell dysfunction; however, approaches and structural bases for selective FZD inhibition remain poorly defined. FZDs interact with Wnt proteins by binding, in part, to their fatty acyl group via a lipid-binding groove, located within the FZD cysteine-rich domain (CRD). Here we identify a highly potent and selective peptide that binds to FZD7 CRD and alters the architecture of its lipid-binding groove. Treatment with FZD7-binding peptide impaired Wnt signaling and down regulated genes primarily expressed in the stem cell compartment of intestinal organoids. According to the data herein, a lipid groove-binding mechanism serves as a basis for isoform-selective FZD inhibition, and implicate a role for the FZD7 CRD lipid-binding groove geometry in intestinal stem cell function.

The preceding Examples are offered for illustrative purposes only, and are not intended to limit the scope of the present invention in any way. Various modifications of the invention in addition to those shown and described herein will become apparent to those skilled in the art from the foregoing description and fall within the scope of the appended claims.

1. A ligand comprising a non-naturally occurring peptide that binds to a cysteine rich domain (CRD) of the Frizzled 7 (FZD7) receptor.

2. The ligand of claim 1, wherein the peptide specifically binds the CRD of FZD7.

3. The ligand of claim 1, wherein the peptide does not bind to a CRD of a FZD receptor selected from:
(a) the group consisting of: FZD3, FZD4, FZD5, FZD6, FZD8, FZD9, or FZD10; or
(b) the group consisting of: FZD1, FZD2, FZD3, FZD4, FZD5, FZD6, FZD8, FZD9, or FZD10.

4. (canceled)

5. The ligand of claim 1, wherein the peptide further binds FZD1 and FZD2.

6. The ligand of claim 1, wherein the peptide is (a) linear or (b) cyclic.

7. (canceled)

8. The ligand of claim 1, wherein the peptide is (a) between 8-16 amino acids in length, or (b) between 11-14 amino acids in length.

9. (canceled)

10. The ligand of claim 1, wherein the FZD7 is hFZD7.

11. The ligand of claim 10, wherein the peptide specifically binds a binding region of hFZD7 CRD comprising at least three amino acids selected from the group consisting of: Leu81, His84, Gln85, Tyr87, Pro88, Phe138, and Phe140.

12. The ligand of claim 1, wherein the peptide comprises an amino acid sequence set forth in:
X₁X₂X₃DDLX₄X₅WCHVMY(SEQ ID NO:100)
(a) wherein each of X₁-X₃ is no amino acid, any amino acid, or an unnatural amino acid, and wherein each of X₄-X₅ is any amino acid or an unnatural amino acid;
(b) X₁ is L, X₂ is P, X₃ is 5, X₄ is E, and X₅ is F; or
(c) X₁ is no amino acid, X₂ is no amino acid, X₃ is 5, X₄ is E, and X₅ is F.

13. (canceled)

14. The ligand of claim 12, wherein the peptide comprises the amino acid sequence set forth in LPSDDLEFWCHVMY (SEQ ID NO: 13) or SDDLEFWCHVMY (SEQ ID NO: 99).

15-16. (canceled)

17. The ligand claim 1 wherein the N-terminal amine of the peptide is acetylated, wherein the C-terminal carboxyl group of the peptide is amidated, or wherein the N-terminal amine of the peptide is acetylated and the C-terminal carboxyl group of the peptide is amidated.

18. The ligand claim 1, wherein the peptide enhances the binding of a Wnt to the CRD of the FZD7 receptor.

19. The ligand of claim 18, wherein the FZD7 receptor is an hFZD7 receptor.

20. The ligand of claim 1, wherein the peptide comprises an amino acid sequence set forth in:
(a) SDDLEFWCHVXY (SEQ ID NO: 114), wherein X is (i) any amino acid, (ii) an unnatural amino acid, or (iii) an unnatural amino acid selected from the group consisting of: 2-amino-3-decyloxy-propionic acid, a derivative of lysine comprising octanoic acid coupled at epsilon amino group, 2-aminodecanoic acid, a derivative of lysine comprising decanoic acid coupled at epsilon amino group, and 6-hydroxy-L-norleucine; or
(b) SDDXEFWCHVMY (SEQ ID NO: 115), wherein X is any amino acid, or an unnatural amino acid, and wherein the unnatural amino acid is selected from the group consisting of: L-homoleucine, L-homophenylalanine, and a derivative of lysine comprising octanoic acid coupled at epsilon amino group;
(c) any one of SEQ ID NOs: 1-31 and 39-99; or
(d) any one of SEQ ID Nos: 32-98.

21-24. (canceled)

25. The ligand of claim 1, wherein the peptide inhibits Wnt signaling with an IC₅₀ of 120 nM or less.

26. The ligand of claim 1, wherein the peptide has an EC₅₀ value of 90 nM or less.

27. The ligand of claim 1, wherein the peptide is conjugated to a lipid.

28-30. (canceled)

31. The ligand of claim 1, wherein the peptide in the ligand is dimerized.

32-33. (canceled)

34. A composition comprising the ligand of claim 1 and a pharmaceutically acceptable carrier.

35. A method of inhibiting Wnt signaling in a cell, comprising contacting the cell with the ligand of claim 1.

36. A method of inhibiting stem cell proliferation, comprising contacting a stem cell with the ligand of claim 1.

37-39. (canceled)

40. A method of killing a cancer cell comprising contacting the cancer cell with the ligand of claim 1.

41. (canceled)

42. A method treating cancer in a subject, comprising administering an effective amount of the composition of claim 34 to the subject.

43-47. (canceled)

48. A kit for treating cancer, comprising:
(a) the ligand of claim 1, and
(b) and instructions for administering the ligand to a subject that has cancer.

49. (canceled)

50. A ligand comprising the non-naturally occurring peptide set forth in LPSDDLEFWSHVMY (SEQ ID NO: 113).


SEQUENCE LISTING (156 sequences)

SEQ ID NO: 1 - protein, 14 aa, Artificial Sequence
  Synthetic Construct
YEHLHDLMDLIRPW

SEQ ID NO: 2 - protein, 16 aa, Artificial Sequence
  Synthetic Construct
TYFDDICNLILPWANP

SEQ ID NO: 3 - protein, 16 aa, Artificial Sequence
  Synthetic Construct
PQDLLDWCHYMIVSSD

SEQ ID NO: 4 - protein, 14 aa, Artificial Sequence
  Synthetic Construct
ACSYVIDLWNQCLT

SEQ ID NO: 5 - protein, 16 aa, Artificial Sequence
  Synthetic Construct
PCSVICLPDWSSLLFI

SEQ ID NO: 6 - protein, 12 aa, Artificial Sequence
  Synthetic Construct
DTDLHQWCLWFT

SEQ ID NO: 7 - protein, 14 aa, Artificial Sequence
  Synthetic Construct
FWMLLQEGFAFWFP

SEQ ID NO: 8 - protein, 14 aa, Artificial Sequence
  Synthetic Construct
FELLLDLGDLIRLW

SEQ ID NO: 9 - protein, 14 aa, Artificial Sequence
  Synthetic Construct
ACSYVIDLWNLCLR

SEQ ID NO: 10 - protein, 14 aa, Artificial Sequence
  Synthetic Construct
ASELHDWCRMMFPW

SEQ ID NO: 11 - protein, 14 aa, Artificial Sequence
  Synthetic Construct
ISLIEAMIALDRVF

SEQ ID NO: 12 - protein, 14 aa, Artificial Sequence
  Synthetic Construct
PPNVHEGCWSMFPW

SEQ ID NO: 13 - protein, 14 aa, Artificial Sequence
  Synthetic Construct
LPSDDLEFWCHVMY

SEQ ID NO: 14 - protein, 12 aa, Artificial Sequence
  Synthetic Construct
DTDLLQWCLWFT

SEQ ID NO: 15 - protein, 14 aa, Artificial Sequence
  Synthetic Construct
FWMQLQDGFAIWFP

SEQ ID NO: 16 - protein, 16 aa, Artificial Sequence
  Synthetic Construct
PCSVICLPDWSSLLFI

SEQ ID NO: 17 - protein, 14 aa, Artificial Sequence
  Synthetic Construct
GDFWPGSLLWEILV

SEQ ID NO: 18 - protein, 14 aa, Artificial Sequence
  Synthetic Construct
ILTFEYFWILGLIL

SEQ ID NO: 19 - protein, 10 aa, Artificial Sequence
  Synthetic Construct
LPLFFLSYVL

SEQ ID NO: 20 - protein, 16 aa, Artificial Sequence
  Synthetic Construct
FLPDQHSHLFLPWGEP

SEQ ID NO: 21 - protein, 16 aa, Artificial Sequence
  Synthetic Construct
SCQMWSNLRVLFLSYW

SEQ ID NO: 22 - protein, 12 aa, Artificial Sequence
  Synthetic Construct
VFVPFSELTSLC

SEQ ID NO: 23 - protein, 14 aa, Artificial Sequence
  Synthetic Construct
IWFKGRFVEFSSLV

SEQ ID NO: 24 - protein, 16 aa, Artificial Sequence
  Synthetic Construct
NAFWRDQCLEWFIICL

SEQ ID NO: 25 - protein, 16 aa, Artificial Sequence
  Synthetic Construct
EHDLLLRAMNSFVLIF

SEQ ID NO: 26 - protein, 10 aa, Artificial Sequence
  Synthetic Construct
FCENPYIICW

SEQ ID NO: 27 - protein, 10 aa, Artificial Sequence
  Synthetic Construct
NPPPECFLSK

SEQ ID NO: 28 - protein, 16 aa, Artificial Sequence
  Synthetic Construct
VFFYHSLFFIKLILDP

SEQ ID NO: 29 - protein, 14 aa, Artificial Sequence
  Synthetic Construct
ERRVCYPWFEVSQP

SEQ ID NO: 30 - protein, 16 aa, Artificial Sequence
  Synthetic Construct
LSSGKKVSSYWFNFWF

SEQ ID NO: 31 - protein, 8 aa, Artificial Sequence
  Synthetic Construct
FWFDFWFG

SEQ ID NO: 32 - protein, 16 aa, Artificial Sequence
  Synthetic Construct
SSDFSGCLSWCDLIFG

SEQ ID NO: 33 - protein, 16 aa, Artificial Sequence
  Synthetic Construct
FDFCSVMPQFIYCPGD

SEQ ID NO: 34 - protein, 16 aa, Artificial Sequence
  Synthetic Construct
HLSDVCCSDWCDLVFW

SEQ ID NO: 35 - protein, 16 aa, Artificial Sequence
  Synthetic Construct
TSDFSWCLSWCDLIFW

SEQ ID NO: 36 - protein, 16 aa, Artificial Sequence
  Synthetic Construct
FDFCTVMPHFIYCPGD

SEQ ID NO: 37 - protein, 16 aa, Artificial Sequence
  Synthetic Construct
FDFCSVMPHFIYCPGD

SEQ ID NO: 38 - protein, 16 aa, Artificial Sequence
  Synthetic Construct
HLSDVFCSDWCDLVFW

SEQ ID NO: 39 - protein, 14 aa, Artificial Sequence
  Synthetic Construct
VAADDLAAWCHVMY

SEQ ID NO: 40 - protein, 14 aa, Artificial Sequence
  Synthetic Construct
AASDDLEFWCHVMY

SEQ ID NO: 41 - protein, 14 aa, Artificial Sequence
  Synthetic Construct
AASDDLEFWCHVMY

SEQ ID NO: 42 - protein, 14 aa, Artificial Sequence
  Synthetic Construct
APSDDVAFWCHVMY

SEQ ID NO: 43 - protein, 14 aa, Artificial Sequence
  Synthetic Construct
APADDVEFWCHVMY

SEQ ID NO: 44 - protein, 14 aa, Artificial Sequence
  Synthetic Construct
APSDDLEFWCHVMY

SEQ ID NO: 45 - protein, 14 aa, Artificial Sequence
  Synthetic Construct
APADDLEAWCHVMY

SEQ ID NO: 46 - protein, 14 aa, Artificial Sequence
  Synthetic Construct
APSDDLEFWCHAMY

SEQ ID NO: 47 - protein, 14 aa, Artificial Sequence
  Synthetic Construct
VASDDLEAWCHVMY

SEQ ID NO: 48 - protein, 14 aa, Artificial Sequence
  Synthetic Construct
AAADDLEFWCHVMY

SEQ ID NO: 49 - protein, 14 aa, Artificial Sequence
  Synthetic Construct
AASDDLAAWCHVMY

SEQ ID NO: 50 - protein, 14 aa, Artificial Sequence
  Synthetic Construct
AASDDLESWCHVMY

SEQ ID NO: 51 - protein, 14 aa, Artificial Sequence
  Synthetic Construct
APADDLAFWCHVMY

SEQ ID NO: 52 - protein, 14 aa, Artificial Sequence
  Synthetic Construct
LPADDLAVWCDVMY

SEQ ID NO: 53 - protein, 14 aa, Artificial Sequence
  Synthetic Construct
LPSDDLESWCHVMY

SEQ ID NO: 54 - protein, 14 aa, Artificial Sequence
  Synthetic Construct
AAADDLEVWCHVMY

SEQ ID NO: 55 - protein, 14 aa, Artificial Sequence
  Synthetic Construct
VAADALEFWCHVMY

SEQ ID NO: 56 - protein, 14 aa, Artificial Sequence
  Synthetic Construct
APSDDLAAWCHVVY

SEQ ID NO: 57 - protein, 14 aa, Artificial Sequence
  Synthetic Construct
AAADDLAAWCDVMY

SEQ ID NO: 58 - protein, 14 aa, Artificial Sequence
  Synthetic Construct
VASDDLEFWCHVMY

SEQ ID NO: 59 - protein, 14 aa, Artificial Sequence
  Synthetic Construct
AAADDLEAWCAVMY

SEQ ID NO: 60 - protein, 14 aa, Artificial Sequence
  Synthetic Construct
LAADDLESWCHVMY

SEQ ID NO: 61 - protein, 14 aa, Artificial Sequence
  Synthetic Construct
APADDLASWCHVMY

SEQ ID NO: 62 - protein, 14 aa, Artificial Sequence
  Synthetic Construct
VASDDLASWCHAMY

SEQ ID NO: 63 - protein, 14 aa, Artificial Sequence
  Synthetic Construct
VPADDLASWCHVMY

SEQ ID NO: 64 - protein, 14 aa, Artificial Sequence
  Synthetic Construct
APADDLEFWCHVVY

SEQ ID NO: 65 - protein, 14 aa, Artificial Sequence
  Synthetic Construct
VPSDDLAFWCHVMY

SEQ ID NO: 66 - protein, 14 aa, Artificial Sequence
  Synthetic Construct
VPADALAVWCDVMY

SEQ ID NO: 67 - protein, 14 aa, Artificial Sequence
  Synthetic Construct
VPADDLAFWCHVMY

SEQ ID NO: 68 - protein, 14 aa, Artificial Sequence
  Synthetic Construct
VPSDDLASWCHVMY

SEQ ID NO: 69 - protein, 14 aa, Artificial Sequence
  Synthetic Construct
VPSADLESWCHVMY

SEQ ID NO: 70 - protein, 14 aa, Artificial Sequence
  Synthetic Construct
AASDDLEAWCHVMY

SEQ ID NO: 71 - protein, 14 aa, Artificial Sequence
  Synthetic Construct
APSDDLASWCHVMY

SEQ ID NO: 72 - protein, 14 aa, Artificial Sequence
  Synthetic Construct
APADDLEFWCHVMY

SEQ ID NO: 73 - protein, 14 aa, Artificial Sequence
  Synthetic Construct
APSDDLAAWCHVMY

SEQ ID NO: 74 - protein, 14 aa, Artificial Sequence
  Synthetic Construct
APADDLAFWCHVVY

SEQ ID NO: 75 - protein, 14 aa, Artificial Sequence
  Synthetic Construct
APSDDLEAWCDVMY

SEQ ID NO: 76 - protein, 14 aa, Artificial Sequence
  Synthetic Construct
VPSDDLEAWCDVMY

SEQ ID NO: 77 - protein, 14 aa, Artificial Sequence
  Synthetic Construct
AASDDLAFWCHVMY

SEQ ID NO: 78 - protein, 14 aa, Artificial Sequence
  Synthetic Construct
VPADDLASWCDVMY

SEQ ID NO: 79 - protein, 14 aa, Artificial Sequence
  Synthetic Construct
LPSADLESWCHVMY

SEQ ID NO: 80 - protein, 14 aa, Artificial Sequence
  Synthetic Construct
AAADDLAFWCHVMY

SEQ ID NO: 81 - protein, 14 aa, Artificial Sequence
  Synthetic Construct
LASDDLEFWCHVMY

SEQ ID NO: 82 - protein, 14 aa, Artificial Sequence
  Synthetic Construct
LPAADLAAWCHVMY

SEQ ID NO: 83 - protein, 14 aa, Artificial Sequence
  Synthetic Construct
VPSADLETWCHVMY

SEQ ID NO: 84 - protein, 14 aa, Artificial Sequence
  Synthetic Construct
LPADDLAAWCHVMY

SEQ ID NO: 85 - protein, 14 aa, Artificial Sequence
  Synthetic Construct
PPADDLAFWCDVMY

SEQ ID NO: 86 - protein, 14 aa, Artificial Sequence
  Synthetic Construct
VASDDLASWCHVVY

SEQ ID NO: 87 - protein, 14 aa, Artificial Sequence
  Synthetic Construct
AAADDVASWCHVMY

SEQ ID NO: 88 - protein, 14 aa, Artificial Sequence
  Synthetic Construct
VAADDLAFWCDVMY

SEQ ID NO: 89 - protein, 14 aa, Artificial Sequence
  Synthetic Construct
APADDLEFWCHAMY

SEQ ID NO: 90 - protein, 14 aa, Artificial Sequence
  Synthetic Construct
APADDLAFWCDVMY

SEQ ID NO: 91 - protein, 14 aa, Artificial Sequence
  Synthetic Construct
APSDDLAFWCDVMY

SEQ ID NO: 92 - protein, 14 aa, Artificial Sequence
  Synthetic Construct
LPADDLAFWCDVMY

SEQ ID NO: 93 - protein, 14 aa, Artificial Sequence
  Synthetic Construct
AAADDLAFWCDVMY

SEQ ID NO: 94 - protein, 14 aa, Artificial Sequence
  Synthetic Construct
LPADDLEFWCHVMY

SEQ ID NO: 95 - protein, 14 aa, Artificial Sequence
  Synthetic Construct
VPSDDLEFWCAVMY

SEQ ID NO: 96 - protein, 14 aa, Artificial Sequence
  Synthetic Construct
APADDLESWCHVMY

SEQ ID NO: 97 - protein, 14 aa, Artificial Sequence
  Synthetic Construct
APSDDLAFWCHVVY

SEQ ID NO: 98 - protein, 14 aa, Artificial Sequence
  Synthetic Construct
AAADDLAAWCHVVY

SEQ ID NO: 99 - protein, 12 aa, Artificial Sequence
  Synthetic Construct
SDDLEFWCHVMY

SEQ ID NO: 100 - protein, 14 aa, Artificial Sequence
  Synthetic Construct
  VARIANT 1, 2, 3: Xaa = Any Amino Acid or An Unnatural Amino Acid and can be present or absent
  VARIANT 7, 8: Xaa = Any Amino Acid or An Unnatural Amino Acid
XXXDDLXXWCHVMY

SEQ ID NO: 101 - protein, 13 aa, Artificial Sequence
  Synthetic Construct
  VARIANT 1, 2: Xaa = Any Amino Acid or An Unnatural Amino Acid and can be present or absent
  VARIANT 6, 7: Xaa = Any Amino Acid or An Unnatural Amino Acid
XXDDLXXWCHVMY

SEQ ID NO: 102 - protein, 12 aa, Artificial Sequence
  Synthetic Construct
  VARIANT 1: Xaa = Any Amino Acid or An Unnatural Amino Acid and can be present or absent
  VARIANT 5, 6: Xaa = Any Amino Acid or An Unnatural Amino Acid
XDDLXXWCHVMY

SEQ ID NO: 103 - protein, 11 aa, Artificial Sequence
  Synthetic Construct
  VARIANT 4, 5: Xaa = Any Amino Acid or An Unnatural Amino Acid
DDLXXWCHVMY

SEQ ID NO: 104 - protein, 27 aa, Artificial Sequence
  Synthetic Construct
GALFLGFLGAAGSTMGAWSQPKKKRKV

SEQ ID NO: 105 - protein, 21 aa, Artificial Sequence
  Synthetic Construct
KETWWETWWTEWSQPKKKRKV

SEQ ID NO: 106 - protein, 20 aa, Artificial Sequence
  Synthetic Construct
  ACETYLATION 1
  VARIANT 20: Cya modification
GLWRALWRLLRSLWRLLWRA

SEQ ID NO: 107 - protein, 8 aa, Artificial Sequence
  Synthetic Construct
RRRRRRRR

SEQ ID NO: 108 - protein, 14 aa, Artificial Sequence
  Synthetic Construct
LPSDNLEFWCHVMY

SEQ ID NO: 109 - protein, 14 aa, Artificial Sequence
  Synthetic Construct
LPSDDAEFWCHVMY

SEQ ID NO: 110 - protein, 14 aa, Artificial Sequence
  Synthetic Construct
LPSDDLEFACHVMY

SEQ ID NO: 111 - protein, 14 aa, Artificial Sequence
  Synthetic Construct
LPSDDLEFWCHVAY

SEQ ID NO: 112 - protein, 14 aa, Artificial Sequence
  Synthetic Construct
LPSDDLEFWCHVMA

SEQ ID NO: 113 - protein, 14 aa, Artificial Sequence
  Synthetic Construct
LPSDDLEFWSHVMY

SEQ ID NO: 114 - protein, 12 aa, Artificial Sequence
  Synthetic Construct
  VARIANT 11: Xaa = Any Amino Acid or An Unnatural Amino Acid
SDDLEFWCHVXY

SEQ ID NO: 115 - protein, 12 aa, Artificial Sequence
  Synthetic Construct
  VARIANT 4: Xaa = Any Amino Acid or An Unnatural Amino Acid
SDDXEFWCHVMY

SEQ ID NO: 116 - protein, 12 aa, Artificial Sequence
  Synthetic Construct
  VARIANT 4, 11: Xaa = Any Amino Acid or An Unnatural Amino Acid
SDDXEFWCHVXY

SEQ ID NO: 117 - protein, 12 aa, Artificial Sequence
  Synthetic Construct
  VARIANT 10: Xaa = Any Amino Acid or An Unnatural Amino Acid
SDDLEFWCHXMY

SEQ ID NO: 118 - protein, 14 aa, Artificial Sequence
  Synthetic Construct
  VARIANT 14: Xaa = Any Amino Acid or An Unnatural Amino Acid
LPSDDLEFWCHVMX

SEQ ID NO: 119 - protein, 12 aa, Artificial Sequence
  Synthetic Construct
  VARIANT 3: Xaa = Any Amino Acid or An Unnatural Amino Acid
SDXLEFWCHVMY

SEQ ID NO: 120 - protein, 14 aa, Artificial Sequence
  Synthetic Construct
  VARIANT 5: Xaa = Any Amino Acid or An Unnatural Amino Acid
LPSDXLEFWCHVMY

SEQ ID NO: 121 - protein, 12 aa, Artificial Sequence
  Synthetic Construct
  VARIANT 6: Xaa = Any Amino Acid or An Unnatural Amino Acid
SDDLEXWCHVMY

SEQ ID NO: 122 - protein, 12 aa, Artificial Sequence
  Synthetic Construct
  VARIANT 1: Xaa = Any Amino Acid or An Unnatural Amino Acid
XDDLEFWCHVMY

SEQ ID NO: 123 - protein, 14 aa, Artificial Sequence
  Synthetic Construct
  VARIANT 10: Xaa = Any Amino Acid or An Unnatural Amino Acid
LPSDDLEFWXHVMY

SEQ ID NO: 124 - protein, 12 aa, Artificial Sequence
  Synthetic Construct
  VARIANT 8: Xaa = L-selenocysteine
SDDLEFWXHVMY

SEQ ID NO: 125 - protein, 12 aa, Artificial Sequence
  Synthetic Construct
SDDLEFWCHVEY

SEQ ID NO: 126 - protein, 12 aa, Artificial Sequence
  Synthetic Construct
SDDLEFWCHLMY

SEQ ID NO: 127 - protein, 12 aa, Artificial Sequence
  Synthetic Construct
SDDLEFWCHIMY

SEQ ID NO: 128 - protein, 12 aa, Artificial Sequence
  Synthetic Construct
SDDLEFWCHTMY

SEQ ID NO: 129 - protein, 12 aa, Artificial Sequence
  Synthetic Construct
SDDFEFWCHVMY

SEQ ID NO: 130 - protein, 12 aa, Artificial Sequence
  Synthetic Construct
SDELEFWCHVMY

SEQ ID NO: 131 - protein, 14 aa, Artificial Sequence
  Synthetic Construct
LPSDQLEFWCHVMY

SEQ ID NO: 132 - protein, 12 aa, Artificial Sequence
  Synthetic Construct
TDDLEFWCHVMY

SEQ ID NO: 133 - protein, 14 aa, Artificial Sequence
  Synthetic Construct
LPSDDLEFWAHVMY

SEQ ID NO: 134 - protein, 13 aa, Artificial Sequence
  Synthetic Construct
  VARIANT 13: Xaa = L-homopropargylglycine or L-bishomopropargylglycine
SDDLEFWCHVMYX

SEQ ID NO: 135 - protein, 12 aa, Artificial Sequence
  Synthetic Construct
  VARIANT 5: Xaa = 5-azido-L-ornithine or S-acetaminomethyl- L-cysteine or azido-homoalanine or S-acetaminomethyl-L-cysteine
SDDLXFWCHVMY

SEQ ID NO: 136 - protein, 190 aa, Artificial Sequence
  Synthetic Construct
AGSQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGGGSGGSGGSGGSGGSGGSGGSGGSLPSDDLEFWCHVMYGSGSGNSHHHHHH

SEQ ID NO: 137 - protein, 65 aa, Artificial Sequence
  Synthetic Construct
DAGLEVHQFYPLVKVQCSPELRFFLCSMYAPVCTVLDQAIPPCRSLCERARQGCEALMNKFGFQW

SEQ ID NO: 138 - protein, 65 aa, Artificial Sequence
  Synthetic Construct
DAGLEVHQFYPLVKVQCSPELRFFLCSMYAPVCTVLEQAIPPCRSICERARQGCEALMNKFGFQW

SEQ ID NO: 139 - protein, 65 aa, Artificial Sequence
  Synthetic Construct
DAGLEVHQFYPLVKVQCSAELKFFLCSMYAPVCTVLEQALPPCRSLCERARQGCEALMNKFGFQW

SEQ ID NO: 140 - protein, 66 aa, Artificial Sequence
  Synthetic Construct
EAGLEVHQFWPLVEIQCSPDLRFFLCSMYTPICLPDYHKPLPPCRSVCERAKAGCSPLMRQYGFAW

SEQ ID NO: 141 - protein, 66 aa, Artificial Sequence
  Synthetic Construct
EAGLEVHQFWPLVEIQCSPDLKFFLCSMYTPICLEDYKKPLPPCRSVCERAKAGCAPLMRQYGFAW

SEQ ID NO: 142 - protein, 66 aa, Artificial Sequence
  Synthetic Construct
DAELQLTTFTPLIQYGCSSQLQFFLCSVYVPMCTEKINIPIGPCGGMCLSVKRRCEPVLKEFGFAW

SEQ ID NO: 143 - protein, 66 aa, Artificial Sequence
  Synthetic Construct
EAAAELAEFAPLVQYGCHSHLRFFLCSLYAPMCTDQVSTPIPACRPMCEQARLRCAPIMEQFNFGW

SEQ ID NO: 144 - protein, 66 aa, Artificial Sequence
  Synthetic Construct
EAAIQLHEFAPLVEYGCHGHLRFFLCSLYAPMCTEQVSTPIPACRVMCEQARLKCSPIMEQFNFKW

SEQ ID NO: 145 - protein, 65 aa, Artificial Sequence
  Synthetic Construct
IAAVEMEHFLPLANLECSPNIETFLCKAFVPTCIEQIHVVPPCRKLCEKVYSDCKKLIDTFGIRW

SEQ ID NO: 146 - protein, 65 aa, Artificial Sequence
  Synthetic Construct
TAALAMEPFHPMVNLDCSRDFRPFLCALYAPICMEYGRVTLPCRRLCQRAYSECSKLMEMFGVPW

SEQ ID NO: 147 - protein, 109 aa, Homo sapiens
PDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFP

SEQ ID NO: 148 - protein, 109 aa, Homo sapiens
PDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFP

SEQ ID NO: 149 - protein, 109 aa, Homo sapiens
PDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFP

SEQ ID NO: 150 - protein, 110 aa, Homo sapiens
SKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLP

SEQ ID NO: 151 - protein, 110 aa, Homo sapiens
AKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLP

SEQ ID NO: 152 - protein, 110 aa, Homo sapiens
EEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFP

SEQ ID NO: 153 - protein, 110 aa, Homo sapiens
RGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLP

SEQ ID NO: 154 - protein, 110 aa, Homo sapiens
PGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLP

SEQ ID NO: 155 - protein, 109 aa, Homo sapiens
HSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQ

SEQ ID NO: 156 - protein, 109 aa, Homo sapiens
HSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGYPWPEDMECSRFP